SEQUENCE LISTING (203 sequences)

SEQ ID NO: 1 - DNA, 528 bp, Homo sapiens
  CDS (1)..(528)
ctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaacgaagaaaagaaggagaaggggaagaaaagaaggggaagaagatcaaaacccaccatgccccaggctcagcggtgtgctgatggaagtggaagagcctgaaatcttgcaggactcactggatagatgttattcgactccgtcaatgttctttgaactacctgactcattccagcactacagaagtgtgttttactcatttgaggaacagcacatcagcttcgcccttgacgtggacaataggtttcttactttgatgggaacaagtctccacctggtcttccagatgggagtcatattcccacagtaa

SEQ ID NO: 2 - protein, 175 aa, Homo sapiens
LSRELLEAVEPEVLQDSLDRCYSTPSSCLEQPDSCLPYGSSFYALEEKHVGFSLDVGEIEKKGKGKKRRGRRSTKKRRRRGRKEGEEDQNPPCPRLSGVLMEVEEPEILQDSLDRCYSTPSMFFELPDSFQHYRSVFYSFEEQHISFALDVDNRFLTLMGTSLHLVFQMGVIFPQ

SEQ ID NO: 3 - DNA, 3033 bp, Homo sapiens
  exon (1)..(171)
  exon (886)..(999): exon y
  exon (1611)..(1853): exon z
ctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggaggtgagtacctttctatgaaggtgataaggatccactgagtcttccatataaagatcatattcctgctccaagtggccattactgagctgagagatgtcattgctgcagtgaggacctataggcacatgtaggttgaatgaaactctagttctaactggaagcccagacatgggatgggtcagtgagcatggctctcttcctagtctcaggccatgcctgtggcactctgattctactctcatgacattggacctgggcagatgtgacaaattcagagaactatgattttgactcaagggtttgtagatttcctttttcactctaatttcagtgtctaaagtcctcacaaccatgaacaatctgagtatttgatgagacagggctaaatattgcagtttttctcctagaaatcatttgagggtatttgctttaaattgattggaaaaatatggcataactgtttgcacaaactcgggacaaatgatattgggataacgatctactagaatagggacattttacccacagtttctgggagaaaaaccgaggaatttctatcatgaccagccttcaggcctcctgaaatatatctctcacagtctcctattcttatgctgaggagcctgaggtccctgtgtgaggattagacagtggattgttatgtgtgtaggggaatcagcttaatgtgtctgtccatgtctgaatttattgcagaaattgaaaagaaggggaaggggaagaaaagaaggggaagaagatcaacgaagaaaagaaggagaaggggaagaaaagaaggggaagaagatcaaaacccaccatgccccaggtaactttcagcaattgtggatgcttaattctgtgttaacacctggaggcaacagattcagggaaaccagagtgtgtttgatttcatgttttcaacgaaggctgaattactcctactgtcattgctgttggttttcattgcagtagatgtttaggtttccatttcttcctccccttatcatttactaacgtaccataggatgaccatacttcaaaagctgtactctcgtggccactgcatcgaattttgagcatattttatggaaaactattgagctcactcttttcatgatcgcagtttgctgtgtgtcatgagggcactaactcagagtgtccttttactcccttaccagtatgtcacctggccaattcactagctcactttctctctgtctctgtctctgtctctgtctctctgtctttctctttcattgttttctacctggccctgttctatcccaacataaaggcaataattttttttttttacctcattaatggatctatccttttcttttcttaccacttccttacgttacttctgaaatctagtggggctctgtggtgtctgattttccctggctgcttctttagttttgtctgcttttccaggctcagcggtgtgctgatggaagtggaagagcctgaaatcttgcaggactcactggatagatgttattcgactccgtcaatgttctttgaactacctgactcattccagcactacagaagtgtgttttactcatttgaggaacagcacatcagcttcgcccttgacgtggacaataggtttcttactttgatgggaacaagtctccacctggtcttccagatgggagtcatattcccacagtaagcagcccttactaagccgagagatgtcattcctgcaggcaggacctataggcacgtgaagatttgaatgaaactatagttccatttggaagcccagacataggatgggtcagtgggcatggctctattcctattctcagaccatgccagtggcaacctgtgctcagtctgaagacaatggacccaagttaggtgtgacacgttcacataactgtgcagcacatgccgggagtgatcagtcagacattttaatttgaaccacgtatctctgggtagctacaaagttcctcagggatttcattttgcaggcatgtctctgagcttctatacctgctcaaggtcagtgtcatctttgtgtttagctcatccaaaggtgttaccctggtttcaatgaacctaacctcattctttgtatcttcagtgttgaattgttttagctgatccatctttaacacaggagggatccttggctgaggattgtatttcagaaccaccaactgctcttgacaattgttaacccgctaggctcctttggttagagaagccacagtccttcagcctccaattggtgtcagtacttaggaagaccacagctagatggacaaacagcattgggaggccttagccctgctcctctcgattccatcctgtagagaacaggagtcaggagccgctggcaggagacagcatgtcacccaggactctgccggtgcagaatatgaacaacgccatgttcttgcagaaaacgcttagcctgagtttcataggaggtaatcaccagacaactgcagaatgtagaacactgagcaggacaactgacctgtctccttcacatagtccatatcaccacaaatcacacaacaaaaaggagaagagatattttgggttcaaaaaaagtaaaaagataatatagctgcatttctttagttattttgaaccccaaatatttcctcatctttttgttgttgtcattgatggtggtgacatggacttgtttatagaggacaggtcagctgtctggctcaatgatctacattctgaagttgtctgaaaatgtcttcatgattaaattcagcctaaacgttttgccgggaacactgcagagacaatgctgtgagtttccaaccttagcccatctgcgggcagagaaggtctagtttgtccatcagcattatcatgat

SEQ ID NO: 4 - DNA, 3743 bp, Homo sapiens
  exon (711)..(881): Exon x
  exon (1596)..(1709): Exon y
  exon (2321)..(2563): Exon z
cccctcagctctgtgcattttgtctacaagcaaggtgactttgggtttagtgtgttcatgtttcaacaagaatgtaagtttgggatcagacatatgttacccctgttggatagtagctttgcaatcagtctttgcaaaaaatatccagaacaaggctagaacaaggtggtacagaaacaaataacacgtgttacagttacatatgagcttggattacctcctttatagaaggaagtggagacatccttcatgcagagatttaggatctctcactagcactaaccctcttgcccccacctagtgtttatttcaatcacccctgttgctttttatcaaatgaaagatacagatccacttttaagagtaaagaagagaggaagcacagtttattaattacccattggtatcccaaggacttgtgcttccattatctcattgttttattcattcattaatccaaacactcattcagcagggatttcttgggggcctaccttgtactgggctctgtggtcctgggagatgcagcagggaatagagtcacaggcttgcactcatagagagaaccttctagctggggagacagacaacaatgacaaaaaggaagtgctttttcaaccacttccttatgctacccatgaaacctagttggggctctgttgtgtctgatttcccctggcttattctttactttttcctccttttccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggaggtgagtacctttctatgaaggtgataaggatccactgagtcttccatataaagatcatattcctgctccaagtggccattactgagctgagagatgtcattgctgcagtgaggacctataggcacatgtaggttgaatgaaactctagttctaactggaagcccagacatgggatgggtcagtgagcatggctctcttcctagtctcaggccatgcctgtggcactctgattctactctcatgacattggacctgggcagatgtgacaaattcagagaactatgattttgactcaagggtttgtagatttcctttttcactctaatttcagtgtctaaagtcctcacaaccatgaacaatctgagtatttgatgagacagggctaaatattgcagtttttctcctagaaatcatttgagggtatttgctttaaattgattggaaaaatatggcataactgtttgcacaaactcgggacaaatgatattgggataacgatctactagaatagggacattttacccacagtttctgggagaaaaaccgaggaatttctatcatgaccagccttcaggcctcctgaaatatatctctcacagtctcctattcttatgctgaggagcctgaggtccctgtgtgaggattagacagtggattgttatgtgtgtaggggaatcagcttaatgtgtctgtccatgtctgaatttattgcagaaattgaaaagaaggggaaggggaagaaaagaaggggaagaagatcaacgaagaaaagaaggagaaggggaagaaaagaaggggaagaagatcaaaacccaccatgccccaggtaactttcagcaattgtggatgcttaattctgtgttaacacctggaggcaacagattcagggaaaccagagtgtgtttgatttcatgttttcaacgaaggctgaattactcctactgtcattgctgttggttttcattgcagtagatgtttaggtttccatttcttcctccccttatcatttactaacgtaccataggatgaccatacttcaaaagctgtactctcgtggccactgcatcgaattttgagcatattttatggaaaactattgagctcactcttttcatgatcgcagtttgctgtgtgtcatgagggcactaactcagagtgtccttttactcccttaccagtatgtcacctggccaattcactagctcactttctctctgtctctgtctctgtctctgtctctctgtctttctctttcattgttttctacctggccctgttctatcccaacataaaggcaataattttttttttttacctcattaatggatctatccttttcttttcttaccacttccttacgttacttctgaaatctagtggggctctgtggtgtctgattttccctggctgcttctttagttttgtctgcttttccaggctcagcggtgtgctgatggaagtggaagagcctgaaatcttgcaggactcactggatagatgttattcgactccgtcaatgttctttgaactacctgactcattccagcactacagaagtgtgttttactcatttgaggaacagcacatcagcttcgcccttgacgtggacaataggtttcttactttgatgggaacaagtctccacctggtcttccagatgggagtcatattcccacagtaagcagcccttactaagccgagagatgtcattcctgcaggcaggacctataggcacgtgaagatttgaatgaaactatagttccatttggaagcccagacataggatgggtcagtgggcatggctctattcctattctcagaccatgccagtggcaacctgtgctcagtctgaagacaatggacccaagttaggtgtgacacgttcacataactgtgcagcacatgccgggagtgatcagtcagacattttaatttgaaccacgtatctctgggtagctacaaagttcctcagggatttcattttgcaggcatgtctctgagcttctatacctgctcaaggtcagtgtcatctttgtgtttagctcatccaaaggtgttaccctggtttcaatgaacctaacctcattctttgtatcttcagtgttgaattgttttagctgatccatctttaacacaggagggatccttggctgaggattgtatttcagaaccaccaactgctcttgacaattgttaacccgctaggctcctttggttagagaagccacagtccttcagcctccaattggtgtcagtacttaggaagaccacagctagatggacaaacagcattgggaggccttagccctgctcctctcgattccatcctgtagagaacaggagtcaggagccgctggcaggagacagcatgtcacccaggactctgccggtgcagaatatgaacaacgccatgttcttgcagaaaacgcttagcctgagtttcataggaggtaatcaccagacaactgcagaatgtagaacactgagcaggacaactgacctgtctccttcacatagtccatatcaccacaaatcacacaacaaaaaggagaagagatattttgggttcaaaaaaagtaaaaagataatatagctgcatttctttagttattttgaaccccaaatatttcctcatctttttgttgttgtcattgatggtggtgacatggacttgtttatagaggacaggtcagctgtctggctcaatgatctacattctgaagttgtctgaaaatgtcttcatgattaaattcagcctaaacgttttgccgggaacactgcagagacaatgctgtgagtttccaaccttagcccatctgcgggcagagaaggtctagtttgtccatcagcattatcatgat

SEQ ID NO: 5 - DNA, 48 bp, Homo sapiens
  GenomeWalker adaptor
gtaatacgactcactatagggcacgcgtggtcgacggcccgggctggt

SEQ ID NO: 6 - DNA, 22 bp, Homo sapiens
gtaatacgactcactatagggc

SEQ ID NO: 7 - DNA, 26 bp, Homo sapiens
cccctcagctctgtgcattttgtcta

SEQ ID NO: 8 - DNA, 27 bp, Homo sapiens
cctcttgcccccacctagtgtttattt

SEQ ID NO: 9 - DNA, 20 bp, Homo sapiens
ggccatgtgaaccaattctg

SEQ ID NO: 10 - DNA, 20 bp, Homo sapiens
ggagagctgagtgaggaggg

SEQ ID NO: 11 - DNA, 20 bp, Homo sapiens
cgcctggcctatttacatgt

SEQ ID NO: 12 - DNA, 20 bp, Homo sapiens
tgcagcccataaatacccac

SEQ ID NO: 13 - DNA, 20 bp, Homo sapiens
cctcgagctgaagccaaatt

SEQ ID NO: 14 - DNA, 20 bp, Homo sapiens
tggcctccatgctgtaggat

SEQ ID NO: 15 - DNA, 20 bp, Homo sapiens
tcaaaccccgactttcagat

SEQ ID NO: 16 - DNA, 20 bp, Homo sapiens
tctgtgctgcctggattcac

SEQ ID NO: 17 - DNA, 21 bp, Homo sapiens
gcctgtttcatcaaatgcctg

SEQ ID NO: 18 - DNA, 21 bp, Homo sapiens
ggagaaatggcacagaggtga

SEQ ID NO: 19 - DNA, 20 bp, Homo sapiens
gagaccaagttctttcccag

SEQ ID NO: 20 - DNA, 20 bp, Homo sapiens
gagaccaagttctttcccag

SEQ ID NO: 21 - DNA, 21 bp, Homo sapiens
acaagcaagactgtgaagccc

SEQ ID NO: 22 - DNA, 21 bp, Homo sapiens
ggcttcctgtttctcctccag

SEQ ID NO: 23 - DNA, 20 bp, Homo sapiens
gcctatcaggccatgatcca

SEQ ID NO: 24 - DNA, 20 bp, Homo sapiens
ggactccaggacagtggcat

SEQ ID NO: 25 - DNA, 20 bp, Homo sapiens
accccatctctttccagcat

SEQ ID NO: 26 - DNA, 20 bp, Homo sapiens
ttgcctggcattcatctaga

SEQ ID NO: 27 - DNA, 20 bp, Homo sapiens
gggtccctggtagttaacga

SEQ ID NO: 28 - DNA, 19 bp, Homo sapiens
ccaactcctgaccccagaa

SEQ ID NO: 29 - DNA, 21 bp, Homo sapiens
atggatgggtaaactgaggcc

SEQ ID NO: 30 - DNA, 20 bp, Homo sapiens
gctgacttcctgagggaggc

SEQ ID NO: 31 - DNA, 21 bp, Homo sapiens
cacccaactttccatgagctc

SEQ ID NO: 32 - DNA, 20 bp, Homo sapiens
tggtggtgtacccccttgac

SEQ ID NO: 33 - DNA, 20 bp, Homo sapiens
gaactgtcgccactcgtctg

SEQ ID NO: 34 - DNA, 20 bp, Homo sapiens
acaccacagcgggatgatgt

SEQ ID NO: 35 - DNA, 516 bp, Homo sapiens
gaactgtcgccactcgtctggatgtgcattcgcaagctgccctgcgagtgctgtcaattgcaaattcagtcaattaggactcagcatcaggcagctggaatgacacttcctctagtgaagcctcagagagcttacttatgccactcatttcagatggagagcaagggtttgaatttgccccctctagagcccatgctcgcacttctgcaccgtatggcaaggttgagtcaggcagagcttgtcctcaagaagcccgcagtctgctgtggagagaggtgggcagtaaaacacatgctccagctgtagccatgtccctgtctggaccagttaactcaattcagtttcaacttgcagacagctccttcaagccaatcagacatcagagaaataaaaaggatgtgacaaaagagagtgaggtggagaacacagtctccctctcctggacaaactgctctgggaaagtttgcacttggaggtagatggggatgaagccaacacatcatcccgctgtggtgt

SEQ ID NO: 36 - DNA, 20 bp, Homo sapiens
aaccccgagtgttgctgaaa

SEQ ID NO: 37 - DNA, 20 bp, Homo sapiens
ccattccccaagcacctcat

SEQ ID NO: 38 - DNA, 789 bp, Homo sapiens
aaccccgagtgttgctgaaatcaaaaggctatgaagaagcagaagtcttgtgaaggtgttgttgggaaagagaattgttcagatatgggtaagccactgaaaggtgtcttgatggttcatctgaacatgggctgagaacctgaagcagatacagaaagcaagatgaccatgctggaacaatgaggacagggaggatgggcagagtggctagggagaaggaggtcttggatgaacatctgagatagaaaatgcccctctggcggctcatgtgtgctctcctgatcacatgctctgacatggcaggccccagcccattctccttacgaacgacacttgctgggaatccctactgggcacacacaacccagtctacaagatagagtggtccaaaccccccatcctgggctgtggtgcttggaagatgcaacacaccgactggctacgctgcagttactgcatctttctggctggctggcaagcaaacttcccactgcctttctccccaggcctgaccccattatgacaatggagccattgttctgaacaggggccatgaggcagctgggactggccgaccctatgctggctcctcacatctcagaagtcaagccctcagacagatcaaggaaagtgataaaattcagcgggaagcaggacagagtattgggggtcctgagccagcttcttcctctcagttctgtatcgtgatgggaatgaataatcaccccaattacagagcaacctgaaggagacaaaatcacttaactaaatgaggtgcttggggaatgg

SEQ ID NO: 39 - DNA, 20 bp, Homo sapiens
agagctgcacggcacttctc

SEQ ID NO: 40 - DNA, 20 bp, Homo sapiens
cacgatgcaaaacgaactac

SEQ ID NO: 41 - DNA, 465 bp, Homo sapiens
agagctgcacggcacttctcagcccacaagcccactttccatgcaccaagcccaccccaccttacacttggcccctggctccttaaccttgagctcagcctccatcttaaggtttcaccaagctgcccttcactatccggtctgttggagattttccagctagactctgctagccacgctctgactctgtttgacccagctcccggggagaagccaagccgactcccttccactccctctgcccccgctgctgtcttacacagccgtaccttcctttcctttgccccctctcttggctggtgctgagccacatttagcaccagcctccaagagcaagcaccctgtgctaacttgggctcgtgtctcgcaagctaccaccctgatcttagaaacccgaagctcagaaaatagttcctatcttatgtgcgttatactcttcaagaaggtagttcgttttgcatcgtg

SEQ ID NO: 42 - DNA, 22 bp, Homo sapiens
gtttggggagattttgagtgtg

SEQ ID NO: 43 - DNA, 21 bp, Homo sapiens
gggcagcagaccttagcaact

SEQ ID NO: 44 - DNA, 478 bp, Homo sapiens
gtttggggagattttgagtgtgtgggtggcagaaggtacttttaggttggggtcacagtgcagcgggccgagcaccaaagcacagatattctagaactggggttctacatcagactactactgtgagagccaagaggctcaaagcccccagagggtgactcattcttccagccctccgcctcctcctgcctgtccaggtcatcttgatcatcccattatcagaactgctggaggaatcatctggaaggcttgaagtgactggggggcatccgctgggttgtgagagctgctcggagggtcccttgtctgtcccagtccacctcctccacccaccttatgctgtcaccccctgcccaggactggacctccattcaactcttctgagcagaaggagcagaaaccatcctatctctgcactggcctggaatggacagtgctccccagcatggttctagggagttgctaaggtctgctgccc

SEQ ID NO: 45 - DNA, 22 bp, Homo sapiens
tcccaaaaggccagtttcacac

SEQ ID NO: 46 - DNA, 22 bp, Homo sapiens
tctgcaggcgtctcatctcaac

SEQ ID NO: 47 - DNA, 678 bp, Homo sapiens
tcccaaaaggccagtttcacacactccattaatgctggctgccactgggacaattacagacagacagcttttcccatttgagcaacttcttaaatttaaaaagcaactgtgcaggaaggaaggagacatgtggctgctctactacagaccttgtaaaaggaggttcttctatgctattacccaccccctcccatacctacctttcctaccacacacaagagctgaaataaaagttgcatttcacttgtgttgtggctgttattttattttacactccttgcttaaaaaaaaaaaaagtcttcacttaaaaaaaaatacatcacccaaacatgtctgagattttttgcaaggcttgggtctgaacccagtgcctttgataggggcatttttcttagcctgctgtggctaaagatgggcatggtcggatttcactcgcttggattaggatggacttggccctaatattttgtttcttaatgattcttccaatttttaagtgtcccaagagatagctttgactttccaccccagcctgctgatgttatcttcctaggaactgctacatcttttaatacaacctgtattcaagttctccttataggacctgagagattcaactggcagcatttaggactaccaggaatctccagtctggcgttgagatgagacgcctgcaga

SEQ ID NO: 48 - DNA, 20 bp, Homo sapiens
cccctcagctctgtgcattt

SEQ ID NO: 49 - DNA, 21 bp, Homo sapiens
tccctgctgaatgagtgtttg

SEQ ID NO: 50 - DNA, 478 bp, Homo sapiens
cccctcagctctgtgcattttgtctacaagcaaggtgactttgggtttagtgtgttcatgtttcaacaagaatgtaagtttgggatcagacatatgttacccctgttggatagtagctttgcaatcagtctttgcaaaaaatatccagaacaaggctagaacaaggtggtacagaaacaaataacacgtgttacagttacatatgagcttggattacctcctttatagaaggaagtggagacatccttcatgcagagatttaggatctctcactagcactaaccctcttgcccccacctagtgtttatttcaatcacccctgttgctttttatcaaatgaaagatacagatccacttttaagagtaaagaagagaggaagcacagtttattaattacccattggtatcccaaggacttgtgcttccattatctcattgttttattcattcattaatccaaacactcattcagcaggga

SEQ ID NO: 51 - DNA, 22 bp, Homo sapiens
gaacctggtctacaaattatac

SEQ ID NO: 52 - DNA, 22 bp, Homo sapiens
gactgggaccaatttgttcttc

SEQ ID NO: 53 - DNA, 673 bp, Homo sapiens
gaacctggtctacaaattatacaccctggcatgtccaaaagcaaaatgctgcctacatctcctgaaaatgcaggaatgtcagaaaagctgctgaagtggtggctaccaggagctccaggggcctagcacccactcccagcccgaccctggggtggggagggtggtgctcttcagagtggctcattttagagctgactacccaagtgcagaagaaacaacgttttaaatatatcgggcagcagagcagctagatattaaggttcttctttgcaggacatcatgaaatattgttcatgacgaccctgaccctgggttgggcgcgggtgctcttcagagtggctcattttagagctgacttcccaagtgcagaagaaacaactttttaaatacgtcgggtaacagagcagctagacattacggttcttctttgcagaacatcatgaaatattgatgttaagctaaaacgaatataataaatgtatgattgcctgctaaattggtcattatcttttttagattctggctttgacagaaagtagcaggccccaatggtttaagaaagaaaggaaaaaatttttttaattgaggaaatcatatccacgtctttttttttttaattgttgaaaagatgtccacagtcagatttcagaggaagaacaaattggtcccagtc

SEQ ID NO: 54 - DNA, 21 bp, Homo sapiens
gtacacccactcacctgtgat

SEQ ID NO: 55 - DNA, 22 bp, Homo sapiens
tctaagggggaaaagtaaacaa

SEQ ID NO: 56 - DNA, 761 bp, Homo sapiens
gtacacccactcacctgtgatgtacttaatggagcaggagggaacaggctgccatcaaagctaactcctggaaatgacctagaacccacagcctgtctcagccgtgctgcatgttccagcagggaacaaagcgactgttgtattatgtaaaattacttacctgataataaaatgtttgtggattggataaatttgaacggatatattgtttccttcaccccacccccactagtggcaaaagctgctctgtatgaatctgagtacatttctgtgacagtattcctcatatgtacagcattctatgtgaatcatctcccaaaaattgccatggggtcattatccccagttcttaactagagaaactgagattctggaagtccctctgaatggcccatacttccccaggtgtctctgcaggaaacaatgtggacaatcacagtagttggaaggacaatgactgacctaatggagttcaggacgctatgactgttggcacctgatgtgcacagctgctcccatctgcgcagagagaatataaaggcagtggcgctgaaacatctgctgctttcactcaccatcaagatggaactgactgtacatgcaagcaaattttgatgaagagagtgatctcagagtgcgaagataagttgtttctccttgacacctaggaatgcacctggccaagaccctcagttgaaagacaatatcaaataaatgcaaaatctaaatatccatgtgcttgtttacttttcccccttaga

SEQ ID NO: 57 - protein, 11 aa, Homo sapiens
SAVGSDHIFHN

SEQ ID NO: 58 - protein, 13 aa, Homo sapiens
SAVGSDHIFHNSK

SEQ ID NO: 59 - protein, 11 aa, Homo sapiens
SAAGTDHIFLN

SEQ ID NO: 60 - protein, 70 aa, Homo sapiens
VPGPTSSATNVSMVVSAGPLSSEKAEMNILEMNEKLRPQLAEKKQQFRNLKEKCFVTQLAGFLANQQNKY

SEQ ID NO: 61 - protein, 70 aa, Homo sapiens
VPGPTSSATNVSMVVSAGPLSSEKAEMNILEINEKLHPQLAEKKQQFRNLKERCFVTQLAGFLANQQKKY

SEQ ID NO: 62 - protein, 70 aa, Homo sapiens
VPGPTSSATNVSMVVSAGPLSSEKAEMNILEINEKLRPQLAEKKQQFRSLKEKCFVTQLAGFLAKQQNKY

SEQ ID NO: 63 - protein, 70 aa, Homo sapiens
VPDSTSSATNVSMVVSAGPWSSEKAEMNILEINEKLRPQLAENKQQFGNLKERCFVTQLAGFLANQQKKY

SEQ ID NO: 64 - protein, 70 aa, Homo sapiens
IPDSTSSATNVSMVVSAGPWSSEKAEMNILEINEKLRPQLAENKQQFRNLKERCFLTQLAGFLANRQKKY

SEQ ID NO: 65 - protein, 70 aa, Homo sapiens
VPGSTSSATNVSMVVSAGPWSSEKAETNILEINEKLRPQLAENKQQFRNLKEKCFVTQLAGFLANRQKKY

SEQ ID NO: 66 - protein, 70 aa, Homo sapiens
IPGSTSSATNVSMVVSAGPWSSEKAETNILKMNEKLRPQLAEKKQQFRNLKEKCFVTQLAGFLANRQKKY

SEQ ID NO: 67 - protein, 67 aa, Homo sapiens
IPGPTSSATNVSMVVSAGPWSGEKAEMNILEINKKSRPQLAENKQQFRNLKQKCLVTQVAYFLANRQ

SEQ ID NO: 68 - protein, 35 aa, Homo sapiens
KYEECKDLIKSMLRNELQFKEEKLAEQLKQAEELR

SEQ ID NO: 69 - protein, 35 aa, Homo sapiens
KYEECKDLIKSMLRNERQFKEEKLAEQLKQAEELR

SEQ ID NO: 70 - protein, 35 aa, Homo sapiens
KYEECKDLIKFMLRNERQFKEEKLAEQLKQAEELR

SEQ ID NO: 71 - protein, 29 aa, Homo sapiens
DLIKSMLRNERQFKEEKLAEQLKQAEELR

SEQ ID NO: 72 - protein, 35 aa, Homo sapiens
EYEECKDLIKSMLRNERQFKEEKLAEQLKQAEELR

SEQ ID NO: 73 - protein, 35 aa, Homo sapiens
EYEECKDLIKCMLRNERQFKEEKLAEQLKQAEELR

SEQ ID NO: 74 - protein, 35 aa, Homo sapiens
KYEECKDVIKFMLRNERQFKEEKLAEQLKQAEELR

SEQ ID NO: 75 - protein, 71 aa, Homo sapiens
QYKVLVHSQERELTQLREKLREGRDASRSLNEHLQALLTPDEPDKSQGQDLQEQLAEGCRLAQHLVQKLSP

SEQ ID NO: 76 - protein, 71 aa, Homo sapiens
QYKVLVHSQERELTQLREKLREGRDASRSLNEHLQALLTLDEPDKSQGQDLQEQLAEGCRLAQHLVQKLSP

SEQ ID NO: 77 - protein, 71 aa, Homo sapiens
QYKVLVHAQERELTQLREKLREGRDASRSLNEHLQALLTPDEPDKSQGQDLQEQLAEGCRLAQHLVQKLSP

SEQ ID NO: 78 - protein, 71 aa, Homo sapiens
QYKVLVHSQERELTQLREKLREGRDASRSLNEHLQALLTPDEPDKSQGQDLQEQLAEGCRLAQQLVQKLSP

SEQ ID NO: 79 - protein, 71 aa, Homo sapiens
QYKVLVHSQERELTQLREKLREGRDASRSLNQHLQALLTPDEPDKSQGQDLQEQLAEGCRLAQHLVQKLSP

SEQ ID NO: 80 - protein, 71 aa, Homo sapiens
QYKVLVHSQERELTQLREKLREGRDASRSLNQHLQALLTPDEPDKSQGQDLQEQLAEGCRLAQQLFQKLSP

SEQ ID NO: 81 - protein, 25 aa, Homo sapiens
ENDEDEDEDVQVEEAEKVLESSAPR

SEQ ID NO: 82 - protein, 25 aa, Homo sapiens
ENDNDDDEDVQVEVAEKVQKSSAPR

SEQ ID NO: 83 - protein, 25 aa, Homo sapiens
ENDNDDDEDVQVEVAEKVQKSSSPR

SEQ ID NO: 84 - protein, 25 aa, Homo sapiens
ENDNDDDEDVQVELAEKVQKSSAPR

SEQ ID NO: 85 - protein, 25 aa, Homo sapiens
ENDEDEDEDVQVEEAEKVQKSSAPR

SEQ ID NO: 86 - protein, 25 aa, Homo sapiens
ENDEDEDEDVQVEEDEKVLESSAPR

SEQ ID NO: 87 - protein, 25 aa, Homo sapiens
ENDEDEDEDVQVEEDEKVLESSSPR

SEQ ID NO: 88 - protein, 25 aa, Homo sapiens
ENDNDHDEDVQVEVAEKVQKSSAPR

SEQ ID NO: 89 - protein, 70 aa, Homo sapiens
EVQKAEESKVPEDSLEECAITCSNSHGPCDSNQPHKNINITFEEDKVNSALVVDRESSHDECQDAVNILP

SEQ ID NO: 90 - protein, 70 aa, Homo sapiens
EMQKAEESKVPEDSLEECAITCSNSHGPCDSNQPHKNIKITFEEDKVNSSLVVDRESSHDECQDALNILP

SEQ ID NO: 91 - protein, 70 aa, Homo sapiens
EVQKTEESKVPEDSLEECAITCSNSHGPCDSNQPHKNIKITFEEDEVNSTLVVDRESSHDECQDALNILP

SEQ ID NO: 92 - protein, 70 aa, Homo sapiens
EVQKAEESKVPEDSLEECAITCSNSHGPCDSNQPHKNIKITFEEDKVNSTVVVDRKSSHDECQDALNILP

SEQ ID NO: 93 - protein, 68 aa, Homo sapiens
EMQKAEEKEVPEDSLEECAITCSNSHGPYDSNQPHRKTKITFEEDKVDSTLIGSSSHVEWEDAVHIIP

SEQ ID NO: 94 - protein, 68 aa, Homo sapiens
EMQKAEEKEVPEDSQEECAITYSNSHGPYDSNQPHRKTKITFEEDKVDSTLIGSSSHVEWEDAVHIIP

SEQ ID NO: 95 - protein, 68 aa, Homo sapiens
EMPKAEEKEVPEDSLEECAITCSNSHGPYDSNQPHRKTKITFEEDKVDSTLIGSSSHVEWEDAVHIIP

SEQ ID NO: 96 - protein, 18 aa, Homo sapiens
ENESDDEEEEEKGPVSPR

SEQ ID NO: 97 - protein, 54 aa, Homo sapiens
NLQESEEEEVPQESWDEGYSTLSIPPEMLASYQSYSSTFHSLEEQQVCMAVDIG

SEQ ID NO: 98 - protein, 54 aa, Homo sapiens
NLQESEEEEVPQESWDEGYSTLSIPPEMLASYKSYSSTFHSLEEQQVCMAVDIG

SEQ ID NO: 99 - protein, 27 aa, Homo sapiens
NLQESEEEEVPQESWDEGYSTLSIPPE

SEQ ID NO: 100 - protein, 54 aa, Homo sapiens
NLQESEEEEVPQESWDEGYSTLSIPPEMLASYQSYSGTFHSLEEQQVCVAVDIG

SEQ ID NO: 101 - protein, 54 aa, Homo sapiens
NLQESEEEEVPQESWDEGYSTLSIPPEMLASYQSYSGTFHSLEEQQVCMAVDIG

SEQ ID NO: 102 - protein, 18 aa, Homo sapiens
RHRWDQVKKEDQEATGPR

SEQ ID NO: 103 - protein, 18 aa, Homo sapiens
RHRWDQVKKEDHEATGPR

SEQ ID NO: 104 - protein, 18 aa, Homo sapiens
GHRWDQVKKEDQEATGPR

SEQ ID NO: 105 - protein, 57 aa, Homo sapiens
LSRELLAEKEPEVLQDSLDRWYSTPSVYLGLTDPCQPYRSAFYVLEQQRVGLAVDMD

SEQ ID NO: 106 - protein, 57 aa, Homo sapiens
LSRELLAEKEPEVLQDSLDRCYSTPSVYLGLTDSCQPYRSAFYVLEQQRVGLAVDMD

SEQ ID NO: 107 - protein, 57 aa, Homo sapiens
LSRELLDEKGPEVLQDSLDRCYSTPSGCLELTDSCQPYRSAFYVLEQQRVGLAVNMD

SEQ ID NO: 108 - protein, 57 aa, Homo sapiens
LSRELLDEKGPEVLQDSLDRCYSTPSGCLELTDSCQPYRSAFYILEQQRVGLAVDMD

SEQ ID NO: 109 - protein, 57 aa, Homo sapiens
  misc_feature (47)...(47): the ′Xaa′ at position 47 may be any amino acid
LSRELLDEEGPEVLQDSLDRCYSTPSGCLELTDSCQPYRSAFYVLEXQRVGLAVDMD

SEQ ID NO: 110 - protein, 57 aa, Homo sapiens
LSGELLDEKEPEVLQESLDRCYSTPSGCLELTDSCQPYRSAFYILEQQRVGLAVDMD

SEQ ID NO: 111 - protein, 57 aa, Homo sapiens
LSRELLDEKGPEVLQDSLDRCYSTPSGYLELTDSCQPYRSAFYILEQQRVGWALDMD

SEQ ID NO: 112 - protein, 56 aa, Homo sapiens
  misc_feature (25)...(25): the ′Xaa′ at position 25 may be any amino acid
LNRELLDEKGPEVLQDSLDRCYSTXSGYLLTDSCQPYRSAFYILEQQRVGWALDLV

SEQ ID NO: 113 - protein, 57 aa, Homo sapiens
LSRELLDEKEPEVLQDSLGRCYSTPSGYLELPDLGQPYSSAVYSLEEQYLGLALDVD

SEQ ID NO: 114 - protein, 57 aa, Homo sapiens
LSRELLAEKEPEVLQDSLDRCYSTPSGYLELPDLGQPYSSAVYSLEEQYLGLALDVD

SEQ ID NO: 115 - protein, 57 aa, Homo sapiens
LSRELLDEKEPEVLQDSLDRCYSTPSGYLELPDLGQPYRSAVYSLEEQYLGLALDVD

SEQ ID NO: 116 - protein, 57 aa, Homo sapiens
LSRELLAEKEPEVLQDSLDRCYSTPSGYLELPDLGQPYRSAVYSLEEQYLGLALDVD

SEQ ID NO: 117 - protein, 57 aa, Homo sapiens
LSRELLAEKEPEVLQDSLDRCYSTPSGYLELPDLGQPYRSAAYSLEEQYLGLALDVD

SEQ ID NO: 118 - protein, 57 aa, Homo sapiens
LSRELLDEKGPEVLQDSLDRCYSTPSGCLELTDSCQPYRSAFYVLEQQRVGLAVDMD

SEQ ID NO: 119 - protein, 57 aa, Homo sapiens
LSRELLDEKEPEVLQDSLDRCYSTPSGYLELPDLGQPYSSAVYSLEEQYLGLALDVD

SEQ ID NO: 120 - protein, 57 aa, Homo sapiens
LSRELLDEKGPEVLQDSLDRCYSTPSGYLELTDSCQPYRSACYILEQQRVGWALDMD

SEQ ID NO: 121 - protein, 57 aa, Homo sapiens
LSRELLDEKEPEVLQDSLDRCYSTPSGYLELPDLGQPYRSAVHSLEEQYLGLALDVD

SEQ ID NO: 122 - protein, 57 aa, Homo sapiens
LSRELLDEKGPEVLQDSLDRCYSTPSGCLELTDSCQPYRSAVYILEQQCVGLAVDMD

SEQ ID NO: 123 - protein, 57 aa, Homo sapiens
LSRELLEAVEPEVLQDSLDRCYSTPSSCLEQPDSCLPYGSSFYALEEKHVGFSLDVG

SEQ ID NO: 124 - protein, 57 aa, Homo sapiens
LSRELLRAVEPEVLQDSLDRCYSTPSSCLEQPDSCLPYGSSFYALEEKHVGFSLDVG

SEQ ID NO: 125 - protein, 57 aa, Homo sapiens
LSRELLEVVEPEVLQDSLDRCYSTPSSCLEQPDSCQPYGSSFYALEEKHVGFSLDVG

SEQ ID NO: 126 - protein, 57 aa, Homo sapiens
LSRELLEVVEPEVLQGSLDRCYSTPSSCLEQPDSCQPYGSSFYALEEKHVGFSLDVG

SEQ ID NO: 127 - protein, 57 aa, Homo sapiens
LSRELLEVVEPEVLQDSLDRCYSTPSSCLEQPDSCQPYRSSFYALEEKHVGFSLDVG

SEQ ID NO: 128 - protein, 57 aa, Homo sapiens
LSRELLEVVEPEVLQDSLDRCYSTPSSYLELPDSCQPYGSSFYALEEKHVGFSLDVG

SEQ ID NO: 129 - protein, 35 aa, Homo sapiens
EIEKKGKGKKRRGRRSKKKRRRGRKEGEDDNPPCP

SEQ ID NO: 130 - protein, 37 aa, Homo sapiens
EIEKKGKGKKRRGRRSKKKRRRGRKEGEEDQNPPCPR

SEQ ID NO: 131 - protein, 37 aa, Homo sapiens
EIEKKGKGKKRRGRRSKKERRRGRKEGEEDQNPPCPR

SEQ ID NO: 132 - protein, 38 aa, Homo sapiens
EIEKKGKGKKRRGRRSMKKRRRRGRKEGEEDQNPPCPR

SEQ ID NO: 133 - protein, 38 aa, Homo sapiens
EIEKKGKGKKRRGRRSTKKRRRRGRKEGEEDQNPPCPR

SEQ ID NO: 134 - protein, 38 aa, Homo sapiens
EIEKKGKGKKRRGRRSTKKRRRRGSKEGEEDQNPPCPR

SEQ ID NO: 135 - protein, 25 aa, Homo sapiens
EIEKKGKGKKRRGRRSTKERRRRGR

SEQ ID NO: 136 - protein, 18 aa, Homo sapiens
EIEKSQKWKKTQNPSCPR

SEQ ID NO: 137 - protein, 18 aa, Homo sapiens
EIEKYQEVEEDQDPSCPR

SEQ ID NO: 138 - protein, 18 aa, Homo sapiens
RIKKDQEEEEDQGPPCPR

SEQ ID NO: 139 - protein, 18 aa, Homo sapiens
RTKKDQEEEEDQGPPCPR

SEQ ID NO: 140 - protein, 18 aa, Homo sapiens
RIKKDQEEEEDQGPPCSR

SEQ ID NO: 141 - protein, 23 aa, Homo sapiens
  misc_feature (2)...(2): the ′Xaa′ at position 2 may be any amino acid
EXSEQEIKKYQEEEEDQDPSCPR

SEQ ID NO: 142 - protein, 80 aa, Homo sapiens
LNGVLMEVEEREVLQDSLDRCYSTPSMYFELPDSFQHYRSVFYSFEEQHISFALYVDNRFFTLTVTSLHLVFQMGVIFPQ

SEQ ID NO: 143 - protein, 80 aa, Homo sapiens
LNGVLMEVEEREVLQDSLDRCYSTPSMYFELPDSFQHYRSVFYSFEEQHITFALYVDNRFFTLTVTSLHLVFQMGVIFPQ

SEQ ID NO: 144 - protein, 80 aa, Homo sapiens
LNGVLMEVEEPEVLQDSLDGCYSTPSMYFELPDSFQHYRSVFYSFEEQHISFALYVDNRFFTLTVTSLHLVFQMEVIFPQ

SEQ ID NO: 145 - protein, 80 aa, Homo sapiens
LNSVLMEVEEPEVLQDSLDGCYSTPSMYFELPDSFQHYRSVFYSFEEEHISFALYLDNRFFTLTVTSLHLVFQMLVIFPQ

SEQ ID NO: 146 - protein, 80 aa, Homo sapiens
LNSMLMEVEEPEVLQDSLDICYSTPSMYFELPDSFQHYRSVFYSFEEEHISFALYVDNRFFTLTVTSLHLVFQMGVIFPQ

SEQ ID NO: 147 - protein, 80 aa, Homo sapiens
LYGVLMEVEEPEVLQDSLDRCYSTPSMYFEQPDSFQHYRSVFYSFEEEHISFALYVDNRFFTLTVTSLHLVFQMGVIFPQ

SEQ ID NO: 148 - protein, 80 aa, Homo sapiens
SYGVLMEVEEPEVLQDSLDRCYSTPSMYFEQPDSFQHYRSVFYSFEEEHISFALYVDNRFFTLTVTSLHLVFQMGVIFPQ

SEQ ID NO: 149 - protein, 80 aa, Homo sapiens
LSGVLMEVEEPEILQDSLDRCYSTLSLFFELPDSFQHYRSVFYSFEEQHISFALDVDNRFLTLMGTSLHLVFQMGVIFPQ

SEQ ID NO: 150 - protein, 80 aa, Homo sapiens
  misc_feature (47)...(47): the ′Xaa′ at position 47 may be any amino acid
  misc_feature (55)...(55): the ′Xaa′ at position 55 may be any amino acid
  misc_feature (73)...(73): the ′Xaa′ at position 73 may be any amino acid
FSGVLMEVEEPEILQDSLDRCYSTLSLFFELPDSFQHYRSVFYSFEXQHISFALXVDNRVLTLMGTSLHRGFXMGVIFPQ

SEQ ID NO: 151 - protein, 80 aa, Homo sapiens
LSGVLMEVEEPEILQDSLDRCYSTPSLFFELPDSFQHYRSVFYSFEEQHISFALDVDNRFLTLMGTSLHLVFQMGVIFPQ

SEQ ID NO: 152 - protein, 80 aa, Homo sapiens
LSGVLMEVEEPEILQDSLDRCYSTPSMFFELPDSFQHYRSVFYSFEEQHISFALDVDNRFLTLMGTSLHLVFQMGVIFPQ

SEQ ID NO: 153 - protein, 80 aa, Homo sapiens
LNSVLMEVEEPEVLQDSLDRCYSTPSMYCELRDSFQHYRSVFYSFEEQHISFALDMDNRFFTLTVTSLYLVFQMGVIFPQ

SEQ ID NO: 154 - protein, 80 aa, Homo sapiens
LSSVLMEVEVPEVLQDSLDRYYSTPSMYFELPDSFQHYRSVFYSFEEQHISFALTWTIGFFTLTVRSLHLVFQMGVIFPQ

SEQ ID NO: 155 - protein, 80 aa, Homo sapiens
LNSVLMEVEEPEVLQDSLDRCYSTPSMYFELPDSFQHYRSVFYSFEEQHITFALDMDNSFFTLTVTSLHLVFQMGVIFPQ

SEQ ID NO: 156 - protein, 80 aa, Homo sapiens
LSGVLMEVEEPEVLQDSLDRCYSTPSMYFELPDSFQHYRSVFYSFEEQHISFALDVDNRFLTLMGTSLHLVFQMGVIFPQ

SEQ ID NO: 157 - DNA, 20 bp, Homo sapiens
gttgtacccccttgacttca

SEQ ID NO: 158 - DNA, 21 bp, Homo sapiens
gtgcccagcaggagattcaat

SEQ ID NO: 159 - DNA, 568 bp, Homo sapiens
ggtgtacccccttgacttcacctgaatgtcagggcaaggccaggagcatgcaggaggcttaaagattgggaacccctattcttgggccttctccccattctttctggagcaattggaagaggcccatgggagggaggtgactgtcatgatgtcccccactatgaactatgggaatggctggagggtcactgggcctggtgctccagctgcctgacctgcagggaagtggtacctgcaggacctacagtgagggagtgttgacaccctggctctgggaaggagctcatggaaagttgggtgaacttcttgttagactacgcaggctttcagaagtgcattccatacggagtggggtggcctccccagggaggagcagagggcagccactggaacaagctacagctacaaaagaccacctttatccctctcaagttctcccaggggcccctgtgtctgcctgtactagaggaagtttccaaaggattttcctttccccaggatgaagaatgaggccacactgccaaagcaagatctcgtgctatgtgtgattgaatctcctgctgggcac

SEQ ID NO: 160 - DNA, 4512 bp, Homo sapiens
  CDS (206)..(409)
  CDS (1717)..(1770)
  der1 breakpoint-overlapping; Genbank AF379606
ctgctctcttcctctctggttcccatggcagccatgctctgttgcagagagaacaggattgcatgtaccctcttaatgggaacctccagtttgctttctgggaccactctcttaatgccgcctgtcaaaaccagctaggactccctggggtccaatccctctgtgtttaatcttctgtcatctctgtcccacctggctcatcagggacatgcagaaggctgaagaaaaggaagtccctgaggactcactggaggaatgtgccatcacttgttcaaatagccatggcccttatgactccaaccagccacataggaaaaccaaaatcacatttgaggaagacaaagtcgactcaactctcattggctcatcctctcatgttgaatgggaggatgctgtacacattatcccaggtagcctctgttttccttgtgtctcatacctctctctaggctgaggaagataaactctgaagacaggctctataaacacaaattcatttgaataaaaaactatgatgggtttctaaacagatatcagggagtttttttgtccttctcagctaatgtcatgcctttgtctgccagtccccagtatcaagttactcgaccccaggcaagtgtgacaatctcatagtcacctgagtgcaggaggtgcacaggcagtatctgtcaggcctcctagcttcgattcagtatctcttgtcatctgtgattaagtcatctgtccctgaacaatgtccatggagtttctatgcctgtttcaggaagctggcagccttgcctttgtatttggaaatattgttccccaggcttcactgctctcagctttcatctggatctcctttaagtcagcttgcttagctgcacagtcaccctgaaatcaggatggaaacttttcttctttactttgctgatatatttccataaagcaaggctggaccctggttctccaccctgtcaatgcaatggctgatccaatgtttctttgtagcatcgtggattttttttttttttttttttttttttttttttttttttttttttttgcgatggagtcttgctctgtcacccaggctagagtgcagttgcaccatcttggcttggtgcaacctctgcctcccagattcaagtgattctcctgcctcagcctcctgagttgctgggaccacaggtgcacaacatcacatctggctaatttttgtatttttagtagagacagtgtttccccatattggccagggtagtcctgaactcatgacctcaaatgattcacctgtcttggcctcccaaatcacagattctttttaaagcaagagttgttcaaatttatctatcagtcgtgtttcatgtatagatgcctctaaacatttaatgtccatgttatctggtgatataagtccgtattgcagcaacactcttagaaaatggaccaatttttggagatttttttggggaaaaaattttgtttaactttgactcaggcagggaatatggcattatggtctacacgtagagggagattttggcctgtgggtctggaaagcagggtcatctaattctcaccaaagttaatctagggcaccctagaatattcctgtcagaatccttattcttgcactgagaatagttatgtccttgtgctatgactggacagtgatttggtcatatgtgaagtatgaattgcttaatgtgacctgcttctctgaatttatttacagaaaatgaaagtgatgatgaggaagaggaagaaaaagggccagtgtctcccaggtaatgttgtggaattgttggctgttaattcagtagtgacatctggagattgtagatttagggaaaatgaggaagtgatgaatagaactatttcttccattcacccagctacaaattgtgctgatttacaatgttgtatgttatttgtgacacttgtattggttttaatttcatagtcctctcaagataggaacttgccatcagatgagccaggtgaactagccaaacagggttttcttgttgatcttttcaaaaaaccagccctggattcattgattttttgaagggttttttgtgtctctatctcctttagttctgctctgatcttagttacttcttgtcttctgctagcttttgaatttgtttgctttgcttctctagttattttaattgtgatgttagggtgtcaattttagatcttttctgctttctcttgtgggcatttagtgctataattttccctctacacattgctttaaatgtgtcccagagattctggtatgttgtgtctttgttctcattggtttcaaagaacatctttatttctgccttcattttgttattttcccagtagtcattcaggagcaggttgttcagtttccatgtagttgtgcggttttgagtgagttccttaatcctgagttctaatttgactgcactgtggtctgacagtttgttgtggtttccattcttttacatttgctgaggagtgctttacctccaactatgtggtcaattttggaataagtgtgatgtggtgctgagaagaatgtatattctgttgatttggggtggagagttctgtagatgtcttttaggtctgcttggtggagagctgagttcaagtcctggatatccttgtaaagcttctgtctcattgatctgtctaatattgacagtggggtgttaaagtctcccattatgattgtgtggagtctaaatctctttgtaggtctctcaggacttgctttatgaatctgggtgctcctgtatagggtgcatatatatttaggatagttaactcttcttgttgaattgatccctttaccattatgtagtggccttctttgtctcttttgatctttgttggtttaaagtctgttttatcagagactaggattgcaacccctgcatttttttgctttccatttgcttggtagatcttcctccatccctttattttgagcctatgtgtgtctctgcatgtgagataggtttcctgagtacagcacactgatgggtcttgactctttgtccaatttgccattctgtgttttttaactggggcatttagcccatttacatttaaggttaatattgttatgtgtgaatttgagcctgtcgttatgatgttagctggttatttcgcccgttagttgatgcagtttcttcctagcgtcaatggtctttacagtttggcatgtttttgcagtggctggtaccggttgttcctttccatgtttagtgcttcctttaggagctcttgtaaggcaggcctggtggtgacaaaatctctcagcatttgcttctctgtaaaggatttatttctccttcacttatgaagctttgtttggctggatatgaaattctgggttgaaaattcttttctttaagaatgttgaagatgctggagaggatgtggagaaataggaacacttttacactgttggtgagagtgtaaactagttcaacgattgtggaaggcagtgtggcaattcctcagggatctagaactagaaatagcatttgacccagccatcccattactgggtgtatacccaaaggattataaatcatgctgctgtaaagacacatgcacacatatgtttattgcggcactattcacaatagcaaagacttggaaccaagccaaatatccagcaatgatagactggattaagaaaatgtggcacgtatacaccatggaatactatgcagctataaaaaatgatgagttcatgtcctttgtaggggcatggatgaagctggaaaccatcattctcagcaaactatcgcaaggacaaaaaaccaagtaccgcatgttcttactcacaggtggaaattgaacaatgagaaaaagcacatagcactaggaagggagccagtagaatttcaggtgaagagtaatgggagatgggaacctacacaaactggagagacccaggaaagcctcctgagaaggtcctttaagatgagctgtgaggccaaggaagagcaggccaggcgaaggcagaggggacagccttccagtgagtggaaacagcaaatgcaaaggcctcgaggcggggcagggcttgtgcttttggggagcagaaagaaggtcacggaaaaaaaacagcgtgattcatggagagaggcccagggctgaaaagggagaggcaggagtggggctcatggtctctagaaaacagactgggtttgagtttgtgtaatagcctggtgaggacggtattattatttctgcttccattttacagttacagaaacagagggcagcatatgatactggggggtactgggatagggctgtgttattgattgatgggggacctttgacggtcagaaatggagggaggctggtggtgtacccccttgacttcacctgaatgtcagggcaaggccaggagcatgcaggaggcttaaagattgggaacccctattcttgggccttctccccattctttctggagcaattggaagaggcccatgggagggaggtgactgtcatgatgtcccccactat

SEQ ID NO: 161 - protein, 86 aa, Homo sapiens
  der1 breakpoint-overlapping; Genbank AF379606
DMQKAEEKEVPEDSLEECAITCSNSHGPYDSNQPHRKTKITFEEDKVDSTLIGSSSHVEWEDAVHIIPENESDDEEEEEKGPVSPR

SEQ ID NO: 162 - DNA, 420 bp, Homo sapiens
  chimera 1; Genbank AF420438
  CDS (1)..(207)
attggctcatcctctcatgttgaatgggaggatgctgtacacattatcccagaaaatgaaagtgatgatgaggaagaggaagaaaaagggccagtgtctcccaggatgaagaatgaggccacactgccaaagcaagatctcgtgctatgtgtgattgaatctcctgctgggcgccaagcaggaatctcatcatttccccacaggccctgagagcacacaggcctcatccccaccccaccacagtgcagtgcactcactgcagagccaatgaggcctgagctcagggtctgcgtctgcctgcatctgcatggccacttccatctgtggtacttgttagtttattttaaatgtgcgatttgtttggttgcattttctccttctaaatatacagtagtttttgcaaaaaaaaaaaaaaaaaaa

SEQ ID NO: 163 - protein, 69 aa, Homo sapiens
  chimera 1; Genbank AF420438
IGSSSHVEWEDAVHIIPENESDDEEEEEKGPVSPRMKNEATLPKQDLVLCVIESPAGRQAGISSFPHRP

SEQ ID NO: 164 - DNA, 457 bp, Homo sapiens
  chimera 2; Genbank AF420439
  CDS (1)..(138)
attggctcatcctctcatgttgaatgggaggatgctgtacacattatcccagaaaatgaaagtgatgatgaggaagaggaagaaaaagggccagtgtctcccaggtgtctctgcaggaaacaatgtggacaatcacagtagttggaaggacaatgactgacctaatggagttcaggacgctatgactgttggcacctgatgtgcacagctgctcccatctgcgcagagagaatataaaggcagtggcgctgaaacatctgctgctttcactcaccatcaagatggaactgactgtacatgcaagcaaattttgatgaagagagtgatctcagagtgcgaagataagttgtttctccttgacacctaggaatgcacctggccaagaccctcagttgaaagacaatatcaaataaatgcaaaattgtaaatcaaaaaaaaaaaaaaaaaaaaaaaaaaa

SEQ ID NO: 165 - protein, 46 aa, Homo sapiens
  chimera 2; Genbank AF420439
IGSSSHVEWEDAVHIIPENESDDEEEEEKGPVSPRCLCRKQCGQSQ

SEQ ID NO: 166 - DNA, 1677 bp, Homo sapiens
  image: 2550162; Genbank AF379636; cDNA clone AG11
  CDS (1)..(1647)
gtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgagaaagggcctgaagtcttgcaggactcactggatagatgttattcaactccttcaggttgtcttgaactgactgactcatgccagccctacagaagtgccttttacatattggagcaacagtgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgagaaagggcctgaagtcttgcaggactcactggatagatgttattcaactccttcaggttgtcttgaactgactgactcatgccagccctacagaagtgccttttacatattggagcaacagtgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaagaaggaaagaaaaaaaaaaaaaaaaaaa

SEQ ID NO: 167 - protein, 549 aa, Homo sapiens
  image: 2550162; Genbank AF379636; cDNA clone AG11
VLQDSLDRCYSTPSSCLEQPDSCQPYGSSFYALEEKHVGFSLDVGEIEKKGKGKKRRGRRSKKERRRGRKEGEEDQNPPCPRLSRELLDEKGPEVLQDSLDRCYSTPSGCLELTDSCQPYRSAFYILEQQCVGLAVDMDEIEKYQEVEEDQDPSCPRLSRELLDEKEPEVLQDSLDRCYSTPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRSKKERRRGRKEGEEDQNPPCPRLSRELLDEKGPEVLQDSLDRCYSTPSGCLELTDSCQPYRSAFYILEQQCVGLAVDMDEIEKYQEVEEDQDPSCPRLSRELLDEKEPEVLQDSLDRCYSTPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRS

SEQ ID NO: 168 - DNA, 1865 bp, Homo sapiens
  image: 2663915; Genbank AF379635; cDNA clone AG10; before frameshift
  CDS (3)..(416)
acaaagtcaactcagctctggttgtagacagagaatcctctcatgatgaatgtcaggatgctgtaaacattctcccagtccctggccccacctcttctgccacaaacgtcagcatggtggtatcagccggccctttgtccagcgagaaggcagagatgaacattctagaaatgaatgagaaattgcgcccccagctggcagagaagaaacagcagttcagaaacctcaaagagaaatgttttgtaactcaactggcctgcttcctggccaaccagcagaacaaatacaaatatgaagagtgcaaagacctcataaaatctgtgctgaggaatgagcgacagttcaaggaggagaagcttgcagagcagctcaagcaagctgaggagctcaggcaatataaagtcctggttcactcaggaacgagagctgacccagttaagggagaagttacgggaagggagagatgcctcccgctcattgaatcagcatctccaggccctcctcactccggatgagccagacaagtcccaggggcaggacctccaagaacagctggctgaggggtgtagactggcacagcaacttttccaaaagctcagcccagaaaatgacaatgatcacgatgaagatgttcaagttgaggtggctgagaaagtgcagaaatcgtctgcccccagggagatgcagaaggctgaagaaaaggaagtccctgaggactcactggaggaatgtgccatcacttgttcaaatagccatggcccttatgactccaaccagccacataggaaaaccaaaatcacatttgaggaagacaaagtcgactcaactctcattggctcatcctctcatgttgaatgggaggatgctgtacacattatcccagaaaatgaaagtgatgatgaggaagaggaagaaaaagggccagtgtctcccaggaatctgcaggagtctgaagaggaggaagtcccccaggagtcctgggatgaaggttattcgactctctcaattcctcctgaaatgttggcctcgtaccagtcttacagcggcacatttcactcattagaggaacagcaagtctgcatggctgttgacataggcggacatcggtgggatcaagtgaaaaaggaggaccaagaggcaacaggtcccaggctcagcagggagctgctggatgagaaagggcctgaagtcttgcaggactcactggatagatgttattcaactccttcaggttatcttgaactgactgactcatgccagccctacagaagtgccttttacatattggagcaacagcgtgttggctgggctcttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagaagtgctgtttactcattggaggaacagtaccttggcttggctcttgacgtggacagaattaaaaaggaccaggaagaggaagaagaccaaggcccaccatgccccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaacgaagaaaagaaggagaaggggaagaaaagaaaaaaaaaaaaa

SEQ ID NO: 169 - protein, 138 aa, Homo sapiens
  image: 2663915; Genbank AF379635; cDNA clone AG10; before frameshift
KVNSALVVDRESSHDECQDAVNILPVPGPTSSATNVSMVVSAGPLSSEKAEMNILEMNEKLRPQLAEKKQQFRNLKEKCFVTQLACFLANQQNKYKYEECKDLIKSVLRNERQFKEEKLAEQLKQAEELRQYKVLVHS

SEQ ID NO: 170 - DNA, 1865 bp, Homo sapiens; identical to SEQ ID NO: 168
  image: 2663915; Genbank AF379635; cDNA clone AG10; after frameshift
  CDS (415)..(1863)

SEQ ID NO: 171 - protein, 483 aa, Homo sapiens
  image: 2663915; Genbank AF379635; cDNA clone AG10; after frameshift
QERELTQLREKLREGRDASRSLNQHLQALLTPDEPDKSQGQDLQEQLAEGCRLAQQLFQKLSPENDNDHDEDVQVEVAEKVQKSSAPREMQKAEEKEVPEDSLEECAITCSNSHGPYDSNQPHRKTKITFEEDKVDSTLIGSSSHVEWEDAVHIIPENESDDEEEEEKGPVSPRNLQESEEEEVPQESWDEGYSTLSIPPEMLASYQSYSGTFHSLEEQQVCMAVDIGGHRWDQVKKEDQEATGPRLSRELLDEKGPEVLQDSLDRCYSTPSGYLELTDSCQPYRSAFYILEQQRVGWALDMDEIEKYQEVEEDQDPSCPRLSRELLDEKEPEVLQDSLDRCYSTPSGYLELPDLGQPYRSAVYSLEEQYLGLALDVDRIKKDQEEEEDQGPPCPRLSRELLEAVEPEVLQDSLDRCYSTPSSCLEQPDSCLPYGSSFYALEEKHVGFSLDVGEIEKKGKGKKRRGRRSTKKRRRRGRKEKKK

SEQ ID NO: 172 - DNA, 2594 bp, Homo sapiens
  image: 2186481; Genbak AF379633; cDNA clone AG06
  CDS (3)..(2579)
gttattcgactccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgagaaagggcctgaagtcttgcaggactcactggatagatgttattcaactccttcaggttgtcttgaactgactgactcatgccagccctacagaagtgccttttacatattggagcaacagtgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgagaaagggcctgaagtcttgcaggactcactggatagatgttattcaactccttcaggttgtcttgaactgactgactcatgccagccctacagaagtgccttttacatattggagcaacagtgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgattccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgagaaagggcctgaagtcttgcaggactcactggatagatgttattcaactccttcaggttgtcttgaactgactgactcatgccagccctacagaagtgccttttacatattggagcaacagtgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaaaaaaaaaaaaaaa

SEQ ID NO: 173 - protein, 859 aa, Homo sapiens
  image: 2186481; Genbak AF379633; cDNA clone AG06
YSTPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRSKKERRRGRKEGEEDQNPPCPRLSRELLDEKGPEVLQDSLDRCYSTPSGCLELTDSCQPYRSAFYILEQQCVGLAVDMDEIEKYQEVEEDQDPSCPRLSRELLDEKEPEVLQDSLDRCYSTPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRSKKERRRGRKEGEEDQNPPCPRLSRELLDEKGPEVLQDSLDRCYSTPSGCLELTDSCQPYRSAFYILEQQCVGLAVDMDEIEKYQEVEEDQDPSCPRLSRELLDEKEPEVLQDSLDRCYSIPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRSKKERRRGRKEGEEDQNPPCPRLSRELLDEKGPEVLQDSLDRCYSTPSGCLELTDSCQPYRSAFYILEQQCVGLAVDMDEIEKYQEVEEDQDPSCPRLSRELLDEKEPEVLQDSLDRCYSTPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRS

SEQ ID NO: 174 - DNA, 915 bp, Homo sapiens
  image: 1700501; Genbank AF379632; cDNA clone AG04
  CDS (1)..(897)
ggggaaaaacatgttggcttttctcttgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgagaaagggcctgaagtcttgcaggactcactggatagatgttattcaactccttcaggttgtcttgaactgactgactcatgccagccctacagaagtgccttttatgtattggagcaacagcgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaagaagatcaaagaagaaggggaagaaaaaaaggggaagaaaagaaggggaagaagatcaaagaaggaaagaagaaggggaagaaaaaaaaaaaaaaaa

SEQ ID NO: 175 - protein, 299 aa, Homo sapiens
  image: 1700501; Genbank AF379632; cDNA clone AG04
GEKHVGFSLDVGEIEKKGKGKKRRGRRSKKERRRGRKEGEEDQNPPCPRLSRELLDEKGPEVLQDSLDRCYSTPSGCLELTDSCQPYRSAFYVLEQQRVGLAVDMDEIEKYQEVEEDQDPSCPRLSRELLDEKEPEVLQDSLDRCYSTPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRSKKIKEEGEEKKGKKRRGRRSKKERRRG

SEQ ID NO: 176 - DNA, 582 bp, Homo sapiens
  image: 450510; Genbank AF379631; cDNA clone AE05
  CDS (1)..(522)
ggcacgagcaaagtcgactcaactctcattggctcatcctctcatgttgaatgggaggatgctgtacacattattccagaaaatgaaagtgatgatgaggaagaggaagaaaaaggaccagtgtctcccaggaatctgcaggagtctgaagaggaggaagtcccccaggagtcctgggatgaaggttattcgactctctcaattcctcctgaaatgttggcctcgtacaagtcttacagcagcacatttcactcattagaggaacagcaagtctgcatggctgttgacataggcagacatcggtgggatcaagtgaaaaaggaggaccacgaggcaacaggtcccaggctcagcagagagctgctggatgagaaagggcctgaagtcttgcaggactcactggatagatgttattcaactccttcaggttgtcttgaactgactgactcatgccagccctacagaagtgccttttacgtattggagcaacagcgtgttggcttggctgttgacatggatgaaattgaaaagtaccaagaaataaactcgtaaccctgtccttcaaaaaaaaaaaaaaaaaaa

SEQ ID NO: 177 - protein, 174 aa, Homo sapiens
  image: 450510; Genbank AF379631; cDNA clone AE05
GTSKVDSTLIGSSSHVEWEDAVHIIPENESDDEEEEEKGPVSPRNLQESEEEEVPQESWDEGYSTLSIPPEMLASYKSYSSTFHSLEEQQVCMAVDIGRHRWDQVKKEDHEATGPRLSRELLDEKGPEVLQDSLDRCYSTPSGCLELTDSCQPYRSAFYVLEQQRVGLAVDMDE

SEQ ID NO: 178 - DNA, 1555 bp, Homo sapiens
  image: 44185; Genbank AF379630; cDNA clone AE03
  CDS (3)..(1520)
aagatctcataaaatttatgctgaggaatgagcgacagttcaaggaggagaagcttgcagagcagctcaagcaagctgaggagctcaggcaatataaagtcctggttcacgctcaggaacgagagctgacccagttaagggagaagttacgggaagggagagatgcctcccgctcattgaatgagcatctccaggccctcctcactccggatgagccggacaagtcccaggggcaggacctccaagaacagctggctgaggggtgtagactgacacagcaccttgtccaaaagctcagcccagaaaatgacaacgatgacgatgaagatgttcaagttgaggtggctgagaaagtgcagaaatcgtctgcccccagggagatgcagaaggctgaagaaaaggaagtccctgaggactcactggaggaatgtgccatcacttgttcaaatagccatggcccttatgactccaaccagccacataagaaaaccaaaatcacatttgaggaagacaaagtcgactcaactctcattggctcatcctctcatgttgaatgggaggatgctgtacacattattccagaaaatgaaagtgatgatgaggaagaggaagaaaaagggccagtgtctcccaggaatctgcaggagtctgaagaggaggaagtcccccaggagtcctgggatgaaggttattcgactctctcaattcctcctgaaatgttggcctcgtaccagtcttacagcagcacatttcactcattagaggaacagcaagtctgcatggctgttgacataggcagacatcggtgggatcaagtgaaaaaggaggaccaagaggcaacaggtcccaggctcagcagggagctgctggatgagaaagagcctgaagtcttgcaggactcactggatagatgttattcaactccttcaggttgtcttgaactgactgactcatgccagccctacagaagtgccttttacgtattggagcaacagcgtgttggcttggctattgacatggatgaaattgaaaagtaccaagaagtggaagaagaccaagacccatcatgccccaggctcagcagggagctgctggatgagaaagagcctgaagtcttgcaggactcactggatagatgttattcaactccttcagattatcttgaactgcctgacttaggccagccctacagcagtgctgtttactcattggaggaacagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaaaaaaaaaaaaaaaaaaaaaaaaaaaaaaaaaaa

SEQ ID NO: 179 - protein, 506 aa, Homo sapiens
  image: 44185; Genbank AF379630; cDNA clone AE03
DLIKFMLRNERQFKEEKLAEQLKQAEELRQYKVLVHAQERELTQLREKLREGRDASRSLNEHLQALLTPDEPDKSQGQDLQEQLAEGCRLTQHLVQKLSPENDNDDDEDVQVEVAEKVQKSSAPREMQKAEEKEVPEDSLEECAITCSNSHGPYDSNQPHKKTKITFEEDKVDSTLIGSSSHVEWEDAVHIIPENESDDEEEEEKGPVSPRNLQESEEEEVPQESWDEGYSTLSIPPEMLASYQSYSSTFHSLEEQQVCMAVDIGRHRWDQVKKEDQEATGPRLSRELLDEKEPEVLQDSLDRCYSTPSGCLELTDSCQPYRSAFYVLEQQRVGLAIDMDEIEKYQEVEEDQDPSCPRLSRELLDEKEPEVLQDSLDRCYSTPSDYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRS

SEQ ID NO: 180 - DNA, 444 bp, Homo sapiens
  image: 341197; Genbank AF379629; cDNA clone AE02
  CDS (269)..(433)
ggcacgaggattgatccagtcctccttccttcactaccacatgaatgctgggcagcccaggatcacactcactgcaccctcaactcagaccgttacctggcacactggcctcactcttgtcggagactgagctattggcagtgccttcagctctgagctcaggcacctcgaacattgtttttgtcgttaaggatcctaaagtgctgcggggactgatcacatttttctcaacatccctggccccacctcttctgccacaaacgtcagcatggtggtatctgccggcccttggtccggtgagaaggcagagatgaacattctagaaatcaacaagaaatcgcgcccccagctggcagagaacaaacagcagttcagaaacctcaaacagaaatgtcttgtaactcaagtggcctacttcctggccaaccggcaaaaaaaaaaaaa

SEQ ID NO: 181 - protein, 55 aa, Homo sapiens
  image: 341197; Genbank AF379629; cDNA clone AE02
MVVSAGPWSGEKAEMNILEINKKSRPQLAENKQQFRNLKQKCLVTQVAYFLANRQ

SEQ ID NO: 182 - DNA, 2630 bp, Homo sapiens
  DKFZp434D0623; Genbank AF379626; cDNA clone AC03
  CDS (2)..(1507)
ccaagacccatcatgccccaggctcagcagggagctgctggatgagaaagagcctgaagtcttgcaggactcactggatagatgttattcgactccttcaggttatcttgaactgcctgacttaggccagccctacagaagtgctgtttactcattggaggaacagtaccttggcttggctcttgacgtggacagaattaaaaaggaccaagaagaggaagaagaccaaggcccaccatgccccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaatgaagaaaagaaggagaaggggaagaaaagaaggggaagaagatcaaaacccaccatgccccaggctcaacagggagctgctggatgagaaagggcctgaagtcttgcaggactcactggatagatgttattcaactccttcaggttatcttgaactgactgactcatgccagccctacagaagtgccttttacatattggagcaacagcgtgttggctgggctcttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagaagtgctgtttactcattggaggaacagtaccttggcttggctcttgacgtggacagaattaaaaaggaccaagaagaggaagaagaccaaggcccaccatgccccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaatgaagaaaagaaggagaaggggaagaaaagaaggggaagaagatcaaaacccaccatgccccaggctcagcggcatgctgatggaagtggaagagcctgaagtcttgcaggactcactggatagatgttattcgactccgtcaatgtactttgaactacctgactcattccagcactacagaagtgtgttttactcatttgaggaacagcacatcagcttcgcccttgacgtggacaataggtttcttactttgatgggaacaagtctccacctggtcttccagatgggagtcatattcccacagtaagcagcccttactaagccgagagatgtcattcctgcaggcaggacctataggcacgtgaagatttgaatgaaactatagttccatttggaagcccagacataggatgggtcagtgggcatggctctattcctattctcagaccatgccagtggcaacctgtgctcagtctgaagacaatggacccaagttaggtgtgacacgttcacataactgtgcagcacatgccgggagtgatcagtcagacattttaatttgaaccacttatctctgggtagctacaaagttcctcagggatttcattttgcagtcatgtctctgagcttctatacctgctcaaggtcagtgtcatctttgtgtttagctcatccaaaggtgttaccctggtttcaatgaacctaacctcattctttgtatcttcagtgttgaattgttttagctgatccatctttaacacaggagggatccttggctgaggattgtatttcagaaccaccaactgctcttgacaattgttaacccgctaggctcctttggttagagaagccagtccttcagcctccaattggtgtcagtacttaggaagaccacagctagatggacaaacagcattgggaggccttagccctgctcctctcgattccatcctgtagagaacaggagtcaggagccgctggcaggagacagcatgtcacccaggactctgccggtgcagaatatgaacaacgccatgttcttgcagaaaacgcttagcctgagtttcataggaggtaattaccagacaactgcagaatgtagaacactgagcaggacaactgacctgtctccttcacatagtccatatcaccacaaatcacacaacaaaaaggagaagagatattttgggttcaaaaaaagtaaaaagataatatagctgcatttctttagttattttgaaccccaaatatttcctcatctttttgttgttgtcattgatggtggtgacatggacttgtttatagaggacaggtcagctgtctggctcaatgatctacattctgaagttgtctgaaaatgtcttcatgattaaattcagcctaaacgttttgccgggaacactgcagaagacaatgctgtgagtttc

SEQ ID NO: 183 - protein, 502 aa, Homo sapiens
  DKFZp434D0623; Genbank AF379626; cDNA clone AC03
QDPSCPRLSRELLDEKEPEVLQDSLDRCYSTPSGYLELPDLGQPYRSAVYSLEEQYLGLALDVDRIKKDQEEEEDQGPPCPRLSRELLEAVEPEVLQDSLDRCYSTPSSCLEQPDSCLPYGSSFYALEEKHVGFSLDVGEIEKKGKGKKRRGRRSMKKRRRRGRKEGEEDQNPPCPRLNRELLDEKGPEVLQDSLDRCYSTPSGYLELTDSCQPYRSAFYILEQQRVGWALDMDEIEKYQEVEEDQDPSCPRLSRELLDEKEPEVLQDSLDRCYSTPSGYLELPDLGQPYRSAVYSLEEQYLGLALDVDRIKKDQEEEEDQGPPCPRLSRELLEAVEPEVLQDSLDRCYSTPSSCLEQPDSCLPYGSSFYALEEKHVGFSLDVGEIEKKGKGKKRRGRRSMKKRRRRGRKEGEEDQNPPCPRLSGMLMEVEEPEVLQDSLDRCYSTPSMYFELPDSFQHYRSVFYSFEEQHISFALDVDNRFLTLMGTSLHLVFQMGVIFPQ

SEQ ID NO: 184 - DNA, 4440 bp, Homo sapiens
  DKFZp434G2022
  CDS (3)..(2903)
aagaaaaggaagtccctgaggactcactggaggaatgtgccatcacttgttcaaatagccatggcccttattactccaaccagccacataggaaaaccaaaatcacatttgaggaagacaaagtcgactcaactctcattggctcatcctctcatgttgaatgggaggatgctgtacacattatcccagaaaatgaaagtgatgatgaggaagaggaagaaaaagggccagtgtctcccaggaatctgcaggagtctgaagaggaggaagtcccccaggagtcctgggatgaaggttattcgactctctcaattcctcctgaaatgttggcctcgtaccagtcttacagcggcacatttcactcattagaggaacagcaagtctgcatggctgttgacataggcggacatcggtgggatcaagtgaaaaaggaggaccaagaggcaacaggtcccaggctcagcagggagctgctggatgagaaagggcctgaagtcttgcaggactcactggatagatgttattcaactccttcaggttatcttgaactgactgactcatgccagccctacagaagtgccttttacatattggagcaacagcgtgttggctgggctcttgacatggatgaaattgaaaagtaccaagaagtggaagaagaccaagacccatcatgccccaggctcagcagggagctgctggatgagaaagagcctgaagtcttgcaggactccctggatagatgttattcgactccttcaggttatcttgaactgcctgacttaggccagccctacagaagtgctgtttactcattggaggaacagtaccttggcttggctcttgacgtggacagaattaaaaaggaccaagaagaggaagaagaccaaggcccaccatgccccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaacgaagaaaagaaggagaaggggaagaaaagaaggggaagaagatcaaaacccaccatgccccaggctcagcagggagctgctggatgagaaagggcctgaagtcttgcaggactcactggatagatgttattcaactccttcaggttatcttgaactgactgactcatgccagccctacagaagtgccttttacatattggagcaacagcgtgttggctgggctcttgacatggatgaaattgaaaagtaccaagaagtggaagaagaccaagacccatcatgccccaggctcagcagggagctgctggatgagaaagagcctgaagtcttgcaggactccctggatagatgttattcgactccttcaggttatcttgaactgcctgacttaggccagccctacagaagtgctgtttactcattggaggaacagtaccttggcttggctcttgacgtggacagaattaaaaaggaccaggaagaggaagaagaccaaggcccaccatgccccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaacgaagaaaagaaggagaaggggaagaaaagaaggggaagaagatcaaaacccaccatgccccaggctcagcagggagctgctggatgagaaagggcctgaagtcttgcaggactcactggatagatgttattcaactccttcaggttatcttgaactgactgactcatgccagccctacagaagtgccttttacatattggagcaacagcgtgttggctgggctcttgacatggatgaaattgaaaagtaccaagaagtggaagaagaccaagacccatcatgccccaggctcagcagggagctgctggatgagaaagagcctgaagtcttgcaggactccctggatagatgttattcgactccttcaggttatcttgaactgcctgacttaggccagccctacagaagtgctgtttactcattggaggaacagtaccttggcttggctcttgacgtggacagaattaaaaaggaccaggaagaggaagaagaccaaggcccaccatgccccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaacgaagaaaagaaggagaaggggaagaaaagaaggggaagaagatcaaaacccaccatgccccaggctcagcggtgtgctgatggaagtggaagagcctgaaatcttgcaggactcactggatagatgttattcgactccgtcaatgttctttgaactacctgactcattccagcactacagaagtgtgttttactcatttgaggaacagcacatcagcttcgcccttgacgtggacaataggtttcttactttgatgggaacaagtctccacctggtcttccagatgggagtcatattcccacagtaagcagcccttactaagccgagagatgtcattcctgcaggcaggacctataggcacatgaagatttgaatgaaactatagttccatttggaagcccagacataggatgggtcagtgggcatggctctattcctattctcagaccatgccagtggcaacctgtgctcagtctgaagacaatggacccaagttaggtgtgacacgttcacataactgtgcagcacatgccgggagtgatcagtcagacatttcaatttgaaccacgtatctctgggtagctacaaagttcctcagggatttcattttgcaggcatgtctctgagcttctatacctgctcaaggtcagtgtcatctttgtgtttagctcatccaaaggtgttaccctggtttcaatgaacctaacctcattctttgtatcttcagtgttgaattgttttagctgatccatctttaacacaggagggatccttggctgaggattgtatttcagaaccaccaactgctcttgacaattgttaacccgctaggctcctttggttagagaagccacagtccttcagcctccaattggtgtcagtacttaggaagaccacagctagatggacaaacagcattgggaggccttagccctgctcctctcgattccatcctgtagagaacaggagtcaggagccgctggcaggagacagcatgtcacccaggactctgccggtgcagaatatgaacaacgccatgttcttgcagaaaacgcttagcctgagtttcataggaggtaatcaccagacaactgcggaatgtagagcactgagcaggacagctggcctgtctccttcacatagtccatgtcaaccacaaatcacacaacaaaaaggagaggagatattttgggttcaaaaaaagtaaaaagataatgtagctgcatttctttggttattttgggccccaaatatttcctcatctttttgttgttgtcattgatggtggtgacatggacttgtttatagaggacaggtcagctgtctggctcagtgatctacattctgaagttgtctgaaaatgtcttcatgattaaattcagcctaaacgttttgccgggaacactgcagagacaatgctgtgagtttccaaccttagcccatctgcgggcagagaaggtctagtttgtccatcagcattatcatgatatcaggactggttacttggttaaggaggggtctaggagatctgtcccttttagagacaccttacttataatgaagtatttgggagggtggttttcaaaagtagaaatgtcctgtattccgatgatcatcctgtaaacattttatcatttattaatcatccctgcctgtgtctattattatattcatatctctacgctggaaactttctgcctcaatgtttactgtgcctttgtttttgctagtgtgtgttgttgaaaaaaaaaacattctctgcctgagttttaatttttgtccaaagttattttaatctatacaattaaaagcttttgcctatcaaaaaaaaaaaaaaaaaa

SEQ ID NO: 185 - protein, 967 aa, Homo sapiens
  DKFZp434G2022
EKEVPEDSLEECAITCSNSHGPYYSNQPHRKTKITFEEDKVDSTLIGSSSHVEWEDAVHIIPENESDDEEEEEKGPVSPRNLQESEEEEVPQESWDEGYSTLSIPPEMLASYQSYSGTFHSLEEQQVCMAVDIGGHRWDQVKKEDQEATGPRLSRELLDEKGPEVLQDSLDRCYSTPSGYLELTDSCQPYRSAFYILEQQRVGWALDMDEIEKYQEVEEDQDPSCPRLSRELLDEKEPEVLQDSLDRCYSTPSGYLELPDLGQPYRSAVYSLEEQYLGLALDVDRIKKDQEEEEDQGPPCPRLSRELLEAVEPEVLQDSLDRCYSTPSSCLEQPDSCLPYGSSFYALEEKHVGFSLDVGEIEKKGKGKKRRGRRSTKKRRRRGRKEGEEDQNPPCPRLSRELLDEKGPEVLQDSLDRCYSTPSGYLELTDSCQPYRSAFYILEQQRVGWALDMDEIEKYQEVEEDQDPSCPRLSRELLDEKEPEVLQDSLDRCYSTPSGYLELPDLGQPYRSAVYSLEEQYLGLALDVDRIKKDQEEEEDQGPPCPRLSRELLEAVEPEVLQDSLDRCYSTPSSCLEQPDSCLPYGSSFYALEEKHVGFSLDVGEIEKKGKGKKRRGRRSTKKRRRRGRKEGEEDQNPPCPRLSRELLDEKGPEVLQDSLDRCYSTPSGYLELTDSCQPYRSAFYILEQQRVGWALDMDEIEKYQEVEEDQDPSCPRLSRELLDEKEPEVLQDSLDRCYSTPSGYLELPDLGQPYRSAVYSLEEQYLGLALDVDRIKKDQEEEEDQGPPCPRLSRELLEAVEPEVLQDSLDRCYSTPSSCLEQPDSCLPYGSSFYALEEKHVGFSLDVGEIEKKGKGKKRRGRRSTKKRRRRGRKEGEEDQNPPCPRLSGVLMEVEEPEILQDSLDRCYSTPSMFFELPDSFQHYRSVFYSFEEQHISFALDVDNRFLTLMGTSLHLVFQMGVIFPQ

SEQ ID NO: 186 - DNA, 2647 bp, Homo sapiens
  DKFZp434M0628; Genbank AF379622; cDNA clone AB18
  CDS (1)..(1074)
gggaaggggaagaaaagaaggggaagaagatcaatgaagaaaagaaggagaaggggaagaaaagaaggggaagaagatcaaaacccaccatgccccaggctcaacagggagctgctggatgagaaagggcctgaagtcttgcaggactcactggatagatgttattcaactccttcaggttatcttgaactgactgactcatgccagccctacagaagtgccttttacatattggagcaacagcgtgttggctgggctcttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagaagtgctgtttactcattggaggaacagtaccttggcttggctcttgacgtggacagaattaaaaaggaccaagaagaggaagaagaccaaggcccaccatgccccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaatgaagaaaagaaggagaaggggaagaaaagaaggggaagaagatcaaaacccaccatgccccaggctcagcggcatgctgatggaagtggaagagcctgaagtcttgcaggactcactggatagatgttattcgactccgtcaatgtactttgaactacctgactcattccagcactacagaagtgtgttttactcatttgaggaacagcacatcagcttcgcccttgacgtggacaataggtttcttactttgatgggaacaagtctccacctggtcttccagatgggagtcatattcccacagtaagcagcccttactaagccgagagatgtcattcctgcaggcaggacctataggcacgtgaagatttgaatgaaactatagttccatttggaagcccagacataggatgggtcagtgggcatggctctattcctattctcagaccatgccagtggcaacctgtgctcagtctgaagacaatggacccaagttaggtgtgacacgttcacataactgtgcagcacatgccgggagtgatcagtcagacattttaatttgaaccacttatctctgggtagctacaaagttcctcagggatttcattttgcagtcatgtctctgagcttctatacctgctcaaggtcagtgtcatctttgtgtttagctcatccaaaggtgttaccctggtttcaatgaacctaacctcattctttgtatcttcagtgttgaattgttttagctgatccatctttaacacaggagggatccttggctgaggattgtatttcagaaccaccaactgctcttgacaattgttaacccgctaggctcctttggttagagaagccagtccttcagcctccaattggtgtcagtacttaggaagaccacagctagatggacaaacagcattgggaggccttagccctgctcctctcgattccatcctgtagagaacaggagtcaggagccgctggcaggagacagcatgtcacccaggactctgccggtgcagaatatgaacaacgccatgttcttgcagaaaacgcttggcctgagtttcataggaggtaattaccagacaactgcagaatgtagaacactgagcaggacaactgacctgtctccttcacatagtccatatcaccacaaatcacacaacaaaaaggagaagagatattttgggttcaaaaaaagtaaaaagataatatagctgcatttctttagttattttgaaccccaaatatttcctcatctttttgttgttgtcattgatggtggtgacatggacttgtttatagaggacaggtcagctgtctggctcaatgatctacattctgaagttgtctgaaaatgtcttcatgattaaattcagcctaaacgttttgccgggaacactgcagagacaatgctgtgagtttccaaccttagcccatctgcgggcagagaaggtctagtttgtccatcagcattatcatgatatcaggactggttacttggttaaggaggggtctaggagatctgtcccttttagagacaccttacttataatgaagtatttgggagggtggttttcaaaagtagaaatgtcctgtattccgatgatcatcctgtaaacattttatcatttattaatcatccctgcctgtgtctattattatattcatatctctacgctggaaactttctgcctcaatgtttactgtgcctttgtttttgctagtgtgtgttgttgaaaaaaaaaacattctctgcctgagttttaatttttgtccaaagttattttaatctatacaattaaaagcttttgcctatcaaaaaaaaaaaaaaaaaaaaaaaaaaaaaaaaaaaaaaaaaaaaaaaaaaaaaaaaa

SEQ ID NO: 187 - protein, 358 aa, Homo sapiens
  DKFZp434M0628; Genbank AF379622; cDNA clone AB18
GKGKKRRGRRSMKKRRRRGRKEGEEDQNPPCPRLNRELLDEKGPEVLQDSLDRCYSTPSGYLELTDSCQPYRSAFYILEQQRVGWALDMDEIEKYQEVEEDQDPSCPRLSRELLDEKEPEVLQDSLDRCYSTPSGYLELPDLGQPYRSAVYSLEEQYLGLALDVDRIKKDQEEEEDQGPPCPRLSRELLEAVEPEVLQDSLDRCYSTPSSCLEQPDSCLPYGSSFYALEEKHVGFSLDVGEIEKKGKGKKRRGRRSMKKRRRRGRKEGEEDQNPPCPRLSGMLMEVEEPEVLQDSLDRCYSTPSMYFELPDSFQHYRSVFYSFEEQHISFALDVDNRFLTLMGTSLHLVFQMGVIFPQ

SEQ ID NO: 188 - DNA, 2982 bp, Homo sapiens
  DKFZp434J205; Genbank AF379616; cDNA clone AB06
  CDS (152)..(1411)
ccaacatgaaggcaataatttgttacctcattaatggatctctccttttactttttcaaccacttccttatgctacccatgaaacctagttggggctctgttgtgtctgatttcccctggcttattctttactttttcctccttttccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaacgaagaaaagaaggagaaggggaagaaaagaaggggaagaagatcaaaacccaccatgccccaggctcagcagggagctgctggatgagaaagggcctgaagtcttgcaggactcactggatagatgttattcaactccttcaggttatcttgaactgactgactcatgccagccctacagaagtgccttttacatattggagcaacagcgtgttggctgggctcttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagaagtgctgttcactcattggaggaacagtaccttggcttggctcttgacgtggacagaattaaaaaggaccaggaagaggaagaagaccaaggcccaccatgctccaggctcagcagggagctgctggaggcagtagagcctgaagtcttgctggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaacgaagaaaagaaggagaaggggaagtaaagaaggggaagaagatcaaaacccaccatgccccaggctcagcggtgtgctgatggaagtggaagagcctgaagtcttacaggactcactggatagatgttattcgactccgtcaatgtactttgaactacctgactcattccagcactacagaagtgtgttttactcatttgaggaacagcacatcagcttcgcccttgacgtggacaataggtttcttactttgatgggaacaagtctccacctggtcttccagatgggagtcatattcccacagtaagcagccctttctaagccgagagatgtcattcctgcaggcaggacctataggcacgtgaagatttgaatgaaactatagttccatttggaagcccagacataggatgggtcagtgggcgtggctctattcctattctcagaccatgccagtggcaacctgtgctcagtctgaagacaatggacccaagttaggtgtgacacgttcacataactgtgcagcacatgccgggagtgatcagtcagacattttaatttgaaccacgtatctctgggtagctacaaagttcctcagggatttcattttgcaggcatgtctctgagcttctatacctgctcaaggtcagtgtcatctttgtgtttagctcatccaaaggtgttaccctggtttcaatgaacctaacctcattctttgtatcttcagtgttgaattgttttagctgatccatctttaacacaggagggatccttggctgaggattgtatttcagaaccaccaactgctcttgacaattgttaacccgctaggctcctttggttagagaagccacagtccttcagcctccaattggtgttagtacttaagaagaccacagctagatggacaaacagcattgggaggccttagccctgctcctctcgattccatcctgtagagaacaggagtcaggagccgctggcaggagacagcatgtcacccaggactctgccggtgcagaatatgaacaacgccatgttcttgcagaaaacgcttagcctgagtttcataggaggtaatcaccagacaactgcagaatgtggaacactgagcaggacaactgacctgtctccttcacatagtccatatcaccacaaatcacacaacagaaaggagaagagatattttgggttcaaaaaaagtaaaaagataatatagctgcatttctttagttattttgaaccccaaatatttcctcatctttttgttgttgtcattgatggtggtgacatggacttgtttatagaggacaggtcagctgtctggctcagtgatctacattctgaagttgtctgaaaatgtcttcatgattaaattcagcctaaacgttttgccgggaacactgcagagacaatgctgtgagtttccaaccttagcccatctgcgggcagagaaggtctagtttgtccatcagcattatcatgatatcaggactggttacttggttaaggaggggtctaggagatgtgtcccttttagagacaccttacttataatgaagtatttgggagggtggttttcaaaagtagaaatgtcctgtattccgatgatcatcctgtaaacattttatcatttattaatcatccctgcctgtgtctattattatattcatatctctacgctggaaactttctgcctctatgtttactgtgcctttgtttttgctagtgtgtgttgttgaaaaaaaaaacattctctgcctgagttttaatttttgtccaaagttattttaatctatacaattaaaagcttttgcctatcaaaaaaaaaaaaaaaaaaaaaaaaaaaaaaaaaaaaaaaaaaaaaaaaaaaaa

SEQ ID NO: 189 - protein, 420 aa, Homo sapiens
  DKFZp434J205; Genbank AF379616; cDNA clone AB06
LSRELLEAVEPEVLQDSLDRCYSTPSSCLEQPDSCLPYGSSFYALEEKHVGFSLDVGEIEKKGKGKKRRGRRSTKKRRRRGRKEGEEDQNPPCPRLSRELLDEKGPEVLQDSLDRCYSTPSGYLELTDSCQPYRSAFYILEQQRVGWALDMDEIEKYQEVEEDQDPSCPRLSRELLDEKEPEVLQDSLDRCYSTPSGYLELPDLGQPYRSAVHSLEEQYLGLALDVDRIKKDQEEEEDQGPPCSRLSRELLEAVEPEVLLDSLDRCYSTPSSCLEQPDSCLPYGSSFYALEEKHVGFSLDVGEIEKKGKGKKRRGRRSTKKRRRRGSKEGEEDQNPPCPRLSGVLMEVEEPEVLQDSLDRCYSTPSMYFELPDSFQHYRSVFYSFEEQHISFALDVDNRFLTLMGTSLHLVFQMGVIFPQ

SEQ ID NO: 190 - DNA, 1217 bp, Homo sapiens
  image: 2920609; Genbank AF380581; cDNA clone AG03
  CDS (2)..(1201)
tgaagatgttcaagttgaggtggctgagaaagtgcagaaatcgtctgcccccagggagatgccgaaggctgaagaaaaggaagtccctgaggactcactggaggaatgtgccatcacttgttcaaatagccatggcccttatgactccaaccagccacataggaaaaccaaaatcacatttgaggaagacaaagtcgactcaactctcattggctcatcctctcatgttgaatgggaggatgctgtacacattatcccagaaaatgaaagtgatgatgaggaagaggaagaaaaagggccagtgtctcccaggaatctgcaggagtctgaagaggaggaagtcccccaggagtcctgggatgaaggttattcgactctctcaattcctcctgaaatgttggcctcgtaccagtcttacagcagcacatttcactcattagaggaacagcaagtctgcatggctgttgacataggcggacatcagtgggatcaagtgaaaaaggaggaccaagaggcaacaggtcccaggctcagcagggagctgctggatgagaaagggcctgaagtcttgcaggactcactggatagatgttattcaactccttcaggttatcttgaactgcctgactcatgccagccctacagaagtgccttttacgtattggagcaacagcgtgttggcttgactcttgacatggatgaaattgaaaagtaccaagaagtggaagaagaccaagacccatcatgccccaggctcagcagggagctgctggatgagaaagagcctgaagtcttgcaggactccctggatagatgttattcgactccttcaggttatcttgaactgcctgacttaggccagccctacagaagtgctgtttactcattggaggaacagtaccttggcttggctcttgacgtggatagaattaaaaaggaccaagaagaggaagaagaccagggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaacgaaaaaaaaaaaaaaaa

SEQ ID NO: 191 - protein, 400 aa, Homo sapiens
  image: 2920609; Genbank AF380581; cDNA clone AG03
EDVQVEVAEKVQKSSAPREMPKAEEKEVPEDSLEECAITCSNSHGPYDSNQPHRKTKITFEEDKVDSTLIGSSSHVEWEDAVHIIPENESDDEEEEEKGPVSPRNLQESEEEEVPQESWDEGYSTLSIPPEMLASYQSYSSTFHSLEEQQVCMAVDIGGHQWDQVKKEDQEATGPRLSRELLDEKGPEVLQDSLDRCYSTPSGYLELPDSCQPYRSAFYVLEQQRVGLTLDMDEIEKYQEVEEDQDPSCPRLSRELLDEKEPEVLQDSLDRCYSTPSGYLELPDLGQPYRSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRST

SEQ ID NO: 192 - DNA, 1885 bp, Homo sapiens
  DKFZp434F2323; Genbank AF380580; cDNA clone AG01
  CDS (3)..(1847)
aaaaacatgttggcttttctcttgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgagaaagggcctgaagtcttgcaggactcactggatagaagttattcaactccttcaggttgtcttgaactgactgactcatgccagccctacagaagtgccttttacgtattggagcaacagcgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagcagtgctgtgtactcattggaggaacagtaccttggcttggctcttgacgtggacagaactaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctctcgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgagaaagggcctgaagtcttgcaggactcactggatagatgttattcaactccttcaggttgtcttgaactgactgactcatgccagccctacagaagtgccttttacgtattggagcaacagcgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagcagtgctgtttactcattggaggaacagtaccttggcttggctcttgacgtggacagaactaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaagaagaaaagaagaaggggaagaaaagaaggggaagaagatcaaaacccaccatgccccaggctcaacggcgtgctgatggaagtggaagagcctgaagtcttacaggactcactggatggatgttattctactccgtcaatgtactttgaactacctgactcattccagcactacagaagtgtgttttactcatttgaggaacagcacatcagcttcgccctttacgtggacaataggttttttactttgacggtgacaagtctccacctggtgttccagatgggagtcatattcccacaataaacagcccttactaaaaaaaaaaaaaaaaaaaaaaa

SEQ ID NO: 193 - protein, 615 aa, Homo sapiens
  DKFZp434F2323; Genbank AF380580; cDNA clone AG01
KHVGFSLDVGEIEKKGKGKKRRGRRSKKERRRGRKEGEEDQNPPCPRLSRELLDEKGPEVLQDSLDRSYSTPSGCLELTDSCQPYRSAFYVLEQQRVGLAVDMDEIEKYQEVEEDQDPSCPRLSRELLDEKEPEVLQDSLDRCYSTPSGYLELPDLGQPYSSAVYSLEEQYLGLALDVDRTKKDQEEEEDQGPPCPRLSRELLEVVEPEVLQDSLDRCYSTPSSCLEQPDSCQPYGSSFYALEEKHVGFSLDVGEIEKKGKGKKRRGRRSKKERRRGRKEGEEDQNPPCPRLSRELLDEKGPEVLQDSLDRCYSTPSGCLELTDSCQPYRSAFYVLEQQRVGLAVDMDEIEKYQEVEEDQDPSCPRLSRELLDEKEPEVLQDSLDRCYSTPSGYLELPDLGQPYSSAVYSLEEQYLGLALDVDRTKKDQEEEEDQGPPCPRLSRELLEVVEPEVLQDSLDRCYSTPSSCLEQPDSCQPYGSSFYALEEKHVGFSLDVGEIEKKGKGKKRRGRRSKKKRRRGRKEGEEDQNPPCPRLNGVLMEVEEPEVLQDSLDGCYSTPSMYFELPDSFQHYRSVFYSFEEQHISFALYVDNRFFTLTVTSLHLVFQMGVIFPQ

SEQ ID NO: 194 - DNA, 1528 bp, Homo sapiens
  image: 2782170 (5′ end); Genbank AF419618; cDNA clone AE01
  CDS (2)..(964)
gaacaaatacaagtatgaagagtgtaaagacctcataaaatttatgctgaggaatgagcgacagttcaaggaggagaagcttgcagagcagctgaagcaagctgaggagctcaggcaatataaagtcctggttcacgctcaggaacgagagctgacccagttaagggagaagttgcgggaagggagagatgcctcccgctcattgaatgagcatctccaggccctcctcactccggatgagccggacaagtcccaggggcaggacctccaagaacagctggctgaggggtgtagactggcacagcaccttgtccaaaagctcagcccagaaaatgacaacgatgacgatgaagatgttcaaattgaggtggctgagaaagtgcagaaatcgtctgcccccagggagatgcagaaggctgaagaaaaggaagtccctgaggactcactggaggaatgtgccatcacttattcaaatagccatggcccttatgactccaaccagccacataggaaaaccaaaatcacatttgaggaagacaaagtcgactcaactctcattggctcatcctctcatgttgaacgggaagatgctgtacacattattccagaaaatgaaagtgatgatgaggaagaggaagaaaaagggccagtgtctcccaggaatctgcaggagtctgaagaggaggaagtcccccaggagtcctgggatgaaggttattcgactccctcaattcctcctgaaatgttggcctcgtacaagtcttacagcagcacatttcactcattagaggaacagcaagtctgcatggctgttgacataggcagacatcggtgggatcaagtgaaaaaggaggaccaagaggcaacaggtcccaggctcagcagggagctgctggatgagaaagggcctgaagtcttgcaggactcactggatagatgtttattcaactccttcaggttgtcttgaactgactgactcatgccagccctacagaagtgccttttacgtattggagcaacagcgtgttggcttggctgttgacatggatgaaattgaaaagtaccaagaagtggaagaagaccaagacccatcatgccccaggctcagcagggagctgctggatgagaaagagcctgaagtcttgcaggactcactgggtagatggtattcgactccttcaggttatcttgaactgcctgacttaggccagccctacagcagtgctgtttactcattggaggaacagtaccttggcttggctcttgacttggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaa

SEQ ID NO: 195 - protein, 321 aa, Homo sapiens
  image: 2782170 (5′ end); Genbank AF419618; cDNA clone AE01
NKYKYEECKDLIKFMLRNERQFKEEKLAEQLKQAEELRQYKVLVHAQERELTQLREKLREGRDASRSLNEHLQALLTPDEPDKSQGQDLQEQLAEGCRLAQHLVQKLSPENDNDDDEDVQIEVAEKVQKSSAPREMQKAEEKEVPEDSLEECAITYSNSHGPYDSNQPHRKTKITFEEDKVDSTLIGSSSHVEREDAVHIIPENESDDEEEEEKGPVSPRNLQESEEEEVPQESWDEGYSTPSIPPEMLASYKSYSSTFHSLEEQQVCMAVDIGRHRWDQVKKEDQEATGPRLSRELLDEKGPEVLQDSLDRCLFNSFRLS

SEQ ID NO: 196 - DNA, 2991 bp, Homo sapiens
  image: 2226413; Genbank AF419617; cDNA clone AG09
  CDS (542)..(715)
  CDS (718)..(2979)
attgataacagtaaagccacactcctcaagtgcctgaaatacccctcattgtcttcttcaggtggcaagggctctggaacagccacataaagcccttgaagataaggatggtcaaacaaaataatatcatacctggagaaactcagatcttgctaagatttactggttgggaatccaaagttaatgccaagaagcagctgccagttgggatcaaatgtgagcctatggatcaaggaatgtctggacacagtagacaaaggtttttcaactggatgccttaggatacatgcttccaaaaacaaagtagccaaaaagaaaccagagtcacagaatatcagagccagaggaacatttggagacggttacctggcacgctggccacaatctacctcactcttatcagagtctgagcagtgctttcagctctgagttgaggcacctcgaaccttgtttttgtggtgaaggatcctaaagtgctgtggggagtgatcacatttttcacaacatccctgactccacctcttctgccacaaacgtcagcatggtggtatcagccggcccttggtccagcgagaaggcagagatgaacattctagaaatcaacgagaaattgcgcccccagttggcagagaacaaacagcagttcagaaacctcaaagagagatgttttctaactcaactggccggcttcctggccaaccgacagaagaaatacagcaatataaagtcctggttcactctcaggaacgagagctgacccagttaagggagaagttacgggaagggagagatgcctcccgctcattgaatgagcatctccaggccctcctcactccggatgagccggacaagtcccaggggcaggacctccaagaacagctggctgaggggtgtagactggcacagcaccttgtccaaaagctcagcccagaaaatgacaaagatgaggatgaagatgttcaagttgaggaggatgagaaagtactggaatcatctgcccccagggaggtgcagaaggctgaagagagcaaagtccctgaggactcactggaggaatgtgccatcacttgttcaaatagccacggcccttgtgactccatccagcctcacaagaacatcaaaatcacatttgaggaagacaaagtcaactcatctctggttgtagacagagaatcctctcatgatggatgtcaggatgctctaaacattctcccagtccctggccccacctcttctgccacaaacgtcagcatggtggtatcagccggccctttgtccagcgagaaggcagagatgaacattctagaaatcaatgagaaattgtgcccccagctggcagagaagaaacagcagttcagaagcctcaaagagaaatgttttgtaactcaagtggcctgcttcctggccaagcagcagaacaaatacaaatatgaagagtgcaaagacctcataaaatctatgctgaggaatgagcgacagttcaaggaggagaagcttgcagagcagctgaagcaagctgaggagctcaggcaatataaagtcctggttcactctcaggaacgagagctgacccagttaagggagaagttacgggaagggagagatgcctcccgctcattgaatgagcatctccaggccctcctcactccggatgagccggacaagtcccaggggcaggacctccaagaacagctggctgaggggtgtagactggcacagcaccttgtccaaaagctcagcccagaaaatgataacgatgacgatgaagatgttcaagttgaggtggctgagaaagtgcagaaatcgtcttcccccagggagatgcagaaggctgaagaaaaggaagtccctgaggactcactggaggagtgtgccatcacttgttcaaatagccatggcccttatgactccaaccagccacatagaaaaaccaaaatcacatttgaggaagacaaagtcgactcaactctcattggctcatcctctcatgttgaatgggaggatgctgtacacattatcccagaaaatgaaagtgatgatgaggaagaggaagaaaaagggccagtgtctcccaggaatctgcaggagtctgaagaggaggaagtcccccaggagtcctgggatgaaggttattcgactctctcaattcctcctgaaaggttggcctcgtaccagtcttacagcagcacatttcactcattagaggaacagcaagtctgcatggctgttgacataggcagacatcggtgggatcaagtgaaaaaggaggaccaagaggcaacaggtcccaggctcagcagggagctgctggatgagaaagagcctgaagtcttgcaggactcactggatagatgttattcaactccttcagtttatcttggactgactgactcatgccagccctacagaagtgccttttacgtattggagcaacagcgtattggcttggctgttgacatggatgaaattgaaaagtgccaagaagtggaagaagaccaagacccatcatgccccaggctcagcagggagctgctggctgagaaagagcctgaagtcttgcaggactcactggatagatgttattcgactccttcaggttatcttgaactgcctgacttaggccagccctacagcagtgctgtttactcattggaggaacagtaccttggcttggctcttgacgtggacagaattaaaaaggaccaagaagaggaagaagaccaaggcccaccgtgccccaggctcagcagggagctgctggaggtagtagagcctgaagtcttgcaggactcactggatagatgttattcaactccttccagttgtcttgaacagcctgactcctgccagccctacggaagttccttttatgcattggaggaaaaacatgttggcttttctcttgacgtgggagaaattgaaaagaaggggaaggggaagaaaagaaggggaagaagatcaaagaagaaaagaagaaaaaaaaaaaaaaaa

SEQ ID NO: 197 - protein, 58 aa, Homo sapiens
  image: 2226413; Genbank AF419617; cDNA clone AG09
MVVSAGPWSSEKAEMNILEINEKLRPQLAENKQQFRNLKERCFLTQLAGFLANRQKKY

SEQ ID NO: 198 - protein, 754 aa, Homo sapiens
  image: 2226413; Genbank AF419617; cDNA clone AG09
QYKVLVHSQERELTQLREKLREGRDASRSLNEHLQALLTPDEPDKSQGQDLQEQLAEGCRLAQHLVQKLSPENDKDEDEDVQVEEDEKVLESSAPREVQKAEESKVPEDSLEECAITCSNSHGPCDSIQPHKNIKITFEEDKVNSSLVVDRESSHDGCQDALNILPVPGPTSSATNVSMVVSAGPLSSEKAEMNILEINEKLCPQLAEKKQQFRSLKEKCFVTQVACFLAKQQNKYKYEECKDLIKSMLRNERQFKEEKLAEQLKQAEELRQYKVLVHSQERELTQLREKLREGRDASRSLNEHLQALLTPDEPDKSQGQDLQEQLAEGCRLAQHLVQKLSPENDNDDDEDVQVEVAEKVQKSSSPREMQKAEEKEVPEDSLEECAITCSNSHGPYDSNQPHRKTKITFEEDKVDSTLIGSSSHVEWEDAVHIIPENESDDEEEEEKGPVSPRNLQESEEEEVPQESWDEGYSTLSIPPERLASYQSYSSTFHSLEEQQVCMAVDIGRHRWDQVKKEDQEATGPRLSRELLDEKEPEVLQDSLDRCYSTPSVYLGLTDSCQPYRSAFYVLEQQRIGLAVDMDEIEKCQEVEEDQDPSCPRLSRELLAEKEPEVLQDSLDRCYSTPSGYLELPDLGQPYSSAVYSLEEQYLGLALDVDRIKKDQEEEEDQGPPCPRLSRELLEVVEPEVLQDSLDRCYSTPSSCLEQPDSCQPYGSSFYALEEKHVGFSLDVGEIEKKGKGKKRRGRRSKKKRRK

SEQ ID NO: 199 - DNA, 4444 bp, Homo sapiens
  DKFZp564A057; Genbank AF419616; cDNA clone AG02
  CDS (1)..(2796)
cttggcttggctcctgacgtggacagaactaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctctcgacgtgggagaaattgaaaagaaggggaaggggaagaaaagaaggggaagaagatcaaagaaggaaagaagaaggggaagaaaagaaggggaagaagatcaaaacccaccatgccccaggctcagcagggagctgctggatgaggaagggcctgaagtcttgcaggactcactggatagatgttattcaactccttcaggttgtcttgaactgactgactcatgccagccctacagaagtgccttttacgtattggagcaacagcgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttgtcttgaactgcctgacttaggccagccctacagcagtgctgtttactcattggaggaacagtaccttggcttggctcttgacgtggacagaactaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaagaaggaaagaagagggggaagaaaagaaggggaagaagatcaaaacccaccatgccccaggctcagcagggagctgctggatgagaaagggcctgaagtcttgcaggactcactggatagatgttattcaactccttcaggttgtcttgaactgactgactcatgccagccctacagaagtgccttttacgtattggagcaacagcgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagcagtgctgagtactcattggaggaacagtaccttggcttggctcttgacgtggacagaactaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctctcgacgtgggagaaattgaaaagaaggggaaggggaagaaaagagggggaagaagatcagagaaggaaagaagaaggggaagaaaagaaggggaagaagatcaaaacccaccatgccccaggctcagcagggagctgctggatgagaaagggcctgaagtcttgcaggactcactggatagatgttattcaactccttcaggttgtcttgaactgactgactcatgccagccctacagaagtgccttttacgtattggagcaacagcgtgttggcttggctgttgacatggatgaaattgaaaagtaccaagaagtggaagaagaccaagacccatcatgccccaggctcagcagggagctgctggatgagaaagagcctgaagtcttgcaggactcactggatagatgttattcgactccttcaggttatcttgaactgcctgacttaggccagccctacagcagtgctgtttactcattggaggaacagtaccttggcttggctcttgacgtggacagaactaaaaaggaccaagaagaggaagaagaccaaggcccaccatgccccaggctcagcagggagctgctggaggtagtagagcctgaagtcttgcaggactcactgaatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagatcaaagaagaaaagaagaaggggaagaaaagaaggggaagaagatcaaaacccaccatgccccaggcccaacggcgtgctgatggaagtggaagggcctgaagtcttacaggactcactggatggatgttattctactccgtcaatgtactttgaactacctgactcattccagcactacagaagtgtgttttactcatttgaggaacagcacatcagcttcgccctttacgtggacaataggttttttactttgacggtgacaagtctccacctggtgttccagatgggagtcatattcccacaataaacagcccttactaagccgagagatgtcattcctgcaggcaggacctataggcacgtgaagatttgaatgaaagtacagttccatttggaagcccagacataggatgggtcagtgggcatggctgtattcctattctcaaaccatgccagtggcaacctgtgctcagtctgaagacaatggacccacgttaggtgtgacacgttcacataactgtgcagcacatgccgggagtgatcagtcagacattttaatttgaaccacgtatctctgggtagctacaaaattcctcagggatttcattttgcaggcatgtctctgagcttctatacctgctcaaggtcattgtcatctttgtgtttagctcatccaaaggtgttaccctggtttcaatgaacctaacctcattctttgtgtcttcagtgttggcttgttttagctgatccatctgtaacacaggagggatccctggttgaggattgtatttcagaaccaccaactgctcttgacaattgttaacccgctaggctcctttggttagagaagccacagtccttcagcctccaattggtgtcagtacttaggaagaccacagctagatggacaaacagcattgggaggccttagccctgctcctctcaattccatcctgtagagaacaggagtcaggagccgctggcaggagacagcatgtcacccaggactctgccggtgcagaatatgaacagtgccatgttcttgcagaaaacgcttagcctgagtttcataggaggtaatcaccagacaactgcagaatgtggaacactgagcgggacagctgacctgtctccttcacatagtccatgtcaccacaaatcacacaacaaaaaggagaagagatattttgggttcaaaaaacgtaaaaagataatgtagctgcatttctttagttattttgggccccaaatatttcctcatctttttgttgttgtcatggatggtggtgacatggacttgtttatagaggacaggtcagctgtctggctcagtgatctacattctgaagttgtctgaaaatgtcttcatgattaaattcagcctaaacgttttgccgggaacactgcagagacaatgctgtgagtttccaacctcagcccatctgcgggcagagaaggtctagtttgtccatcaccattatgatatcaggactggttacttggttaaggaggggtctaggagatctgtcccttttagagacaccttacttataatgaagaagtacttgggaaagcggttttcaagagtataaatatcctgtattctaatgatcatcctctaaacattttatcatttattaatcctccctgcctgtgtctattattatattcatatctctacactgcaaattttgggtctcaatttttactgtgcctttgtttttactagtgtctgctgttgcaaaaagaaggagacattctctgcctgagttttaatttttgtccaaagttaattttaatctatacaattaaaaccttttgcctatcactctggacttctggattgttttttacattcagtgttataatatttgattatgctgattggttttggtgggtactgatgcgaattaataaaaacatttcatttccaaaaaaaaaaaaaaaaaaaa

SEQ ID NO: 200 - protein, 932 aa, Homo sapiens
  DKFZp564A057; Genbank AF419616; cDNA clone AG02
LGLAPDVDRTKKDQEEEEDQGPPCPRLSRELLEVVEPEVLQDSLDRCYSTPSSCLEQPDSCQPYGSSFYALEEKHVGFSLDVGEIEKKGKGKKRRGRRSKKERRRGRKEGEEDQNPPCPRLSRELLDEEGPEVLQDSLDRCYSTPSGCLELTDSCQPYRSAFYVLEQQRVGLAVDMDEIEKYQEVEEDQDPSCPRLSRELLDEKEPEVLQDSLDRCYSTPSGCLELPDLGQPYSSAVYSLEEQYLGLALDVDRTKKDQEEEEDQGPPCPRLSRELLEVVEPEVLQDSLDRCYSTPSSCLEQPDSCQPYGSSFYALEEKHVGFSLDVGEIEKKGKGKKRRGRRSKKERRGGRKEGEEDQNPPCPRLSRELLDEKGPEVLQDSLDRCYSTPSGCLELTDSCQPYRSAFYVLEQQRVGLAVDMDEIEKYQEVEEDQDPSCPRLSRELLDEKEPEVLQDSLDRCYSTPSGYLELPDLGQPYSSAEYSLEEQYLGLALDVDRTKKDQEEEEDQGPPCPRLSRELLEVVEPEVLQDSLDRCYSTPSSCLEQPDSCQPYGSSFYALEEKHVGFSLDVGEIEKKGKGKKRGGRRSEKERRRGRKEGEEDQNPPCPRLSRELLDEKGPEVLQDSLDRCYSTPSGCLELTDSCQPYRSAFYVLEQQRVGLAVDMDEIEKYQEVEEDQDPSCPRLSRELLDEKEPEVLQDSLDRCYSTPSGYLELPDLGQPYSSAVYSLEEQYLGLALDVDRTKKDQEEEEDQGPPCPRLSRELLEVVEPEVLQDSLNRCYSTPSSCLEQPDSCQPYGSSFYALEEKHVGFSLDVGEIEKKGKGKKRRGRRSKKKRRRGRKEGEEDQNPPCPRPNGVLMEVEGPEVLQDSLDGCYSTPSMYFELPDSFQHYRSVFYSFEEQHISFALYVDNRFFTLTVTSLHLVFQMGVIFPQ

SEQ ID NO: 201 - DNA, 5166 bp, Homo sapiens
  Full-size DNA sequence of representative NBG gene
  CDS (55)..(5166)
gatcacatttttcacaacatccctgactccacctcttctgccacaaacgtcagcatggtggtatcagccggcccttggtccagcgagaaggcagagatgaacattctagaaatcaacgagaaattgcgcccccagttggcagagaacaaacagcagttcagaaacctcaaagagagatgttttctaactcaactggccggcttcctggccaaccgacagaagaaatacaagtatgaagagtgtaaagacctcataaaatttatgctgaggaatgagcgacagttcaaggaggagaagcttgcagagcagctgaaacaagctgaggagctcaggcaatataaagtcctggttcactctcaggaacgagagctgacccagttaagggagaagttacgggaagggagagatgcctcccgctcattgaatgagcatctccaggccctcctcactccggatgagccggacaagtcccaggggcaggacctccaagaacagctggctgaggggtgtagactggcacagcaacttgtccaaaagctcagcccagaaaatgatgaagatgaggatgaagatgttcaagttgaggaggatgagaaagtactggaatcatctgcccccagggaggtgcagaaggctgaagagagcaaagtccctgaggactcactggaggaatgtgccatcacttgttcaaatagccacggcccttgtgactccaaccagcctcacaagaacatcaaaatcacatttgaggaagacaaagtcaactcaactgtggttgtagacagaaaatcctctcatgatgaatgtcaggatgctctaaacattctcccagtccctggccccacctcttctgccacaaacgtcagcatggtggtatcagccggccctttgtccagcgagaaggcagagatgaacattctagaaatcaatgagaagttgcgcccccagctggcagagaagaaacagcagttcagaagcctcaaagagaaatgttttgtaactcaactggccggcttcctggccaagcagcagaacaaatacaaatatgaagagtgcaaagacctcataaaatctatgctgaggaatgagctacagttcaaggaggagaagcttgcagagcagctgaagcaagctgaggagctcaggcaatataaagtcctggttcactctcaggaacgagagctgacccagttaagggagaagttacgggaagggagagatgcctcccgctcattgaatgagcatctccaggccctcctcactccggatgagccggacaagtcccaggggcaggacctccaagaacagctggctgaggggtgtagactggcacagcaccttgtccaaaagctcagcccagaaaatgatgaagatgaggatgaagatgttcaagttgaggaggatgagaaagtgctggaatcatcttcccccagggagatgcagaaggctgaagaaagcaaagtccctgaggactcactggaggaatgtgccatcacttgttcaaatagccacggcccttgtgactccaaccagcctcacaagaacatcaaaatcacatttgaggaagacaaagtcaactcatctctggttgtagacagagaatcctctcatgatgaatgtcaggatgctctaaacattctcccagtccctggccccacctcttctgccacaaacgtcagcatggtggtatcagccggccctttgtccagcgagaaggcagagatgaacattctagaaatcaatgagaagttgcgcccccagctggcagagaagaaacagcagttcagaagcctcaaagagaaatgttttgtaactcaagtggcctgcttcctggccaagcagcagaacaaatacaaatatgaagagtgcaaagacctcataaaatctatgctgaggaatgagctacagttcaaggaggagaagcttgcagagcagctgaagcaagctgaggagctcaggcaatataaagtcctggttcactctcaggaacgagagctgacccagttaagggagaagttacgggaagggagagatgcctcccgctcattgaatgagcatctccaggccctcctcactccggatgagccggacaagtcccaggggcaggacctccaagaacagctggctgaggggtgtagactggcacaacaccttgtccaaaagctcagcccagaaaatgataacgatgacgatgaagatgttcaagttgaggtggctgagaaagtgcagaaatcgtcttcccccagggagatgcagaaggctgaagaaaaggaagtccctgaggactcactggaggagtgtgccatcacttgttcaaatagccatggcccttattactccaaccagccacataggaaaaccaaaatcacatttgaggaagacaaagtcgactcaactctcattggctcatcctctcatgttgaatgggaggatgctgtacacattatcccagaaaatgaaagtgatgatgaggaagaggaagaaaaagggccagtgtctcccaggaatctgcaggagtctgaagaggaggaagtcccccaggagtcctgggatgaaggttattcgactctctcaattcctcctgaaatgttggcctcgtaccagtcttacagcggcacatttcactcattagaggaacagcaagtctgcatggctgttgacataggcggacatcggtgggatcaagtgaaaaaggaggaccaagaggcaacaggtcccaggctcagcagggagctgctggatgagaaagggcctgaagtcttgcaggactcactggatagatgttattcaactccttcaggttatcttgaactgactgactcatgccagccctacagaagtgccttttacatattggagcaacagcgtgttggctgggctcttgacatggatgaaattgaaaagtaccaagaagtggaagaagaccaagacccatcatgccccaggctcagcagggagctgctggatgagaaagagcctgaagtcttgcaggactccctggatagatgttattcgactccttcaggttatcttgaactgcctgacttaggccagccctacagaagtgctgtttactcattggaggaacagtaccttggcttggctcttgacgtggacagaattaaaaaggaccaagaagaggaagaagaccaaggcccaccatgccccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaacgaagaaaagaaggagaaggggaagaaaagaaggggaagaagatcaaaacccaccatgccccaggctcagcagggagctgctggatgagaaagggcctgaagtcttgcaggactcactggatagatgttattcaactccttcaggttatcttgaactgactgactcatgccagccctacagaagtgccttttacatattggagcaacagcgtgttggctgggctcttgacatggatgaaattgaaaagtaccaagaagtggaagaagaccaagacccatcatgccccaggctcagcagggagctgctggatgagaaagagcctgaagtcttgcaggactccctggatagatgttattcgactccttcaggttatcttgaactgcctgacttaggccagccctacagaagtgctgtttactcattggaggaacagtaccttggcttggctcttgacgtggacagaattaaaaaggaccaggaagaggaagaagaccaaggcccaccatgccccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaacgaagaaaagaaggagaaggggaagaaaagaaggggaagaagatcaaaacccaccatgccccaggctcagcagggagctgctggatgagaaagggcctgaagtcttgcaggactcactggatagatgttattcaactccttcaggttatcttgaactgactgactcatgccagccctacagaagtgccttttacatattggagcaacagcgtgttggctgggctcttgacatggatgaaattgaaaagtaccaagaagtggaagaagaccaagacccatcatgccccaggctcagcagggagctgctggatgagaaagagcctgaagtcttgcaggactccctggatagatgttattcgactccttcaggttatcttgaactgcctgacttaggccagccctacagaagtgctgtttactcattggaggaacagtaccttggcttggctcttgacgtggacagaattaaaaaggaccaggaagaggaagaagaccaaggcccaccatgccccaggctcagcagggagctgctggaggcagtagagcctgaagtcttgcaggactcactggatagatgttattcaactccttccagttgtcttgaacagcctgactcctgcctgccctatggaagttccttttatgcattggaggaaaaacatgttggcttttctcttgacgtgggagaaattgaaaagaaggggaaggggaagaaaagaaggggaagaagatcaacgaagaaaagaaggagaaggggaagaaaagaaggggaagaagatcaaaacccaccatgccccaggctcagcggtgtgctgatggaagtggaagagcctgaaatcttgcaggactcactggatagatgttattcgactccgtcaatgttctttgaactacctgactcattccagcactacagaagtgtgttttactcatttgaggaacagcacatcagcttcgcccttgacgtggacaataggtttcttactttgatgggaacaagtctccacctggtcttccagatgggagtcatattcccacag

SEQ ID NO: 202 - protein, 1704 aa, Homo sapiens [sequence omitted]

SEQ ID NO: 203 - DNA, 263 bp, Artificial Sequence
  Description of Artificial Sequence: cDNA of exon A and B
tcagggacatgcagaaggctgaagaaaaggaagtccctgaggactcactggaggaatgtgccatcacttgttcaaatagccatggcccttatgactccaaccagccacataggaaaaccaaaatcacatttgaggaagacaaagtcgactcaactctcattggctcatcctctcatgttgaatgggaggatgctgtacacattatcccagaaaatgaaagtgatgatgaggaagaggaagaaaaagggccagtgtctcccagg

(19) United States
(12) Patent Application Publication
(10) Pub. No.: US 2004/0132030 A1
(43) Pub. Date: Jul. 8, 2004

(54) Identification of neuroblastoma tumor suppressor genes

(76) Inventors: Frans Van Roy, Destelbergen (BE); Frank Speleman, Antwerpen (BE); Nadine Van Roy, Oostakker (BE); Katrien Staes, Moerzeke (BE); Karl Vaderpoele, Gent (BE)

Correspondence Address: Laurence B Bond, Traskbritt, PO Box 2550, Salt Lake City, UT, 84110, US

(21) Appl. No.: 10/381,906

(22) PCT Filed: Sep. 27, 2001
(86) PCT No.: PCT/EP01/11199

(30) Foreign Application Priority Data
Sep. 27, 2000 (EP) 00870219.3

(51) Int. Cl. C12Q 1/68; C07H 21/04; C07K 14/52

(52) U.S. Cl. 435/6; 435/69.1; 435/320.1; 435/325; 530/351; 536/23.5

(57) ABSTRACT

The present invention relates to a new tumor suppressor gene family. More specifically the present invention relates to a tumor suppressor involved in suppression of neuroblastoma. Said tumor suppressor is involved in the generation of micronuclei and in the removal of acentric chromosomal fragments that might contain amplified DNA.

The present invention relates to a new tumor suppressor gene family. More specifically the present invention relates to a tumor suppressor involved in suppression of neuroblastoma. Said tumor suppressor is involved in the generation of micronuclei and in the removal of acentric chromosomal fragments that might contain amplified DNA.

Non-random structural rearrangements of the short arm of chromosome 1 (1p) are among the most common abnormalities in solid tumors as well as in hematological malignancies (for review, see Schwab et al. 1996). The most common structural rearrangements affecting 1p are deletions, thus suggesting the implication of tumor suppressor gene(s) located in this region. For several tumor types including neuroblastoma (Caron, 1995), meningioma (Cai et al., 2001), colorectal cancer (De Angelis et al., 2001), gastric carcinoma (Igarashi et al., 2000), and breast cancer (Ragnarsson et al., 1999), it has been shown that loss of 1p is correlated with poor prognosis or with tumor progression. The presence of a tumor suppressor gene on chromosome 1p is further supported by somatic cell fusion experiments. The delivery of chromosome 1p into the neuroblastoma cell line NGP by microcell-mediated chromosome transfer resulted in complete differentiation of the cells (Bader et al., 1991) and the introduction of the 1p36 chromosomal region in a colon carcinoma cell line suppressed tumorigenicity in nude mice (Tanaka et al., 1993). Deletion mapping in breast tumor cell lines has shown that one of the two SROs (shortest regions of overlaps) involving chromosome I overlaps with the consensus region of loss of heterozygosity (LOH) in neuroblastoma (Bieche et al., 1998). For malignant melanoma, frequent alterations of three evolutionary conserved regions on chromosome 1 (1p11-12, 1q21 and 1p36) were described (Zhang et al., 1999). Also metastatic retinoblastoma, which occasionally shows MYCN amplification, has been shown to have a recurrent LOH of chromosome 1p (Doz et al., 1996). Other LOH studies have identified several critical regions on 1p in various cancers including colon cancer, breast cancer, lung cancer, hepatocellular carcinoma, parathyroid adenoma, melanoma, and others. For different tumor types including neuroblastoma some SRO's (shortest regions of overlaps) for 1p deletions are overlapping indicating that the same tumor suppressor genes may be implicated in the different tumor types.

Tumor cells arise from normal tissues through the progressive accumulation of several mutations. These genetic changes consist of point mutations, deletions, inversions, translocations and DNA amplifications and result in the activation of proto-oncogenes or the inactivation of tumor suppressor genes. Mutations in DNA repair genes can significantly speed up this process. DNA amplification is often detected in high-grade tumors where the amplified DNA is either incorporated in a chromosome and is apparent as a homogenously staining region (HSR) or where it is present on double minute chromosomes (DMs). These DMs are small, acentric, paired structures that replicate autonomously. In many cases, it has been shown that these structures contain cellular proto-oncogenes.

The amplified genomic region is often tumor-specific and is correlated with a poor prognosis. For example, the MYC gene is amplified in several tumor types such as breast carcinoma, lung carcinoma and colon carcinoma. The related MYCN gene shows amplification in neuroblastoma and less frequently in retinoblastoma and small cell lung cancer (reviewed in Knuutila et al., 1998).

Double minutes are acentric and therefore they are not equally distributed between both daughter cells at, mitosis. It has been shown that DMs are lost from cells if their presence does not result in a selective advantage (Shimizu et al., 1994). Several drugs such as hydroxyurea and etoposide lead to a significant drop in the number of DMs in several human cell lines (Von Hoff et al., 1991; Von Hoff et al., 1992, Canute et al., 1996) and elimination of DMs can lead to a reversion of a malignant phenotype or to cellular differentiation of Colo320DM cells (Von Hoff et al., 1992), HL-60 cells (Shimizu et al., 1994; Eckhardt et al., 1994) and a number of neuroblastoma cell lines (Ambros et al., 1997).

Micronuclei are small, sphere-like structures, which are detected in the cytoplasm and are surrounded by a nuclear membrane. They are generated around acentric chromosomal fragments, such as DMs, when the nuclear membrane is reformed after mitosis. DMs can avoid this encapsulation by what is known as the ‘hitchhike’ mechanism (Levan and Levan, 1978; Kanda et al., 2001). Recently, a new mechanism of micronucleation has been described, involving the generation of micronuclei through budding from the main nucleus during S phase (Shimizu et al., 1998). It was also shown that the DMs are preferentially located at the periphery of the nucleus. Since the contents of the formed micronuclei consist mostly of amplified DNA, located on DMs, this is a mechanism through which the cell can remove amplified DNA fram the nucleus (Shimizu et al., 1998). The micronuclei are then removed via extrusion into the extracellular environment (Shimizu et al., 2000).

These results show that the elimination of DMs can be a new target in the development of tumor-selective chemotherapeutics. The treatment with hydroxyurea of patients with high-grade ovarian carcinomas led to a decrease in the number of DMs and to a better prognosis in several patients (Raymond et al., 2001).

Neuroblastoma is the most common extracranial solid tumor of childhood, originating from neuroectodermal cells. One of the hallmarks of neuroblastoma is the clinical and genetic heterogeneity. Brodeur et al. (1977) found that deletions of the short arm of chromosome 1 are a typical karyotypic finding in neuroblastoma cell lines and primary tumors. This observation has been extended by molecular genetic studies, which demonstrated LOH in 27% of primary tumors (Fong et al., 1989; Fong et al., 1992). The majority of 1p deletions are large, and virtually all 1p deletions encompass a common region within chromosome band 1p36 (Brodeur, 1998). The finding of 1p deletions together with the fact that introduction of chromosome 1p sequences in a neuroblastoma cell line induces differentiation and/or cell death (Bader et al., 1991), suggests that one or more tumor suppressor genes are located on the short arm of chromosome 1. Evidence for involvement of multiple tumor suppressor genes were provided by several studies. Schleiermacher et al. (1994) and Takeda et al. (1994) reported a case with interstitial deletions of the proximal part of 1p, suggesting the existence of a putative tumor suppressor gene in this proximal region. Furthermore, Caron et al. (1993) found that tumors with amplification of the MYCN gene generally had large deletions, whereas in single-copy MYCN cases small terminal deletions were also observed. The latter tumors showed preferential deletion of the maternal allele, which is suggestive for the presence of an imprinted gene. Several genes have been analyzed as candidate tumor suppressor genes, but until now no mutations have been found in the non-deleted allele of any of these candidate genes. The occurrence of tumor predisposing constitutional chromosome rearrangements may be helpful in positional cloning of tumor suppressor genes as was exemplified for retinoblastoma. In neuroblastoma, however, germline chromosomal abnormalities have been rarely described. Two neuroblastoma patients with constitutional interstitial deletions within chromosome band 1p36 have been identified (Biegel et al., 1993; White et al, 2001). In addition, our research group (Laureys et al., 1990) described a neuroblastoma patient with a constitutional t(1;17)(p36.2;q11.2) chromosomal translocation. In addition to the well-established recurrent occurrence of 1p-deletions, more recently the possible role of 17q in neuroblastoma came into focus.

Recurrent abnormalities of the long arm of chromosome 17 were already reported by Gilbert et al. (1984), but were only confirmed with the advent of molecular cytogenetic techniques which showed that 17q gain is the most frequently occurring structural rearrangement in high stage neuroblastomas (Van Roy et al., 1994; Van Roy et al., 1995; Brinkschmidt et al., 1997; Lastowska et al., 1997; Plantaz et al., 1997; Van Roy et al., 1997a; Vandesompele et al., 1998). Gain of 17q was also shown to be the most important independent prognostic marker in neuroblastoma, indicating that 17q gain itself or tightly associated accompanying genetic changes have an important impact on the biological characteristics of neuroblastoma (Bown et al., 1999). These accompanying genetic changes may well be the partial losses in the partner chromosomes, which participate in the formation of the unbalanced 17q translocations. This lead us to suggest that both copy number gain of genes on 17q and loss of putative tumor suppressor genes on the partner chromosome could be of functional significance. In this respect, the frequent involvement of 1p in unbalanced 17q translocations (Savelyeva et al., 1994; Van Roy et al., 1994) is of relevance as 1p36 is the presumed location for one or more neuroblastoma tumor suppressor genes (Versteeg et al., 1995; Schwab et al., 1996). Molecular analyses in neuroblastoma cell lines and primary tumors revealed that 1p and 17q breakpoints were scattered over a region of several megabases on both chromosomes, thus excluding the possibility of the classical mechanism of recurrent activation of the same proto-oncogene or recurrent formation of a specific oncogenic hybrid gene (Van Roy et al., 1995; Van Roy et al., 1997b; Lastowska et al., 1998). In view of these observations, the finding of a constitutional t(1;17) translocation in a neuroblastoma patient is intriguing. In order to obtain insight into the role of this translocation in the development of neuroblastoma in this unique patient, positional cloning and sequence analysis of the regions covering both breakpoints was performed. The 1p breakpoint was previously mapped to 1p36.2 within a large cluster containing multiple copies of genes including snRNA and tRNAs (van der Drift et al., 1994; Laureys et al., 1995; van der Drift et al., 1995). Notwithstanding this substantial amount of research by several research groups, and although initial efforts allowed to locate the constitutional 17q breakpoint between the NF1 gene and SCYA7 gene (Van Roy et al., 1997b), up to now, no neuroblastoma tumor suppressor gene could be isolated.

Surprisingly, we were able to demonstrate that the breakpoint of the constitutional t(1;17) translocation is situated within the transcribed sequence of a novel gene on chromosome 1. Analysis shows that this gene belongs to a highly conserved gene family, of which several members function as tumor suppressors. Even more surprisingly, these tumor suppressor genes were involved in the generation of micronuclei and the removal of amplified DNA. Gene disruption, caused by an event such as chromosomal translocation, may lead to a gene product that is less active, or not able to induce micronuclei, and therefore losing its tumor suppressor activity. It is a first aspect of the invention to provide an isolated tumor suppressor gene product, comprising SEQ ID N° 202 or a functional fragment, variant or fusion protein thereof. Preferentially, said tumor suppressor gene product is essentially consisting of SEQ ID N° 202, more preferentially said tumor suppressor gene product is consisting of SEQ ID 202. One preferred embodiment is a tumor suppressor gene product fragment comprising SEQ ID N° 2, preferably essentially consisting of SEQ ID N° 2, more preferentially preferably consisting of SEQ ID N° 2. Another preferred embodiment is tumor suppressor gene product fragment comprising SEQ ID N° 161, preferentially essentially consisting of SEQ ID N° 161, more preferentially consisting of SEQ ID N° 161. Still another preferred embodiment is a variant selected from the group of consisting of SEQ ID N° 175, 177, 181, 187, 189, 191 and 195, preferably essentially consisting of SEQ ID N° 175, 177, 181, 187, 189, 191 and 195, more preferably consisting of SEQ ID N° 175, 177, 181, 187, 189, 191 and 195. An even more preferred embodiment is a variant selected from the group of consisting of SEQ ID N° 167, 169, 171, 179, 183 and 193, preferably essentially consisting of SEQ ID N° 167, 169, 171, 179, 183 and 193, more preferably consisting of SEQ ID N° 167,169, 171, 179, 183 and 193. A most preferred embodiment is a variant selected from the group of consisting of SEQ ID N° 173, 185, 197, 198 and 200, preferably essentially consisting of SEQ ID N° 173, 185, 197, 198 and 200, more preferably consisting of SEQ ID N° 173,185, 197, 198 and 200. Still another preferred embodiment is a fusion protein of a tumor suppressor gene, consisting of SEQ ID N° 163 or SEQ ID N° 165 Preferentially, said tumor suppressor gene product is a tumor suppressor of meningioma, colorectal cancer, gastric carcinoma and/or breast cancer. Even more preferentially, said tumor suppressor is a neuroblastoma tumor suppressor.

It is another aspect of the invention to provide a nucleic acid encoding a tumor suppressor gene product or a tumor suppressor gene product fragment, variant or fusion product according to the invention. Said nucleic acid can be, amongst others, mRNA, cDNA or genomic DNA. A preferred embodiment is a nucleic acid, comprising SEQ ID N° 1, preferably essentially consisting of SEQ ID N° 1, more preferably consisting of SEQ ID N° 1. Another preferred embodiment is a nucleic acid, comprising SEQ ID N° 3, preferably essentially consisting of SEQ ID N° 3, more preferably consisting of SEQ ID N° 3. Still another preferred embodiment is a nucleic acid, comprising SEQ ID N° 4, preferably essentially consisting of SEQ ID N° 4, more preferably consisting of SEQ ID N°4. Still another preferred embodiment is a nucleic acid, comprising SEQ ID N° 201, preferably essentially consisting of SEQ ID N° 201, more preferably consisting of SEQ ID N° 201. Still another preferred embodiment is a nucleic acid, comprising SEQ ID N° 203,preferably essentially consisting of SEQ ID N° 203, more preferably consisting of SEQ ID N° 203. Still another preferred embodiment is a nucleic acid, comprising the sequence selected from a group consisting of SEQ ID N° 174, 176, 180, 186, 188, 190 and 194, preferably essentially consisting of SEQ ID N° 174, 176, 180, 186, 188, 190 and 194, more preferably consisting of SEQ ID N° 174, 176, 180, 186, 188, 190 and 194. An even more preferred embodiment is a variant selected from the group of consisting of SEQ ID N° 166, 168, 178, 182 and 192, preferably essentially consisting of SEQ ID N° 166, 168, 178, 182 and 192, more preferably consisting of SEQ ID N° 166, 168, 178, 182 and 192. A most preferred embodiment is a variant selected from the group of consisting of SEQ ID N° 172, 184, 196, and 199, preferably essentially consisting of SEQ ID N° 172, 184, 196, and 199, more preferably consisting of SEQ ID N° 172, 184, 196, and 199. Still another preferred embodiment is a nucleic acid, comprising the SEQ ID N°160, preferably essentially consisting of SEQ ID N° 160, more preferably consisting of SEQ ID N° 160. Still another preferred embodiment is a nucleic acid, consisting of SEQ ID N° 162 or SEQ ID N° 164.

Still another aspect of the invention is the use of a nucleic acid encoding a tumor suppressor gene product, or a functional fragment, variant or fusion product thereof according to the invention, or a nucleic acid with at least 60%, preferably 70%, more preferably 80%, most preferably 90% identity to said nucleic acid as measured by a BLASTN search (Altschul et al., 1997), or a functional fragment thereof in diagnosis of cancer and/or prediction of the likelihood of developing cancer. A preferred embodiment is the use of said nucleic acid, whereby said cancer is meningioma, colorectal cancer, gastric carcinoma and/or breast cancer. An even more preferred embodiment is the use of said nucleic acid, whereby said cancer is neuroblastoma.

Said diagnosis and/or prediction can be based on the detection of mutations, comprising point mutations, deletions, insertions and rearrangements, in the tumor suppressor gene or in a translocation target sequence such as the translocation target sequence on chromosome 17, and/or by measuring the transcription level of the tumor suppressor gene. This analysis can be performed by techniques such as, as a non-limiting example, DNA/DNA hybridization, DNA/RNA hybridization, fluorescent in situ hybridization (FISH) or PCR reaction, all known to the person skilled in the art.

Another aspect of the invention is the use of a nucleic acid encoding a tumor suppressor gene product or a functional fragment or variant thereof, according to the invention, or a nucleic acid with at least 60%, preferably 70%, more preferably 80%, most preferably 90% identity to said nucleic acid as measured by a BLASTN search (Altschul et al., 1997), or a functional fragment thereof in the treatment of cancer. A preferred embodiment is the use of said nucleic acid in gene therapy, to restore the defective function of the tumor suppressor gene. Vectors for gene therapy are known to the person skilled in the art, and include, but are not limited, retroviral vectors, adenovirus-associated vectors and lentiviral vectors. Suitable vector systems have been described, amongst others, in WO9822143, WO9812338 and WO9817816. A preferred embodiment is said use, whereby said cancer is meningioma, colorectal cancer, gastric carcinoma and/or breast cancer. An even more preferred embodiment is said use, whereby said cancer is neuroblastoma.

Still another aspect of the invention is the use of a tumor suppressor gene product, or a functional fragment or variant thereof, according to the invention, or a protein with at least 60% identity, preferably 70% identity, more preferably 80% identity, most preferably 90% identity to said tumor suppressor, as measured by a BLASTP or TBLASTN search (Altschul et al., 1997) for the manufacture of a medicament to treat cancer. A preferred embodiment is said use, whereby said cancer is meningioma, colorectal cancer, gastric carcinoma and/or breast cancer. An even more preferred embodiment is said use, whereby said cancer is neuroblastoma.

Still another aspect of the invention is the use of a tumor suppressor gene product, or a functional fragment or variant thereof, according to the invention, or a protein with at least 60% identity, preferably 70% identity, more preferably 80% identity, most preferably 90% identity to said tumor suppressor, as measured by a BLASTP or TBLASTN search (Altschul et al., 1997), for the generation of micronuclei and/or the removal of amplified DNA. Said generation of micronuclei may be useful in the suppression of different forms of cancer.

Still another aspect of the invention is a method to produce antibodies, using a tumor suppressor gene product or a functional fragment or variant or fusion protein thereof, according to the invention, or a protein with at least 60% identity, preferably 70% identity, more preferably 80% identity, most preferably 90% identity to said tumor suppressor, as measured by a BLASTP or TBLASTN search (Altschul et al., 1997), or using nucleic acid encoding such tumor suppressor gene product or a functional fragment or variant or fusion protein thereof, according to the invention, or a protein with at least 60% identity, preferably 70% identity, more preferably 80% identity, most preferably 90% identity to said tumor suppressor, as measured by a BLASTP or TBLASTN search (Altschul et al., 1997),. Antibodies include polyclonal, monoclonal and synthetic antibodies. Methods to produce such antibodies are known to the person skilled in the art.

A further aspect of the invention is an antibody obtainable by said method.

Still a further aspect of the invention is the use of said antibody in diagnosis of cancer and/or prediction of likelihood of developing cancer. A preferred embodiment is said use whereby said cancer is meningioma, colorectal cancer, gastric carcinoma or breast cancer. An even more preferred embodiment is said use whereby said cancer is neuroblastoma. Said antibody may be used in assays such as, but not limited to, Western blot or ELISA, known to the person skilled in the art.

Another aspect of the invention is use of a tumor suppressor gene product, or a functional fragment, variant or fusion product thereof, according to the invention, or a protein with at least 60% identity, preferably 70% identity, more preferably 80% identity, most preferably 90% identity to said tumor suppressor, as measured by a BLASTP or TBLASTN search (Altschul et al., 1997), for the isolation of an interacting compound. Several methods have been described to detect protein—compound interactions and to select the interacting compound. These methods include, but are not limited to, phage display, yeast two-hybrid assay, coimmunoprecipitation, DNase protection assay, electrophoretic mobility shift assay, or mass spectrometric analyses, all known to the person skilled in the art, fluorescence resonance energy transfer (FRET, WO9918124) and bioluminescence resonance energy transfer (BRET, WO9966324).

### Definitions

A functional fragment of a tumor suppressor gene product, means any proteineous molecule that retains its tumor suppression activity, and preferably its micronuclei inducing activity and/or the activity to remove amplified DNA.

A variant of a tumor suppressor gene product according to the invention is a gene product with at least 60% identity, preferably 70% identity, more preferably 80% identity, most preferably 90% identity to said tumor suppressor gene product, as measured by a BLASTP or TBLASTN search (Altschul et al., 1997), and retaining its tumor suppression activity, and preferably its micronuclei inducing activity and/or the activity to remove amplified DNA.

An isolated nucleic acid encoding a tumor suppressor gene product means that said nucleic acid comprises partly or totally the coding sequence of said tumor suppressor gene. The definition covers, but is not limited to genomic DNA and messenger RNA. It does however not implicate that said genomic DNA is transcribed and translated into said tumor gene product.

A functional fragment of a nucleic acid for use in diagnosis of and/or prediction of the likelihood of means any fragment that can be used as specific probe in hybridization or as primer in PCR reactions.

A functional fragment of a nucleic acid for use in the treatment of cancer means any fragment that can be transcribed and/or translated into a functional tumor suppressor, which preferably retains its micronuclei inducing activity and or the activity to remove amplified DNA.

A functional fragment of a tumor suppressor gene product for the manufacture of a medicament to treat cancer is any fragment of said tumor suppressor gene product that retains its tumor suppression function and preferably its micronuclei inducing activity and or the activity to remove amplified DNA

A functional fragment of a tumor suppressor gene product in the production of antibodies is an immunogenic fragment that comprises at least one epitope and can be used for the production of antibodies against said tumor suppressor gene product.

A functional fragment of a tumor suppressor gene product in the isolation of an interacting compound is any fragment that can be used in an interaction screening assay, such as, but not limited to, a yeast two-hybrid assay, a phage display assay, coimmunoprecipitation, a DNase protection assay, an electrophoretic mobility shift assay, or mass spectrometric analyses.

The terms protein and polypeptide as used in this appliction are interchangeable. Polypeptide refers to a polymer of amino acids and does not refer to a specific length of the molecule. This term also includes post-translational modifications of the polypeptide, such as glycosylation, phosphorylation and acetylation

Compound as used here means any chemical or biological compound, including simple or complex organic or inorganic molecules, peptides, peptido-mimetics, proteins, antibodies, carbohydrates, nucleic acids or derivatives thereof.

Interacting compound with a protein means any compound that can bind, covalently or not, with said protein in a specific way.

### BRIEF DESCRIPTION OF THE FIGURES

FIG. 1

Physical map of PAC/BAC contig covering the constitutional 17q11.2 breakpoint based on fibre FISH, FIGE and content mapping of STS and EST clones. The orientation of the map is centromere to telomere, left to right. Markers on the top row are cDNA/EST clones, polymorphic markers and STSs. Known genes are indicated by arrowheads. Partially sequenced BACs (with GenBank accession numbers) are indicated by dashed lines. Three breakpoint overlapping cosmid clones are shown, as indicated by ICRF numbers. The shaded bars indicate the approximate location of the 17q breakpoint and the distal breakpoints of NF1 deletion patients UWA 155-1 and UWA 106-3 (for details on the latter two patients, see Dorschner et al., 2000).

FIG. 2

Southern blot analysis of the t(1;17) translocation breakpoint by use of probe #6 (Table 4). Genomic DNAs extracted from a normal human placenta (normal) and from the Chinese hamster ovary cell line a3 served as controls in comparison to genomic DNAs from somatic cell hybrids 32-7A and 32-2F53VIII. Following exhaustive digestion with the restriction enzymes indicated on top, DNA fragments were subjected to Southern blot analysis. Probe #6 was prepared by PCR on the basis of the breakpoint-overlapping cosmid contig sequence (as described in Material and methods to the examples). The bands corresponding to the normal, non-rearranged DNA are indicated by arrows, whereas the rearranged bands are indicated by arrowheads. Approximate sizes are indicated in bp.

FIG. 3

Schematic representation of the normal and derivative chromosomal breakpoint region in the 32-2F53VIII somatic cell line hybrid. The location of the t(1;17) translocation breakpoint is indicated in each panel by a vertical double-pointed arrow. Restriction enzyme sites indicated are BamHI (B), PvuII (P), DraI (D), StuI (S) and EcoRV (E).

A. Schematic overview of the fragment of normal human chromosome 17 encompassing the chromosomal t(1;17) breakpoint. The horizontal bar represents the sequence (51,050 bp; GenBank Acc. No. AF148647) of a cosmid contig spanning the breakpoint in cell line 32-2F53VIII. The physical ends of the insert of cosmid ICRFc105F1060D1 were determined as indicated. An upward arrow shows the location of the SP6 end sequence of clone 841C13. Nine repeat-free probes (black boxes) were selected for use in the identification of the breakpoint by Southern hybridizations.

B. Schematic overview of a fragment of normal human chromosome 17. The breakpoint (Brkpt) in the chromosome-17 derivative of 32-2F53VIII cells was mapped to a 1,539-bp PvuII fragment. The GenomeWalker PCR products extending from primer. GSP1 to either StuI, PvuII or DraI sites each comprise the location of the t(1;17) breakpoint as evidenced by a comparison with the scheme depicted in (C).

C. Schematic overview of a fragment of derivative chromosome 17, corresponding to the fragment of normal chromosome 17 depicted in (B). Cloned and sequenced GenomeWalker PCR products spanning the t(1;17) breakpoint are as indicated. The sequence of the (GSP-1—EcoRV) fragment is shown in FIG. 5.

FIG. 4

Cloning of the t(1;17) breakpoint of chromosome der17 by GenomeWalker PCR. Genomic DNA from the 32-2F53VIII cell line was digested as described in the text and summarized in FIG. 3. GenomeWalker PCR was then performed using the following primer pairs (PR): either GSP1+AP1 (four lanes at the left), or GSP1′ plus AP1 (four lanes at the right). Restriction enzymes (RE) used are indicated on top: DraI (D), EcoRV (E), PvuII (P), and StuI (S). M, Lambda BstEII molecular weight marker. Bands were stained by ethidium bromide. The bands corresponding to normal chromosome 17 are indicated by *, whereas the bands rearranged by the t(1;17) translocation are indicated by arrowheads. The latter were cloned and sequenced (FIG. 5). Fragment sizes (FS) are indicated at the bottom in bp.

FIG. 5

Genomic DNA sequence (3,743 bp; GenBank Acc. No. AF379607) of the der17 chromosome overlapping the t(1;17)(p36.2;q11.2-q12.1) translocation breakpoint. Chromosome-17 specific sequences (602 bp) flanking the breakpoint are underlined. A spacer region of 7 bp (bold and underlined capitals) is of unknown origin. The remaining sequence of 3,134 bp represents chromosome-1 specific sequences flanking the breakpoint. Three exons, two internal ones (designated x and y) and one 3′ terminal (designated z), were predicted in the latter sequence. They are indicated by capitals; the predicted ORF is given in bold single-letter codes under the DNA sequence; the flanking splice donor and acceptor sites are underlined; a stop codon (indicated by *) in the 3′ terminal exon z is in bold and underlined.

FIG. 6

Sequence of a cDNA fragment (528 nucleotides) with corresponding amino acid sequence (175 amino acid residues), predicted to be encoded by the chromosome-1 specific genomic DNA sequence, flanking the t(1;17)(p36.2;q11.2-q12.1) translocation breakpoint in the der17 chromosome (depicted in FIG. 5). A stop codon (indicated by *) is in bold and underlined.

FIG. 7

Schematic overview of selected human cDNA (EST) sequences highly homologous or identical to exons x to z (exons 11.1, 12.5 and 14.12), flanking the t(1;17)(p36.2;q11.2-q12.1) translocation breakpoint and depicted on top as Breakpoint der17. Sequences are identified by their respective cDNA code, as summarized in Tables 5 and 6. EST cDNA sequences as deposited in GenBank were confirmed, improved and often extended on the appropriate cDNA clones. The predicted protein fragments, encoded by separate exons, were aligned as shown in FIGS. 9 to 15. Asterisks refer to sequences corresponding to incomplete exons. Thin lines correspond either to sequences predicted to be intronic, or to sequences corresponding to the 3′UTR (size given in nucleotides). If the 3′UTR is thought to be complete, it is followed by AAAA. Dotted lines represent unfinished sequences. Sizes of separate exon-encoded domains are indicated in amino acid residues (aa). Some sequence abnormalities (frameshift, internal deletion) are as indicated.

FIG. 8

Schematic overview of selected human cDNA sequences highly homologous or identical to either exons A and B (exons 6.12 and 7.1) or exons x to z (exons 11.1, 12.5 and 14.12). These exons are flanking the t(1;17)(p36.2;q11.2-q12.1) translocation breakpoints of chromosomes der1 and der17 as depicted at the bottom. Sequences are identified by their respective GenBank Acc. No, as summarized in Table 5. The predicted protein fragments were aligned as shown in FIGS. 9 to 15. Sizes of separate exon-encoded domains are indicated in amino acid residues (aa). The double broken lines in the centre of the AB033071 molecule point at a single-nucleotide frameshift and an apparent deletion as compared to the other molecules depicted. Asterisks refer to sequences corresponding to incomplete exons. Thin lines in the scheme of cDNA clone AI050141 correspond to sequences predicted to be intronic. The unlabeled black box (60 aa) in the scheme of cDNA clone AL136890 is of unknown origin.

FIGS. 9 to 15

Alignments of protein fragments predicted to be encoded by exons identified in genomic and cDNA clones and belonging to a novel gene family, comprising exons exons A, B and x to z, flanking the t(1;17)(p36.2;q11.2-q12.1) translocation breakpoints in chromosomes der1 or der17. The source of these sequences is summarized in Table 5 and partly depicted in FIGS. 7, 8, 21, 22-25, 28 and 37. On top of each sequence alignment is a corresponding phylogenetic tree (scales indicate % of non-similarity).

FIG. 9 represents exons of type 0, probably comprising highly conserved 5′UTR sequences, and exons of type 1, encoding both putative amino-terminal and internal domains (NBG proteins are probably starting with the highly conserved sequence MWSAGP . . . ).

FIG. 10 represents exons of types 2 and 3.

FIG. 11 represents exons of types 4, 5 and 6. Exon 6.12 corresponds to exon A.

FIG. 12 represents exons of types 7, 8 and 9. Exon 7.1 corresponds to exon B.

FIG. 13 represents exons of type 10 and 11. Exon 11.1 corresponds to exon x.

FIG. 14 represents exons of type 12 and 13. Exon 12.5 corresponds to exon y.

FIG. 15 represents exons of type 14, encoding carboxy-terminal protein domains. Exon 14.12 corresponds to exon z.

FIG. 16

Southern blot analysis of the t(1;17) translocation breakpoint by use of probe #9 (Table 4). Genomic DNAs extracted from a normal human placenta (normal) and from the Chinese hamster ovary cell line a3 served as controls in comparison to genomic DNAs from somatic cell hybrids 32-2F53VIII and 32-7A. Following exhaustive digestion with the restriction enzymes indicated on top, DNA fragments were subjected to Southern blot analysis. Probe #9 was prepared by PCR on the basis of the breakpoint-overlapping cosmid contig sequence (as described in Material and methods to the examples). Rearranged bands in the 32-7A genomic DNA are indicated by arrows. Size markers (DNA molecular weight marker 11, Roche) are indicated in bp.

FIG. 17

Schematic representation of the normal and derivative chromosomal breakpoint region in the 32-7A somatic cell line hybrid. The location of the t(1;17) translocation breakpoint is indicated in each panel by a vertical double-pointed arrow. Restriction enzyme sites indicated are BamHI (B), PvuII (P), DraI (D), SspI (S) and EcoRV (E).

A. Schematic overview of the fragment of normal human chromosome 17 encompassing the chromosomal t(1;17) breakpoint. The horizontal bar represents the sequence (51,050 bp; GenBank Acc. No. AF148647) of a cosmid contig spanning the breakpoint in cell line 32-7A. The physical ends of the insert of cosmid ICRFc105F1060D1 were determined as indicated. An upward arrow shows the location of the SP6 end sequence of clone 841C13. Nine repeat-free probes (black boxes) were selected for use in the identification of the breakpoint by Southern hybridizations.

B. Schematic overview of a fragment of normal human chromosome 17. The GenomeWalker PCR products extending from primer GSP1″ to a DraI site, or extending from primer GSP2 to PvuII or SspI sites each comprise the location of the t(1;17) breakpoint as evidenced by a comparison with the scheme depicted in (C).

C. Schematic overview of a fragment of derivative chromosome 1, corresponding to the fragment of normal chromosome 17 depicted in (B). Cloned and sequenced GenomeWalker PCR products spanning the t(1;17) breakpoint are as indicated. The sequence of the (GSP1″—PvuII) fragment is shown in FIG. 19.

FIG. 18

Cloning of the t(1;17) breakpoint of chromosome der1 by GenomeWalker PCR. Genomic DNA from the 32-7A cell line was digested as described in the text and summarized in FIG. 17 and GenomeWalker PCR was performed using the following primer pairs (PR): either GSP1″+AP1 (five lanes at the left), or GSP2 plus AP2 (five lanes at the right). Restriction enzymes (RE) used are indicated on top: DraI (D), EcoRV (E), PvuII (P), ScaI (Sc) or SspI (S). M, Lambda BstEII molecular weight marker. Bands were stained by ethidium bromide. The bands corresponding to normal chromosome 17 are indicated by *, whereas the bands rearranged by the t(1;17) translocation are indicated by arrowheads. The latter were cloned and sequenced (FIG. 19). Fragment sizes (FS) are indicated at the bottom in bp. Aspec, aspecifically amplified bands.

FIG. 19

Genomic DNA sequence (4,512 bp; GenBank Acc. No. AF379606) of the der1 chromosome overlapping the t(1;17)(p36.2;q11.2-q12.1) translocation breakpoint. The sequence of the first 3,845 bp represents chromosome-1 specific sequences flanking the breakpoint. This is followed by a spacer region of 4 bp (bold and underlined capitals AAAG), which is of unknown origin. The remaining underlined sequence of 663 bp represents chromosome-17 specific sequences flanking the breakpoint. Two exons (designated A and B) were predicted in the chromosome-1 sequence. They are indicated by capitals; the predicted ORF is given in bold single-letter codes under the DNA sequence; the flanking splice donor and acceptor sites are underlined.

FIG. 20

Sequence of a cDNA fragment (258 nucleotides) with corresponding amino acid sequence (86 amino acid residues), predicted to be encoded by the chromosome-1 specific genomic DNA sequence, flanking the t(1;17)(p36.2;q1.2-q12.1) translocation breakpoint in the der1 chromosome (depicted in FIG. 19).

FIG. 21

Schematic overview of selected human cDNA (EST) sequences highly homologous or identical to exons A and B (exons 6.12 and 7.1), flanking the t(1;17)(p36.2;q11.2-q12.1) translocation breakpoint and depicted on top as Breakpoint der17. Sequences are identified by their respective cDNA code, as summarized in Tables 5 and 6. EST cDNA sequences as deposited in GenBank were confirmed, improved and often extended on the appropriate cDNA clones. The predicted protein fragments, encoded by separate exons, were aligned as shown in FIGS. 9 to 14. Asterisks refer to sequences corresponding to incomplete exons. Thin lines correspond to sequences predicted to be intronic. Sizes of separate exon-encoded domains are indicated in amino acid residues (aa). Some sequence abnormalities (frameshifts) are as indicated. The sequence of cDNA clone AE02 is homologous with the 5′-end of cDNA clone AG09. When domains of type 1 are preceded by one of type 0, they are predicted to contain the start codon.

FIG. 22

Schematic overview of selected human cDNA (EST) sequences highly homologous or identical to exons x and y (exons 11.1 and 12.5), flanking the t(1;17)(p36.2;q11.2-q12.1) translocation breakpoint and depicted on top as Breakpoint der17. Sequences are identified by their respective cDNA code, as summarized in Tables 5 and 6. EST cDNA sequences as deposited in GenBank were confirmed, improved and often extended on the appropriate cDNA clones. The predicted protein fragments, encoded by separate exons, were aligned as shown in FIGS. 13 and 14. Asterisks refer to sequences corresponding to incomplete exons. Thin lines correspond to sequences predicted to be intronic. Sizes of separate exon-encoded domains are indicated in amino acid residues (aa).

FIG. 23

Schematic overview of selected human genomic sequences highly homologous or identical to either exons A and B (exons 6.12 and 7.1) or exons x to z (exons 11.1, 12.5 and 14.12). These exons are flanking the t(1;17)(p36.2;q11.2-q12.1) translocation breakpoints of chromosomes der1 and der17 as depicted at the top. Sequences are identified by their respective GenBank Acc. No, as summarized in Table 5. The predicted protein fragments were aligned as shown in FIGS. 9 to 15. Sizes of separate exon-encoded domains are indicated in amino acid residues (aa). The double-pointed horizontal arrows below the AL354666 and AL356581 molecules point at separate contigs of these unfinished genomic sequences (contigs are identified by #number). The exon types 4.1 and 5.1 between brackets were only detected in previous releases of sequence AL354666, but removed in release 5.

FIG. 24

Schematic overview of the neuroblastoma breakpoint gene (NBG) gene structure at the t(1;17)(p36.2;q11.2-q12.1) translocation breakpoint. Idiograms of normal chromosome 1, normal chromosome 17 (shaded), and derivative chromosomes der1 and der17 are shown. The structure of the chromosome-1 NBG gene, interrupted by the translocation, is predicted on the basis of sequences of cDNA clone DKFZp434G2022 (clone AB25; FIG. 7) and sequences from unfinished genomic clones RP11-284017 (GenBank Acc. No. AL355800) and RP11-4513 (GenBank Acc. No. AC015618). Only exon sequences are represented (typing according to Table 5 and FIGS. 9-15; sizes in aa, amino acid residues). Thin lines correspond to the 3′UTR sequence. Arrows denote the direction of transcription. Upon chromosomal translocation, a chromosome-1 region of about 20 kbp appears to be deleted (indicated by Del). After translocation, the gene on the der1 chromosome is expressed as chimeric transcripts (FIGS. 25-27) as splicing occurs from exon 7.1 to chromosome-17 derived sequences X (as indicated in this figure) or Y (see also FIG. 25). Similar chimeric transcripts for the der17 chromosome are not yet identified.

FIG. 25

Chimeric NBG-related transcripts. The chimera #1 and #2 transcripts were isolated by 3′-RACE on RNA from somatic cell hybrid 32-7A, containing the der1 chromosome (see FIGS. 26 and 27 for the sequences of the 3′-RACE clones). Sequences X and Y are derived from chromosome 17. In addition, two tumor-derived cDNA clones contain NBG-related sequences fused to gene sequences from chromosome 1, as documented in the text.

FIG. 26

Sequence of a chimeric cDNA fragment with corresponding amino acid sequence (GenBank Acc. No. AF420438), cloned by 3′-RACE on RNA from somatic cell hybrid 32-7A (see chimera #1 in FIG. 25). The sequence starts with gene-specific primer GSP2 located in NBG exon 6. Nucleotides from chromosome 1 (part of exon 6 plus exon 7.1) are put in capitals, and the encoded amino acid residues in bold capitals; nucleotides from chromosome 17 are put in lowercase and the encoded amino acid residues (ORF X) in underlined bold italics capitals. Asteriks, stop codon followed by 3′UTR and poly-A stretch.

FIG. 27

Sequence of a chimeric cDNA fragment with corresponding amino acid sequence, (GenBank Acc. No. AF420439) cloned by 3′-RACE on RNA from somatic cell hybrid 32-7A (see chimera #2 in FIG. 25). The sequence starts with gene-specific primer GSP2 located in NBG exon 6. Nucleotides from chromosome 1 (part of exon 6 plus exon 7.1) are put in capitals, and the encoded amino acid residues in bold capitals; nucleotides from chromosome 17 are put in lowercase and the encoded amino acid residues (ORF Y) in underlined bold italics capitals. Asteriks, stop codon followed by 3′UTR and poly-A stretch.

FIG. 28

Overview of cloned NBG-related 5′-RACE products. RNAs from four different cell lines were used as template, as indicated. Nested primers of these reactions were specific for type-11 exons but may anneal also to part of the type-10 exons. The predicted protein fragments, encoded by separate exons, were aligned as shown in FIGS. 13 and 14. Asterisks refer to sequences corresponding to incomplete exons. Thin lines correspond to sequences predicted to be intronic. Sizes of separate exon-encoded domains are indicated in amino acid residues (aa). Double pointed arrows indicate the insert sizes of the fully overlapping shorter cDNA clones indicated.

FIG. 29

Northern blot analysis of NBG-related transcripts in human tumor-derived cell lines as indicated, in somatic cell hybrid 32-7A and in mouse cell lines NMe and Neuro2A. Sequential hybridization was done with a 5′ NBG probe followed by a 3′ NBG probe (see Materials and methods to the examples). Loading of the mRNAs was estimated by hybridization with a mouse GAPDH probe. The graph shows the normalized relative amounts of the different bands (7.2 kb+5.9 kb, 4.1 kb, 0.5 kb) as quantitated by phosphor imaging. The 5′ probe (5′) allowed more efficient detection as compared to the 3′ probe (3′). SH-SY5Y cells expressed the highest levels for all NBG transcripts, except for the 0.5 kb species that was expressed mainly by HCT8/R1 cells.

FIG. 30

Flowchart of the construction of plasmid pBlue-KIAA-AB25, comprising a full-length NBG cDNA. cDNA/protein fragments, corresponding to separate exons, are labeled according to Table 5 and FIGS. 9-15.

FIG. 31

MCF7/AZ cells were transfected with different NBG constructs as indicated. Left panels show either the Myc-tag revealed by anti-Myc antibody (panels A and K) or fluorescence of the GFP-tag (panels C, E, G and I ). Right panels show DNA staining by DAPI. Cells were transfected with a construct encoding a Myc-tagged aminoterminal fragment (A, B), a construct encoding a GFP-tagged aminoterminal fragment (C, D), a construct encoding a GFP-tagged carboxyterminal fragment (E-H), a GFP-tagged full-length construct (I, J), and a Myc-tagged full-length construct (K, L). Arrows point at micronuclei, which are particularly obvious in cells transfected with the full-length constructs (I and K).

FIG. 32

MCF7/AZ cells were transfected with different NBG constructs as indicated. pCS2+MT-KIAA-AB25, a construct encoding a Myc-tagged full-length NBG protein, was used for cells in panels A-F. Cells were simultaneously stained with an anti-Myc antibody (A), a concanavalin-A conjugate to stain the endoplasmic reticulum (B), and with the DNA stain DAPI (C). This shows that the overexpressed protein is not localized in the endoplasmic reticulum. The arrows points at a micronucleus. Transfected cells were also simultaneously stained with the polyclonal NBG-specific antibody #31226 (D), with a mouse anti-lamin B antibody (E) and with DAPI (F). The 2 micronuclei in this cell (arrows) are clearly stained by the anti-lamin antibody and by DAPI, demonstrating that these structures are indeed micronuclei. Cells were also transfected with either the empty vector pEGFP-N3 (G, H) or the empty vector pEF6/Myc-His-A. GFP fluorescence is shown (G), staining with anti-Myc antibody (I) or DAPI staining (H, J).

FIG. 33

HCT8/E8 cells were transfected with constructs encoding a Myc-tagged full-length NBG protein (A, B), a GFP-tagged aminoterminal fragment (C, D), a GFP-tagged carboxyterminal fragment (E, F), or with the empty vector pEGFP (G, H). Several micronuclei are visible in the cytoplasm as indicated by arrows. GFP fluorescence is shown (C, E, G), staining with anti-Myc antibody (A) or DAPI staining (B, D, F, H).

FIG. 34

HEK293T cells were transfected with constructs encoding a Myc-tagged full-length NBG protein (A, B) or a GFP-tagged aminoterminal fragment (C, D). Micronuclei in the cytoplasm are indicated by arrows.

Colo320DM cells were transfected with a construct encoding a Myc-tagged full-length NBG protein (E, F), with the empty control vector (G, H), with a construct encoding a GFP-tagged aminoterminal fragment (I, J), or with a construct encoding a GFP-tagged carboxyterminal fragment (K, L). The cell transfected with the full-length construct shows two micronuclei (arrows), the first one in the cytoplasm and the second still attached to the main nucleus.

GFP fluorescence is shown (C, I, K), staining with anti-Myc antibody (A, E, G) or DAPI staining (B, D, F, H, J, L).

FIG. 35

Mouse NMe cells were transfected with several constructs as indicated. The latter encode a GFP-tagged full-length NBG protein (A, B), a GFP-tagged aminoterminal fragment (C, D), a Myc-tagged full-length NBG protein (E, F). In addition, empty vector pEGFP (G, H) or empty vector pCS2+MT (I, J) were used. When the full-length protein was overexpressed, many micronucleus-like structures became visible (arrows in A and E), which stain negative for DAPI. Upon expression of the aminoterminal fragment, perinuclear fluorescence was apparent, but much less micronucleus-like cytoplasmic structures (C).

GFP fluorescence is shown (A, C, G), staining with anti-Myc antibody (E, I) or DAPI staining (B, D, F, H, J).

FIG. 36

MCF7/AZ cells were transfected with two constructs expressing NBG-related chimeric transcripts as indicated. Chimeric cDNA fragments were cloned by 3′-RACE on mRNA of the 32-7A somatic cell hybrid, followed by ligation to a suitable fragment of plasmid pCS2+MT-KIAA-AB25. The transcripts encode an aminoterminal NBG fragment elongated by chromosome-17 sequences (see FIG. 25). Use of these constructs was featured by the generation of multiple vesicle-like structures in the cytoplasm (A, C). In addition, these cells contain some micronuclei as demonstrated by an arrow in (A). Staining was with anti-Myc antibody (A, C) or with DAPI staining (B, D).

FIG. 37

RT-PCR was performed on RNA from either human fetal brain (HFB) or the somatic cell hybrid 32-2FVIII. Primers used in these experiments are denoted by FVR numbers under typical NBG exonic sequences. Sequences of cloned products are as depicted (see also Table 5 and FIGS. 11-15 for the protein fragments, predicted to be encoded by separate exons). Several new exon subtypes were identified, including novel splice variants each time involving exon 8. Clone names with an asterisk contain a frameshift immediately before exon 9, as they either lack the complete exon 8 or parts of it (deletions indicated by dashed lines). In clones 27 and 28, domain 8 consists of the first 5 amino acid residues and the last 28 amino acid residues of a full-length exon-8 sequence (see also FIG. 12).

FIG. 38

MCF7/AZ cells were transfected with a construct encoding a Myc-tagged full-length NBG protein followed by simultaneous staining with an anti-Myc antibody (A), polyclonal NBG-specific antibody #31226 (B) and DAPI (C). A transfected cell (centre) is stained by both the anti-Myc antibody and the polyclonal antibody, whereas nontransfected cells are not stained by anti-Myc antibody and only very lightly by the polyclonal antibody.

### EXAMPLES

### Material and Methods to the Examples

Library Screening and DNA Isolation

YAC- and PAC-end clones and selected STSs were used to screen for additional PAC clones. These clones were isolated by screening of a gridded RCPI-1 PAC library (loannou et al., 1994). Subsequently, breakpoint-overlapping PAC clones were used to screen a chromosome-17 specific cosmid library. The PAC and cosmid filters and clones were distributed by the Ressourcenzentrum Primärdatenbank in the Max Planck-Institut für Molekulare Genetik in Germany (RZPD) (Zehetner and Lehrach, 1994). In brief, 30 ng probe DNA (YAC- or PAC-end clone, STS) was randomly labeled using the Megaprime DNA labeling kit (AP Biotech) according to the supplier's protocol. Prehybridisation of filters, hybridization and washing steps were done according to standard procedures. BACs were obtained from the Children's Hospital Oakland Research Institute (C.H.O.R.I.) in Oakland, Calif., USA (http://www.chori.org/bacpac).

PACs, BACs and cosmids were cultured in LB-broth medium containing the appropriate antibiotics. DNA was isolated using a rapid alkaline lysis miniprep method (C.H.O.R.I.).

The following databases were consulted for identification of publicly available genetic markers and clones: Whitehead Institute for Biomedical Research (http://www-genome.wi.mit.edu/), Marshfield Medical Research Foundation Center for Medical Genetics (http://www.marshmed.org/genetics), CEPH-Généthon (http://www.cephb.fr/ceph-genethon-map.html), National Center for Biotechnology Information (NCBI, http://www.ncbi.nlm.nih.gov/) and the Human Genome Project Working Draft at UCSC (http://genome.ucsc.edu/).

YAC and PAC End Isolation and Generation of Sequence-tagged Sites

YAC end clones were isolated using an Alu-vector based method (Nelson et al., 1989). PAC end clones were obtained by vectorette PCR (Riley et al., 1990), or were obtained by direct sequencing and subsequent PCR amplification. For direct sequencing, PAC DNA was subjected to cycle sequencing using BigDye chemistry on an ABI377 automatic sequencer (Applied Biosystems) with the following vector based primers: T7 (5′-TAATACGACTCACTATAGGG-3′) and SP6 (5′-CAAGCT ATTTAGGTGACACTATAGA-3′). Screening for repetitive sequences was done using the program ‘Repeat Masker’

(http://ftp.genome.washington.edu/RM/RepeatMasker.html). In total, 17 end sequences were generated of which 9 were submitted as GGS to Genbank [SP6 RPCI-1 105C17 (acc. n° BH021100); SP6 RPCI-1 605A3 (acc. n° BH021101); T7 RPCI-1 1167N14 (acc. n° BH021102); SP6 RPCI-1 835C10 (acc. n° BH021103); T7 RPCI-1 835C10 (acc. n° BH021104); SP6 RPCI-1 55N16 (acc. n° BH021105); SP6 RPCI-1 266J21 (acc. n° BH021106); T7 RPCI-1 118B4 (acc. n° BH021107); T7 RPCI-1 266J21 (acc. n° BH021108)] and 8 were used to develop end STSs. Primers were designed using the PrimerExpress software (Applied Biosystems). The novel STSs and primer sequences have also been submitted to GenBank (see Table 1).

Fluorescence in situ Hybridization (FISH)

PACs, BACs and cosmids were biotinylated (biotin-16-dUTP, Roche) or digoxigenated (digoxigenin-11-dUTP, Roche) by standard nick translation. FISH was performed according to Van Roy et al. (1994). Slides were mounted in Vectashield (Vector laboratories) plus DAPI (4′,6-diamidino-2′-phenylindole dihydrochloride, Roche) for counterstaining and observed under a standard ZEISS epifluorescence microscope equipped with a 100 W Hg lamp. FISH images were recorded using the ISIS digital imaging system (MetaSystems). Verification of the chromosomal localization of the isolated clones was done by hybridization to normal metaphase chromosomes. Subsequently, the position of the probes with respect to the constitutional 17q breakpoint was determined by FISH on hybrid cell lines containing either derivative chromosome 1 or derivative chromosome 17 (Laureys et al., 1995). To facilitate the detection of the respective derivative chromosomes, dual color hybridization was performed with a chromosome-17.specific library pBS-17 (Collins et al., 1991) and a chromosome- 1p36 specific probe p1-79 (D1Z2) (Buroker et al., 1987).

Fiber FISH

Fiber FISH slides were prepared according to Speleman et al. (1997). Prior to hybridization, fiber FISH slides were counterstained with DAPI and evaluated by fluorescence microscopy. Probe order, degree of overlap and estimation of size of gaps between probes was determined by dual color FISH experiments. Hybridization on fiber FISH slides was performed according to Van Roy et al. (1994) with minor modifications. Fiber slides were only pretreated with RNase A. Pepsin treatment and postfixation were omitted. Antibody concentration for the immunohistochemical detection of biotin and digoxigenin labeled probes was doubled in comparison with the standard FISH procedure and incubation of antibodies was performed at 37° C. for 30 min instead of 20 min at room temperature.

The lengths of probe signals, overlaps and gaps were measured from digitized images with the ISIS software program (MetaSystems, Atlussheim, Germany). The gap measurements were normalized based on the signal lengths of probes on both sides of the gap to compensate for the variation in the level of DNA stretching. For the purpose of probe ordering and length measurements, at least ten images were analyzed.

Estimation of Clone Insert Sizes by Field Inversion Gel Electrophoresis (FIGE)

PAC DNA (1 μg) was digested with restriction enzyme NotI according to the suppliers protocol (Gibco BRL). An agarose gel (1%) was run for 16 h, at 180 V forward and at 120 V reverse, at 20° C. in 0.5×TBE buffer on a Biorad FIGE Mapper™. Insert fragment sizes were determined by comparison with size standards (Lambda DNA-PFGE marker; Pharmacia Biotech)(Table 3).

Mapping of STSs and ESTs

Mapping of STSs and ESTs was done by hybridization of PCR products on PAC dot blots. PCR products were purified using a QIAquick PCR purification column (Qiagen). Purified PCR products were labeled using the Megaprime kit. Hybridization was performed according to standard procedures. Furthermore, STSs and ESTs were mapped by PCR on PAC and BAC clones or positions were inferred by BLAST query of the nr and htgs NCBI databases (http://www.ncbi.nlm.nih.gov).

Shotgun DNA Sequencing

Three overlapping cosmids (ICRFc105F1060D1, ICRFc105F1065D1, ICRFc105G3072D1), presumed to span the t(1;17) breakpoint on chromosome 17, were chosen for shotgun DNA sequencing. Cosmids were grown in LB with 25 μg/ml kanamycin to late log phase. Cosmid DNA was isolated using columns (Qiagen) and sheared on ice using an ultrasound sonicator. Treated DNA was analyzed in an agarose gel. Fragments of 0.8-1.0 kbp were cut from the gel, extracted and the ends were polished using T4 DNA polymerase and Klenow polymerase. Fragments were ligated into the pUC18Srf plasmid. A shotgun-sequencing library was generated using DH5α competent cells and checked for insert size by digestion with EcoRI and HindIII enzymes. More than 313 shotgun clones were selected from the three different cosmid fragment libraries and plasmid DNA was prepared using a BioRobot 9600 (Qiagen, Valencia, Calif., USA) and Qiagen Turbo kits. Clones were sequenced using universal M13 primers. In addition, specific walking primers were selected to fill the remaining gaps according to standard procedures.

DNA Sequence Analysis

Dye terminator sequencing (Sanger, 1981) was carried out by use of Big Dye chemistry (Perkin Elmer, Foster City, Calif.). The gels were run on a ABI Prism 377 DNA sequencer (Perkin Elmer, Foster City, Calif.). Nucleotide sequences were edited and assembled using GAP4 of the Staden software package (Bonfield et al., 1995)(URL http://www.mrc-lmb.cam.uk/pubseq/). RepeatMasker programs were used to screen DNA sequences for interspersed repeats known to exist in mammalian genomes (URL http://ftp.genome.washington.edu/RM/RepeatMasker.html).

All DNA sequences were analyzed using BLAST programs (Altschul et al., 1997)(URL http://www.blast.genome.ad.jp) to determine similarities or identities to known genes or EST sequences using non-redundant and EST compilation databases from Genbank and EMBL-EBI. Searches for amino-acid homologies were carried out using non-redundant Swiss-Prot and PIR databases with the BLASTP and FASTA programs. Exons were predicted by use of GENSCAN (URL http://CCR-081.mit.edu/GENSCAN.html) and GeneMark software (URL http://opal.biology.gatech.edu/GeneMark). Alignment of cDNA sequences to the genomic data (DNAstar software, Madison, Wis.) revealed exon-intron boundaries.

Southern Blotting

Genomic DNA from normal human placenta, from the Chinese hamster ovary cell line a3, and from somatic cell hybrids 32-7A and 32-2F53VIII were restricted with BglII, HindIII, SacI, XhoI, XbaI, McoI, PvuII, KpnI or HincII restriction enzyrfies. The restricted DNA was ethanol-precipitated, washed, dissolved in 20 μl, run on an 0.8% agarose gel and transferred to Hybond N⁺ nylon membranes (Amersham) following the recommendations of the manufacturer. Membranes were hybridized, washed and revealed by autoradiography and phosphor imaging using a Molecular Imager Fx (BioRad, Herts, UK). Probes used for hybridization were labeled by incorporation of [α-³²P]dCTP (Amersham) using a random priming kit (GibcoBRL). In order to generate probes adjacent to the t(1;17) breakpoint for Southern blot analysis, the cosmid contigs sequence spanning the breakpoint was screened with Repeatmasker (URL http://ftp.genome.washington.edu/cgi-bin/RepeatMasker) to select 9 repeat-free probes representative for the cosmid sequence contig (Table 4) (depicted in FIGS. 3A and 17A). The resulting PCR products, probe #1 (516 bp), probe #2 (789 bp), probe #3 (465 bp), probe #4 (478 bp), probe #5 (678 bp), probe #6 (478 bp; also depicted in FIG. 3B, FIGS. 3C and 17B); probe #7 (673 bp), probe #8 (761 bp) and probe #9 (567 bp; also depicted in FIGS. B and 17C) were directly sequenced to confirm their identity.

GenomeWalker Libraries

Libraries were generated according to the manufacturer's instructions (Clontech Laboratories, Palo Alto, Calif., USA). For the 32-2F53VIII somatic cell hybrid, 2.5 μg of genomic DNA was digested with blunt-end generating restriction enzymes PvuII, DraI, StuI, and EcoRV, for 2 h at 37° C. in a final reaction volume of 100 μl. After this incubation, the reaction was vortexed at low speed and returned to 37° C. for 16 h. DNA was purified by phenol-chloroform extraction and ethanol precipitation. Then, the digested DNA was dissolved in 20 μl.

Appropriate blunt-ended GenomeWalker adaptors (1.9 μl of 25 μM) with sequence

5′-GTAATACGACTCACTATAGGGCACGCGTGGTCGACGGCCCGGGCTGGT-3′
3′ H₂N-CCCGACCA-PO₄-5′

(Clontech), 0.5 μl of T4 DNA ligase (1 U/μl) and 1.6 μl of 5× ligase buffer were added. The mixture was incubated at 16° C. for 16 h. TE (72 μl; pH 7.4) was added and the obtained libraries, designated 32-2F53VIII/PvuII, 32-2F53VIII/StuI, 32-2F53VIII/EcoRV and 32-2F53VIII/DraI, were stored at −20° C. in 10-μl aliquots until use.

For the 32-7A somatic cell hybrid, 2 μg of genomic DNA was digested with blunt-end generating restriction enzymes DraI, EcoRV, PvuII, ScaI and SspI, for 2 h at 37° C. in a final reaction volume of 80 μl. After this incubation, the reaction was vortexed at low speed: and returned to 37° C. for 16 h. DNA was purified by phenol-chloroform extraction and ethanol precipitation. Then, the digested DNA was dissolved in 16 μl.

Appropriate blunt-ended GenomeWalker adaptors (1.9 μl of 25 μM) with sequence

5′-GTAATACGACTCACTATAGGGCACGCGTGGTCGACGGCCCGGGCTGGT-3′
3′ H₂N-CCCGACCA-PO₄-5′

(Clontech), 0.5 μl of T4 DNA ligase (1 U/μl) and 0.8 μl of 10× ligase buffer were added to 4.8 μl of the digested DNA. The mixture was incubated at 16° C. for 16 h. TE (72 μl; pH 7.4) was added and the obtained libraries, designated 32-7A/DraI, 32-7A/EcoRV, 32-7A/PvuII, 32-7A/ScaI and 32-7A/SspI, were stored at −20° C.

Long-range PCR Amplification of GenomeWalker Products

Takara LA TAQ mix (Takara) was used. Four μl dNTPs (2.5 mM), 5 μl 10×LA reaction buffer, 1 μl adaptor primer 1 (10 μM; 5′-GTAATACGACTCACTATAGGGC-3′), 1 μl Gene-specific primer (10 μM) and 1 μl GenomeWalker library DNA (see above) were mixed in a final volume of 50 μl. DNA was amplified using a two-step PCR with 7 cycles of denaturation at 94° C. for 2 sec plus extension at 72° C. for 3 min, followed by 35 cycles of denaturation at 94° C. for 2 sec plus extension at 67° C. for 3 min. After completion of the reaction, PCR products were analyzed on a 1% agarose gel. For nested PCR reactions, 1/50 μl of the first PCR amplification mixture was used as a template in the next reaction.

For the 32-2F53VIII libraries, gene-specific primers (GSP) were selected on the basis of DNA sequence of probe #6 (Table 4):

| | |
|---|---|
| GSP1: | 5′-CCCCTCAGCTCTGTGCATTTTGTCTA-3′ |
| GSP1′: | 5′-CCTCTTGCCCCCACCTAGTGTTTATTT-3′ |

For the 32-7A libraries, nested PCR reactions were done with adaptor primer 2 (5′-ACTATAGGGCACGCGTGGT-3′) and with gene-specific primers GSP1″ and GSP2, which were selected on the basis of the DNA sequence of probe #9 (Table 4):

GSP1″: 5′-CATAGTGGGGGACATCATGACAGTCAC-3′

GSP2: 5′ ACACCACCAGCCTCCCTCCATTTCTGA-3′ (probe #9 was elongated with 23 bp 5′ to obtain GSP2).

Cloning of PCR Products

PCR products were cloned by the TA cloning procedure in the pGEMTeasy vector (Promega). In the case of long-range PCR products (GenomeWalker experiment), PCR products were treated with normal TAQ polymerase after amplification in order to generate overhanging A ends to enable TA cloning.

Plasmid Constructions

All enzymes were purchased from Promega, New England Biolabs, Roche, Invitrogen or Fermentas.

pBlueKIAA1245 (FIG. 30) stands for cDNA clone hg04073 (GenBank Acc No: AB033071-GI:6330825; backbone vector pBluescriptII SK+); pSport-AB25 (FIG. 30) stands for cDNA clone DKFZp434G2022 (GenBank Acc No: AL042839-GI:5935596; backbone vector pSport).

Construction of pBlue-KIAA-AB25 (FIG. 30)

The pBlue-KIAA1245 plasmid was digested with NotI and NcoI and a fragment of 5,952 bp was isolated. Plasmid pSport-AB25 was digested with NotI, NcoI and BstEII and a NotI-NcoI fragment of 4,385 bp was isolated. These two fragments were ligated together with T4 DNA ligase, which produced pBlue-KIAA-AB25.

Construction of pEF6/Myc-His-A-KIAA-AT

Plasmid pEF6/Myc-His-A (Invitrogen) was digested with BamHI and EcoRV and a fragment of 5,898 bp was isolated. Plasmid pBlue-KIAA1245 was cut with NcoI, blunted with Pfu DNA polymerase (Promega) and cut with BclI resulting in a fragment of 2,326 bp. These fragments were ligated with T4 DNA ligase, which produced plasmid pEF6/Myc-His A-KIAA-AT.

Construction of pCS2+MT-KIAA-AB25

Plasmid pCS2+MT (Roth et al., 1991) was linearized with StuI and dephosphorylated with CIP (calf intestinal phosphatase). Plasmid pBlue-KIAA-AB25 was cut with BclI and a fragment of 5,401 bp was isolated. This fragment was blunted using Pfu DNA polymerase. Vector and insert were ligated using T4 DNA ligase.

Construction of pEF6/Myc-His-B-KIAA-AB25

Plasmid pEF6/Myc-His-B (Invitrogen) was digested with BamHI and EcoRV and a fragment of 5,902 bp was isolated. Plasmid pBlue-KIAA-AB25 was digested with BclI and SapI, which resulted in a fragment of 4,946 bp. PCR was used to mutate a stop codon using primers FVR2803 (5′-CGCATAGTGCGGTGTGCTGATGGAAGT-3′) and FVR2804 (5′-GCGATATCGTACTGTGGGAATATGACTC-3′), which contains an EcoRV recognition site. 40 ng DNA (pSport-AB25) was used as a template in a PCR mix with VENT DNA polymerase. The DNA was denatured at 95° C. for 5 min, followed by 35 cycles of denaturation at 95° C. for 30 sec, annealing at 56° C. for 30 sec and extension at 75° C. for 30 sec. The reaction had a final extension of 10 min at 75° C. After completion of the reaction, PCR products were analyzed on a 1% agarose gel and purified using Concert™ Rapid PCR Purification System (Invitrogen). Subsequently, the DNA was digested with EcoRV and SapI. The three fragments were ligated using T4 DNA ligase.

Construction of pEGFP-C3-AB25-CT

pEGFP-C3 (Clontech) was digested with Ecl13611 and BamHI and a fragment of 4,685 bp was isolated. Plasmid pSport-AB25 was cut with NcoI, blunted with Pfu DNA polymerase (Promega) and digested with BclI. A fragment of 3,079 bp was isolated. These fragments were ligated with T4 DNA ligase, which resulted in plasmid pEGFP-C3-AB25-CT.

Construction of pEGFP-C3-KIAA-AB25

Plasmid pEGFP-C3 (Clontech) was linearized with BamHI and dephosphorylated with calf intestinal phosphatase. Plasmid pBlue-KIAA-AB25 was cut with BclI, which resulted in a fragment of 5,401 bp. This was ligated with the linearized vector using T4 DNA ligase.

Construction of pEGFP-N3-KIAA-AT

Plasmid pEGFP-N3 (Clontech) was linearized with Ecl13611 and dephosphorylated with calf intestinal phosphatase, which resulted in a fragment of 4,729 bp. Plasmid pBlue-KIAA-AB25 was digested with NcoI and BclI and blunted with Pfu DNA polymerase resulting in a fragment of 2,326 bp. These fragments were ligated with T4 DNA ligase, which resulted in plasmid pEGFP-N3-KIAA-AT.

Construction of pCS2+MT-chimera1

Plasmid pCS2+MT-KIAA-AB25 was linearized with XcmI and blunted with T4 DNA polymerase. Subsequently, the DNA was cut with BsrGI and a fragment of 6,939 bp was isolated. Plasmid pGEM-T Easy-chimera1 (see further) was digested with HincII and BsrGI and a fragment of 417 bp was isolated. The fragments were ligated using T4 DNA ligase.

Construction of pCS2+MT-chimera2

Plasmid pCS2+MT-KIAA-AB25 was linearized with XcmI and blunted with T4 DNA polymerase. Subsequently, the DNA was cut with BsrGI and a fragment of 6,939 bp was isolated. Plasmid pGEM-T Easy-chimera2 (see further) was linearized with BstXI, blunted with T4 DNA polymerase and cut with BsrGI. A fragment of 245 bp was isolated and ligated into the vector using T4 DNA ligase.

DNA Transfections

MCF7/AZ cells were maintained in DMEM, 10% FCS, non-essential amino acids, sodium pyruvate and 6 ng/ml bovine insulin and transfected using Fugene6 Reagent (Roche) according to the manufacturer's instructions. Colo320DM cells were maintained in RPMI1640, 10% FCS, L-Gln and penicillin/streptomycin and transfected using Lipofectamine (Invitrogen) according to the manufacturer's instructions. HEK293T cells were maintained in DMEM, 10% FCS, L-Gln, penicillin/streptomycin, sodium pyruvate and non-essential amino acids and transfected using the calcium phosphate method. HCT8 cells were maintained in RPMI1640, 10% FCS, L-Gln, penicillin/streptomycin and sodium pyruvate and transfected using Lipofectamine Plus (Roche) according to the manufacturer's instructions. Mouse NMe cells were maintained in DMEM, 10% FCS, penicillin/streptomycin, L-Gln and 10 μg/ml insulin and transfected using Lipofectamine (Roche) according to the manufacturer's instructions.

Immunofluorescence

After transfection, cells were kept in culture for 48 h. After this period, cells were washed twice with PBS-A and fixed with ice-cold methanol. Incubation with primary antibody was during 1 h for anti-Myc (diluted 1/125) and anti-lamin B1 (diluted 1/60) antibodies (Calbiochem) or during 2 h for the polyclonal antibodies (diluted 1/100). As secondary antibodies anti-mouse IgG Alexa488, Alexa594, anti-rabbit IgG Alexa488 and Alexa594 (Molecular Probes) were used (diluted 1/300). The endoplasmic reticulum of MCF7/AZ cells was stained using 20 μg/ml Concanavalin A Alexa594 conjugate (Molecular Probes) in PBS-A for 30 min, followed by two washes with PBS-A.

Coverslips were mounted using Vectashield (Vector Laboratories) and observations were made through an Axiophot microscope (Zeiss). Images were obtained using MetaMorph software.

Northern Analysis

Total RNA was isolated from cell lines using the RNeasy kit (Qiagen). 23 μg of RNA was loaded on a 1% agarose gel and blotted on Hybond N⁺ Membrane (Amersham Pharmacia) using standard protocols (Sambrook et al., 1989). For generating a 5′-specific probe, PCR was performed on 2 ng of pCS2+MT-KIAA-AB25 DNA using primers 5′-TGAGGAATGAGCGACAGT-3′ and 5′-TTTGAGGCTTCTGAACTG-3′ in a PCR mix with Taq DNA polymerase. The DNA was denatured at 94° C. for 2 min, followed by 35 cycles of denaturation at 94° C. for 20 sec, annealing at 56° C. for 20 sec and extension at 72° C. for 1 min. The products were treated by a final extension of 10 min at 72° C. After completion of the reaction, PCR products were analyzed on a 1% agarose gel and purified using Concert™ Rapid PCR Purification System (Invitrogen). For generation of a 3′-probe, cDNA clone DKFZp434M0628 (clone AB18, Table 6) was digested with EcoRI and PstI and a fragment of 691 bp was isolated. Probes were labelled using RadPrime Labelling Kit (Invitrogen) and alpha-³²P-dCTP (Amersham). Hybridisation was performed overnight in 1% BSA, 7% SDS, 0,5 M Na₂HPO₄, pH 7,2, 1 mM EDTA and 100 μg/ml ssDNA. After hybridisation, filters were washed at 65° C. with a final stringency of 0.1 M sodium phosphate, pH 7.2, 1% SDS and 1 mM EDTA. For detection and quantitation, a Phosphor Imager cassette (Molecular Dynamics, Sunnyvale, Calif.) was exposed and scanned with a Molecular Imager® FX using the Quantity One software (BioRad, Richmond, Calif.).

5′-RACE Reactions

5′-RACE was performed using the 5′-RACE system for Rapid Amplification of cDNA Ends (Invitrogen) according to the manufacturer's instructions. Briefly, 2 μg total RNA, isolated from either 32-7A, or 32-2F53VIII, or SK-N-SH or TR-14 cells was first-strand transcribed using the gene-specific primers FVR2458 (5′-TTGTTCCCATCAAAGTAAGAAAC-3′, located in type-14 exons) or FVR2457 (5′-ATTGACGGAGTCGAATAACATCTA-3, located in type-10, -11 or -14 exons). PCR was executed using the Abridged Anchor Primer (AAP) and a gene-specific primer FVR2456 (5′-CCCCTTCTCCTTCTTTTCTTCGT-3′, located in exons 12.5, 12.6 and 12.7) or FVR2455 (5′-CTCCCACGTCAAGAGAAAAG-3′, located in type-11 exons). Nested PCR was performed using the Unabridged Anchor Primer (UAP) and FVR2455 or FVR2454 (5′-CATAGGGCAGGCAGGAGTCAG-3′, located in type-11 exons but with high homology to part of the type-10 exons). Products were separated on a 1% agarose gel, eluted using the Concert™ Rapid Gel Extraction Purification System (Invitrogen) and cloned into plasmid pGEM-T Easy (Promega).

3′-RACE

3′-RACE was performed using the 3′-RACE System for Rapid Amplification of cDNA Ends (Invitrogen) according to the manufacturer's instructions. Briefly, 2 μg of total RNA isolated from the 32-7A cell line was first-strand transcribed using the AP (Adapter Primer). Two PCRs (PCR A and PCR B) were set up using the UAP (Universal Anchor Primer) and a gene-specific primer (either GSP1 or GSP2):

| | |
|---|---|
| PCR A: GSP1 | (5′-GCCCTTATGACTCCAACCAG-3′), |
| PCR B: GSP2 | (5′-ATTGGCTCATCCTCTCATGTT-3′). |

Nested PCRs were done using the Abridged Universal Anchor Primer (AUAP) and GSP2 (for nested PCR on PCR A), or the AUAP and GSP3 primer pair (for nested PCR on PCR B). GSP3: 5′-TCCCAGAAAATGAAAGTGATG-3′.

Products were separated on a 1% agarose gel and eluted using the Concert™ Rapid Gel Extraction Purification System (Invitrogen) and cloned in pGEM-T Easy (Promega). This yielded plasmids pGEM-T Easy-chimera1 and -chimera2 in the case of 32-7A RNA as template.

RT-PCR

RT-PCR was performed on RNA from the 32-2F53VIII somatic hybrid cell line and from human fetal brain (HFB). Total RNA was isolated from 32-2F53VIII cells using RNeasy RNA Extraction Kit (Qiagen), whereas HFB RNA was purchased (Clontech). First-strand synthesis was done using SuperScript II reverse transcriptase (Invitrogen) and oligo(dT) primers. Primers for RT-PCR on cDNA from 32-2F53VIII cells were FVR2457 (5′-ATTGACGGAGTCGAATAACATCTA-3′) as reverse primer and FVR2511 (5′-TTGGCTCTTGACGTGGACAGAATTA-3′) or FVR2512 (5′-AAGGACCAGGAAGAGGAAGAAGA-3′) as forward primer. For RT-PCR on HFB cDNA, primers FVR2686 (5′-CTCAACTCTCATTGGCTCATC-3′) and FVR2687 (5′-GTCCTCCTTTTTCACTTGATC-3′) were used. PCR fragments were separated on a 1% agarose gel and purified using the Concert™ Rapid Gel Extraction Purification System (Invitrogen) and cloned in pGEM-T Easy (Promega).

Antibodies

Antibodies specific for NBG proteins were raised by immunisation of rabbits with 200 μg of synthetic peptides with sequence NH₂-VGEIEKKGKGKKRRG-COOH (peptide #1059), NH₂-GEEDQNPPSPRLSGVLM-COOH (peptide #1060) or NH₂-PEILQDSLDRSYSTPSM-COOH (peptide #1061). These peptides were coupled to keyhole limpet hemocyanin via an additional cysteine residue at the NH₂-terminal end using the Imject® Maleimide Activated mcKLH Kit (Pierce). Boosts were given with intervals of minimum two weeks. Sera were tested by ELISA on the peptides used for injection, using nonrelevant peptide as a negative control. Sera were also tested on lysates of HEK293 cells transfected with pEGFP-C3-AB25-CT encoding the GFP-tagged carboxyterminal part of the NBG protein. The specificity of the antibodies was also evaluated by use of immunofluorescence. To this end, MCF7/AZ cells were transfected with the pCS2+MT-KIAA-AB25 construct encoding the Myc-tagged full-length NBG-protein.

### Example 1

### Construction of a 1.4 Mb PAC/BAC Contig Covering the Constitutional 17q Breakpoint

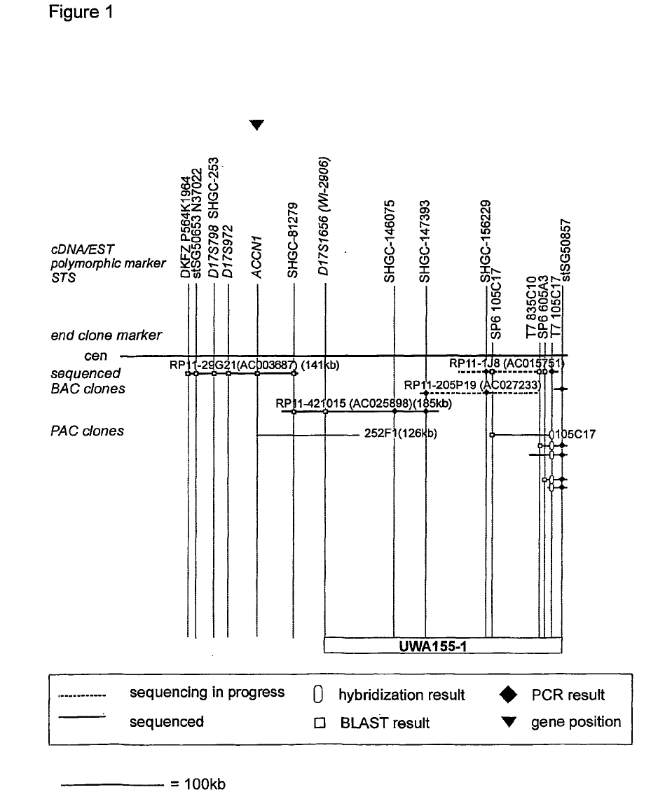

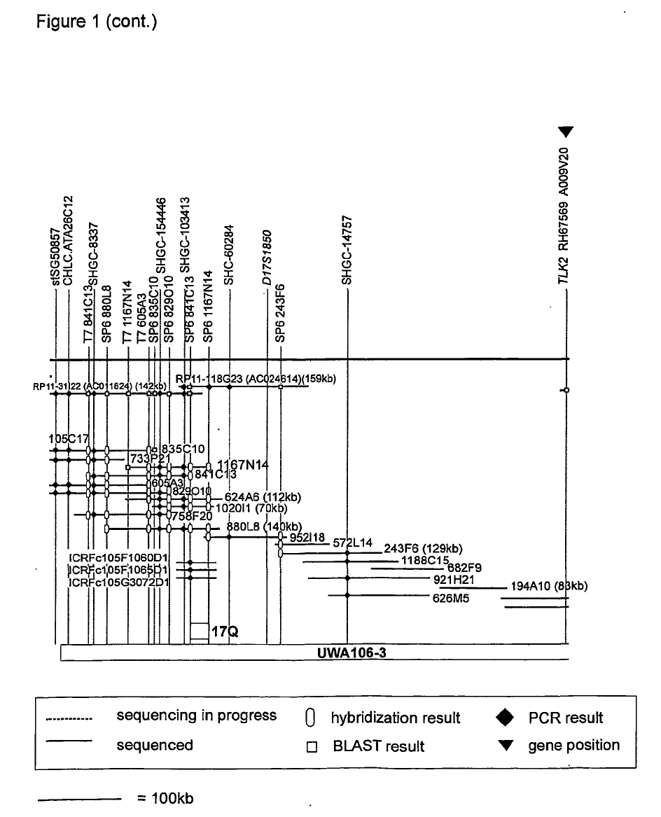

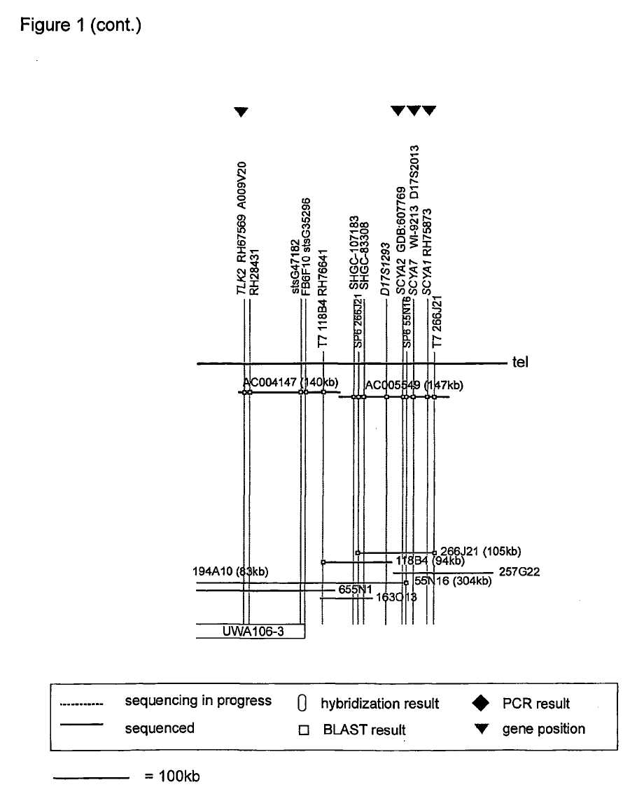

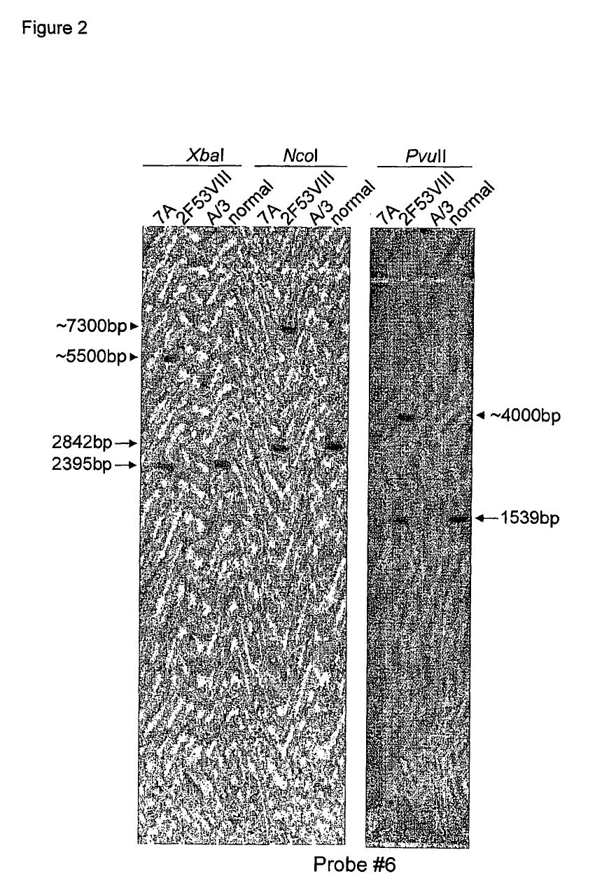

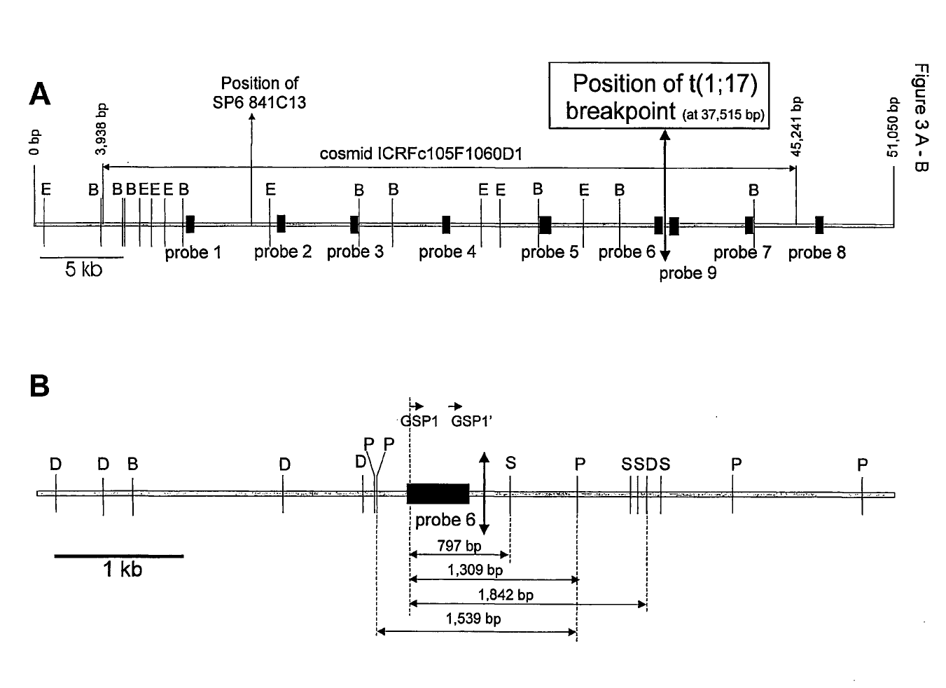

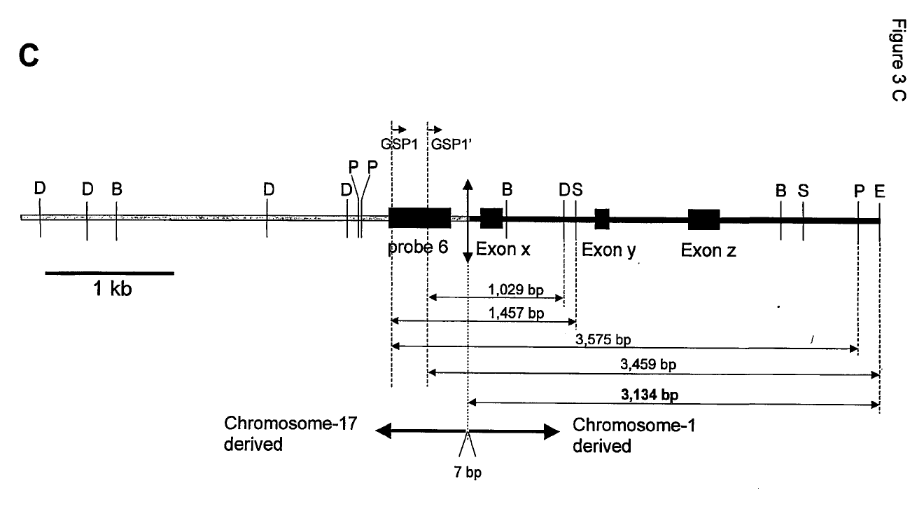

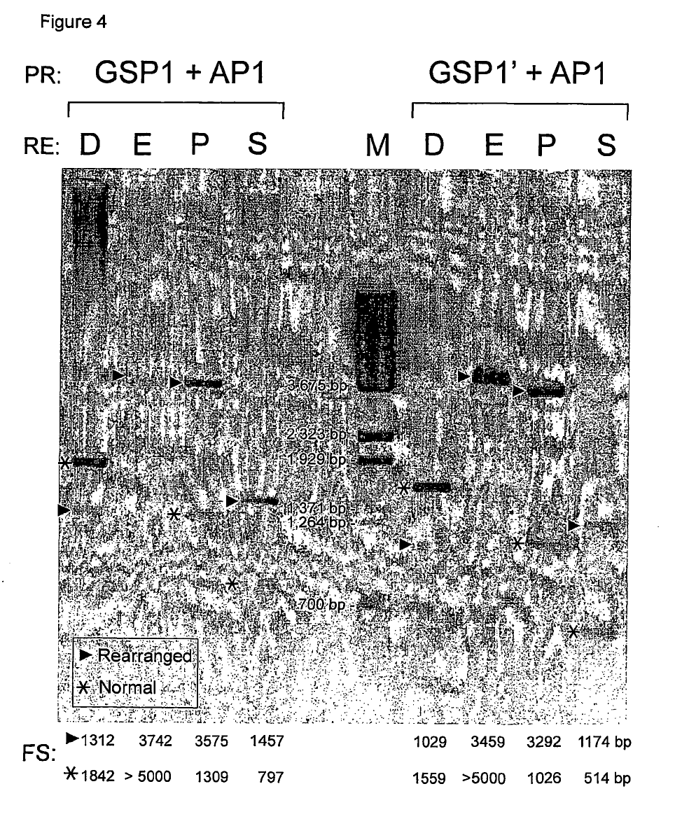

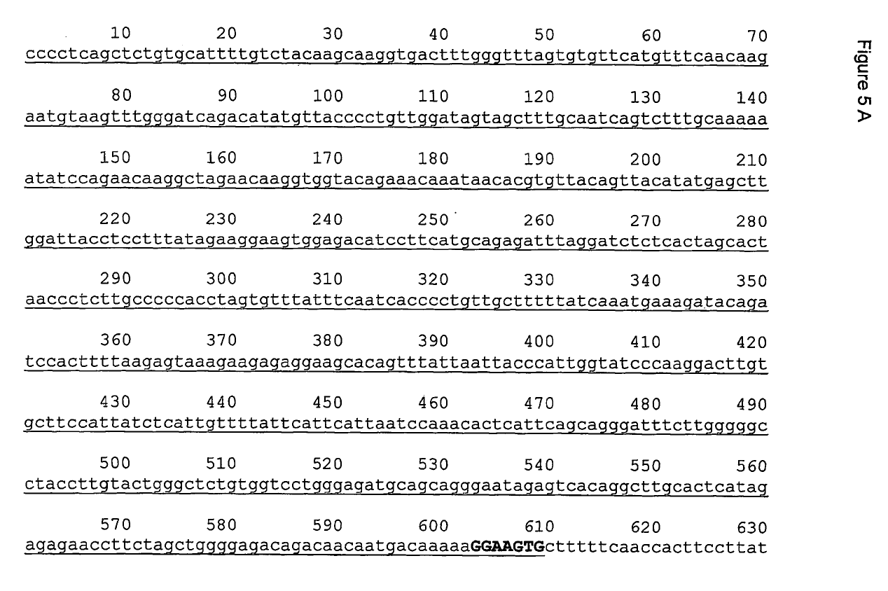

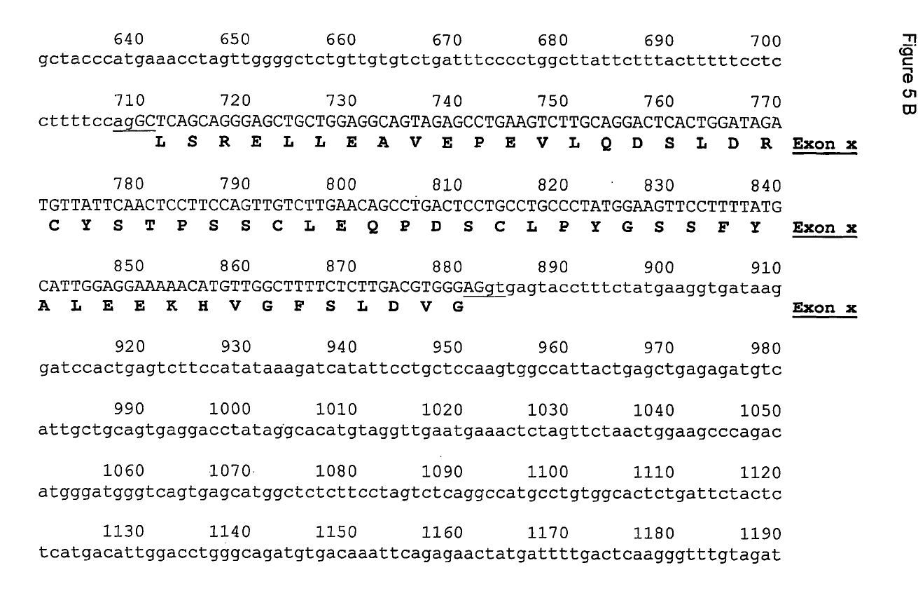

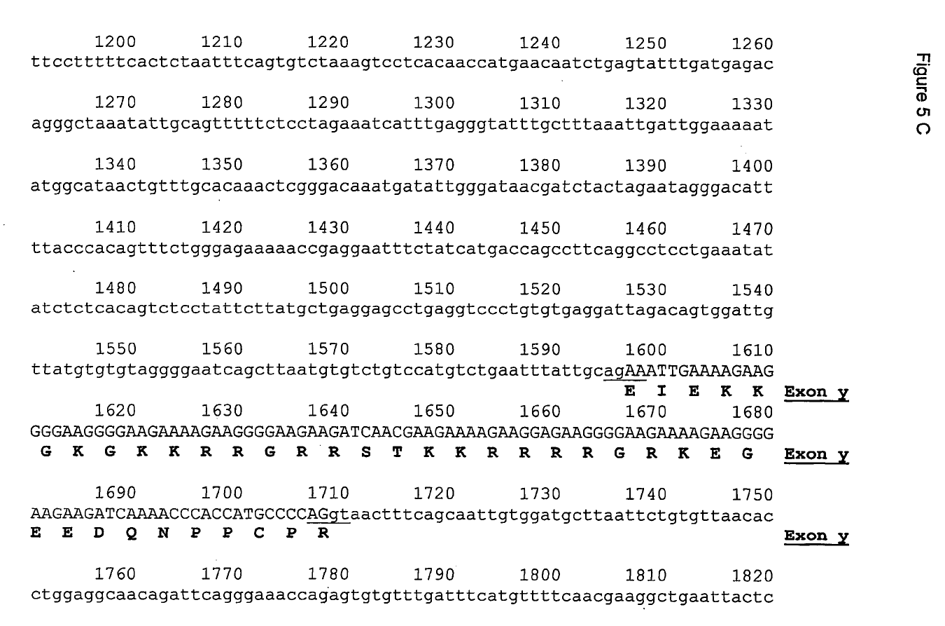

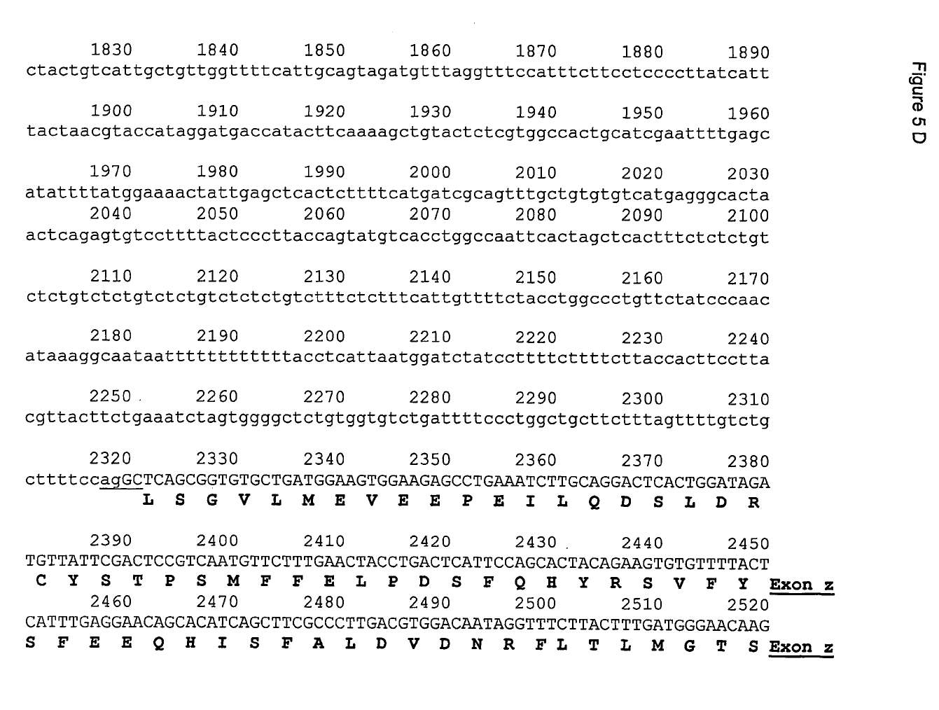

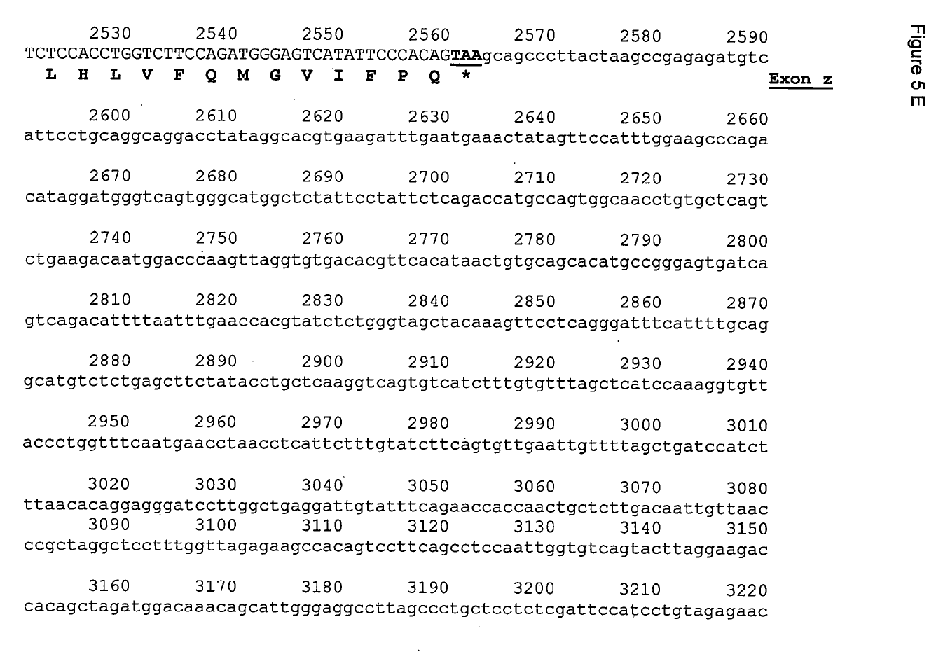

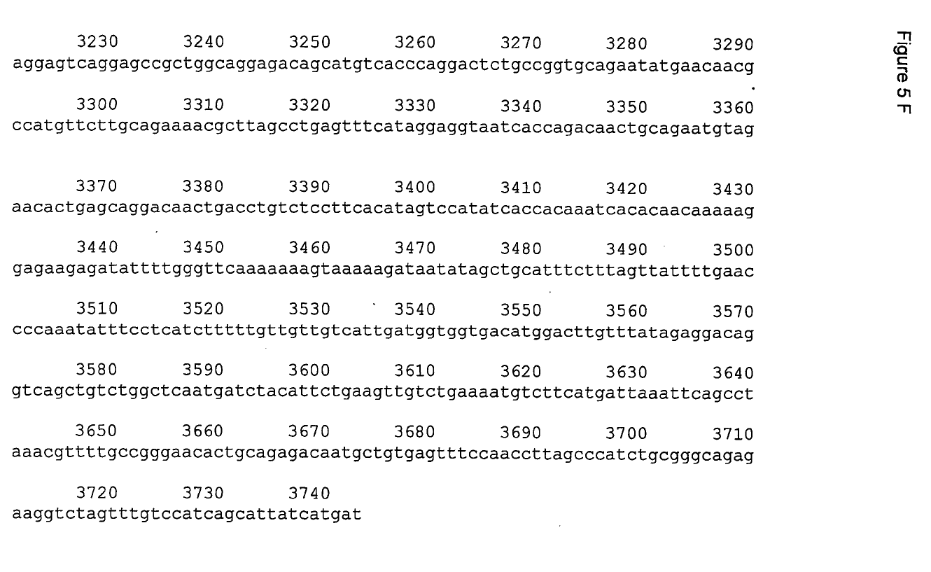

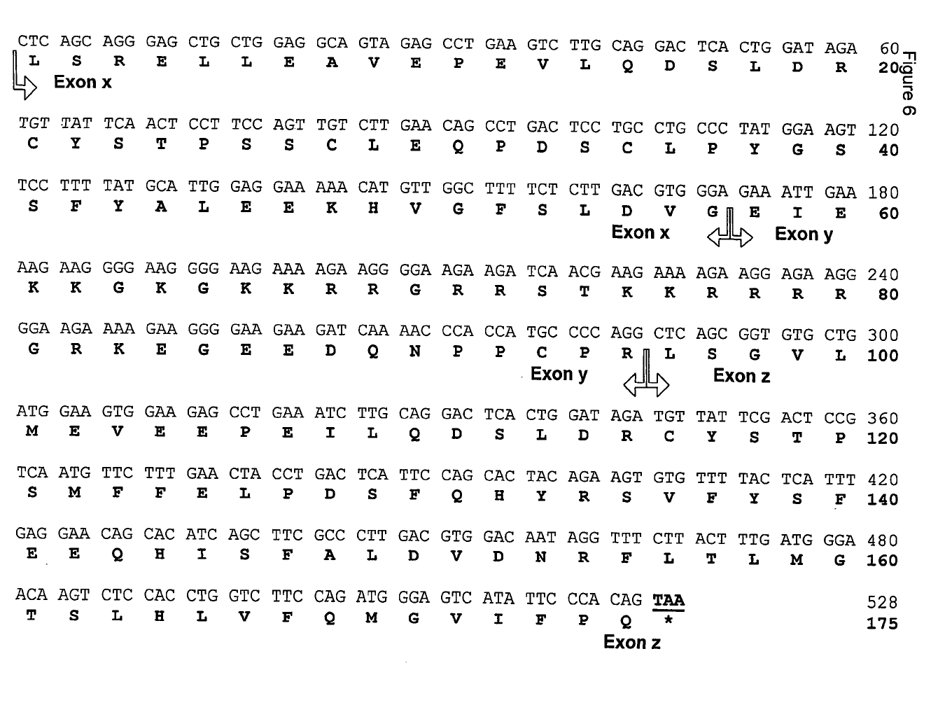

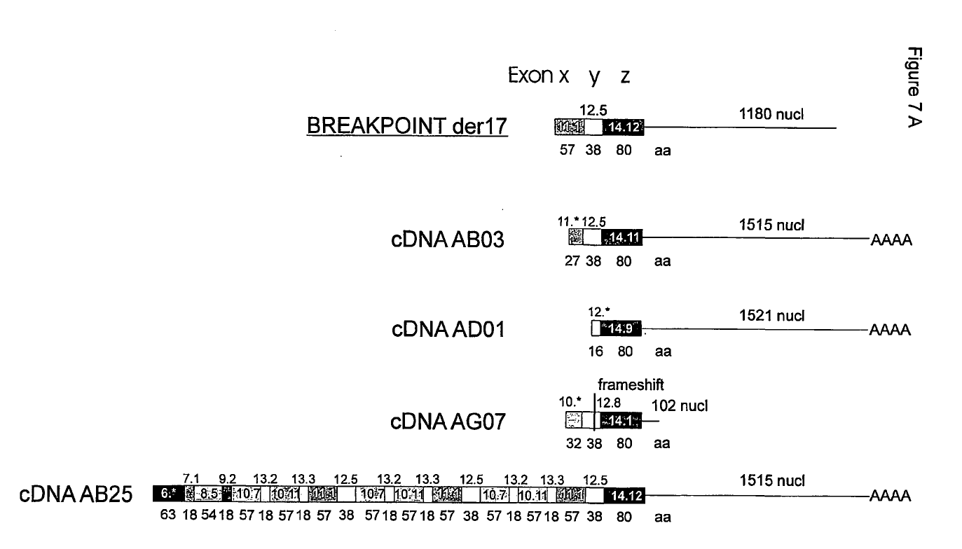

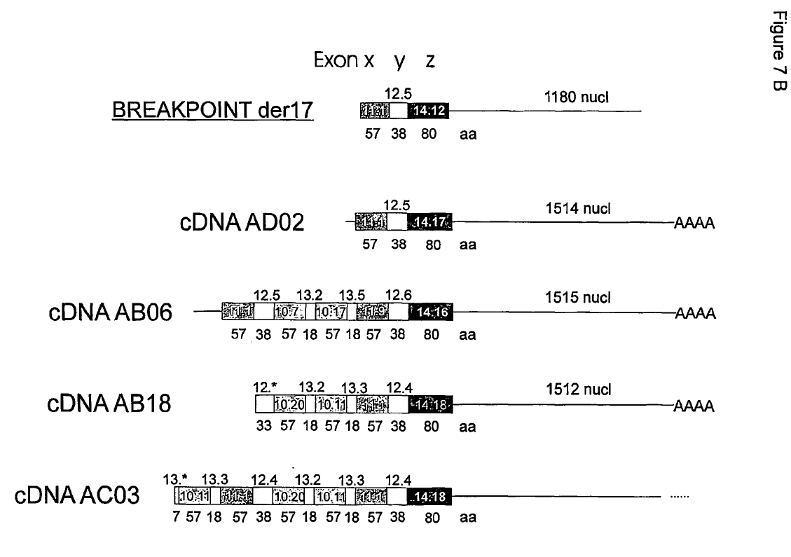

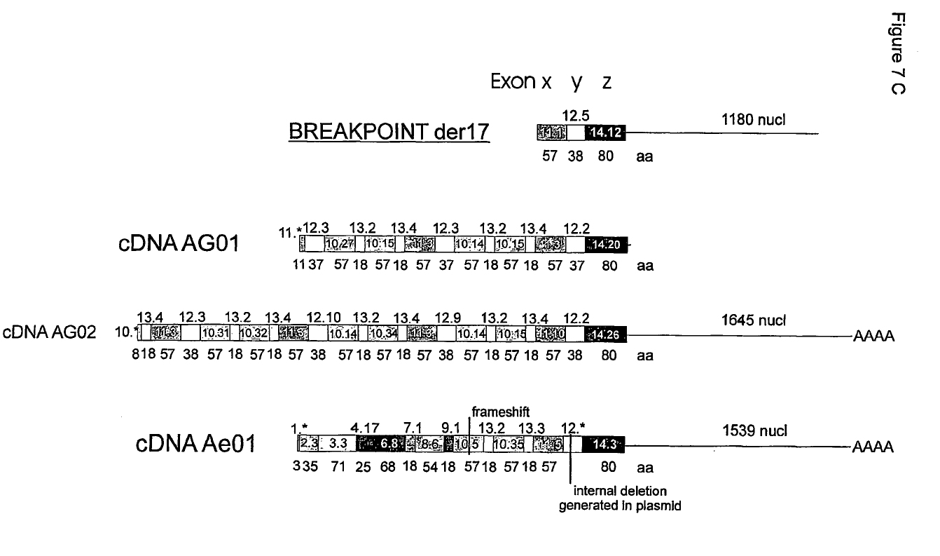

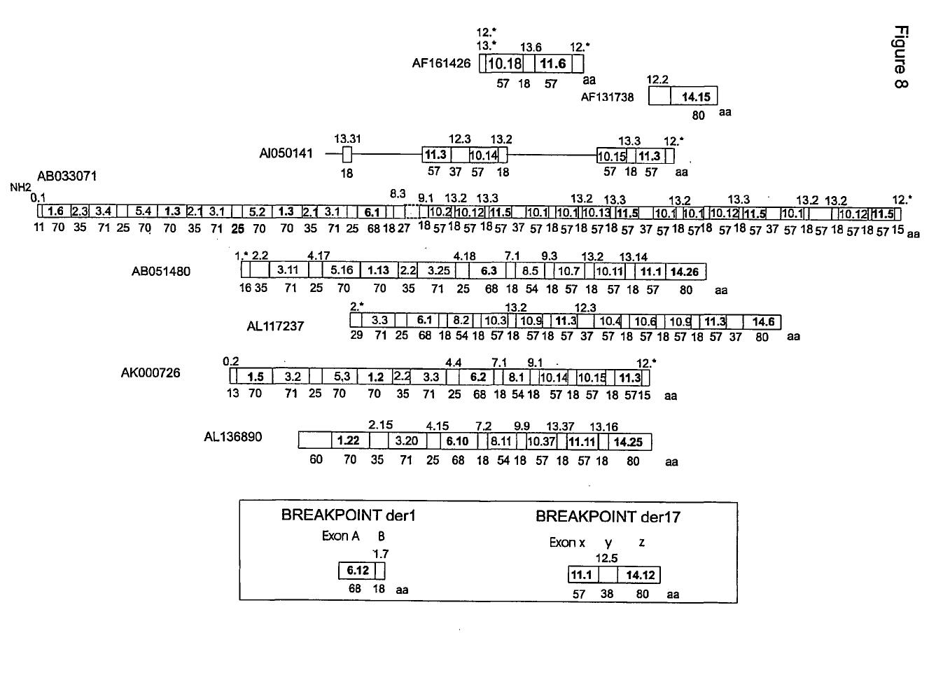

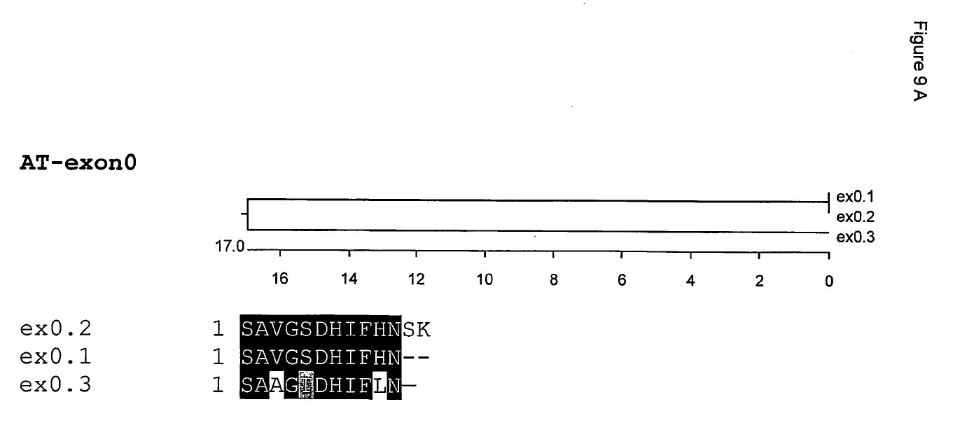

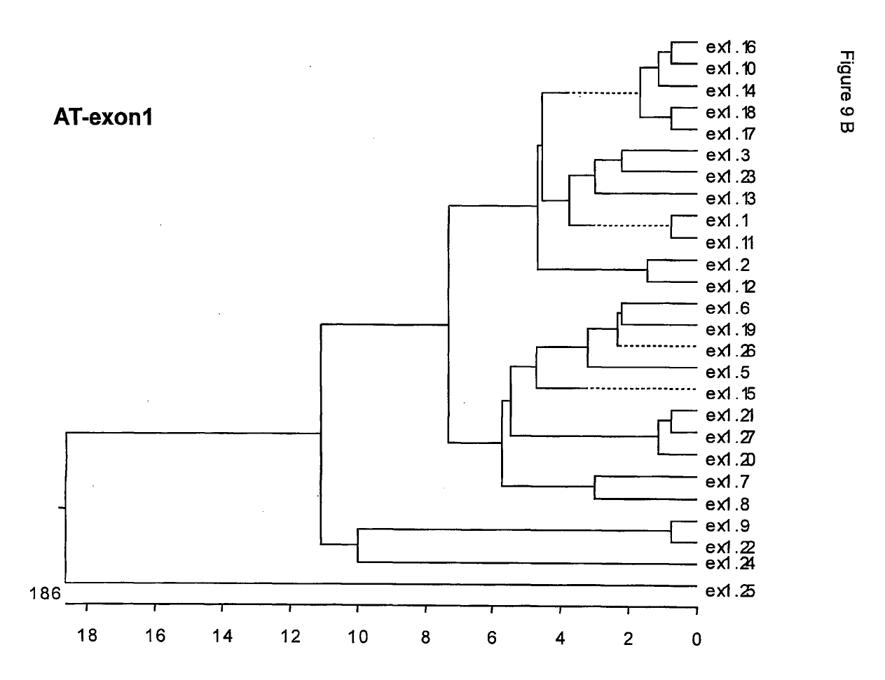

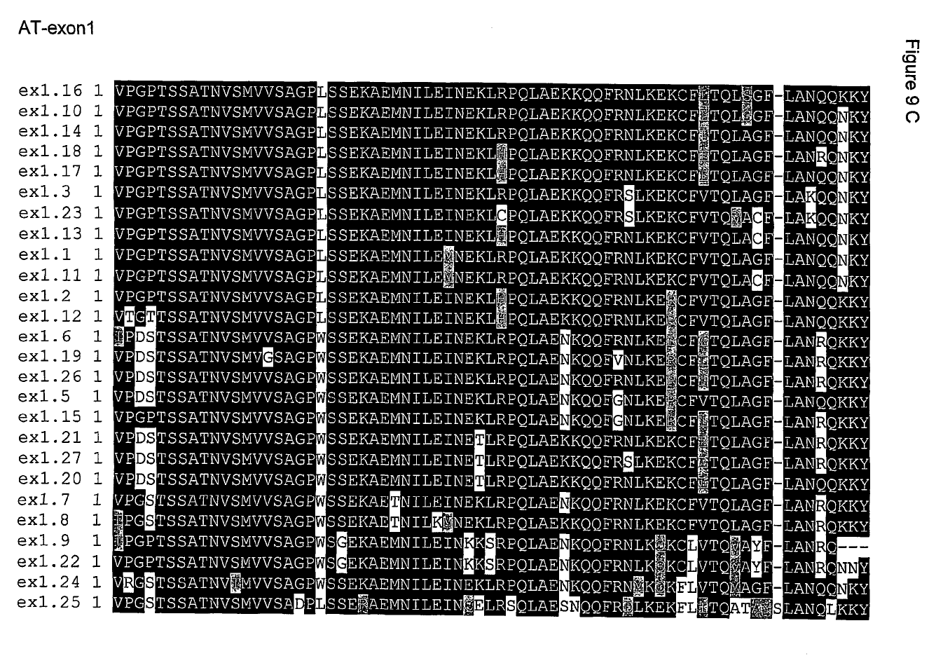

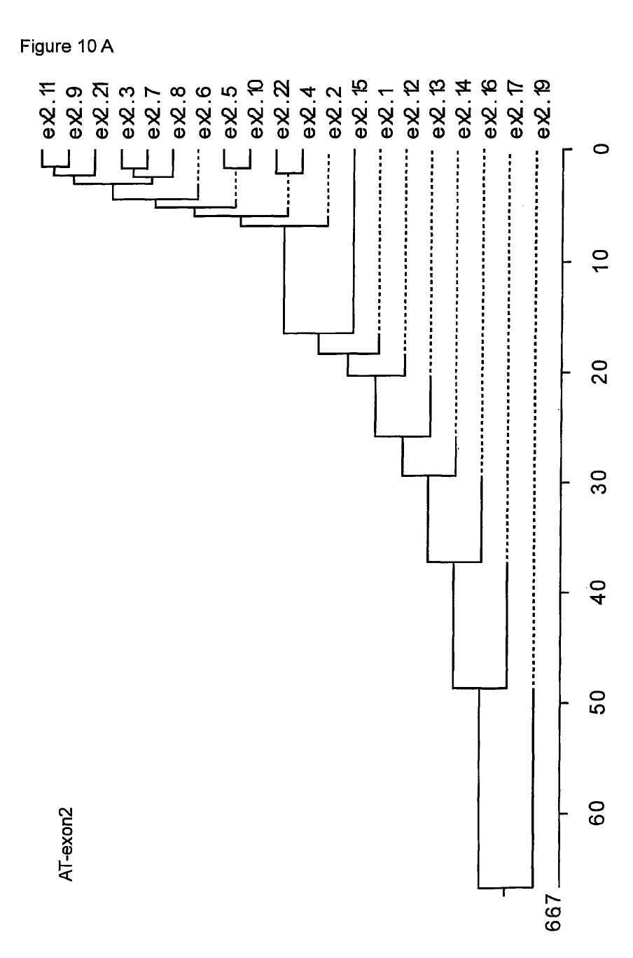

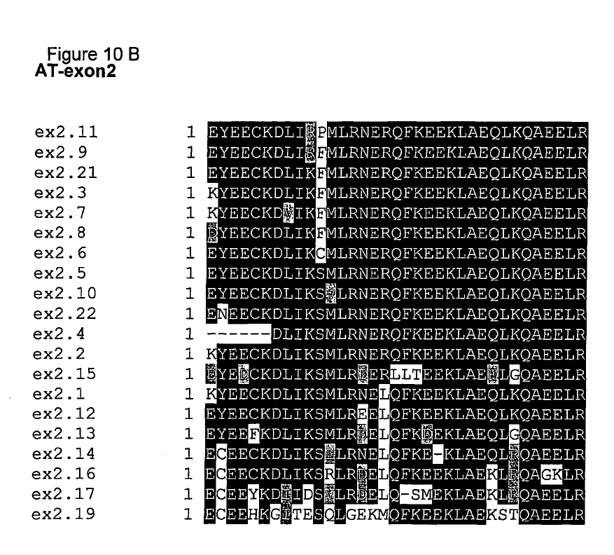

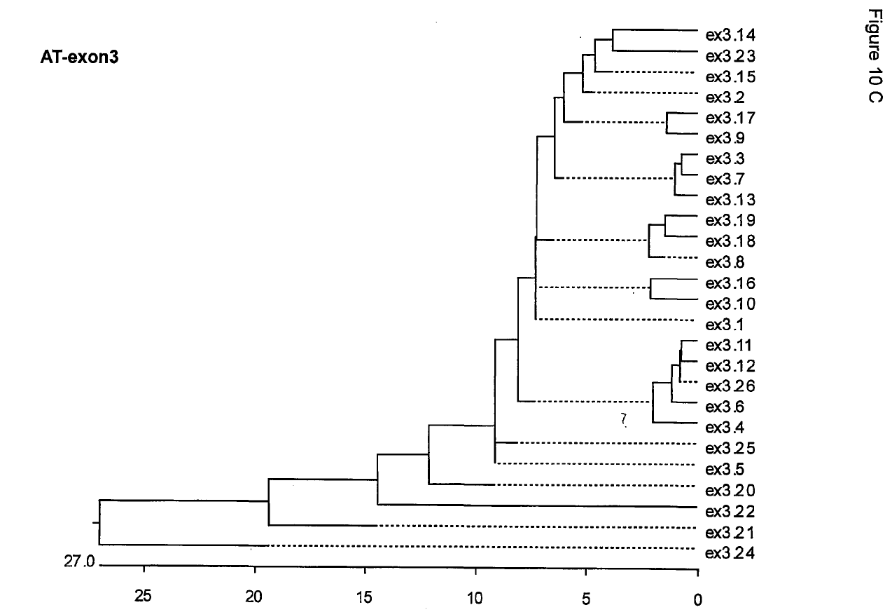

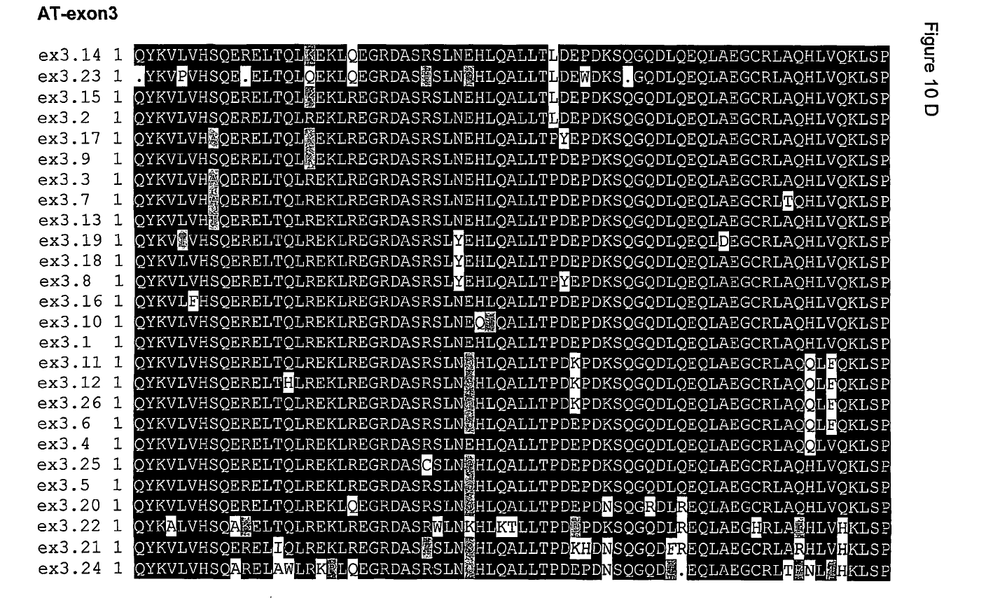

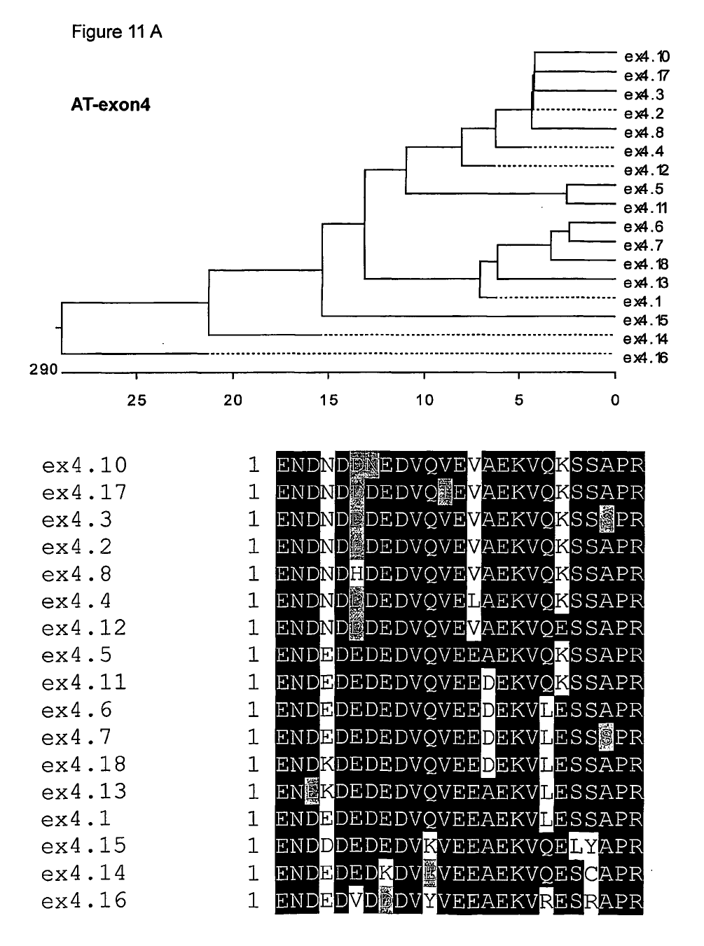

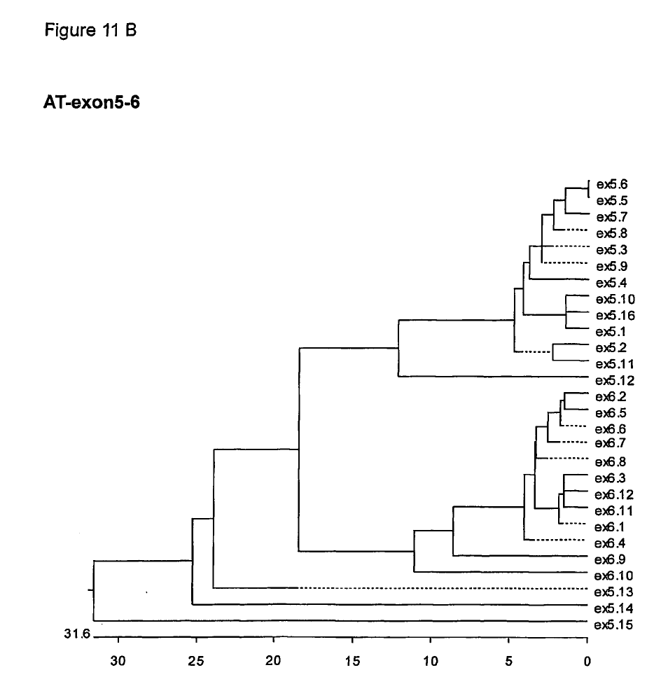

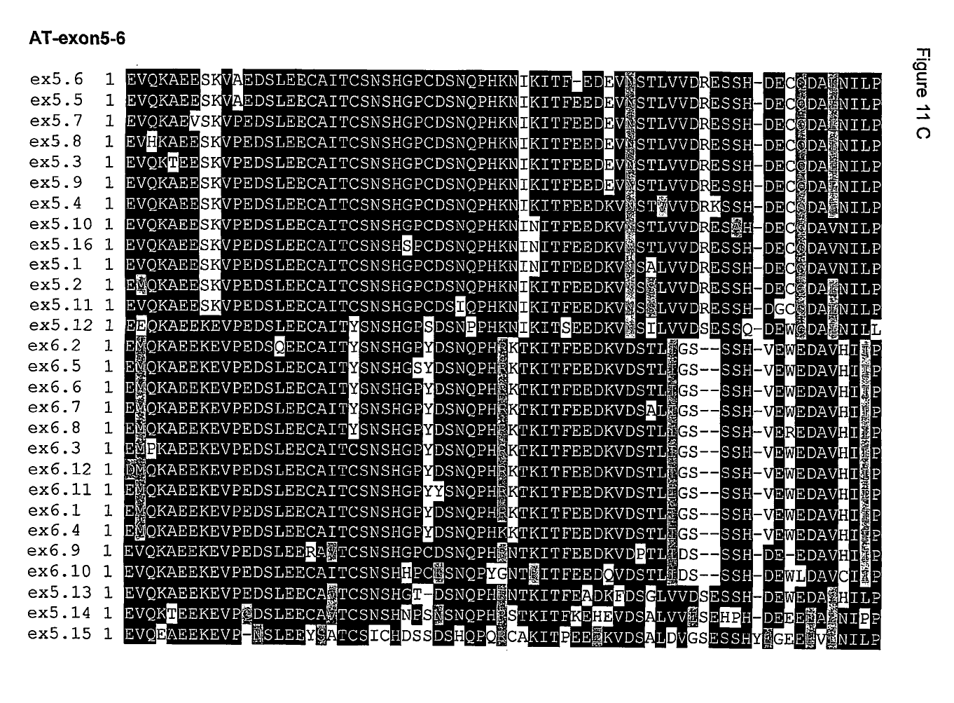

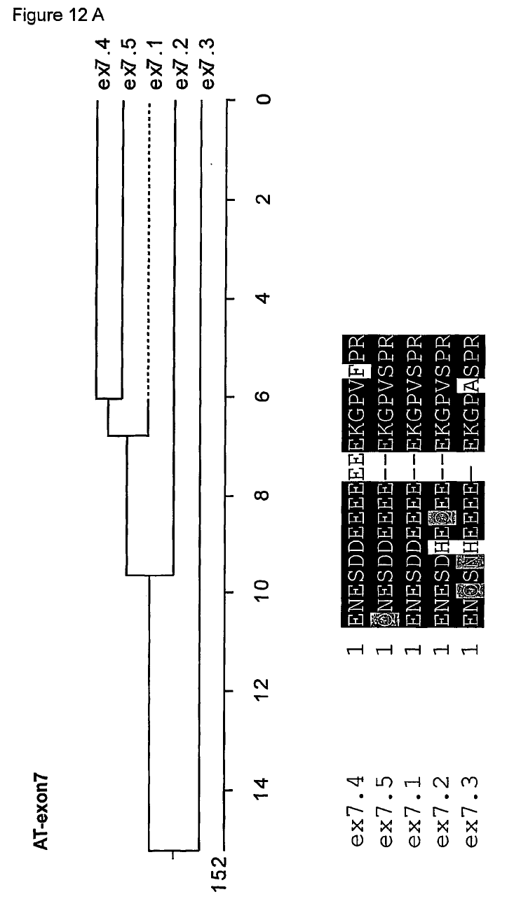

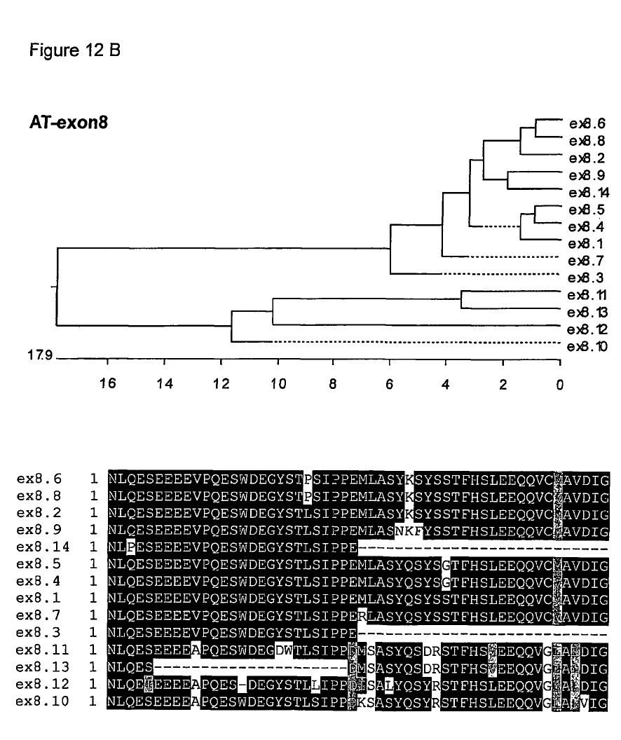

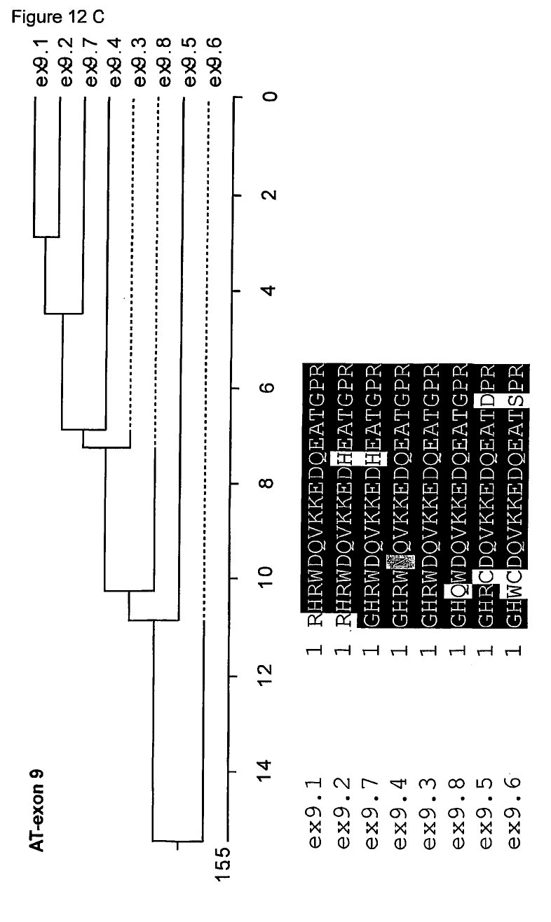

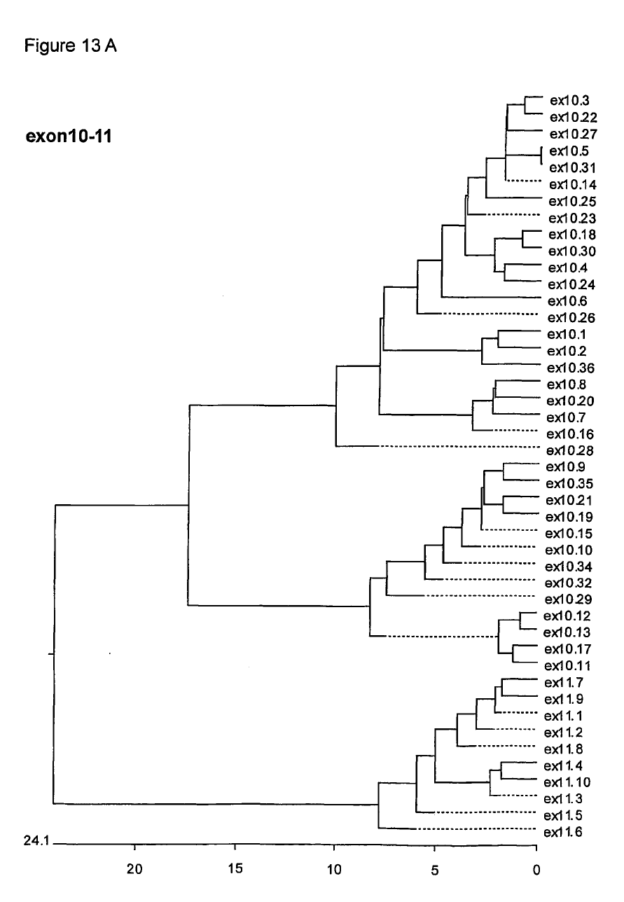

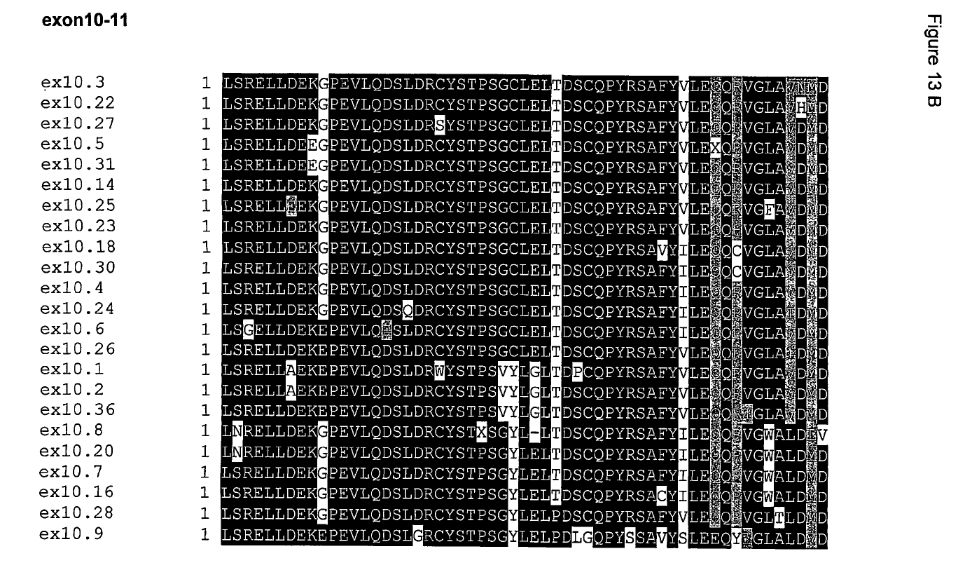

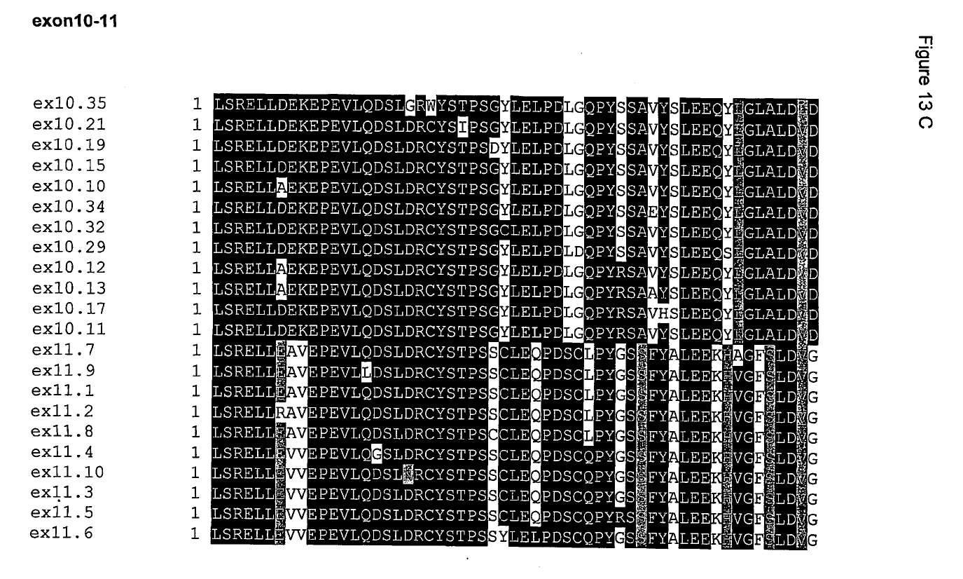

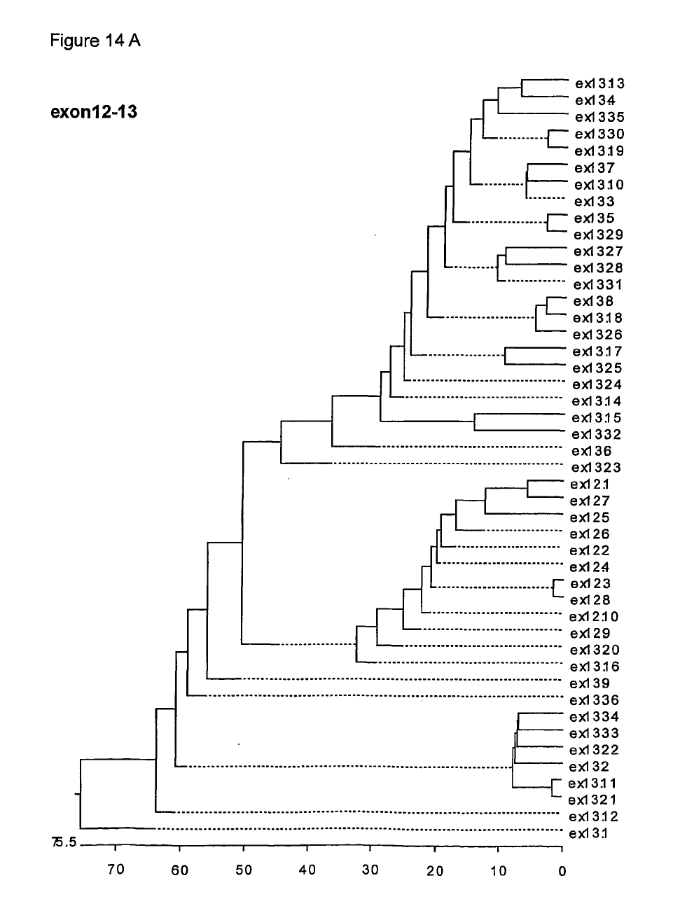

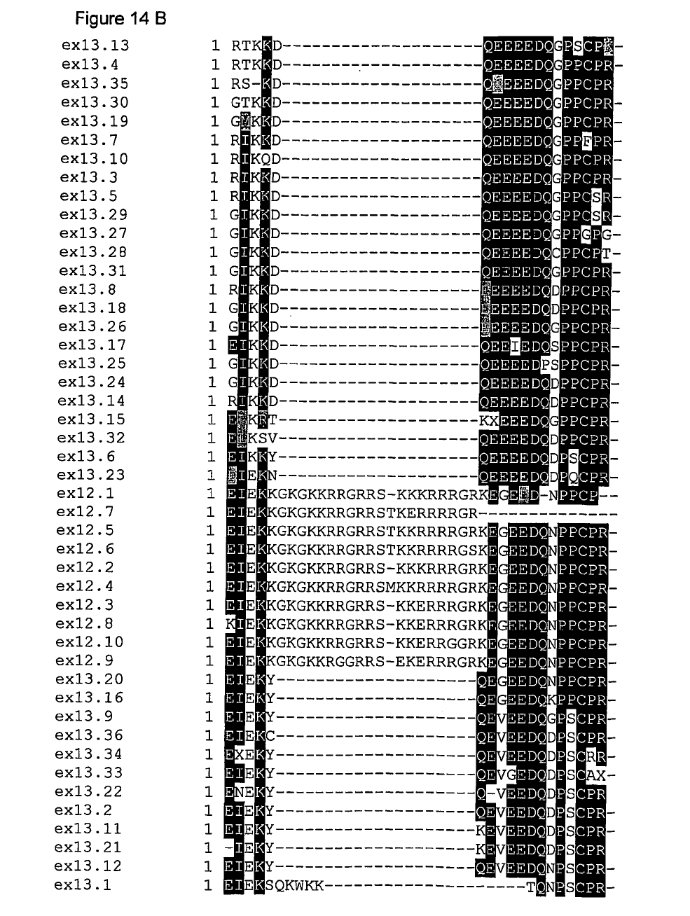

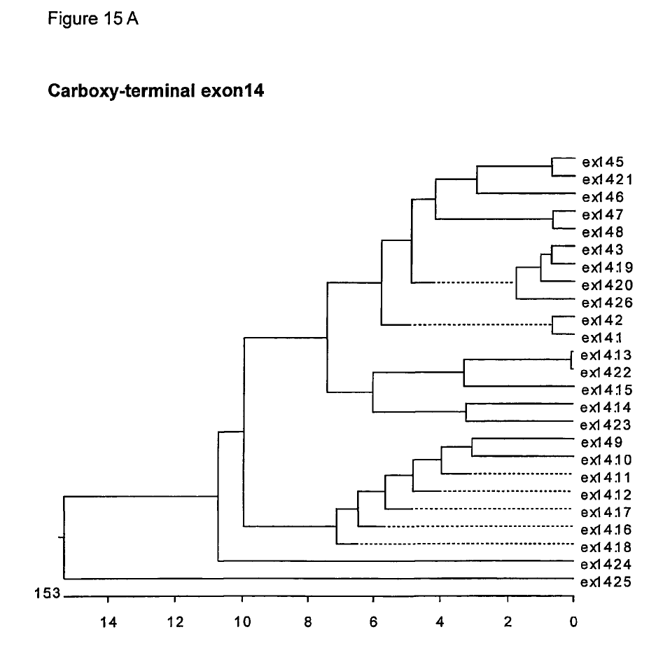

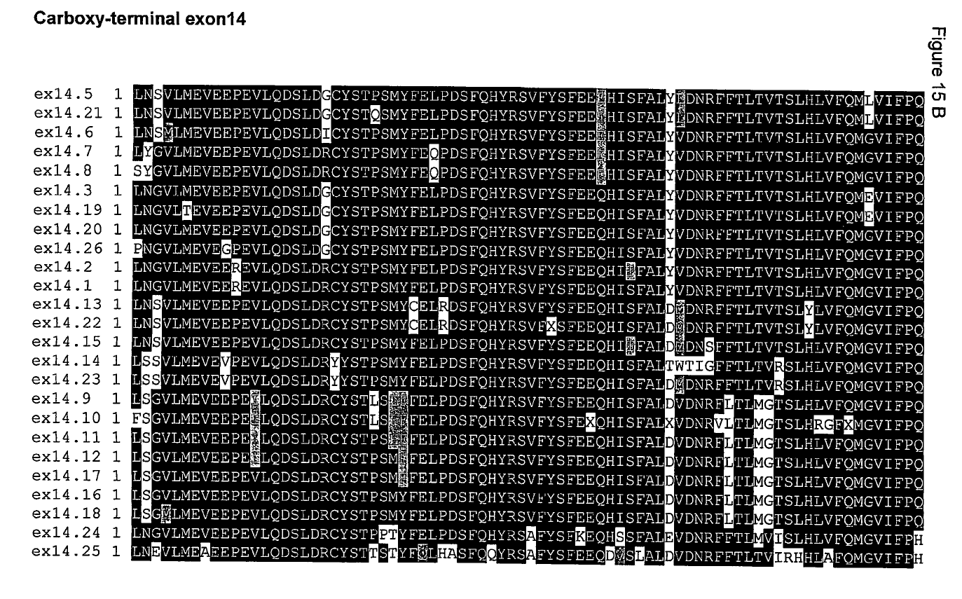

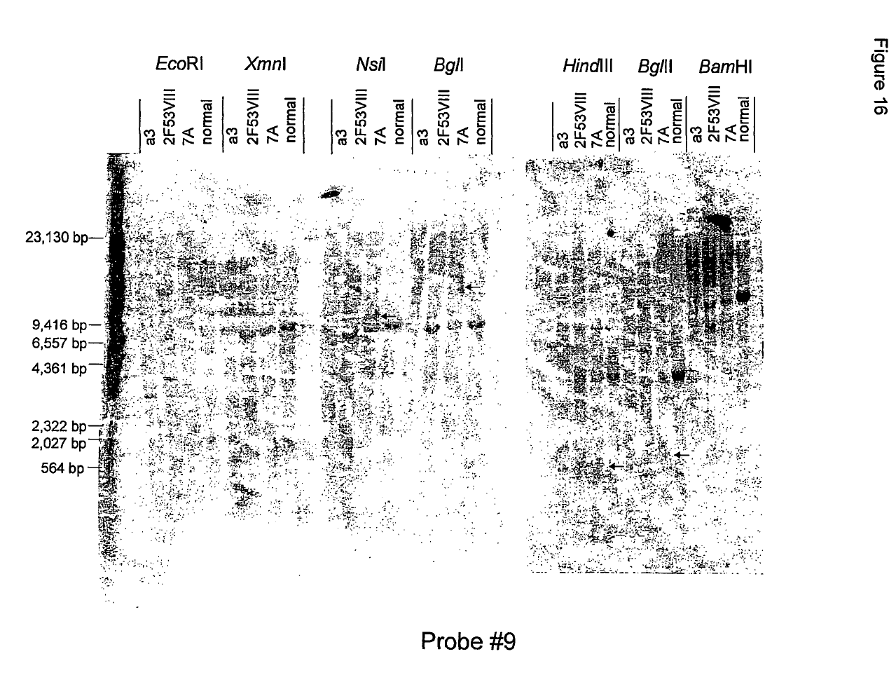

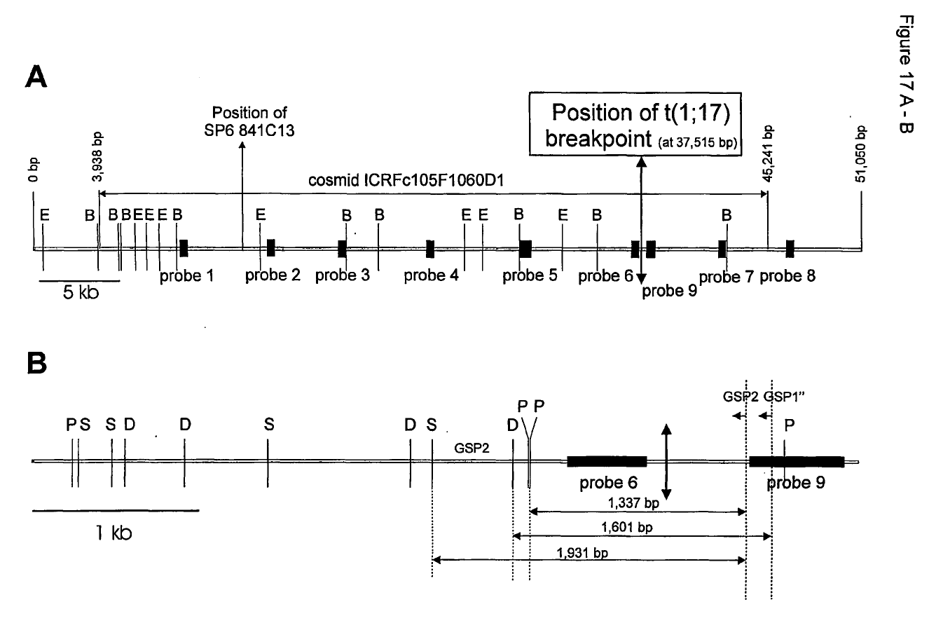

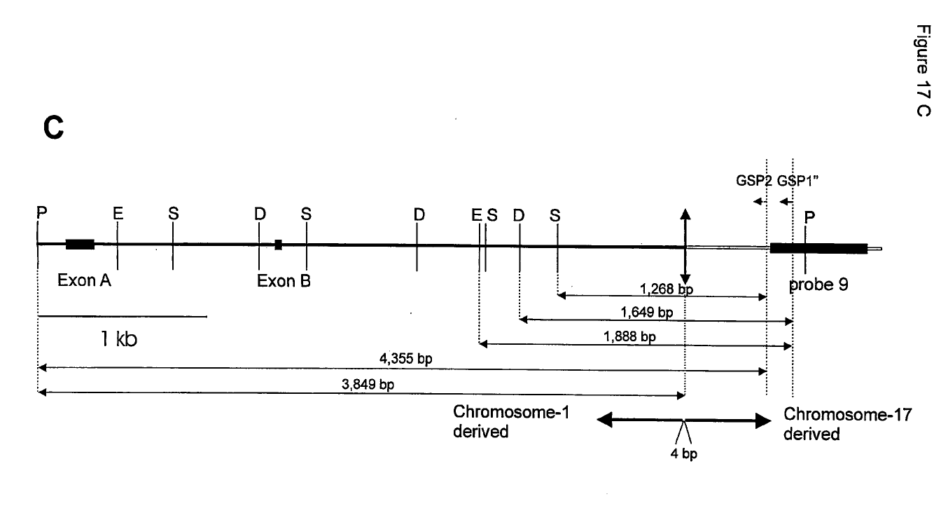

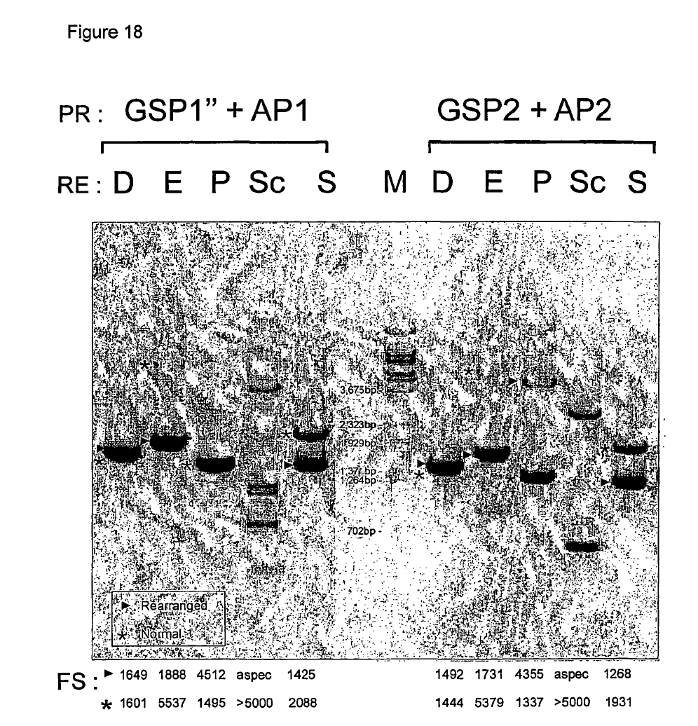

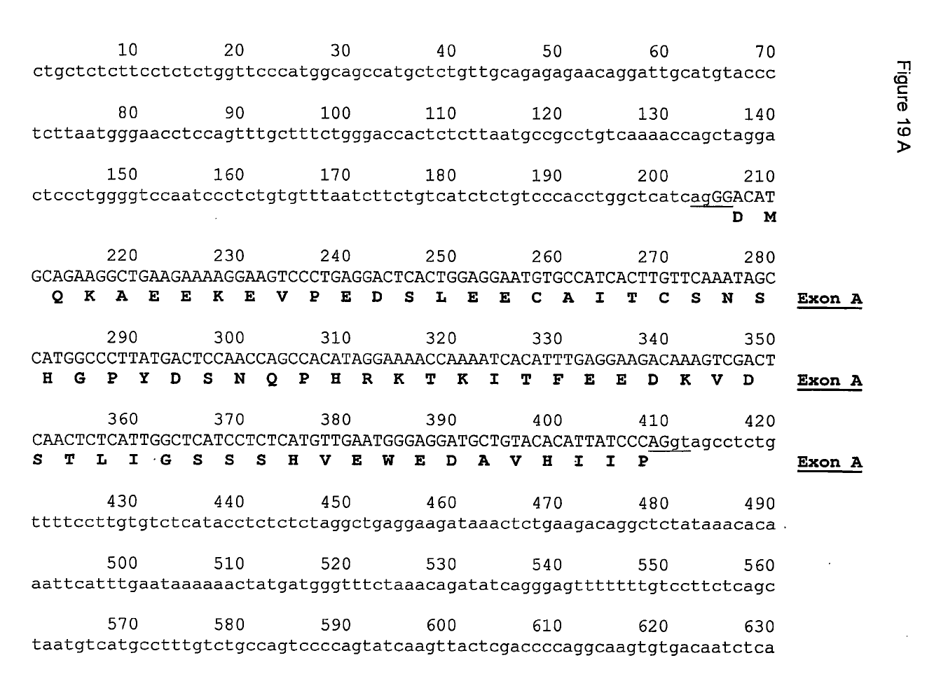

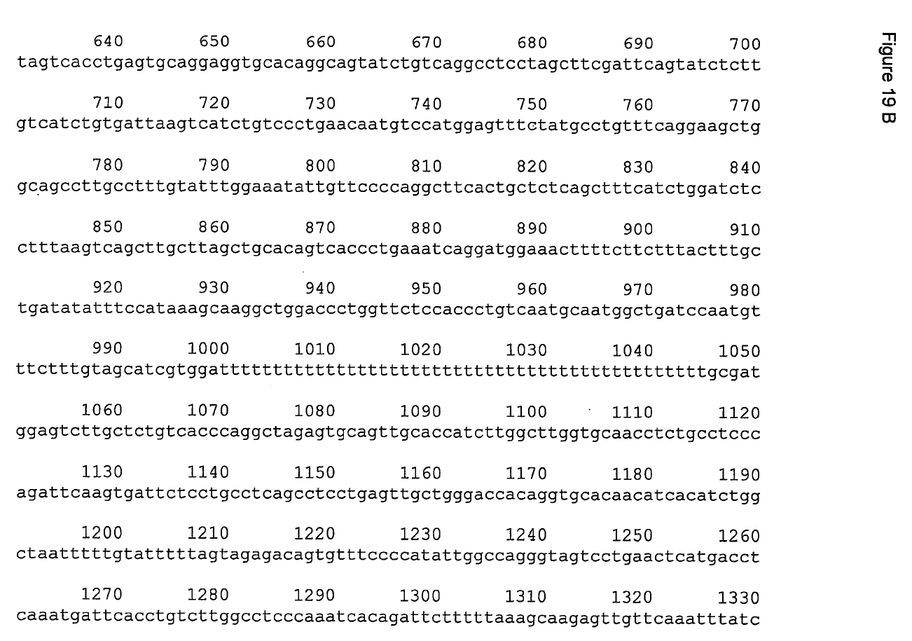

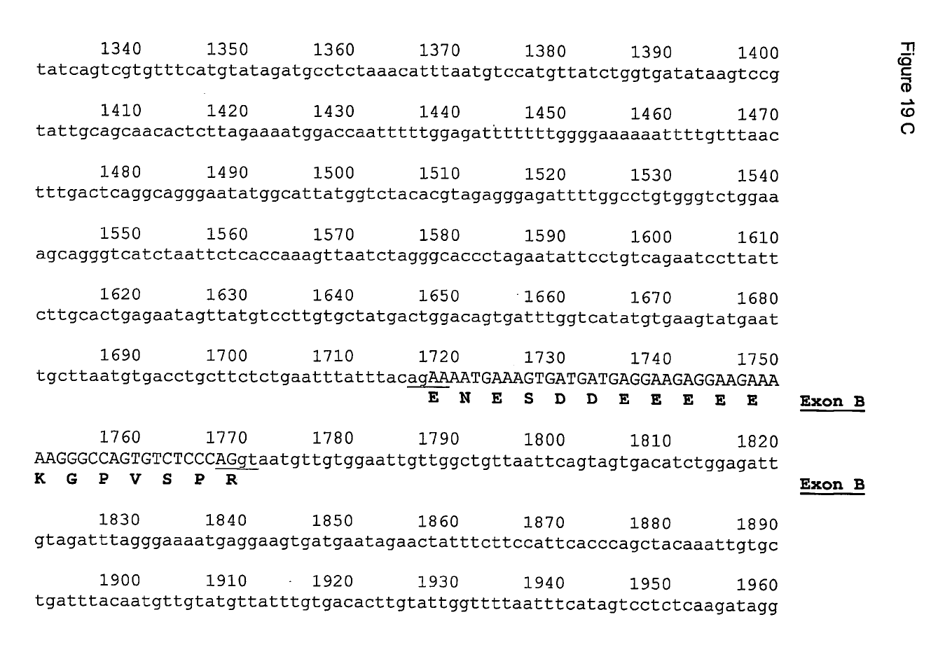

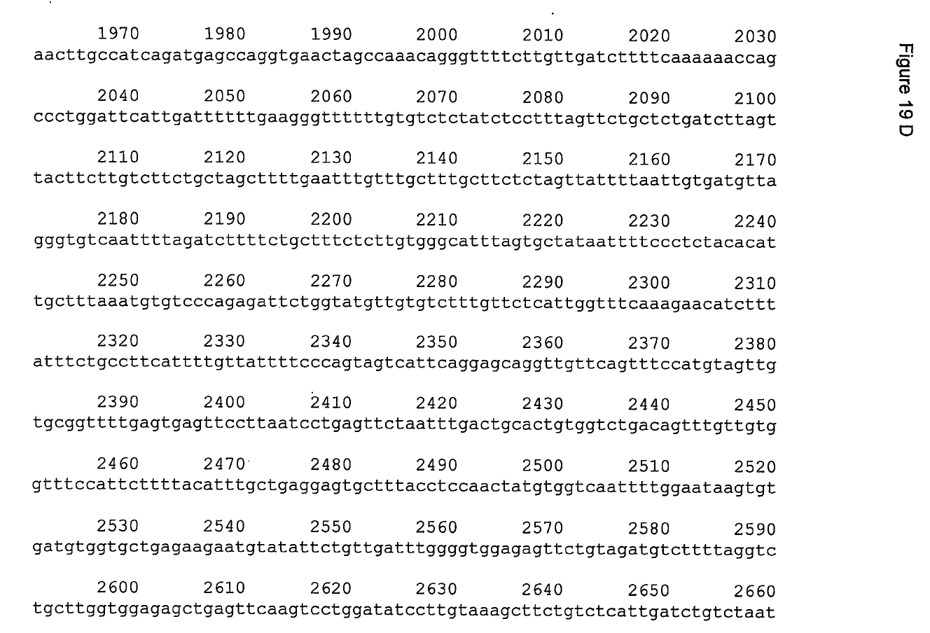

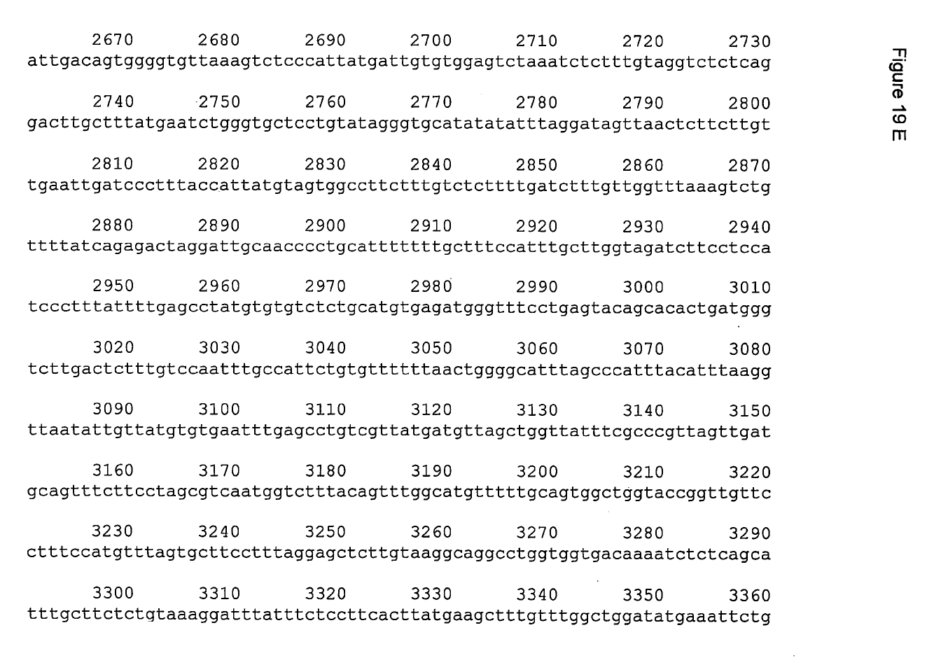

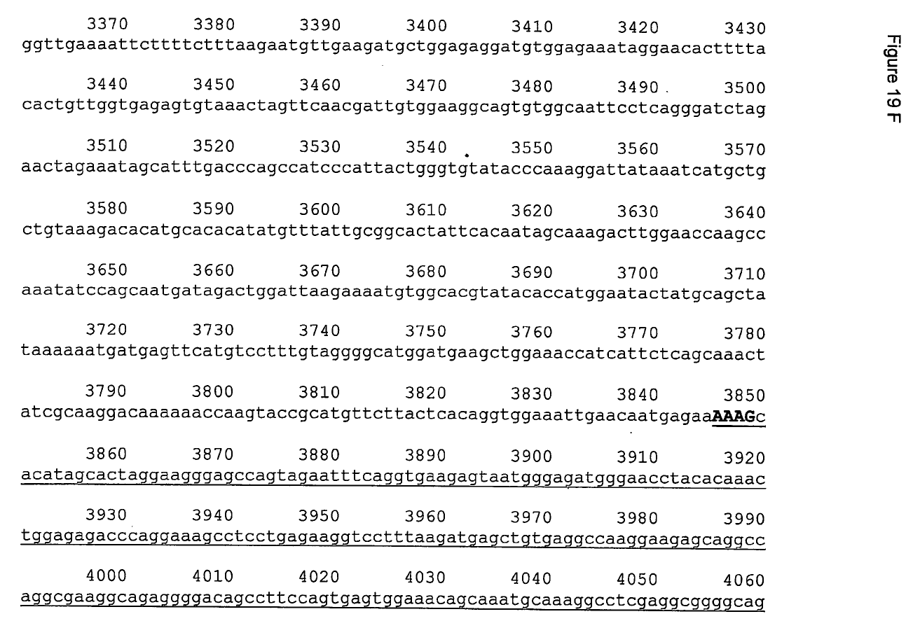

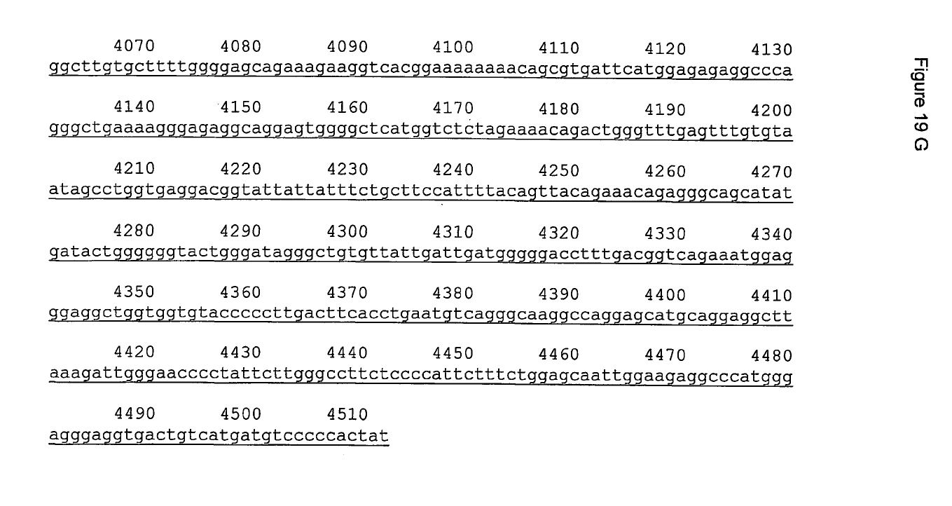

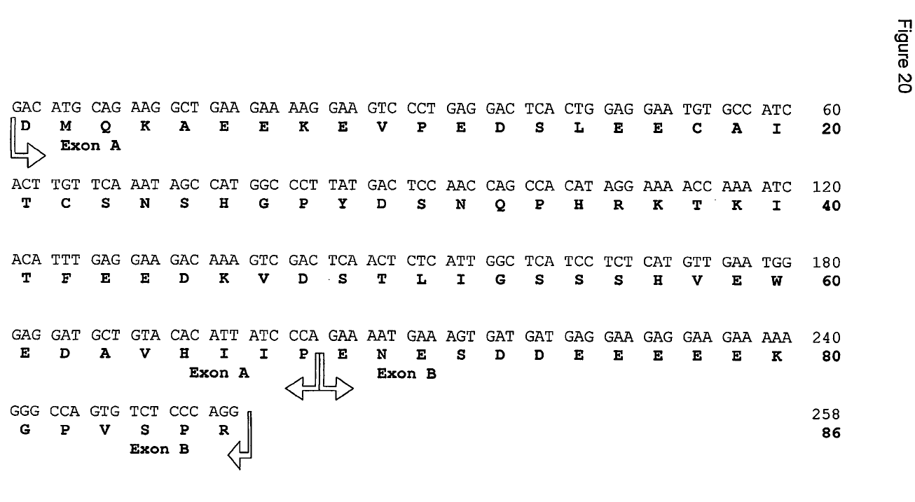

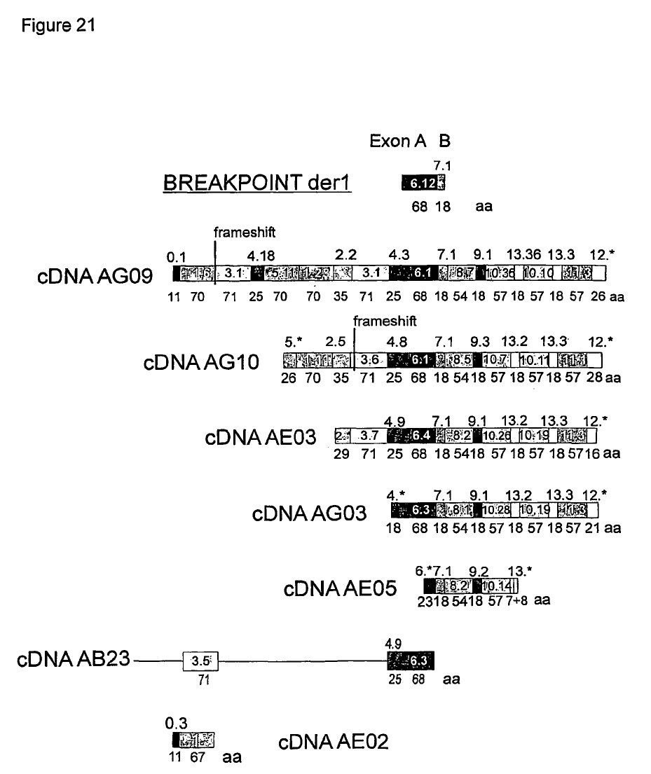

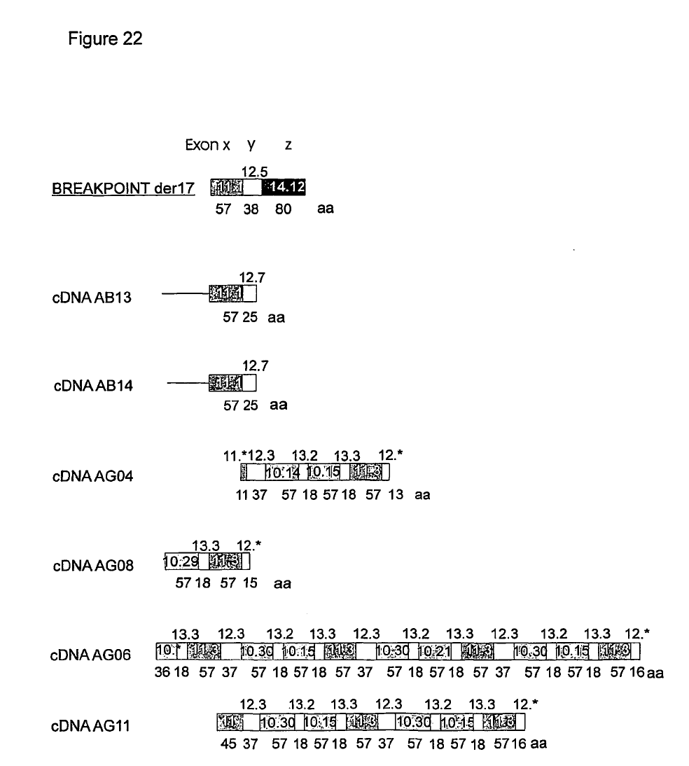

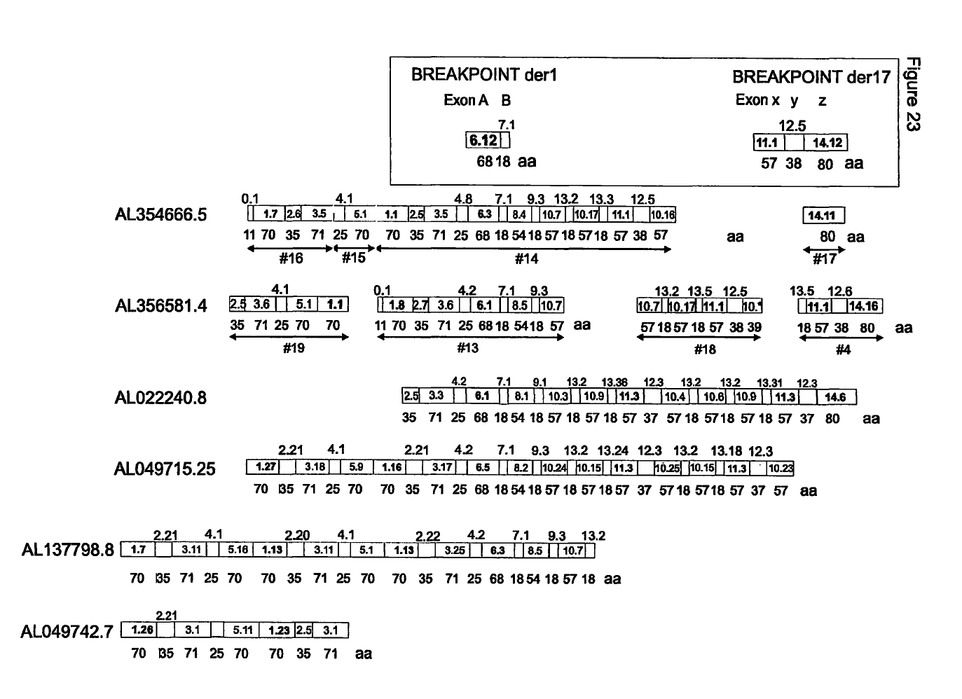

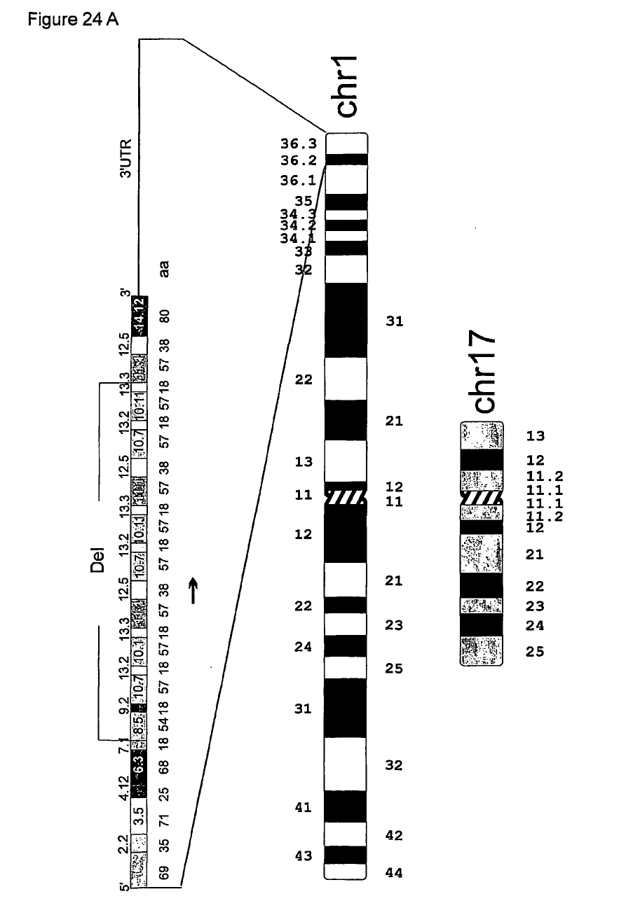

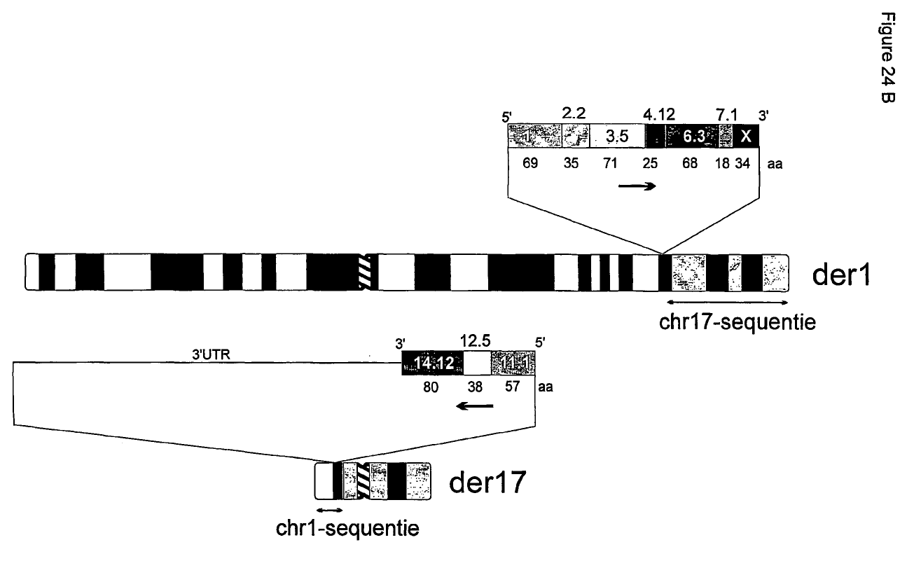

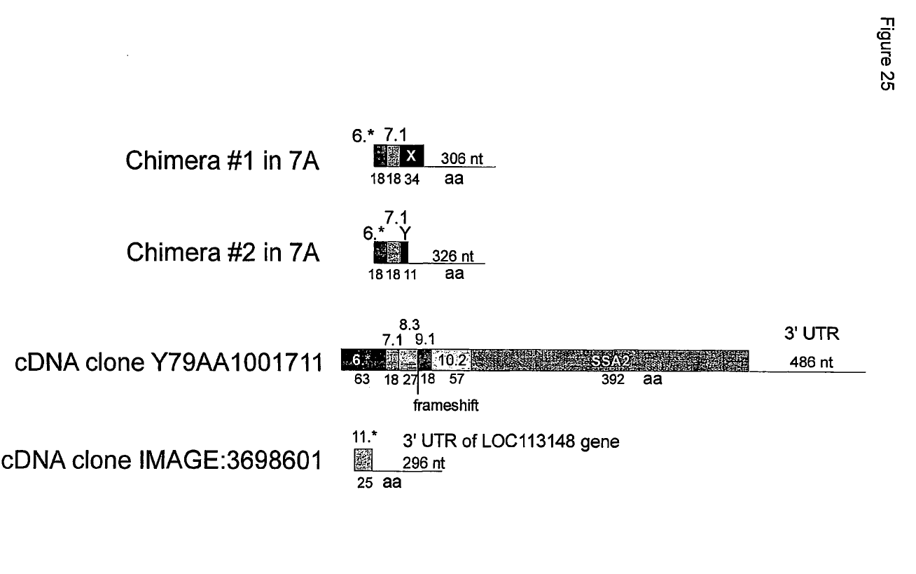

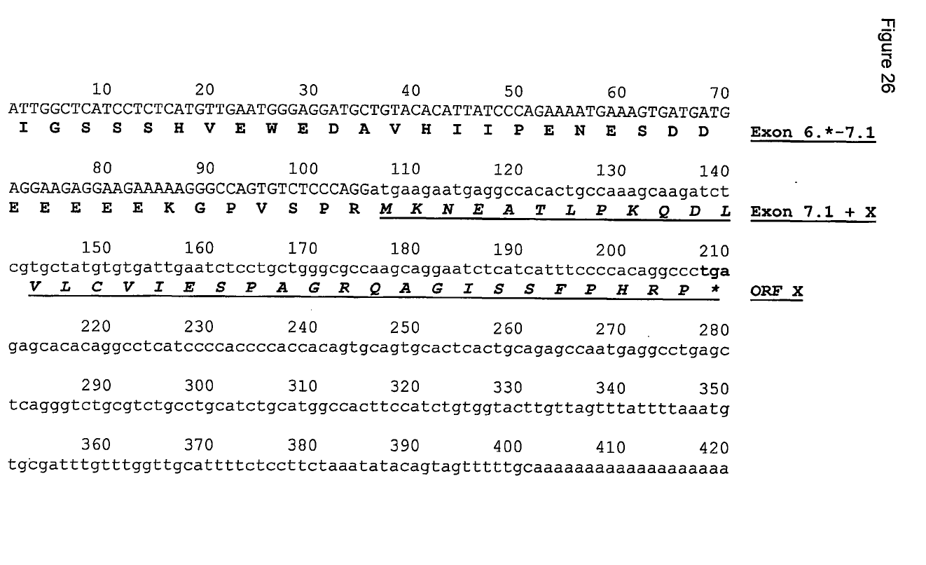

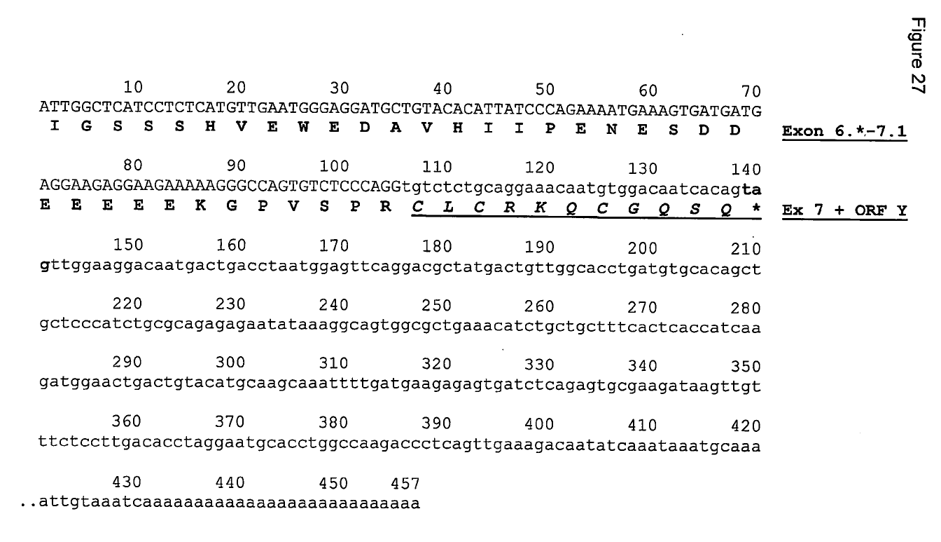

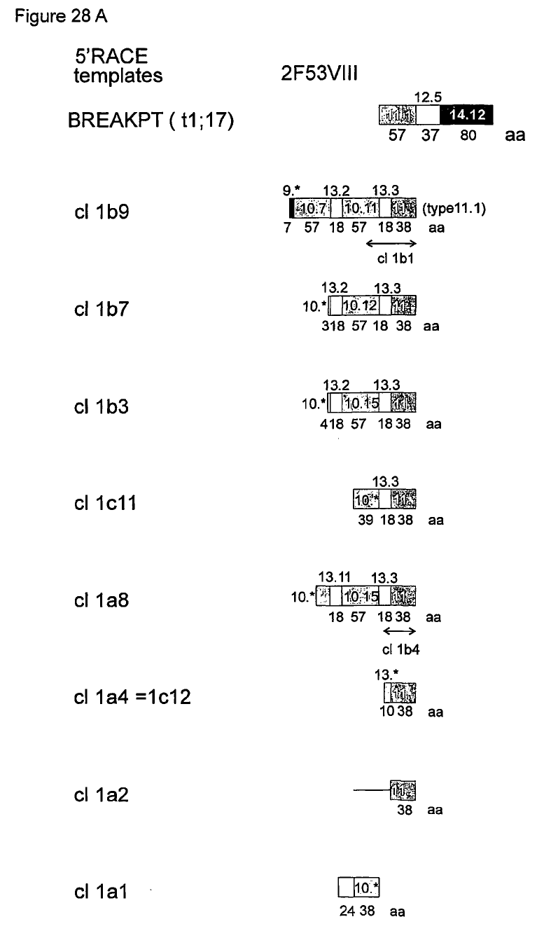

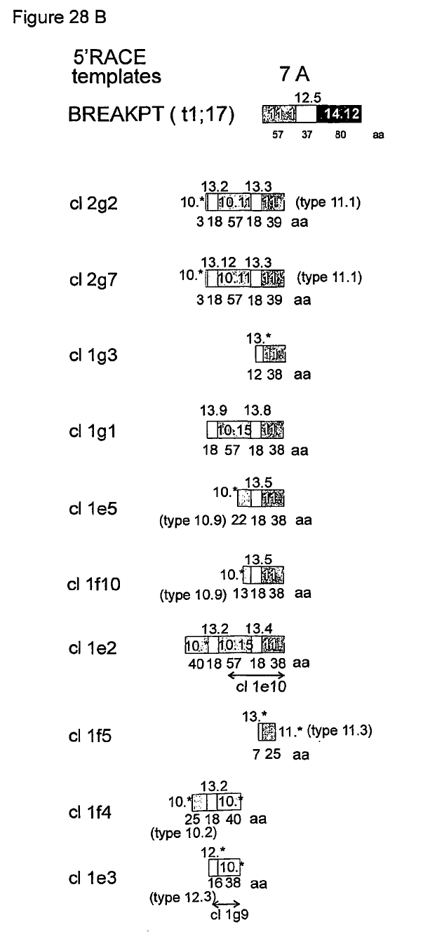

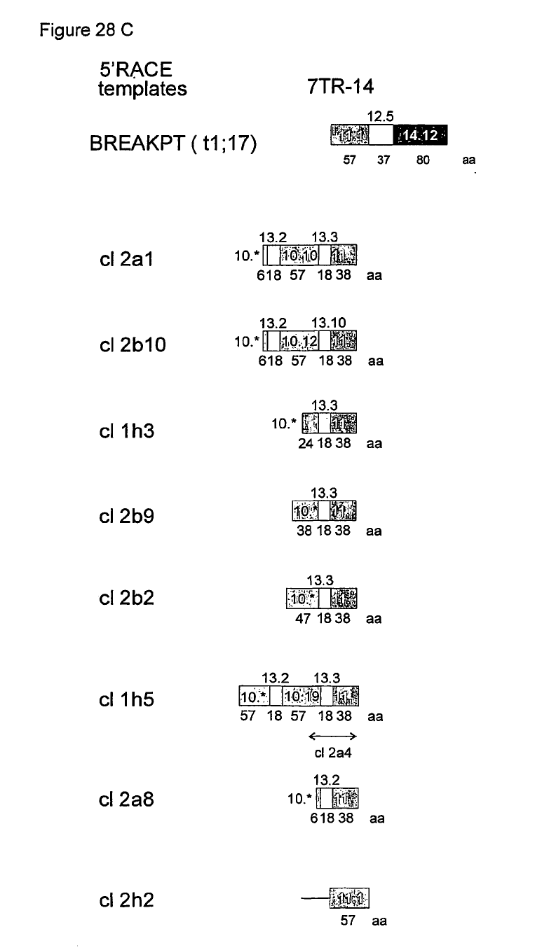

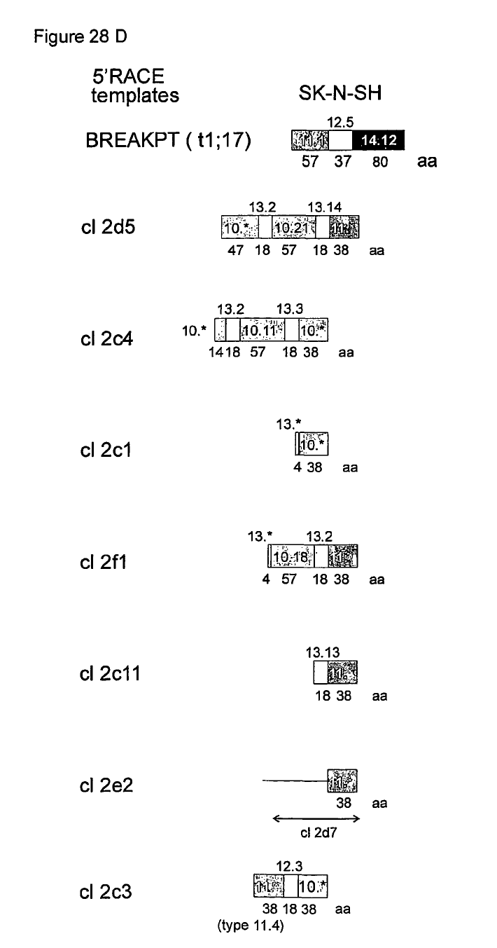

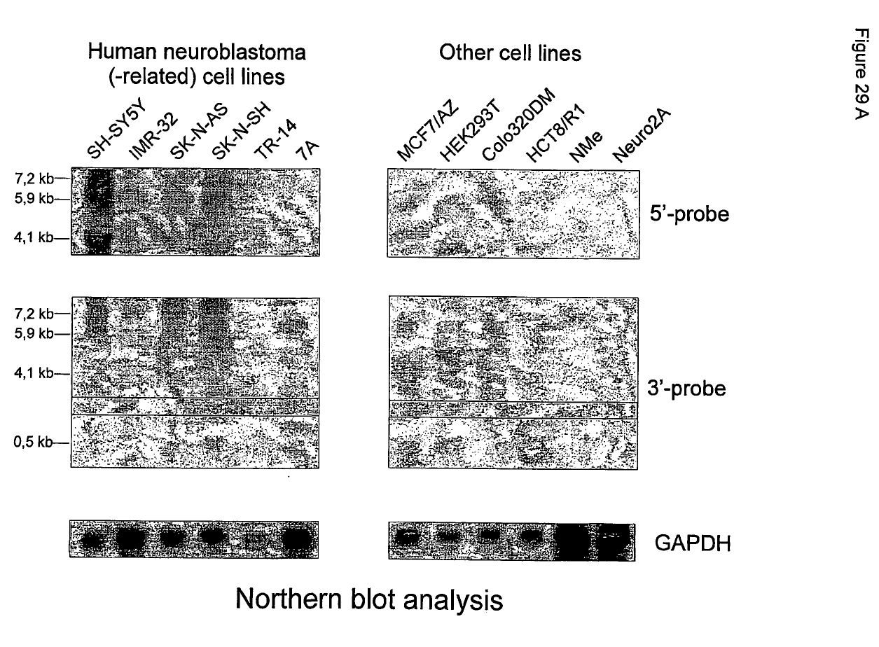

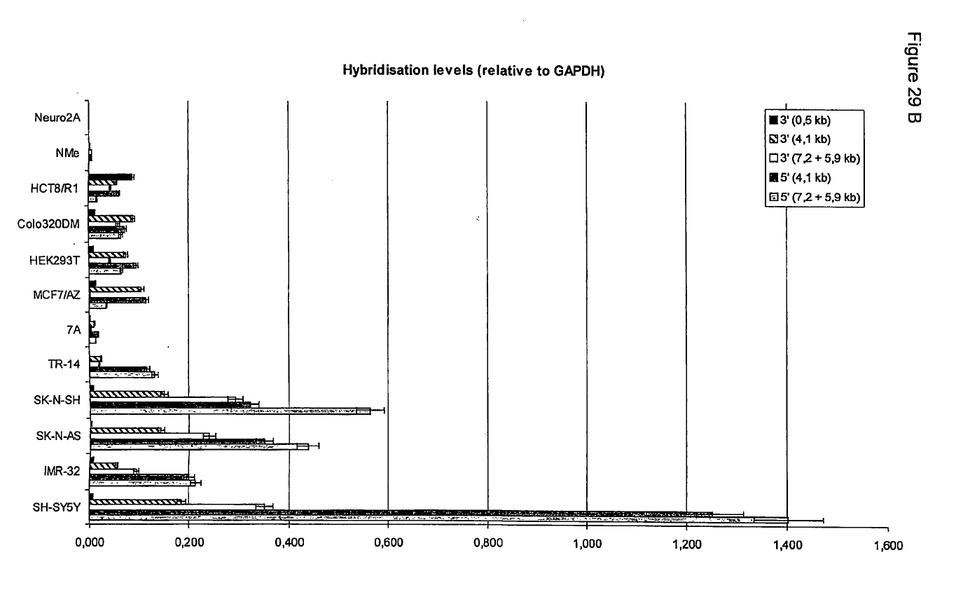

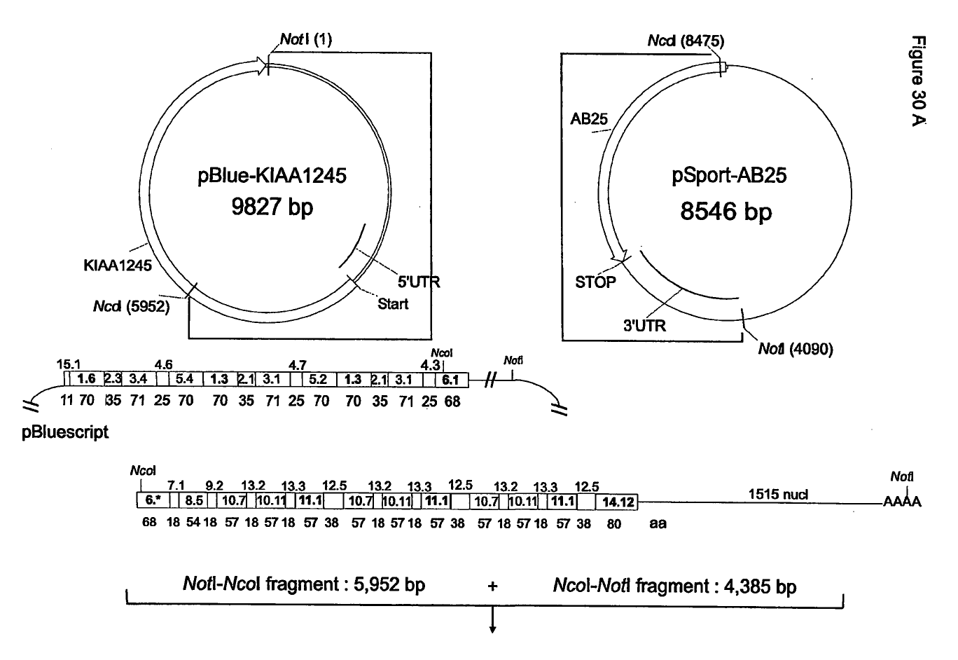

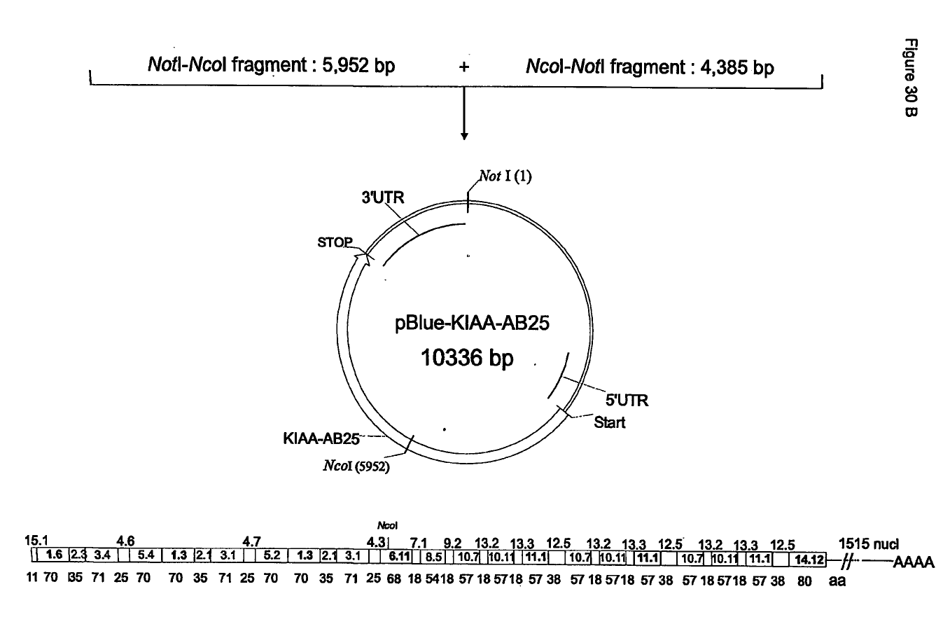

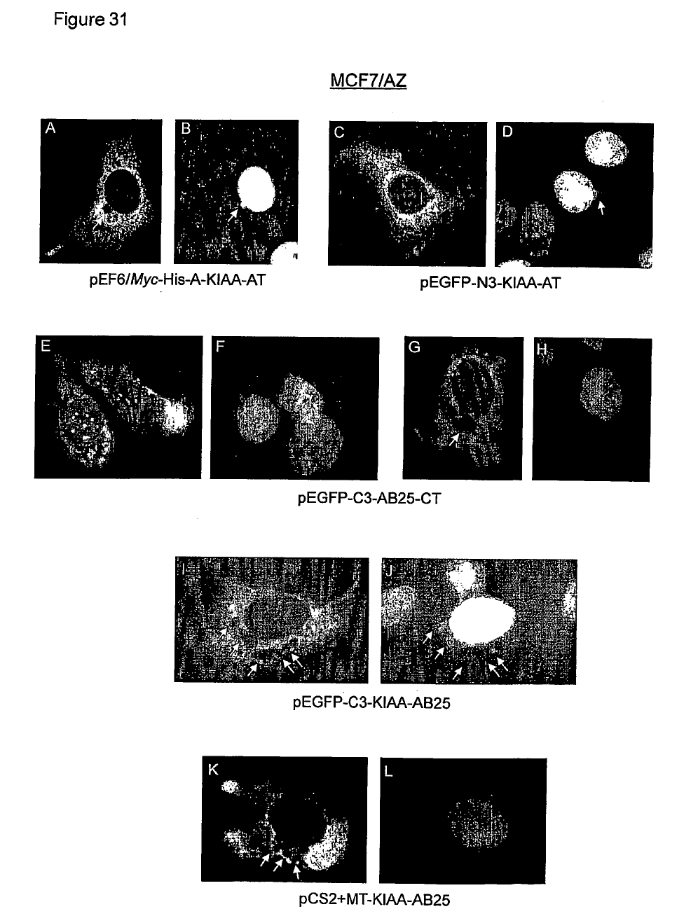

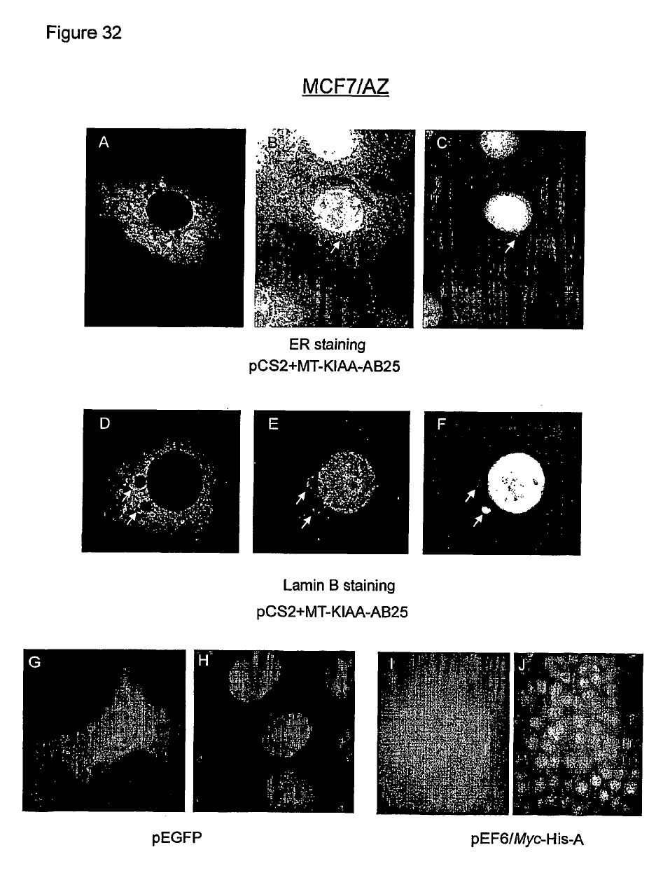

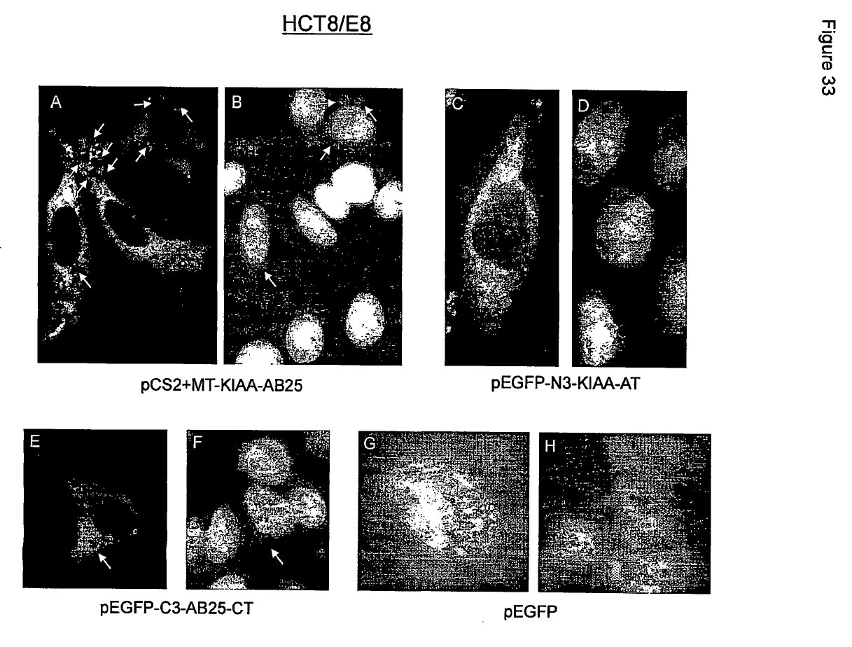

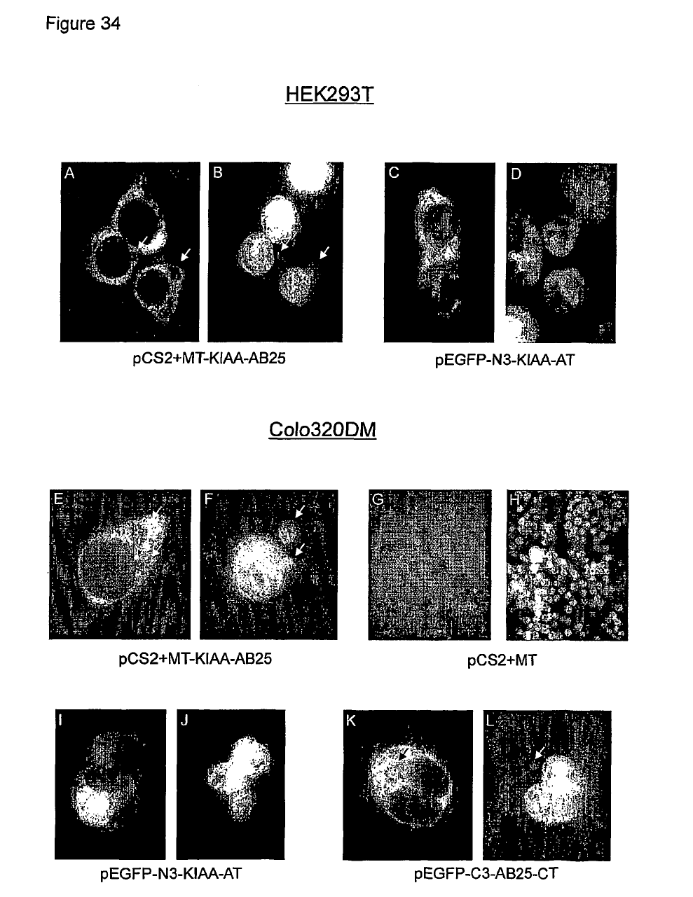

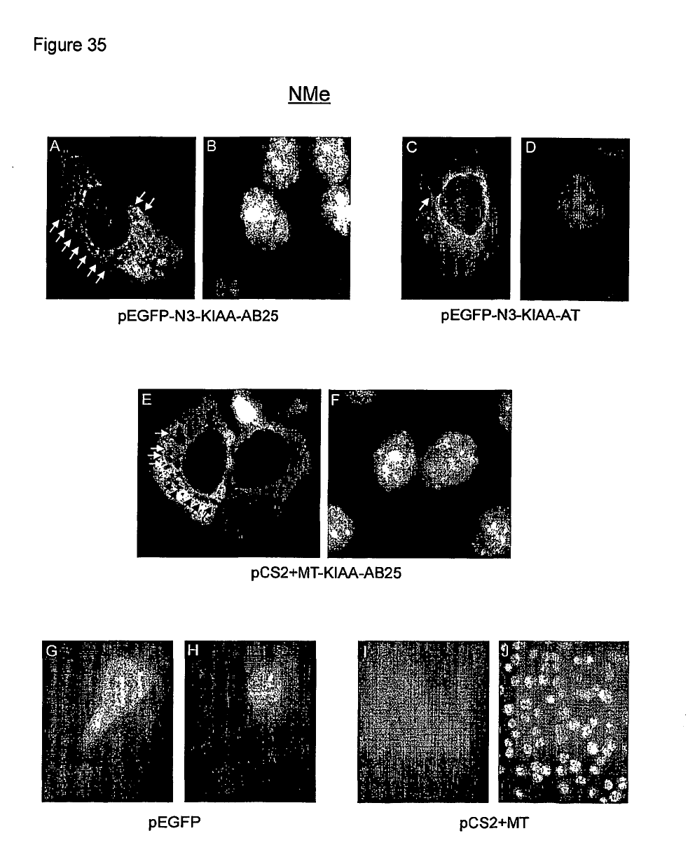

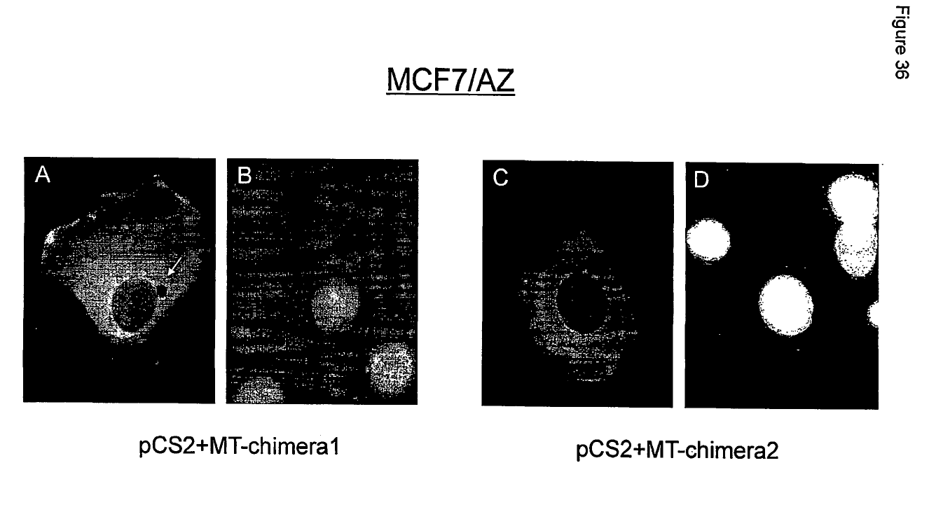

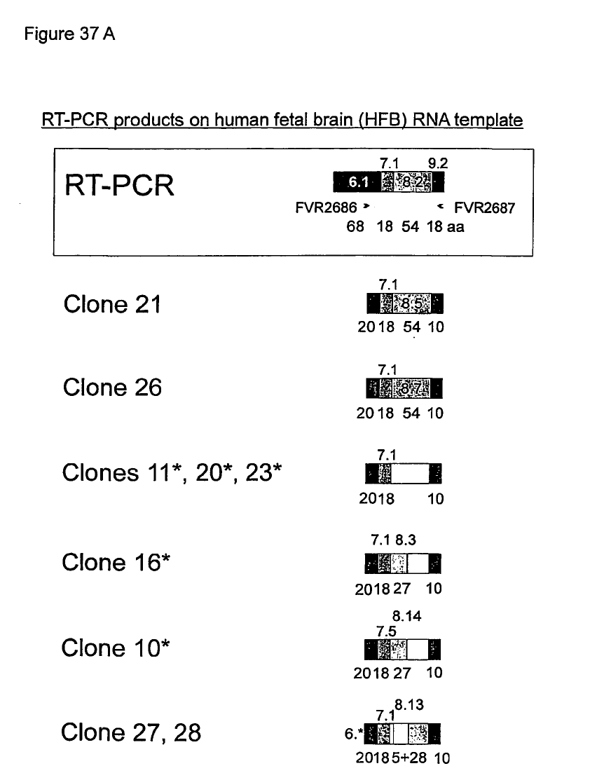

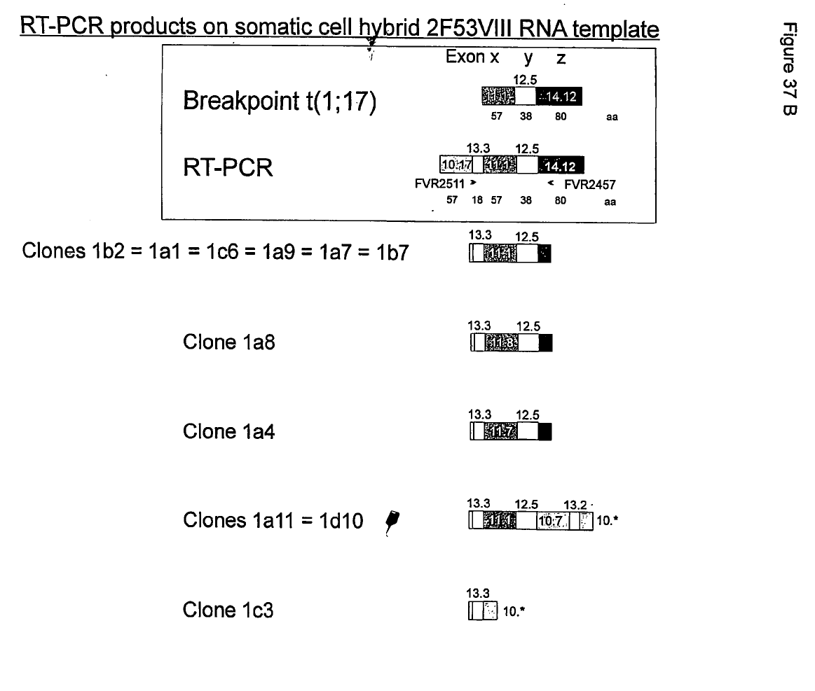

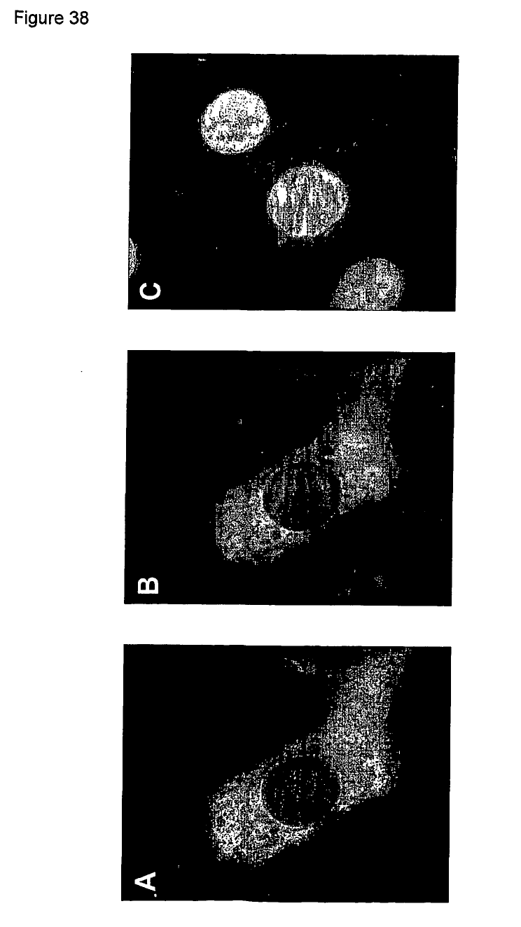

Previous YAC mapping lead to the identification of apparently breakpoint overlapping clones (954-e-11 and 936-g-11) (Van Roy et al., 1997b). However, upon subsequent screening of a chromosome-17 specific cosmid library no breakpoint-overlapping cosmid clones could be identified suggesting that the region covering the breakpoint region was not present in any of the analyzed YAC clones. To overcome this problem, a PAC/BAC contig covering the 17q breakpoint region was constructed (FIG. 1). The RCPI-1 PAC library was screened with end clones (Table 2) from YACs 725-g-2, 728-f-1, 15FD11, 776-d-7 and 681-c-3, cosmid clone cCI17-1079 (Van Roy et al., 1997b) and STS markers D17S1850, D17S1656 (WI-2906), D17S798 and PAC end clones to further close the remaining gaps. Additional STSs were generated from end sequences of the PACs, that covered the breakpoint or were located within its immediate vicinity (Table 1). BLAST searches with PAC end sequences allowed the identification of partially or completely sequenced BAC clones. Further analysis of the partial maps from the Map Viewer at NCBI (http://www.ncbi.nlm.nih.gov/) and from the Human Genome Project Working Draft at UCSC (http://genome.ucsc.edu/) allowed the identification of additional BACs in the breakpoint region. A 1.4 Mb contig was constructed by STS (including newly developed STSs) and EST content mapping by hybridization of PCR products on dot blots containing the PAC clones and/or PCR analyses of PAC and BAC clones. Further careful positioning of all clones was done by fiber FISH. The minimum tiling path of the contig was determined as: centromere * RPCI-11 29G21 * RPCI-11 421O15 * RPCI-11 205P19 * RPCI-1 105C17 * RPCI-1 733P21 * RCPI-1 880L8 * RPCI-1 952I18 * RPCI-1 243F6 * RPCI-1 682F9 * RPCI-1 194A10 * RPCI-1 55N16 * RPCI-1 257G22 * telomere (FIG. 1). PCR content mapping showed that the order of the markers assigned to BAC clone AC011824 had to be inversed. Despite the progress in the mapping of the human genome, inconsistencies exist with regard to the position of markers and genes within this region as reflected by the different and changing mapping information available at the NCBI and UCSC databases. Ordering of contigs and closing of gaps between contigs, as described here, will be crucial in the construction of the final human genome map.

### Example 2

### Identification of Breakpoint Overlapping Clones

The position of the PAC/BAC clones with respect to the constitutional 17q breakpoint, was analyzed using FISH on metaphases from somatic hybrid cell lines, containing the derivative chromosomes 1 and 17. A total of 21 and 18 PAC/BAC clones mapped to the derivative chromosome 1 or derivative chromosome 17, respectively. Using this FISH approach, four PACs and one BAC were shown to be breakpoint overlapping (RPCI-1 880L8, RPCI-1 624A6, RPCI-1 1020I1, RPCI-1 1167N14 and RPCI-11 118G23) (Table 2). PAC clone RPCI-1 880L8 was used to screen a chromosome 17 specific cosmid library. Eleven positive cosmid clones including three overlapping cosmids were identified (ICRFc105F1060D1, ICRFc105F1065D1 and ICRFc105G3072D1) (Table 2). The DNA of these three mutually overlapping cosmids was largely sequenced after shotgun subcloning (see Example 3).

### Example 3

### Shotgun Sequencing of a Cosmid Contig on Chromosome 17 Overlapping the Constitutional 17q Breakpoint

After shotgun cloning and sequencing, we constructed a sequence contig of three chromosome-17 specific mutually overlapping cosmids (ICRFc105F1060D1, ICRFc105F1065D1, ICRFc105G0372D1) encompassing the translocation breakpoint. A total contiguous sequence of 51,050 bp was obtained (GenBank Acc. N° AF148647). Within this sequence, the end clone sequence SP6 841C3 was detected (position 13112-13806), but not these of SP6 829O10 or SP6 835C10 (FIG. 1 and FIG. 3A). Further, these cosmid sequences showed identity with one unfinished BAC sequences of the Human Genome Project (Genbank Acc. No. AC013739, version .2) and with two complete BAC sequences (GenBank Acc. No. AC011824, version .8-GI:13940712; and AC024614, version .5-GI:14575793).

So far, BLAST analysis did not show any evidence for the presence of exons or single-exon genes in these chromosome-17 sequences. However, the exon prediction algorithms GeneMark (URL http://opal.biology.gatech.edu/GeneMark) predicted several putative exons of which one was cloned by exon trapping (see annotation in GenBank Acc. No. AF148647). One EST cluster (stSG50857, Unigene cluster Hs.125747) was located at a maximum distance of ˜180 kb proximal to the 17q breakpoint. Sequence analysis of 4 cDNA clones (IMAGE clones 742727, 2356570, 1471272, 1471196; Genbank Acc. No. AF381171-AF381174) showed alignment of three putative exons with the genomic sequence of BAC clone RPCI-11 31122. On the basis of the stop codons observed, several possibilities can be proposed: either these ESTs represent a multi-exonic 3′UTR, or they represent a transcribed multi-exonic pseudogene with ORF disrupting mutations, or a short open reading frame starts in the second exon, encoding a polypeptide of 78 amino acid residues. The latter polypeptide does not show significant homology with hitherto known proteins or protein domains. BLAST analysis, without masking repeat sequences, of the breakpoint overlapping cosmid sequences against the human EST database showed the presence of two partially overlapping EST sequences (GenBank Acc No AI934614 and AI571839). Although both ESTs contained a LTR/MaLR repeat they show 100% sequence similarity to the cosmid sequence.

### Example 4

### Identification of Rearranged Genomic Fragments Comprising the Constitutional t(1;17) Breakpoint

To facilitate the identification and cloning of the t(1;17) breakpoint, genomic DNA from the somatic cell hybrids 32-7A and 32-2F53VIII, the Chinese hamster ovary cell line a3 and a normal human placenta were digested with a panel of restriction enzymes including BglII, HindIII, KpnI, NcoI, PvuII, XbaI, EcoRI, NsiI and BglII. The somatic cell hybrids 32-7A and 32-2F53VIII contain, respectively, derivative chromosomes 1 and 17 from the original neuroblastoma patient. From the cosmid contig sequence, overspanning the translocation breakpoint, 9 DNA probes free of repetitive sequences were selected and PCR amplified. These probes are listed in Table 4. They were then used for hybridization to the digested genomic DNA in order to search for abnormal patient-specific hybridization bands. No aberrant migrating bands could be detected with probes #3, #4 or #5. However, upon using probe #6, additional bands were identified in the genomic DNA of 32-2F53VIII but not in any of the other DNAs, indicating that these fragments spanned the breakpoint on chromosome 17q (FIG. 2). The smallest rearranged band detected with probe #6 was estimated to be approximately 4 kbp long and was found in the PvuII digested genomic DNA.

Upon use of probe #9, additional bands were identified in the genomic DNA of 32-7A but not in any of the other DNAs, indicating that these fragments spanned the breakpoint on chromosome 1p (FIG. 16). Rearranged bands detected with probe #9 were found in genomic DNA of 32-7A cells upon digestion with EcoRI (˜19,500 bp instead of ˜13,500 bp), NsiI (˜8,800 bp instead of ˜7,600 bp), BglI (˜13,200 bp instead of ˜7,950 bp), HindIII (˜1,000 bp instead of ˜3,300 bp) and BglII (˜1,100 bp instead of ˜3,300 bp).

### Example 5

### Cloning of the Constitutional t(1;17) Translocation Breakpoint in the Somatic Cell Hybrid 32-2F53VIII

GenomeWalker PCR (Siebert et al., 1995) was used to clone the unknown genomic chromosome-1 sequences that have been juxtaposed to known, chromosome-17 sequences as a result of the human t(1;17) chromosomal translocation in the somatic cell hybrid line 32-2F53VIII. In brief (FIG. 3), genomic DNA from cell line 32-2F53VIII was digested with blunt-end generating restriction enzymes PvuII, StuI, EcoRV and DraI, and the resulting DNA fragments were then separately ligated to compatible GenomeWalker adaptors to produce four libraries. PCR amplification was performed using gene-specific primers (GSP), localized in the probe #6 sequence in combination with a GenomeWalker adaptor-specific primer (API). As pointed out above, the chromosome-17 specific probe #6 hybridizes to a 4-kbp PvuII fragment encompassing the (1,17) breakpoint. During the first cycle of the PCR, the GSP primer anneals and one strand of the adaptor is copied. Due to the lack of the binding site for the AP1 primer in the 5′-extended adaptor in combination with the 3′ end of the adaptor being blocked with an amine group to prevent extension, the AP1 primer anneals only to the newly synthesized strand and not to the adaptor-ligated primary template genomic DNA. If rare extension of the 3′ end of the adaptor should occur, efficient panhandle formation suppresses nonspecific background amplification. In consequence, only the genomic DNA fragment containing the specific GSP sequence is amplified.

Primer pair GSP1 plus AP1 was used in a first round of amplification. Specific products of 1,457 bp and 3,575 bp were amplified from, respectively, the 32-2F53VIII/StuI and 32-2F53VIII/PvuII GenomeWalker libraries. With the GSP1′ plus AP1 primer combination, products of 1,029 bp and 3,459 bp were amplified from, respectively, the 32-2F53VIII/DraI and 32-2F53VIII/EcoRV.GenomeWalker libraries (FIGS. 3 and 4). All four 32-2F53VIII-specific products mentioned were cloned and fully sequenced. Overlapping novel sequences, derived from chromosome 1, were present in all clones. In total, a maximum of 3,141 bp of novel sequences were identified in the 32-2F53VIII/EcoRV clone (FIG. 5; sequence deposited with GenBank under Acc No AF379607). Of these, 3,134 bp were found to be chromosome-1 specific (FIG. 3; see Example 7). As indicated in FIG. 3, the other clones contained shorter but completely overlapping sequences.

### Example 6

### Cloning of the Constitutional t(1;17) Translocation Breakpoint in the Somatic Cell Hybrid 32-7A

GenomeWalker PCR (Siebert et al., 1995) was used to clone the unknown genomic chromosome-1 sequences that have been juxtaposed to known, chromosome-17 sequences as a result of the human t(1;17) chromosomal translocation in the somatic cell hybrid line 32-7A. In brief (FIG. 17), genomic DNA from cell line 32-7A was digested with blunt-end generating restriction enzymes DraI, EcoRV, PvuII, ScaI and SspI, and the resulting DNA fragments were then separately ligated to compatible GenomeWalker adaptors to produce five libraries. PCR amplification was performed using gene-specific primers (GSP), localized in the probe #9 sequence in combination with a GenomeWalker adaptor-specific primer (API). As pointed out above (FIG. 16), the chromosome-17 specific probe #9 hybridizes to various restriction fragments encompassing the (1,17) breakpoint as their mobility is shifted in DNA digests of the 32-7A cell line as compared to the 32-2F53VIII cell line or normal human placenta.

Primer pair GSP1″ plus AP1 was used in a first round of GenomeWalker amplification, as outlined above in Example 5. Specific products of 1,649 bp and 1,888 bp were amplified from, respectively, the 32-7A/DraI and 32-7A/EcoRV GenomeWalker libraries. With the GSP2 plus AP2 primer combination, products of 1,268 bp and 4,355 bp was amplified from the 32-7A/SspI and 32-7A/PvuII GenomeWalker libraries (FIGS. 17 and 18). All four 32-7A-specific products mentioned were cloned and fully sequenced. Overlapping novel sequences, derived from chromosome 1, were present in all clones. In total, a maximum of 3,853 bp of novel sequences were identified in the 32-7A/PvuII clone (FIG. 19; sequence deposited with GenBank under Acc. No. AF379606). Of these, 3,849 bp were found to be chromosome-1 specific (see FIG. 17; Example 7). As indicated in FIG. 17, the other clones contained shorter but completely overlapping sequences.

### Example 7

### Analysis of Sequences Flanking the Constitutional t(1;17) Translocation Breakpoint

Sequences generated from both sides of the t(1;17)(p36.2;q11.2-q12.1) breakpoint in the derivative chromosome-17 and derivative chromosome-1 were analyzed for the presence of putative protein-encoding gene sequences by using both BLASTN and BLASTX to search public databases. This demonstrated that the breakpoint-flanking sequence adjacent to the chromosome-17 sequences were chromosome-1 derived as they show almost perfect identity with several unfinished or finished sequences of BAC clones (Genbank Acc. N° AL354666, AL356581, AC015618, AL355149, AL355800, AL022240, AL049715, AL137798 and AL049742), all mapping to chromosome 1. Regarding chromosome-1 sequences on derivative chromosome 17 (der17), high to perfect homology was also found between parts of this genomic sequence and at least eleven EST sequences (GenBank Acc. N° AL040932, AL045522, AA322028, AL043132, AA609104, AA350323, AL042839, AL044108, AL043174, AL037724 and AW160820) (Tables 5 and 6, and FIG. 7). This confirmed the prediction of three consecutive exons, designated x, y and z as indicated in FIGS. 3, 5 and 6. These exons x to z encode a carboxy-terminal fragment of a novel protein. This protein shows no identity with so far known gene products. The extended size of this particular protein, and of highly related new proteins, became apparent upon sequencing the full-size inserts of the cDNA clones represented by the EST sequences mentioned (summarized in FIG. 7, Tables 5 and 6). Corresponding nucleotide sequences were deposited with GenBank (Table 6). The predictable sequences of the protein domains, encoded by exons of the x, y or z type, are aligned in FIGS. 13 to 15. Regarding breakpoint-flanking chromosome-1 sequences on derivative chromosome-1, high to perfect homology was found between parts of this genomic sequence and the sequences of at least six cDNA clones (EST sequences deposited with GenBank under Acc. N° AI570017, AW173183, H06312, AW468059, AA704208, AI239884). Full-size insert sequences were determined and deposited with GenBank (summarized in FIG. 21, Tables 5 and 6). This confirmed the prediction of two consecutive exons, designated A and B as indicated in FIGS. 17, 19 and 20. The predictable sequences of the protein domains, encoded by exons of the A or B type, are aligned in FIGS. 11 and 12. These exons A and B encode an internal fragment of a novel protein, belonging to the same family as the protein with the carboxy-terminal sequence encoded by exons x, y and z, discussed above. This is apparent both from cDNA sequences, notably the full-size sequences of the cDNA clones depicted in FIG. 7 and FIG. 21, and from genomic sequences (see Example 8).

### Example 8

### Chromosome-1 Derived Exons Flanking the t(1;17) Translocation Breakpoint Belong to a Large and Novel Gene Family NBG

High homology was found between on the one hand the exons x, y and z (FIGS. 5 and 6) and the exons A and B (FIGS. 19 and 20) and on the other hand many more EST sequences than the one mentioned in Example 7 (e.g. Genbank Acc. N° AL037724, AI909921, AA705685, AA701673, AI570017, AW173183, AA350323, AI372468, F11837, AI049567, AW238577, AI537172, AI953463, . . . )(Tables 5 and 6). This became further clear upon sequencing the full-size inserts of cDNA clones represented by several of the additional EST sequences mentioned (summarized in FIG. 22, Tables 5 and 6). High homology was observed also between on the one hand exons A, B, x and y, and on the other hand two cDNA clones (Acc N° AK000726 and AB033071) and predictable exons of one genomic sequence AL049715 and this was particularly obvious upon comparison of the encoded protein fragments (Table 5, FIGS. 8 and 23). High homology was also found between exons A, B, x and z and the cDNA sequence AB051480; between exons x+y and cDNA sequences AF161426 and AI050141; between exons y-z and cDNA sequence AF131738; and between exons A+B and predictable exons of genomic sequence AL137798 (Table 5, FIGS. 8 and 23). High homology was also found between exons A, B and x to z and the cDNA sequence AL117237, predicted on the basis of the genomic sequence AL022240 (derived from clone 328E19), the cDNA sequence AL136890, and predictable exons of the genomic sequences AL354666, AL356581 and AL022240 (Table 5; FIGS. 8 and 23). When the sequences of the predicted exons in various representative cDNA and genomic clones were aligned with each other, separate classes could be discerned, as shown in Table 5, FIGS. 7 to 15, and FIGS. 21 to 23 (overview in FIGS. 7, 8, 21 to 23; alignments and pedigrees in FIGS. 9 to 15). In these figures, the three exons x to z correspond to, respectively, exon 11.1, exon 12.5 and carboxy-terminal exon 14.12, whereas exons A and B correspond to exons 6.12 and 7.1. On the basis of sequences from cDNA clone DKFZp434G2022 (AB25; Table 6 and FIG. 7) and from genomic clones RP11-284O17 and RP11-45I3 (GeneBank Acc. Nos. AL355800.5 and AC015618.3), we could predict the structure of the transcript interrupted by the chromosomal t(1;17) translocation (FIG. 24). By the translocation event, a genomic segment of about 20 kbp and comprising exons of the types 8 to 13 appears to be deleted (FIG. 24).

Within this new gene family, designated NBG (Neuroblastoma Breakpoint Gene family), 5′ exons of types 0 and 1 (Table 5) were represented by cDNA clone hg04073 (encoding protein KIAA1245; GenBank Acc. No. AB033071; FIG. 8), cDNA clone HEP17004 (GenBank Acc. No. AK000726; FIG. 8), IMAGE cDNA clone 2226413 (clone AG09; Table 6 and FIG. 21), IMAGE cDNA clone 341197 (clone AE02; Table 6 and FIG. 21), genomic clones RP11-87L17 and RP5-1020C22 (GenBank Acc. Nos. AL354666 and AL356581; FIG. 23). A likely start codon is situated in exon type 1 and yields proteins with starting sequence MWSAG . . . (FIG. 9).

Upon comparison of exons A, B, and x to z, which were found to flank the cloned breakpoints of the der1 and der17 chromosomes, with the cDNA and genomic sequences obtained so far, the transcript interrupted by the chromosomal t(1;17) translocation was predicted to have the structure depicted on top in FIG. 24. It corresponds to the sequence of cDNA clone DKFZp434G2022 (clone AB25; FIG. 7 and Table 6; exon types 6.3 and 6.12 are very closely related [cf. FIG. 11] as they differ by only two out of 68 amino acid residues due to two nucleotide differences), completed at the 5′ end by sequences from unfinished genomic clones RP11-284017 (GenBank Acc. No. AL355800) and RP11-45I3 (GenBank Acc. No. AC015618). Upon chromosomal translocation, a genomic region covering one exon of type 8, one exon of type 9, and several exons of type 10, 11, 12 and 13 are lost (FIG. 24). The total sequence that is predicted to be deleted is therefore as large as ˜20 kbp.

The interrupted gene on the der1 chromosome is transcribed into chimeric molecules, as demonstrated by a 3′-RACE experiment on mRNA from the 32-7A somatic cell hybrid. Two chimeric transcripts were cloned in this way. The fusion partner is in both cases located on chromosome 17. In one chimeric transcript, represented by eight distinct clones, the exon-7.1 sequence is spliced to chromosome-17 sequences at 338 nt from the breakpoint, and this yields a transcript extended by 295 additional nucleotides and an ORF extended by 34 additional codons in frame (represented by X in FIGS. 24 and 25; sequence depicted in FIG. 26; deposited with GenBank under Acc. No. AF420438). In a second chimeric transcript, represented by a single clone, the exon-7.1 sequence is spliced to chromosome-17 sequences at 8,602 nt from the breakpoint according to the BAC sequence with GenBank Acc. No. AC024614.3. This yields a transcript extended by 325 additional nucleotides and an ORF extended by 11 additional codons in frame (represented by Y in FIG. 25; sequence depicted in FIG. 27; deposited with GenBank under Acc. No. AF420439). A reciprocal experiment aimed at identifying chimeric transcripts by 5′-RACE from somatic cell hybrid 32-2F53VIII and several more cell lines. Although many gene-specific fragments were detected illustrating the widespread expression of NBG family members (FIG. 28), no chimeric transcripts could be cloned so far from these 5′-RACE experiments.

Unusual features were also observed in several cDNA clones corresponding to this new NBG family: translation frameshifts (e.g. cDNA clones AG07 and AE01 in FIG. 7, cDNA clone AB033071 in FIG. 8, cDNA clones AG09 and AG10 in FIG. 21), and retention of intronic sequences (e.g. cDNA clones AD02 and AB06 in FIG. 7; cDNA clone AI050141 in FIG. 8, cDNA clone AB23 in FIG. 21; cDNA clones AB13 and AB14 in FIG. 22). Such intronic sequences were also found in 5′-RACE products of 32-2F53VIII, TR-14 and SK-N-SH cells (FIG. 28).

We addressed the number of different human NBG genes by checking the occurrence of nonidentical exon-7, -8 and -9 sequences in contiguous genomic sequences of the public-domain human genome resources. To our knowledge, these three exon types occur only once per transcript as exemplified in FIGS. 7, 8 and 21. From the data, listed in Table 7, we conclude that there about 15 different NBG genes, mainly localized on chromosome arms 1p and 1q.

No homologous or orthologous sequences for this novel gene family NBG were found in the genomes of either Saccharomyces cerevisiae, Drosophila melanogaster or Caenorhabditis elegans. The repetitive nature of the transcripts and proteins, encoded by this novel gene family, is particularly striking (FIGS. 7, 8, 22 and 23). Nevertheless, upon screening various protein domain databases, no significant matches were found. These databases include: InterPro (URL http://www.ebi.ac.uk/interpro/), ProSite (URL http://www.expasy.ch/prosite/), Blocks (URL http://www.blocks.fhcrc.org), Prints (URL http://www.biochem.ucl.ac.uk/bsm/dbbrowser/PRINTS/PRINTS.html), PFAM (URL http://qenome.wustl.edu/Pfam), and other databases. In conclusion, this failure to detect known protein domains points at a fully novel protein structure and innovative functions for the gene family members described above.

### Example 9

### Chimeric Transcripts of the Novel Gene Family in Other Tumor Types Besides Neuroblastoma

Analysis of the non-redundant database and the EST-database at NCBI, resulted in the identification of two chimeric transcripts showing homology to to the novel NBG gene family (FIG. 25). The first one, cDNA clone Y79AA1001711 (GenBank Acc. No. AK024044) was derived from the human retinoblastoma cell line Y79, and consists of 552 nucleotides of a NBG family member, followed by 5 unrelated nucleotides and 1,657 nucleotides of the SSA2 transcript (Sjogren syndrome antigen A2; 60-kDa ribonucleoprotein autoantigen SS-A/Ro) (nucleotides 1-1,657 of the GenBank sequence with Acc. No. XM_{—}029851.1), encoded by the Sjogren syndrome gene SSA2 located at chromosome 1q31. The second chimeric transcript, cDNA clone IMAGE:3698601 (complement of sequence with GenBank Acc. No. BF478071.1) was derived from pooled germ cell tumors, and comprises some 75 NBG-specific nucleotides (encoding the exon-11 like peptide sequence TPTSCLEQPDSSQPYGSSFYALEEK), followed by a stretch of 9 A nucleotides, and 288 nucleotides of the 3′ UTR of the transcript encoding human coatomer protein complex, subunit alpha (corresponding to nucleotides 4,735-5,022 of the GenBank sequence with Acc. No. XM_{—}049690.1; gene LOC113148 on chromosome 1q).

### Example 10

### NBG mRNA Expression Analysis

A Northern blot was hybridized with two separate NBG-specific probes. In the first hybridisation, a 5′-specific probe was used. This resulted in a strong signal in the neuroblastoma cell lines SH-SY5Y, IMR-32, SK-N-AS and SK-N-SH, but no detectable signal in TR-14, which contains double minutes (FIG. 29, upper panel). Human non-neuroblastoma cell lines (MCF7/AZ, HEK293T, Colo320DM, and HCT8/R1) showed lower expression levels. No detectable signal was seen for mouse cell lines NMe and Neuro2A. When a 3′-specific probe was used, the signal resembled the signal from the 5′ probe, but with an additional band of approximately 500 bp that was detected in HCT8/E8 (FIG. 29). The signal intensities were measured and normalized on the basis of the GAPDH transcript in the respective cell lines. The normalized values were graphically compared (FIG. 29, lower panel) and this emphasized the high expression level in SH-SY5Y.

The expression analysis of NBG family members was extended by RT-PCR experiments on either human fetal brain (HFB) RNA (using primers in exon 6 and exon 9) or on 32-2F53VIII RNA (using primers in exon 10 and exon 14). The cloned RT-PCR products were sequenced as depicted in FIG. 37. The products were specific for the NBG family but yielded also new exonic variants. Several splice variants of exon 8 were observed (exon 8.3, 8.14, 8.13, as well as removal of exon 8; FIG. 37), and this in line with the presence of a frameshift between exon 8 and exon 9 in cDNA clones AB033071 (FIG. 8) and Y79AA1001711 (see Example 9 and FIG. 25).

### Example 11

### NBG Protein Expression Generates Micronuclei

To study the function of this novel NBG gene family, several eukaryotic expression plasmids were constructed. To generate a representative full-length cDNA clone, sequences from two distinct cDNA clones were fused (FIG. 30). This full-length construct pBlueKIAA-AB25 was then tagged with either an aminoterminal Myc-epitope, or a carboxyterminal Myc-epitope, or an eGFP protein, yielding constructs pCS2+MT-KIAA-AB25, pEF6/Myc-His-B-KIAA-AB25 and pEGFP-C3-KIAA-AB25, respectively. The two distinct parts (aminoterminal and carboxyterminal half) were also separately coupled to eGFP (constructs pEGFP-N3-KIAA-AT and pEGFP-C3-AB25-CT, respectively), whereas the aminoterminal domain was also tagged with a Myc-epitope (construct pEF6/Myc-His-A-KIAA-AT).

These expression vectors were transfected into several human cell lines including mammary gland tumor cells MCF7/AZ (FIGS. 31 and 32), colon tumor cells HCT8/E8 (FIG. 33), embryonic kidney cells HEK293T (FIG. 34, top panels), colon tumor cells Colo320DM (FIG. 34, middle and bottom panels), and also into the mouse mammary gland cell line NMe (FIG. 35). The ectopically expressed proteins were detected by use of an anti-Myc antibody or by GFP fluorescence. These proteins showed a preferential cytoplasmic localization with only 5% of the transfected cells showing nuclear staining (the latter exemplified in FIG. 31E). Comparable results were obtained for constructs expressing either GFP-tagged or Myc-tagged proteins. Staining of the endoplasmic reticulum did not show colocalization with the NBG product (FIGS. 32A-C). Transfection of human cells with the full-size NBG constructs resulted in increased incidence of micronuclei-containing cells (illustrated by arrows in FIGS. 31I-L; FIGS. 32A-F; FIGS. 33A,B; FIGS. 34A,B,E,F). Staining of the nuclear lamina using an anti-lamin B1 antibody confirmed clearly the presence of micronuclei (FIGS. 32,D-F), which were generally also detectable by DAPI-mediated DNA-staining (most right panels of each combination in FIGS. 31-36). Transfections of backbone vectors served as negative controls and did not yield high levels of micronuclei (FIGS. 32G-J; FIGS. 33G,H; FIGS. 34G,H). Transfections of mouse NMe cells (FIG. 35) were particularly useful to demonstrate clearly the potential of the NBG proteins, as these cells lack endogenous NBG proteins. A high number of micronucleus-like structures was induced by full-size NBG protein in NMe cells (illustrated by the arrows FIGS. 35A,B,E,F), whereas such structures were not detectable in control transfectants (FIGS. 35G-J). According to DAPI staining, the NBG-induced cytoplasmic structures lacked DNA and this might point at molecules lacking in mouse and cooperating with NBG proteins in human cells in order to remove excessive DNA from the nucleus. Altogether, these data indicate that the NBG proteins can remove amplified DNA from human cells by inducing or enhancing micronuclei generation.

Transfections of plasmids encoding amino- or carboxyterrninally truncated NBG proteins were also associated with micronuclei generation, although an aminoterminal fragment (illustrated in FIGS. 31A-D; FIGS. 33C,D; FIGS. 34C,D; FIGS. 34I,J; FIGS. 35C,D) appeared to be less efficient than a carboxyterminal fragment (illustrated in FIGS. 31E-H; FIGS. 33E,F; FIGS. 34K,L). Transfection into MCF7/AZ cells of plasmids encoding the two NBG chimeric cDNAs isolated by 3′-RACE from 32-7A cells (see Example 8) was featured by the formation of multiple vesicle-like structures in the cytoplasm, which were apparently lacking DNA (FIGS. 36A,C). Some micronuclei were still visible in these transfected cells (FIG. 36A). From this, we conclude that these chimeric NBG transcripts are reorganizing the cytoplasmic organelles and interfere with the removal of excessive DNA such as amplified oncogenic DNA in cancer cells.

### Example 12

### NBG-Specific Antibodies

Polyclonal antibodies were raised against NBG-specific peptides. For example, rabbit polyclonal antibody #31226 was raised against peptide #1061 with sequence NH₂-PEILQDSLDRSYSTPSM-COOH. MCF7/AZ cells were transfected with a construct encoding a Myc-tagged full-length NBG protein. FIG. 38 illustrates that an anti-Myc antibody (A) and polyclonal antibody #31226 (B) produce overlapping staining patterns, pointing at the specificity of the polyclonal antibody. Non-transfected cells were not stained by anti-Myc antibody and only very lightly stained by polyclonal antibody #31226. The same polyclonal antibody was used in combination with anti-lamin B1 antibodies in order to allow identification of NBG-transfected cells (illustrated in FIG. 32D).

TABLE 1

Newly developed sequence-tagged sites (STS) obtained from sequenced PAC end clones: primer
sequences, product size and annealing temperature

| | | | product | annealing |
|---|---|---|---|---|
| Marker (PAC clone end) | primer 1 (5′-3′ sequence) | primer 2 (5′-3′ sequence) | size (bp) | temperature |
| RPCI-1 880L8 SP6 | GGCCATGTGAACCAATTCTG | GGAGAGCTGAGTGAGGAGGG | 421 | 58° C. |
| RPCI-1 243F6 SP6 | CGCCTGGCCTATTTACATGT | TGCAGCCCATAAATACCCAC | 451 | 55° C. |
| RPCI-1 252F1 T7 | CCTCGAGCTGAAGCCAAATT | TGGCCTCCATGCTGTAGGAT | 151 | 56° C. |
| RPCI-1 59J12 SP6 | TCAAACCCCGACTTTCAGAT | TCTGTGCTGCCTGGATTCAC | 501 | 58° C. |
| RPCI-1 928F18 T7 | GCCTGTTTCATCAAATGCCTG | GGAGAAATGGCACAGAGGTGA | 201 | 59° C. |
| RPCI-1 841C13 T7 | GAGACCAAGTTCTTTCCCAG | GAGACCAAGTTCTTTCCCAG | 251 | 54° C. |
| RPCI-1 841C13 SP6 | ACAAGCAAGACTGTGAAGCCC | GGCTTCCTGTTTCTCCTCCAG | 251 | 60° C. |
| RPCI-1 605A3 T7 | GCCTATCAGGCCATGATCCA | GGACTCCAGGACAGTGGCAT | 151 | 58° C. |
| RPCI-1 829O10 SP6 | ACCCCATCTCTTTCCAGCAT | TTGCCTGGCATTCATCTAGA | 201 | 54° C. |
| RPCI-1 829O10 T7 | GGGTCCCTGGTAGTTAACGA | CCAACTCCTGACCCCAGAA | 481 | 56° C. |
| RPCI-1 105C17 T7 | ATGGATGGGTAAACTGAGGCC | GCTGACTTCCTGAGGGAGGC | 151 | 60° C. |
| RPCI-1 1167N14 SP6 | CACCCAACTTTCCATGAGCTC | TGGTGGTGTACCCCCTTGAC | 301 | 59° C. |

TABLE 2
List of PAC and cosmid clones, screened by hybridization with the YAC, PAC, STS, and end clones
indicated, and confirmed to be positive for hybridization (FISH) on chromosome band 17q11.2

| Screening wlth | Clone type | Positive clones |
|---|---|---|
| cCl17-1079 | cosmid | RPCl-1 55N16, 118B4, 266J21 |
| STS D17S1850 | STS | RPCl-1 243F6 |
| STS Wl-2906 | STS | RPCl-1 243F6, 107O9, 52F12, 59J12, 110l13, 107O9 |
| 728-f-1 | YAC | RPCl-1 101G3, 194A10, 243F6, 107O9, 252F1 |
| 15FD11 | YAC | RPCl-1 257G22 |
| RPCl-1 266J21 | PAC | RPCl-1 266J21, 257G22, 655N1, 163O13 |
| 776-d-7 (end clones) | YAC end | RPCl-1 201O9, 129G2 |
| STS D17S798 | STS | RPCl-1 32H9, 153M23 |
| 681-c-S (end clones) | YAC end | RPCl-1 1027N14, 928F18, 1020l1, 880L8, 1167N14, 710N14, 841C13, 2H10, 863N19, 678O5, 624A6, 314l17 |
| RPCl-1 243F6 | PAC | RPCl-1 1188C15, 572L14, 1105H14, 952l18, 682F9, 921H21, 626M5 |
| RPCl-1 880L8 | PAC | lCRFc105F1060D1, lCRFc105E08156D1, lCRFc105B0832D1, lCRFc105C0471D1, lCRFc105B08115D1, |
| | | lCRFc105F1065D1, lCRFc105B07149D1, lCRFc105H1244D1, lCRFc105G0372D1, lCRFc105C04161D1, |
| | | lCRFc105F09116D1 |
| RPCl-1 880L8 (end clones) | PAC end | RPCl-1 829O10, 733P21, 835C10, 758F20, 605A3, 758E20 |
| RPCl-1 835C10 | PAC | RPCl-1 79l2, 91O8, 105C17, 307B7, 542l11, 1167N14 |

TABLE 3
Determination of PAC clone Insert slzes by FIGE

| PAC clone | Insert size |
|---|---|
| RPCl-1 257G22 | ˜87 kb |
| RPCl-1 55N16 | 304 kb |
| RPCl-1 118B4 | 94 kb |
| RPCl-1 243F6 | 129 kb |
| RPCl-1 252F1 | 126 kb |
| RPCl-1 201O9 | 141 kb |
| RPCl-1 129G2 | 141 kb |
| RPCl-1 1027N14 | ˜130 kb |
| RPCl-1 928F18 | 170 kb |
| RPCl-1 1020l1 | 70 kb |
| RPCl-1 880L8 | 140 kb |
| RPCl-1 710N14 | ˜155 kb |
| RPCl-1 841C13 | ˜98 kb |
| RPCl-1 2H10 | ˜98 kb |
| RPCl-1 863N19 | ˜155 kb |
| RPCl-1 678O5 | ˜100 kb |
| RPCl-1 624A6 | 112 kb |
| RPCl-1 572L14 | ˜55-60 kb |
| RPCl-1 194A10 | 83 kb |
| RPCl-1 32H9 | 88 kb |
| RPCl-1 153M23 | 77 kb |
| RPCl-1 829O10 | 80 kb |
| RPCl-1 266J21 | 105 kb |

TABLE 4

Sequences of 9 hybridization probes, specific for human chromosome
17 and generated by PCR on cosmid DNA template.

| | | |
|---|---|---|
| Probe N°. | #1 | |
| Primer^{a} | (+) 5′-gaactgtcgccactcgtctg-3′ | (−) 5′-acaccacagcgggatgatgt-3′ |
| Position^{b} | 9,359 | 9,874 |

| | |
|---|---|
| Probe | gaactgtcgccactcgtctggatgtgcattcgcaagctgccctgcgagtgctgtcaattgcaaattcagtcaattaggact |
| sequence | cagcatcaggcagctggaatgacacttcctctagtgaagcctcagagagcttacttatgccactcatttcagatggaga |
| | gcaagggtttgaatttgccccctctagagcccatgctcgcacttctgcaccgtatggcaaggttgagtcaggcagagctt |
| | gtcctcaagaagcccgcagtctgctgtggagagaggtgggcagtaaaacacatgctccagctgtagccatgtccctgt |
| | ctggaccagttaactcaattcagtttcaacttgcagacagctccttcaagccaatcagacatcagagaaataaaaagg |
| | atgtgacaaaagagagtgaggtggagaacacagtctccctctcctggacaaactgctctgggaaagtttgcacttgga |
| | ggtagatggggatgaagccaacacatcatcccgctgtggtgt |

| | | |
|---|---|---|
| Probe N° | #2 | |
| Primer^{a} | (+) 5′-aaccccgagtgttgctgaaa-3′ | (−) 5′-ccattccccaagcacctcat-3′ |
| Position^{b} | 14,748 | 15,536 |

| | |
|---|---|
| Probe | aaccccgagtgttgctgaaatcaaaaggctatgaagaagcagaagtcttgtgaaggtgttgttgggaaagagaattgtt |
| sequence | cagatatgggtaagccactgaaaggtgtcttgatggttcatctgaacatgggctgagaacctgaagcagatacagaaa |
| | gcaagatgaccatgctggaacaatgaggacagggaggatgggcagagtggctagggagaaggaggtcttggatg |
| | aacatctgagatagaaaatgcccctctggcggctcatgtgtgctctcctgatcacatgctctgacatggcaggccccag |
| | ccattctccttacgaacgacacttgctgggaatccctactgggcacacacaacccagtctacaagatagagtggtcc |
| | aaaccccccatcctgggctgtggtgcttggaagatgcaacacaccgactggctacgctgcagttactgcatctttctggc |
| | tggctggcaagcaaacttcccactgcctttctccccaggcctgaccccattatgacaatggagccattgttctgaacagg |
| | ggccatgaggcagctgggactggccgaccctatgctggctcctcacatctcagaagtcaagccctcagacagatcaa |
| | ggaaagtgataaaattcagcgggaagcaggacagagtattgggggtcctgagccagcttcttcctctcagttctgtatc |
| | gtgatgggaatgaataatcaccccaattacagagcaacctgaaggagacaaaatcacttaactaaatgaggtgcttg |
| | gggaatgg |

| | | |
|---|---|---|
| Probe N° | #3 | |
| Primer^{a} | (+) 5′-agagctgcacggcacttctc-3′ | (−) 5′-cacgatgcaaaacgaactac-3′ |
| Position^{b} | 18,773 | 19,237 |

| | |
|---|---|
| Probe | agagctgcacggcacttctcagcccacaagcccactttccatgcaccaagcccaccccaccttacacttggcccctgg |
| sequence | ctccttaaccttgagctcagcctccatcttaaggtttcaccaagctgcccttcactatccggtctgttggagattttccagcta |
| | gactctgctagccacgctctgactctgtttgacccagctcccggggagaagccaagccgactcccttccactccctctgc |
| | ccccgctgctgtcttacacagccgtaccttcctttcctttgccccctctcttggctggtgctgagccacatttagcaccagcct |
| | ccaagagcaagcaccctgtgctaacttgggctcgtgtctcgcaagctaccaccctgatcttagaaacccgaagctcag |
| | aaaatagttcctatcttatgtgcgttatactcttcaagaaggtagttcgttttgcatcgtg |

| | | |
|---|---|---|
| Probe N° | #4 | |
| Primer^{a} | (+) 5′-gtttggggagattttgagtgtg-3′ | (−) 5′-gggcagcagaccttagcaact-3′ |
| Position^{b} | 24,246 | 24,723 |

| | |
|---|---|
| Probe | gtttggggagattttgagtgtgtgggtggcagaaggtacttttaggttggggtcacagtgcagcgggccgagcaccaaa |
| sequence | gcacagatattctagaactggggttctacatcagactactactgtgagagccaagaggctcaaagcccccagagggt |
| | gactcattcttccagccctccgcctcctcctgcctgtccaggtcatcttgatcatcccattatcagaactgctggaggaatc |
| | atctggaaggcttgaagtgactggggggcatccgctgggttgtgagagctgctcggagggtcccttgtctgtcccagtcc |
| | acctcctccacccaccttatgctgtcaccccctgcccaggactggacctccattcaactcttctgagcagaaggagcag |
| | aaaccatcctatctctgcactggcctggaatggacagtgctccccagcatggttctagggagttgctaaggtctgctgcc |
| | c |

| | | |
|---|---|---|
| Probe N° | #5 | |
| Primer^{a} | (+) 5′-tcccaaaaggccagtttcacac-3′ | (−) 5′-tctgcaggcgtctcatctcaac-3′ |
| Position^{b} | 30,057 | 30,734 |

| | |
|---|---|
| Probe | tcccaaaaggccagtttcacacactccattaatgctggctgccactgggacaattacagacagacagcttttcccatttg |
| sequence | agcaacttcttaaatttaaaaagcaactgtgcaggaaggaaggagacatgtggctgctctactacagaccttgtaaaa |
| | ggaggttcttctatgctattacccaccccctcccatacctacctttcctaccacacacaagagctgaaataaaagttgcatt |
| | tcacttgtgttgtggctgttattttattttacactccttgcttaaaaaaaaaaaaagtcttcacttaaaaaaaaatacatcacc |
| | caaacatgtctgagattttttgcaaggcttgggtctgaacccagtgcctttgataggggcatttttcttagcctgctgtggcta |
| | aagatgggcatggtcggatttcactcgcttggattaggatggacttggccctaatattttgtttcttaatgattcttccaattttta |
| | agtgtcccaagagatagctttgactttccaccccagcctgctgatgttatcttcctaggaactgctacatcttttaatacaac |
| | ctgtattcaagttctccttataggacctgagagattcaactggcagcatttaggactaccaggaatctccagtctggcgttg |
| | agatgagacgcctgcaga |

| | | |
|---|---|---|
| Probe N° | #6 | |
| Primer^{a} | (+) 5′-cccctcagctctgtgcattt-3′ | (−) 5′-tccctgctgaatgagtgtttg-3′ |
| Position^{b} | 36,914 | 37,391 |

| | |
|---|---|
| Probe | cccctcagctctgtgcattttgtctacaagcaaggtgactttgggtttagtgtgttcatgtttcaacaagaatgtaagtttggg |
| sequence | atcagacatatgttacccctgttggatagtagctttgcaatcagtctttgcaaaaaatatccagaacaaggctagaacaa |
| | ggtggtacagaaacaaataacacgtgttacagttacatatgagcttggattacctcctttatagaaggaagtggagacat |
| | ccttcatgcagagatttaggatctctcactagcactaaccctcttgcccccacctagtgtttatttcaatcacccctgttgctttt |
| | tatcaaatgaaagatacagatccacttttaagagtaaagaagagaggaagcacagtttattaattacccattggtatccc |
| | aaggacttgtgcttccattatctcattgttttattcattcattaatccaaacactcattcagcaggga |

| | | |
|---|---|---|
| Probe N° | #7 | |
| Primer^{a} | (−) 5′-gaacctggtctacaaattatac-3′ | (−) 5′-gactgggaccaatttgttcttc-3′ |
| Position^{b} | 42,102 | 42,774 |

| | |
|---|---|
| Probe | gaacctggtctacaaattatacaccctggcatgtccaaaagcaaaatgctgcctacatctcctgaaaatgcaggaatgt |
| sequence | cagaaaagctgctgaagtggtggctaccaggagctccaggggcctagcacccactcccagcccgaccctggggtg |
| | gggagggtggtgctcttcagagtggctcattttagagctgactacccaagtgcagaagaaacaacgttttaaatatatcg |
| | ggcagcagagcagctagatattaaggttcttctttgcaggacatcatgaaatattgttcatgacgaccctgaccctgggtt |
| | gggcgcgggtgctcttcagagtggctcattttagagctgacttcccaagtgcagaagaaacaactttttaaatacgtcgg |
| | gtaacagagcagctagacattacggttcttctttgcagaacatcatgaaatattgatgttaagctaaaacgaatataataa |
| | atgtatgattgcctgctaaattggtcattatcttttttagattctggctttgacagaaagtagcaggccccaatggtttaagaa |
| | agaaaggaaaaaatttttttaattgaggaaatcatatccacgtctttttttttttaattgttgaaaagatgtccacagtcagattt |
| | cagaggaagaacaaattggtcccagtc |

| | | |
|---|---|---|
| Probe N° | #8 | |
| Primer^{a} | (+) 5′-gtacacccactcacctgtgat-3′ | (−) 5′-tctaagggggaaaagtaaacaa-3′ |
| Position^{b} | 46,377 | 47,137 |

| | |
|---|---|
| Probe | gtacacccactcacctgtgatgtacttaatggagcaggagggaacaggctgccatcaaagctaactcctggaaatga |
| sequence | cctagaacccacagcctgtctcagccgtgctgcatgttccagcagggaacaaagcgactgttgtattatgtaaaattact |
| | tacctgataataaaatgtttgtggattggataaatttgaacggatatattgtttccttcaccccacccccactagtggcaaaa |
| | gctgctctgtatgaatctgagtacatttctgtgacagtattcctcatatgtacagcattctatgtgaatcatctcccaaaaattg |
| | ccatggggtcattatccccagttcttaactagagaaactgagattctggaagtccctctgaatggcccatacttccccagg |
| | tgtctctgcaggaaacaatgtggacaatcacagtagttggaaggacaatgactgacctaatggagttcaggacgctat |
| | gactgttggcacctgatgtgcacagctgctcccatctgcgcagagagaatataaaggcagtggcgctgaaacatctgc |
| | tgctttcactcaccatcaagatggaactgactgtacatgcaagcaaattttgatgaagagagtgatctcagagtgcgaa |
| | gataagttgtttctccttgacacctaggaatgcacctggccaagaccctcagttgaaagacaatatcaaataaatgcaa |
| | aatctaaatatccatgtgcttgtttacttttcccccttaga |

| | | |
|---|---|---|
| Probe N°. | #9 | |
| Primer^{a} | (+) 5′-gttgtacccccttgacttca-3′ | (−) 5′-gtgcccagcaggagattcaat-3′ |
| Position^{b} | 38,018 | 38,584 |

| | |
|---|---|
| Probe | ggtgtacccccttgacttcacctgaatgtcagggcaaggccaggagcatgcaggaggcttaaagattgggaacccct |
| sequence | attcttgggccttctccccattctttctggagcaattggaagaggcccatgggagggaggtgactgtcatgatgtccccca |
| | ctatgaactatgggaatggctggagggtcactgggcctggtgctccagctgcctgacctgcagggaagtggtacctgc |
| | aggacctacagtgagggagtgttgacaccctggctctgggaaggagctcatggaaagttgggtgaacttcttgttagac |
| | tacgcaggctttcagaagtgcattccatacggagtggggtggcctccccagggaggagcagagggcagccactgga |
| | acaagctacagctacaaaagaccacctttatccctctcaagttctcccaggggcccctgtgtctgcctgtactagagga |
| | agtttccaaaggattttcctttccccaggatgaagaatgaggccacactgccaaagcaagatctcgtgctatgtgtgattg |
| | aatctcctgctgggcac |

^{a}The primers were designed on a genomic 51,050-bp sequence of human chromosome 17, obtained by shotgun cloning and DNA sequencing of three overlapping cosmids (Genbank Acc. No. AF148647).
^{b}Position refer to the location in the 51,050-bp sequence (Genbank Acc. No. AF148647).

TABLE 5
Overview of exons of a novel gene family,
comprising tumor suppressor genes

| | | | Source (clone name)³ | | | |
|---|---|---|---|---|---|---|
| Exon type¹ | Exon name² | Size #aa | Type⁴ | Genbank Acc. No. | Clone No. | Remark |
| ex0.1 | B02AT09 | 11 | BAC | AL356581.2 | RP5-1020C22 | |
| | B01AT14 | 11 | BAC | AL354666.3 | RP11-87L17 | |
| | KIA-AT00 | 11 | cDNA | AB033071 | KIAA1245 | |
| ex0.2 | AK0OAT00 | 13 | cDNA | AK00726 | HEP17004 | |
| ex0.3 | AE02AT01 | 11 | EST checked | W58508 | zd20d07.s1 | |
| ex1.1 | B01AT08 | 70 | BAC | AL354666.3 | RP11-87L17 | |
| | B02AT14 | 70 | BAC | AL356581.2 | RP5-1020C22 | |
| ex1.2 | AK00AT05 | 70 | cDNA | AK00726 | HEP17004 | |
| | | 70 | BAC | AC060753.8 | RP11-285C11 | |
| ex1.3 | KIA-AT06 | 70 | cDNA | AB033071 | KIAA1245 | |
| | KIA-AT11 | 70 | cDNA | AB033071 | KIAA1245 | |
| | | 70 | BAC | AL359176.4 | RP11-47D6 | |
| ex1.5 | AK00AT01 | 70 | cDNA | AK00726 | RP11-47D6 | |
| | | 70 | BAC | AL109937.4 | HSJ322E17 | |
| | | 70 | BAC | AC060753.8 | RP11-285C11 | |
| ex1.6 | KIA-AT01 | 70 | cDNA | AB033071 | KIAA1245 | |
| | | 70 | BAC | AL359176.4 | RP11-47D6 | |
| ex1.7 | B01AT13 | 70 | BAC | AL354666.3 | RP11-87L17 | |
| | | 70 | BAC | AL355149 | RP5-875O13 | |
| | | 70 | BAC | L355800.5 | RP11-284O17 | |
| | | 70 | BAC | AC084206.1 | RP11-305B17 | |
| | | 70 | BAC | AC015618.3 | RP11-4513 | |
| | | 70 | BAC | AL137798.8 | RP5-1182A14 | |
| ex1.8 | B02AT08 | 70 | BAC | AL356581.2 | RP5-1020C22 | |
| ex1.9 | AE02AT02 | 67 | EST checked | W58508 | zd20d07.s1 | partial |
| ex1.10 | ex1.A | 70 | BAC | AL109937.4 | HSJ322E17 | |
| | | 70 | BAC | AL138796.3 | RP4-791M13 | |
| ex1.11 | ex1.B | 70 | BAC | AL109937.4 | HSJ322E17 | |
| | AG10AT02 | 70 | EST checked | AW173183 | IMAGp998J126649Q2 | |
| ex1.12 | ex1.C | 70 | BAC | AL355149 | RP5-875O13 | |
| ex1.13 | ex1.D | 70 | BAC | AL355800.5 | RP11-284O17 | |
| | ex1.E | 70 | BAC | AL355800.5 | RP11-284O17 | |
| | ex1.F | 70 | BAC | AC015618.3 | RP11-4513 | |
| | ex1.G | 70 | BAC | AC015618.3 | RP11-4513 | |
| | | 70 | BAC | AL137798.8 | RP5-1182A14 | 2 X in this BAC |
| | | 70 | cDNA | AB051480 | K1AA1693 | |
| ex1.14 | ex1.H | 70 | BAC | AC027727.3 | RP11-18B10 | |
| | | 70 | BAC | AC026900.3 | RP11-798G11 | |
| ex1.15 | ex1.I | 70 | BAC | AC027727.3 | RP11-18B10 | |
| | | 70 | BAC | AC026900.3 | RP11-798G11 | |
| ex1.16 | ex1.M | 70 | BAC | AC018381.3 | RP11-5G21 | |
| | ex1.O | 70 | BAC | AL359093.9 | RP11-35B4 | |
| | ex1.Q | 70 | BAC | AC018729.3 | RP11-1F5 | |
| | ex1.R | 70 | BAC | AC018729.3 | RP11-1F5 | stop codon!! |
| | | 70 | BAC | AL049715.25 | RP4-646P11 | |
| ex1.17 | ex1.J | 70 | BAC | AC027209.2 | RP11-277H3 | |
| | ex1.K | 70 | BAC | AL390296.3 | RP11-555C20 | |
| ex1.18 | ex1.U | 70 | BAC | AC079074.1 | CTD-2386B2 | |
| ex1.19 | ex1.L | 70 | BAC | AL390296.3 | RP11-555C20 | |
| ex1.20 | ex1.N | 70 | BAC | AC018381.3 | RP11-5G21 | |
| | | 70 | BAC | AC007734.3 | RP11-44O1 | |
| ex1.21 | ex1.P | 70 | BAC | AL359093.9 | RP11-35B4 | |
| | | 70 | BAC | AC024119.2 | RP11-542J23 | |
| | | 70 | BAC | AC073223.4 | RP11580F15 | |
| ex1.22 | ex1.S | 70 | BAC | AL391068.7 | RP11-288L16 | |
| | ex1.T | 70 | BAC | AC026565.3 | RP11-702G12 | |
| ex1.23 | ex1.V | 70 | BAC | AC013673.2 | RP11-20I21 | |
| | ex1.W | 70 | BAC | AL356004.4 | RP11-94I2 | |
| | | 70 | BAC | AL049742.7 | RP4-565E6 | |
| ex1.24 | ex1.X | 70 | BAC | AL359752.7 | RP5-1042I8 | |
| ex1.25 | ex1.Y | 71 | BAC | AC013627.3 | RP11-11L4 | 71 AZ!! |
| | ex1.Z | 71 | BAC | AL359258.4 | RP11-483I13 | |
| | ex1.AA | 71 | BAC | AL356110.1 | RP4-562N20 | |
| | ex1.AB | 71 | BAC | AL390038.5 | RP11-131J3 | |
| ex1.26 | | 70 | BAC | AL049742.7 | RP4-565E6 | |
| ex1.27 | | 70 | BAC | AL049715.25 | RP4-646P11 | |
| ex2.1 | KIA-AT07 | 35 | cDNA | AB033071 | KIAA1245 | |
| | KIA-ATI2 | 35 | cDNA | AB033071 | KIAA1245 | |
| | | 35 | BAC | AL359176.4 | RP11-47D6 | |
| ex2.2 | AK00AT06 | 35 | cDNA | AK00726 | HEP17004 | |
| | AC01AT01 | 35 | EST checked | AL042283 | DKFZp434L1620 | |
| | | 35 | cDNA | AB051480 | KIA1693 | |
| ex2.3 | KIA-AT02 | 35 | cDNA | AB033071 | KIAA1245 | 2 X in this cDNA |
| | | 35 | BAC | AC015618.3 | RP11-45I3 | |
| | | 35 | BAC | AL109937.4 | RP3-322E17 | |
| | | 35 | BAC | AL355800.5 | RP11284O17 | |
| | | 35 | BAC | AL356004.4 | RP11-94I2 | |
| | | 35 | BAC | AC024119.2 | RP11-542J23 | |
| | | 35 | BAC | AC023990.1 | RP11-510K4 | |
| | | 35 | BAC | AC018729.3 | RP11-1F5 | |
| | | 35 | BAC | AL138796.3 | RP4-791M13 | |
| | | 35 | BAC | AC073223.4 | RP11-580F15 | |
| | | 35 | BAC | AL359093.9 | RP11-35B4 | 2 X in this BAC |
| | | 35 | BAC | AC018381.3 | RP11-5G21 | 2 X in this BAC |
| | | 35 | BAC | AC027727.3 | RP11-18B10 | |
| | | 35 | BAC | AL359176.4 | RP11-47D6 | |
| | | 35 | BAC | AC026900.3 | RP11-798G11 | 2 X in this BAC |
| | | 35 | BAC | AL355149.4 | RP5-875O13 | |
| | | 35 | BAC | AC007734.3 | RP11-44O1 | 2 X in this BAC |
| ex2.4 | AL117AT1 | 29 | pred. cDNA | AL117237 | HS328E191 | partial |
| ex2.5 | B01AT07 | 35 | BAC | AL354666.3 | RP11-87L17 | |
| | B02AT10 | 35 | BAC | AL356581.2 | RP5-1020C22 | |
| | | 35 | BAC | AC079074.1 | CTD-2386B2 | |
| | | 35 | BAC | AC021397.3 | | |
| | | 35 | BAC | AC015618.3 | RP11-45I3 | 2 X in this BAC |
| | | 35 | BAC | AC060753.8 | RP11-285C11 | |
| | | 35 | BAC | AL109937.4 | RP3-322E17 | |
| | | 35 | BAC | AC084209.1 | RP11-305B17 | 3 X in this BAC |
| | | 35 | BAC | AC027209.2 | RP11-277H3 | |
| | | 35 | BAC | AL355800.5 | RP11284O17 | 2 X in this BAC |
| | | 35 | BAC | AL356004.4 | RP11-94I2 | |
| | | 35 | BAC | AL356581.4 | RP5-1020C22 | |
| | | 35 | BAC | AL022240.8 | RP3-328E19 | |
| | | 35 | BAC | AL049742.7 | RP4-565E6 | |
| | | 35 | BAC | AL137798.8 | RP5-1182A14 | |
| | AG10AT03 | 35 | EST checked | AW173183 | IMAGp998J126649Q2 | |
| ex2.6 | B01AT12 | 35 | BAC | AL354666.3 | RP11-87L17 | |
| ex2.7 | B02AT07 | 35 | BAC | AL356581.2 | RP5-1020C22 | |
| | | 35 | BAC | AL356581.4 | RP5-1020C22 | |
| ex2.8 | ex2.A | 35 | BAC | AC060753.8 | RP11-285C11 | |
| | | 35 | BAC | AL109937.4 | RP3-322E17 | |
| ex2.9 | ex2.B | 35 | BAC | AC027209.2 | RP11-277H3 | |
| ex2.10 | ex2.C | 35 | BAC | AL355149.4 | RP5-875O13 | |
| ex2.11 | ex2.D | 35 | BAC | AL390296.3 | RP11-555C20 | |
| ex2.12 | ex2.E | 35 | BAC | AL359752.7 | RP5-1042I8 | |
| ex2.13 | ex2.F | 35 | BAC | AC024491.4 | RP11-139D23 | |
| ex2.14 | ex2.G | 34 | BAC | AC053486.4 | RP11-100A19 | 34 AZ!!! |
| ex2.15 | ex2.H | 35 | BAC | AC026565.3 | RP11-702G12 | |
| | ex2.I | 35 | BAC | AL391068.7 | RP11-288L16 | |
| ex2.16 | ex2.J | 35 | BAC | AC015706.3 | RP11-325P15 | |
| ex2.17 | ex2.K | 34 | BAC | AL356110.1 | RP4-562N20 | 34 AZ!!! |
| | ex2.L | 34 | BAC | AL359258.4 | RP11-483I13 | |
| | ex2.M | 34 | BAC | AL390038.5 | RP131J3 | |
| | ex2.N | 34 | BAC | AC013627.3 | RP11-11L4 | |
| ex2.18 | ex2.O | 35 | BAC | AC024468.3 | RP11-495P10 | |
| | ex2.P | 35 | BAC | AC018593.3 | RP11-21M21 | |
| | ex2.Q | 35 | BAC | AL162612.3 | RP4-691G10 | |
| | ex2.S | 35 | BAC | AC025992.3 | RP11-277L2 | |
| | ex2.T | 35 | BAC | AL358813.4 | RP11-353N4 | |
| ex2.19 | ex2.R | 35 | BAC | AC068979.9 | RP11-134C18 | |
| ex2.21 | | 35 | BAC | AL049715.25 | RP4-646P11 | 2 X in this BAC |
| | | 35 | BAC | AL137798.8 | RP5-1182A14 | |
| | | 35 | BAC | AL049742.7 | RP4-565E6 | |
| ex2.22 | | 35 | BAC | AL137798.8 | RP5-1182A14 | |
| ex.3.1 | KIA-AT08 | 71 | cDNA | AB033071 | KIAA1245 | |
| | KIA-AT13 | 71 | cDNA | AB033071 | KIAA1245 | |
| | | 71 | BAC | AL356004.4 | RP11-94I2 | 2 X in this BAC |
| | | 71 | BAC | AL359093.9 | RP11-35B4 | |
| | | 71 | BAC | AL359176.4 | RP11-47D6 | |
| | | 71 | BAC | AL049742.7 | RP4-565E6 | 2 X in this BAC |
| ex3.2 | AK00AT02 | 71 | cDNA | AK00726 | HEP17004 | |
| | | 71 | BAC | AC060753.8 | RP11-285C11 | |
| | | 71 | BAC | AL109937.4 | HSJ322E17 | |
| ex3.3 | AK00AT07 | 71 | cDNA | AK00726 | HEP17004 | |
| | AL117AT02 | 71 | pred. cDNA | AL117237 | HS328E191 | |
| | AC01AT02 | 71 | EST checked | AL042283 | DKFZp434L1620 | |
| | | 71 | BAC | AC007734.3 | RP11-44O1 | |
| | | 71 | BAC | AC060753.8 | RP11-285C11 | |
| | | 71 | BAC | AC026900.3 | RP11-798G11 | |
| | | 71 | BAC | AC018381.3 | RP11-5G21 | |
| | | 71 | BAC | AL109937.4 | HSJ322E17 | 2 X in this BAC |
| | | 71 | BAC | AC027727.3 | RP11-18B10 | |
| | | 71 | BAC | AL138796.3 | RP4-791M13 | |
| | | 71 | BAC | AL022240.8 | RP3-328E19 | |
| ex3.4 | KIA-AT03 | 71 | cDNA | AB033071 | KIAA1245 | |
| ex3.5 | B01AT06 | 71 | BAC | AL354666.5 | RP11-87L17 | |
| | B01AT11 | 71 | BAC | AL354666.5 | RP11-87L17 | |
| | AE4AT01′ | 71 | EST checked | AI239884 | IMAGp998N074517 | |
| | AB23AT01 | 71 | EST checked | AI239884 | IMAGp998N074517 | |
| | | 71 | BAC | AC015618.3 | RP11-4513 | |
| | | 71 | BAC | AL355800.5 | RP11-284O17 | |
| ex3.6 | B02AT06 | 71 | BAC | AL356581.4 | RP5-1020C22 | |
| | B02AT11 | 71 | BAC | AL356581.4 | RP5-1020C22 | |
| | | 71 | BAC | AL355149.4 | RP5-875O13 | 2 X in this BAC |
| | AG10AT04 | 71 | EST checked | AW173183 | IMAGp998J126649Q2 | |
| ex3.7 | AE03AT02 | 71 | EST checked | H06312 | IMAGp998I02268 | |
| ex3.8 | ex3.A | 71 | BAC | AC007734.3 | RP11-44O1 | |
| | ex3.D | 71 | BAC | AC018381.3 | RP11-5G21 | |
| ex3.9 | ex3.B | 71 | BAC | AC026900.3 | RP11-798G11 | |
| | ex3.I | 71 | BAC | AC027727.3 | RP11-18B10 | |
| ex3.10 | ex3.C | 71 | BAC | AL359176.4 | RP11-47D6 | |
| ex3.11 | ex3.E | 71 | BAC | AC015618.3 | RP11-45I3 | |
| | ex3.G | 71 | BAC | AL355800.5 | RP11-284O17 | |
| | | 71 | BAC | AL137798.8 | RP5-1182A14 | 2 X in this BAC |
| | | 71 | cDNA | AB051480 | KIAA1693 | |
| ex3.12 | ex3.F | 71 | BAC | AC015618.3 | RP11-45I3 | |
| | ex3.H | 71 | BAC | AL355800.5 | RP11-284O17 | |
| ex3.13 | ex3.J | 71 | BAC | AC027209.2 | RP11-277H3 | |
| | ex3.L | 71 | BAC | AL390296.3 | RP11-555C20 | |
| ex3.14 | ex3.K | 71 | BAC | AC027209.2 | RP11-277H3 | |
| ex3.15 | ex3.M | 71 | BAC | AL390296.3 | RP11-555C20 | |
| ex3.16 | ex3.N | 71 | BAC | AC073223.4 | RP11580F15 | |
| | ex3.O | 71 | BAC | AC024119.2 | RP11-542J23 | |
| ex3.17 | ex3.P | 71 | BAC | AL359093.9 | RP11-35B4 | |
| | | 71 | BAC | AL049715.25 | RP4-646P11 | |
| ex3.18 | ex3.Q | 71 | BAC | AC023783.2 | RP11-759I1 | |
| | | 71 | BAC | AL049715.25 | RP4-846P11 | |
| ex3.19 | ex3.R | 71 | BAC | AC018729.3 | RP11-1F5 | |
| ex3.20 | ex3.S | 71 | BAC | AL391068.7 | RP11-288L16 | |
| | ex3.T | 71 | BAC | AC026565.3 | RP11-702G12 | |
| ex3.21 | ex3.U | 71 | BAC | AL359752.7 | RP5-1042I8 | |
| ex3.22 | ex3.V | 71 | BAC | AC013627.3 | RP11-11L4 | |
| | ex3.W | 71 | BAC | AL359258.4 | RP11-483I13 | |
| | ex3.X | 71 | BAC | AL356110.1 | RP4-562N20 | |
| | ex3.Y | 71 | BAC | AL390038.5 | RP11-131J3 | |
| ex3.23 | ex3.Z | 71 | BAC | AC053486.4 | RP11-100A19 | 3 stop codons |
| ex3.24 | ex3.AA | 71 | BAC | AC024491.4 | RP11-139D23 | 1 stop codon |
| ex3.25 | | 71 | BAC | AL137798.8 | RP5-1182A14 | |
| | | 71 | cDNA | AB051480 | KIAA1693 | |
| ex4.1 | B01AT10 | 25 | BAC | AL354666.3 | RP11-87L17 | deleted in Vs.5 |
| | B02AT12 | 25 | BAC | AL356581.2 | RP5-1020C22 | |
| | | 25 | BAC | AL355149.4 | RP5-875O13 | |
| | | 25 | BAC | AC007734.3 | RP11-44O1 | |
| | | 25 | BAC | AC018381.3 | RP11-5G21 | |
| | | 25 | BAC | AL355800.5 | RP11-284O17 | 2 X in this BAC |
| | | 25 | BAC | AC015618.3 | RP11-45I3 | 2 X in this BAC |
| | | 25 | BAC | AC018729.3 | RP11-1F5 | |
| | | 25 | BAC | AL359093.9 | RP11-35B4 | |
| | | 25 | BAC | AL049715.25 | RP4-646P11 | |
| | | 25 | BAC | AL137798.8 | RP5-1182A14 | 2 X in this BAC |
| ex4.2 | AI117AT3 | 25 | pred. cDNA | AL117237 | HS328E191 | |
| | B02AT05 | 25 | BAC | AL356581.2 | RP5-1020C22 | |
| | | 25 | BAC | AC024119.2 | | |
| | | 25 | BAC | AC007734.3 | RP11-44O1 | |
| | | 25 | BAC | AC018381.3 | RP11-5G21 | |
| | | 25 | BAC | AL390296.4 | RP11-555C20 | |
| | | 25 | BAC | AC073223.4 | RP11-580F15 | |
| | | 25 | BAC | AC027727.3 | RP11-18B10 | |
| | | 25 | BAC | AC026900.3 | RP11-798G11 | |
| | | 25 | BAC | AC027209.2 | RP11-277H3 | |
| | | 25 | BAC | AL022240.8 | RP3-328E19 | |
| | | 25 | BAC | AL049715.25 | RP4-646P11 | |
| | | 25 | BAC | AL137798.8 | RP5-1182A14 | |
| | AE04AT01 | 25 | EST checked | AI239884 | IMAGp998N074517 | |
| | AC01AT03 | 25 | EST checked | AL042283 | DKFZp434L1620 | |
| | AB23AT02 | 25 | EST checked | AI239884 | IMAGp998N074517 | |
| ex4.3 | KIA-AT14 | 25 | cDNA | AB033071 | KIAA1245 | |
| | AE02AT03 | 25 | EST checked | H06312 | IMAGp998I02268 | |
| | | 25 | BAC | AL359176.4 | RP11-47D6 | |
| | | 25 | BAC | AL356004.4 | RP11-9412 | |
| ex4.4 | AK00AT08 | 25 | cDNA | AK00726 | HEP17004 | |
| | | 25 | BAC | AL109937.4 | RP3-322E17 | |
| | | 25 | BAC | AC060753.8 | RP11-285C11 | |
| ex4.5 | AK00AT03 | 25 | cDNA | AK00726 | HEP17004 | |
| | | 25 | BAC | AL109937.4 | RP3-322E17 | |
| | | 25 | BAC | AC060753.8 | RP11-285C11 | |
| ex4.6 | KIA-AT04 | 25 | cDNA | AB033071 | KIAA1245 | |
| | | 25 | BAC | AC027727.3 | RP11-18B10 | |
| | | 25 | BAC | AC026900.3 | RP11-798G11 | |
| | | 25 | BAC | AC027209.2 | RP11-277H3 | |
| | | 25 | BAC | AL359176.4 | RP11-47D6 | |
| | | 25 | BAC | AL356004.4 | RP11-94I2 | |
| | | 25 | BAC | AL049742.7 | RP4-565E6 | |
| ex4.7 | KIA-AT09 | 25 | cDNA | AB033071 | KIAA1245 | |
| ex4.8 | B01AT05 | 25 | BAC | AL354666.3 | RP11-87L17 | |
| | | 25 | BAC | AL355149.4 | RP5-875O13 | |
| | AG10AT05 | 25 | EST checked | AW173183 | IMAGp998J126649Q2 | |
| ex4.10 | ex4.A | 25 | BAC | AL109937.4 | RP3-322E17 | |
| | ex4.E | 25 | BAC | AL138796.3 | RP4-791M13 | |
| ex4.11 | ex4.B | 25 | BAC | AL390296.4 | RP11-555C20 | first a > g (from exon3) |
| ex4.12 | ex4.C | 25 | BAC | AL355800.5 | RP11-284O17 | |
| | ex4.D | 25 | BAC | AC015618.3 | RP11-45I3 | |
| ex4.13 | ex4.F | 25 | BAC | AC018729.3 | RP11-1F5 | |
| ex4.14 | ex4.G | 25 | BAC | AC053486.4 | RP11-100A19 | |
| ex4.15 | ex4.H | 25 | BAC | AC026565.3 | RP11-702G12 | |
| | ex4.I | 25 | BAC | AL391068.7 | RP11-288L16 | |
| ex4.16 | ex4.J | 25 | BAC | AC024491.4 | RP11-139D23 | |
| ex4.17 | AE01AT04 | 25 | EST checked | AW160820 | IMAGp998I196882 | |
| | | 25 | cDNA | AB051480 | KIAA1693 | |
| ex4.18 | AG9AT04 | 25 | EST checked | AI500017 | IMAGp998E065507Q2 | |
| | | 25 | cDNA | AB051480 | KIAA1693 | |
| ex5.1 | B01AT09 | 70 | BAC | AL354666.3 | RP11-87L17 | deleted in Vs.5 |
| | B02AT13 | 70 | BAC | AL356581.4 | RP5-1020C22 | |
| | | 70 | BAC | AC015618.3 | RP11-4513 | |
| | | 70 | BAC | AL355149.4 | RP5-875O13 | |
| | | 70 | BAC | AL355800.5 | RP11-284O17 | 2 X in this BAC |
| | | 70 | BAC | AL137798.8 | RP5-1182A14 | |
| ex5.2 | KIA-AT10 | 70 | cDNA | AB033071 | KIAA1245 | |
| ex5.3 | AK00AT04 | 70 | cDNA | AK00726 | HEP17004 | |
| | | 70 | BAC | AC060753.8 | RP11-285C11 | |
| | | 70 | BAC | AL109937.4 | HSJ322E17 | |
| ex5.4 | KIA-AT05 | 70 | cDNA | AB033071 | KIAA1245 | |
| | | 70 | BAC | AL359176.4 | RP11-47D6 | |
| ex5.5 | ex5 + 6.N | 70 | BAC | AC026900.3 | RP11-798G11 | |
| ex5.6 | ex5 + 6.R | 70 | BAC | AC027727.3 | RP11-18B10 | 1 stop codon |
| ex5.7 | ex5 + 6.L | 70 | BAC | AC027209.2 | RP11-277H3 | |
| ex5.8 | ex5 + 6.H | 70 | BAC | AC018729.3 | RP11-1F5 | |
| ex5.9 | ex5 + 6.C | 70 | BAC | AC023990.1 | RP11-510K4 | |
| | ex5 + 6.D | 70 | BAC | AL390296.3 | RP11-555C20 | |
| | ex5 + 6.F | 70 | BAC | AL359093.9 | RP11-35B4 | |
| | ex5 + 6.G | 70 | BAC | AC018729.3 | RP11-1F5 | |
| | ex5 + 6.I | 70 | BAC | AC018381.3 | RP11-5G21 | |
| | | 70 | BAC | AL049715.25 | RP4-646P11 | |
| ex5.10 | ex5 + 6.B | 70 | BAC | AC015618.3 | RP11-45I3 | |
| ex5.11 | ex5 + 6.K | 70 | BAC | AL356004.4 | RP11-94I2 | |
| | | 70 | BAC | AL049742.7 | RP4-565E6 | |
| ex5.12 | ex5 + 6.V | 70 | BAC | AL359752.7 | RP5-1042I8 | |
| ex5.13 | ex5 + 6.U | 70 | BAC | AC015706.3 | RP11-325P15 | 1 stop codon |
| ex5.14 | ex5 + 6.Y | 70 | BAC | AL359258.4 | RP11-483I13 | |
| | ex5 + 6.Z | 70 | BAC | AL390038.5 | RP11-131J3 | |
| | ex5 + 6.AB | 70 | BAC | AC068979.9 | RP11-134C18 | |
| ex5.15 | ex5 + 6.AA | 70 | BAC | AC013627.3 | RP11-11L4 | 1 stop codon |
| ex5.16 | | 70 | cDNA | AB051480 | KIAA1693 | |
| | | 70 | BAC | AL137798.8 | RP5-1182A14 | |
| ex6.1 | AL117AT4 | 68 | pred. cDNA | AL117237 | HS328E191 | |
| | KIA-AT15 | 68 | cDNA | AB033071 | KIAA1245 | |
| | B02AT04 | 68 | BAC | AL356581.2 | RP5-1020C22 | |
| | AC01AT04 | 68 | EST checked | AL042283 | DKFZp434L1620 | |
| | | 68 | BAC | AL359176.4 | RP11-47D6 | |
| | | 68 | BAC | AL355149.4 | RP5-875O13 | |
| | | 68 | BAC | AL356004.4 | RP11-94I2 | |
| | | 68 | BAC | AL022240.8 | RP3-328E19 | |
| | AG10AT06 | 68 | EST checked | AW173183 | IMAGp998J126649Q2 | |
| ex6.2 | AK00AT09 | 68 | cDNA | AK00726 | HEP17004 | |
| | AE05AT01 | 23 | EST checked | AA704208 | zj17a04.s1 | partial |
| | | 68 | BAC | AL109937.4 | HSJ322E17 | |
| ex6.3 | B01AT04 | 68 | BAC | AL354666.5 | RP11-87L17 | |
| | AE4AT02 | 68 | EST checked | AI239884 | IMAGp998N074517 | |
| | AB23AT03 | 68 | EST checked | AI239884 | IMAGp998N074517 | |
| | | 68 | BAC | AC015618.3 | RP11-4513 | |
| | | 68 | BAC | AL355800.5 | RP11-284O17 | |
| | | 68 | BAC | AL137798.8 | RP5-1182A14 | |
| | AG03AT02 | 68 | EST checked | AW468059 | IMAGp998B027200Q2 | |
| | | 68 | cDNA | AB051480 | KIAA1693 | |
| ex6.4 | AE03AT04 | 68 | EST checked | H06312 | IMAGp998I02268 | |
| ex6.5 | ex5 + 6.J | 68 | BAC | AC018381.3 | RP11-5G21 | |
| | | 68 | BAC | AL049715.25 | RP4-646P11 | |
| ex6.6 | ex5 + 6.P | 68 | BAC | AC024119.2 | RP11-542J23 | |
| | ex5 + 6.Q | 68 | BAC | AC073223.4 | RP11580F15 | |
| ex6.7 | ex5 + 6.E | 68 | BAC | AL390296.3 | RP11-555C20 | |
| | ex5 + 6.M | 68 | BAC | AC027209.2 | RP11-277H3 | |
| ex6.8 | ex5 + 6.A | 68 | BAC | AL109937.4 | HSJ322E17 | |
| | ex5 + 6.O | 68 | BAC | AC026900.3 | RP11-798G11 | |
| | ex5 + 6.S | 68 | BAC | AL138796.3 | RP4-791M13 | |
| ex6.9 | ex5 + 6.T | 68 | BAC | AC053486.4 | RP11-100A19 | 1 stop codon |
| ex6.10 | ex5 + 6.W | 68 | BAC | AL391068.7 | RP11-288L16 | |
| | ex5 + 6.X | 68 | BAC | AC026565.3 | RP11-702G12 | |
| ex6.11 | | 68 | cDNA | Combination of KIAA1245 and AB25 | | |
| ex6.12 | der_1AT01 | 68 | genomic DNA | | | = exon A |
| ex7.1 | B01AT03 | 18 | BAC | AL354666.3 | RP11-87L17 | |
| | AK00AT10 | 18 | cDNA | AK00726 | HEP17004 | |
| | AL117AT5 | 18 | pred. cDNA | AL117237 | HS328E191 | |
| | KIA-AT16 | 18 | cDNA | AB033071 | KIAA1245 | |
| | B02AT03 | 18 | BAC | AL356581.2 | RP5-1020C22 | |
| | E05AT02 | 18 | EST checked | AA704208 | zj17a04.s1 | |
| | AC01AT05 | 18 | EST checked | AL042283 | DKFZp434L1620 | |
| | AB25AT02 | 18 | EST checked | AL042839 | DKFZp434G2022 | |
| | AE03AT05 | 18 | EST checked | H06312 | IMAGp998I02268 | |
| | | 18 | BAC | AL022240.8 | RP3-328E19 | |
| | | 18 | BAC | AL049715.25 | RP4-646P11 | |
| | | 18 | BAC | AL137798.8 | RP5-1182A14 | |
| | | 18 | cDNA | AK024044 | Y79AA1001711 | |
| | | 18 | RT-PCR HFB | | pKNB11 | |
| | | 18 | RT-PCR HFB | | pKNB16 | |
| | | 18 | RT-PCR HFB | | pKNB21 | |
| | | 18 | RT-PCR HFB | | pKNB26 | |
| | | 18 | RT-PCR HFB | | pKNB20 | |
| | | 18 | RT-PCR HFB | | pKNB23 | |
| | | 18 | RT-PCR HFB | | pKNB27 | |
| | | 18 | RT-PCR HFB | | pKNB28 | |
| | AG03AT03 | 18 | EST checked | AW468059 | IMAGp998B027200Q2 | |
| | der_1AT02 | 18 | genomic DNA | | | = exon B |
| | AG10AT07 | 18 | EST checked | AW173183 | IMAGp998J126649Q2 | |
| | | 18 | cDNA | AB051480 | KIAA1693 | |
| ex7.2 | ex7.A | 18 | BAC | AC026565.3 | RP11-702G12 | |
| | ex7.A | 18 | BAC | AL391068.7 | RP11-288L16 | |
| ex7.3 | ex7.B | 18 | BAC | AC015706.3 | RP11-325P15 | |
| ex7.4 | ex7.C | 20 | BAC | AC053486.4 | RP11-100A19 | |
| ex7.5 | | 18 | RT-PCR HFB | | pKNB10 | |
| ex8.1 | AK00AT11 | 54 | cDNA | AK00726 | HEP17004 | |
| | | 54 | BAC | AC018381.3 | RP11-5G21 | |
| | | 54 | BAC | AC024119.2 | RP11-542J23 | |
| | | 54 | BAC | AC109937.4 | HSJ322E17 | |
| | | 54 | BAC | AL359093.9 | RP11-35B4 | |
| | | 54 | BAC | AC060753.8 | RP11-285C11 | |
| | | 54 | BAC | AL022240.8 | RP3-328E19 | |
| | AE03AT06 | 54 | EST checked | H06312 | IMAGp998I02268 | |
| | AG03AT04 | 54 | EST checked | AW468059 | IMAGp998B027200Q2 | |
| ex8.2 | AL117AT6 | 54 | pred. cDNA | AL117237 | HS328E191 | |
| | AE05AT03 | 54 | EST checked | AA704208 | zj17a04.s1 | |
| | AC01AT06 | 54 | EST checked | AL042283 | DKFZp434L1620 | |
| | | 54 | BAC | AC027209.2 | RP11-277H3 | |
| | | 54 | BAC | AL049715.25 | RP4-646P11 | |
| ex8.3 | KIA-AT17 | 27 | cDNA | AB033071 | KIAA1245 | |
| | | 27 | cDNA | AK024044 | Y79AA1001711 | |
| | | 27 | RT-PCR HFB | | pKNB16 | |
| ex8.4 | B01AT02 | 54 | BAC | AL354666.5 | RP11-87L17 | |
| ex8.5 | B02AT02 | 54 | BAC | AL356581.4 | RP5-1020C22 | |
| | | 54 | BAC | AL355149.4 | RP5-875O13 | |
| | | 54 | BAC | AC015618.3 | RP11-45I3 | |
| | | 54 | BAC | AL355800.5 | RP11-284O17 | |
| | | 54 | BAC | AL137798.8 | RP5-1182A14 | |
| | AB25AT03 | 54 | EST checked | AL042839 | DKFZp434G2022 | |
| | | 54 | RT-PCR HFB | | pKNB21 | |
| | AG10AT08 | 54 | EST checked | AW173183 | IMAGp998J126649Q2 | |
| | | 54 | cDNA | AB051480 | KIAA1693 | |
| ex8.6 | ex8.A | 54 | BAC | AL109937.4 | HSJ322E17 | |
| | ex8.B | 54 | BAC | AL138796.3 | RP4-791M13 | |
| | ex8.C | 54 | BAC | AC026900.3 | RP11-798G11 | |
| ex8.7 | ex8.D | 54 | BAC | AL356004.4 | RP11-94I2 | |
| | ex8.E | 54 | BAC | AL359176.4 | RP11-47D6 | |
| | | 54 | RT-PCGR HFB | | pKNB26 | |
| ex8.8 | ex8.F | 54 | BAC | AC027727.3 | RP11-18B10 | |
| ex8.9 | ex8.G | 54 | BAC | AL390296.3 | RP11-555C20 | |
| ex8.10 | ex8.H | 54 | BAC | AC053486.4 | RP11-100A19 | |
| ex8.11 | ex8.I | 54 | BAC | AC026565.3 | RP11-702G12 | |
| | ex8.J | 54 | BAC | AL391068.7 | RP11-288L16 | |
| ex8.12 | ex8.K | 53 | BAC | AC015706.3 | RP11-325P15 | |
| ex8.13 | | 33 | RT-PCR HFB | | pKNB27 | |
| | | 33 | RT-PCR HFB | | pKNB28 | |
| ex8.14 | | 27 | RT-PCR HFB | | pKNB10 | |
| ex9.1 | AK00AT12 | 18 | cDNA | AK00726 | HEP17004 | |
| | KIA-AT18 | 18 | cDNA | AB033071 | KIAA1245 | |
| | | 18 | cDNA | AK024044 | Y79AA1001711 | |
| | AE03AT07 | 18 | EST checked | H06312 | IMAGp998I02268 | |
| ex9.2 | AL117AT7 | 18 | pred. cDNA | AL117237 | HS328E191 | |
| | AE05AT04 | 18 | EST | AA704208 | zj17a04.s1 | |
| | AC01AT07 | 18 | EST checked | AL042283 | DKFZp434L1620 | |
| ex9.3 | B01AT01 | 18 | BAC | AL354666.5 | RP11-87L17 | |
| | B02AT01 | 18 | BAC | AL356581.2 | RP5-1020C22 | |
| | | 18 | BAC | AC015618.3 | RP11-45I3 | |
| | | 18 | BAC | AC027727.3 | RP11-18B10 | |
| | | 18 | BAC | AC026900.3 | RP11-798G11 | |
| | | 18 | BAC | AL355147.2 | RP11-78O9 | |
| | | 18 | BAC | AL356004.4 | RP11-94I2 | |
| | | 18 | BAC | AL109937.4 | HSJ322E17 | |
| | | 18 | BAC | AC018381.3 | RP11-5G21 | |
| | | 18 | BAC | AL355800.5 | RP11-284O17 | |
| | | 18 | BAC | AL355149.4 | RP5-875O13 | |
| | | 18 | BAC | AC024119.2 | RP11-542J23 | |
| | | 18 | BAC | AL138796.3 | RP4-791M13 | |
| | | 18 | BAC | AL359176.4 | RP11-47D6 | |
| | | 18 | BAC | AC060753.8 | RP11-285C11 | |
| | | 18 | BAC | AC073223.4 | RP11580F15 | |
| | | 18 | BAC | AC07734.3 | RP11-4401 | |
| | | 18 | BAC | AC079074.1 | CTD-2386B2 | 2 X in this BAC |
| | | 18 | BAC | AL049715.25 | RP4-646P11 | |
| | | 18 | BAC | AL137798.8 | RP5-1182A14 | |
| | AB25AT04 | 18 | EST checked | AL042839 | DKFZp434G2022 | |
| | AG10AT09 | 18 | EST checked | AW173183 | IMAGp998J126649Q2 | |
| | | 18 | cDNA | AB051480 | KIAA1693 | |
| ex9.4 | ex9.A | 18 | BAC | AC027209.2 | RP11-277H3 | |
| ex9.5 | ex9.B | 18 | BAC | AC053486.4 | RP11-100A19 | |
| ex9.6 | ex9.C | 18 | BAC | AC026565.3 | RP11-702G12 | |
| | ex9.D | 18 | BAC | AL391068.7 | RP11-288L16 | |
| ex9.7 | | 18 | BAC | AL022240.8 | RP3-328E19 | |
| ex9.8 | AG03AT05 | 18 | EST checked | AW468059 | IMAGp998B027200Q2 | |
| ex9.9 | | 18 | cDNA | AL136890 | DKFZp434D177 | |
| ex10.1 | KIAR04A | 57 | cDNA | AB033071 | KIAA1245 | |
| | KIAR05A | 57 | cDNA | AB033071 | KIAA1245 | |
| | KIAR08A | 57 | cDNA | AB033071 | KIAA1245 | |
| | KIAR09A | 57 | cDNA | AB033071 | KIAA1245 | |
| | KIAR12A | 57 | cDNA | AB033071 | KIAA1245 | |
| | KIAR13A | 57 | cDNA | AB033071 | KIAA1245 | |
| ex10.2 | KIAR1A | 57 | cDNA | AB033071 | KIAA1245 | |
| | | 57 | cDNA | AK024044 | Y79AA1001711 | |
| ex10.3 | AL117R1A | 57 | pred. cDNA | AL117237 | HS328E191 | |
| | | 57 | BAC | AL022240.8 | RP3-328E19 | |
| ex10.4 | AL117R4A | 57 | pred. cDNA | AL117237 | HS328E191 | |
| | | 57 | BAC | AL022240.8 | RP3-328E19 | |
| ex10.5 | E05R05SA | 57 | EST | AL037724 | DKFZp564A057 | |
| ex10.6 | AL117R5A | 57 | pred. cDNA | AL117237 | HS328E191 | |
| | | 57 | BAC | AL022240.8 | RP3-328E19 | |
| ex10.7 | E02R02SA | 57 | EST checked | AL045522 | DKFZp434J205 | |
| | B01RA04 | 57 | BAC | AL354666.3 | RP11-87L17 | |
| | B02RA02 | 57 | BAC | AL356581.2 | RP5-1020C22 | |
| | B02RA06 | 57 | BAC | AL356581.2 | RP5-1020C22 | |
| | 1b9RA03 | 57 | 5′RACE 2F53VIII | | | |
| | | 57 | BAC | AL137798.8 | RP5-1182A14 | |
| | R1d10ra2 | 57 | RT-PCR 2F53VIII | | | |
| | R1a11ra2 | 57 | RT-PCR 2F53VIII | | | |
| | AB06RA02 | 57 | EST checked | AL045522 | DKFZp434J205 | |
| | AB25RA01 | 57 | EST checked | AL042839 | DKFZp434G2022 | |
| | AB25RA04 | 57 | EST checked | AL042839 | DKFZp434G2022 | |
| | AB25RA07 | 57 | EST checked | AL042839 | DKFZp434G2022 | |
| | AG10RA01 | 57 | EST checked | AW173183 | IMAGp998J126649Q2 | |
| | | 57 | cDNA | AB051480 | KIAA1493 | |
| ex10.8 | E03R04SA | 56 | EST | AL043132 | DKFZp434D0623 | |
| ex10.9 | AL117R2A | 57 | pred. cDNA | AL117237 | HS328E191 | |
| | AL117R6A | 57 | pred. cDNA | AL117237 | HS328E191 | |
| | AC01RA02 | 57 | EST checked | AL042283 | DKFZp434L1620 | |
| | | 57 | BAC | AL022240.8 | RP3-328E19 | 2 X in this BAC |
| ex10.10 | E09R01A | 57 | EST | AA570017 | nm48h10 | |
| | 2a1RA02 | 57 | 5′RACETR14 | | | |
| ex10.11 | AC03RA01 | 57 | EST checked | AL043132 | DKFZp434D0623 | |
| | AC03RA04 | 57 | EST checked | AL043132 | DKFZp434D0623 | |
| | E10R02SA | 57 | EST | AW173183 | xj84d06.x1 | |
| | 1b9RA02 | 57 | 5′RACE2F53VIII | | | |
| | 2g2Ra02 | 57 | 5′RACE 7A | | | |
| | 2g7Ra02 | 57 | 5′RACE 7A | | | |
| | AB18RA01 | 57 | EST checked | AL044108 | DKFZp434M0628 | |
| | 2c4RA02 | 57 | 5′RACE SK-N-SH | | | |
| | AB25RA02 | 57 | EST checked | AL042839 | DKFZp434G2022 | |
| | AB25RA05 | 57 | EST checked | AL042839 | DKFZp434G2022 | |
| | AB25RA08 | 57 | EST checked | AL042839 | DKFZp434G2022 | |
| | AG01RA02 | 57 | EST checked | AL043174 | DKFZp434F2323Q2 | |
| | AG10RA02 | 57 | EST checked | AW173183 | IMAGp998J126649Q2 | |
| | | 57 | cDNA | AB051480 | KIAA1693 | |
| ex10.12 | KIAR02A | 57 | cDNA | AB033071 | KIAA1245 | |
| | KIAR10A | 57 | cDNA | AB033071 | KIAA1245 | |
| | KIAR14A | 57 | cDNA | AB033071 | KIAA1245 | |
| | 1b7RA02 | 57 | 5′RACE 2F53VIII | | | |
| | 2b10RA02 | 57 | 5′RACE TR14 | | | |
| ex10.13 | KIAR06A | 57 | cDNA | AB033071 | KIAA1245 | |
| ex10.14 | AK00R01A | 57 | cDNA | AK00726 | HEP17004 | |
| | AL050R2A | 57 | cDNA | AL050141 | DKFZp586O031 | |
| | AE05RA01 | 57 | EST checked | AA704208 | zj17a04.s1 | |
| | AG01RA04 | 57 | EST checked | AL043174 | DKFZp434F2323Q2 | |
| | AG04RA02 | 57 | EST checked | AI049567 | IMAGp998M024318Q2 | |
| | AG02ra06 | 57 | EST checked | AL037724 | DKFZp564A057Q3 | |
| | AG02ra09 | 57 | EST checked | AL037724 | DKFZp564A057Q3 | |
| ex10.15 | AK00R02A | 57 | cDNA | AK00726 | HEP17004 | |
| | AL050R3A | 57 | cDNA | AL050141 | DKFZp586O031 | |
| | 1b3RA02 | 57 | 5′RACE 2F53VIII | | | |
| | 1g1RA02 | 57 | 5′RACE 7A | | | |
| | 1aBRA02 | 57 | 5′RACE 2F53VIII | | | |
| | 1e2RA02 | 57 | 5′RACE 7A | | | |
| | 1g1RA02 | 57 | 5′RACE 7A | | | |
| | | 57 | BAC | AL049715.25 | RP4-646P11 | 2 X in this BAC |
| | AG01RA02 | 57 | EST checked | AL043174 | DKFZp434F2323Q2 | |
| | AG01RA05 | 57 | EST checked | AL043174 | DKFZp434F2323Q2 | |
| | AG04RA03 | 57 | EST checked | AI049567 | IMAGp998M024318Q2 | |
| | AG06RA04 | 57 | EST checked | AI537172 | IMAGp998E105403Q2 | |
| | AG06RA010 | 57 | EST checked | AI537172 | IMAGp998E105403Q2 | |
| | AG11RA03 | 57 | EST checked | AI953463 | IMAGp998F196353Q2 | |
| | AG11RA06 | 57 | EST checked | AI953463 | IMAGp998F196353Q2 | |
| ex10.16 | B01RA01 | 57 | BAC | AL354666.3 | RP11-87L17 | |
| ex10.17 | B01RA03 | 57 | BAC | AL354666.3 | RP11-87L17 | |
| | B02RA05 | 57 | BAC | AL356581.2 | RP5-1020C22 | |
| | AB06RA03 | 57 | EST checked | AL045522 | DKFZp434J205 | |
| ex10.18 | AF16R2A | 57 | mRNA | AF161426 | HSPC308 | |
| | 2f1RA02 | 57 | 5′RACE SK-N-SH | | | |
| ex10.19 | 1h5RA02 | 57 | 5′RACE TR14 | | | |
| | AE03RA02 | 57 | EST checked | H06312 | IMAGp998I02268 | |
| ex10.20 | AB18RA01 | 57 | EST checked | AL044108 | DKFZp434M0628 | |
| | AC03RA03 | 57 | EST checked | AL043132 | DKFZp434D0623 | |
| ex10.21 | 2d5RA02 | 57 | 5′RACE SK-N-SH | | | |
| | AG06RA07 | 57 | EST checked | AI537172 | IMAGp998E105403Q2 | |
| ex10.22 | AC01RA01 | 57 | EST checked | AL042283 | DKFZp434L1620 | |
| ex10.23 | | 57 | BAC | AL049715.25 | RP4-646P11 | |
| ex10.24 | | 57 | BAC | AL049715.25 | RP4-646P11 | |
| ex10.25 | | 57 | BAC | AL049715.25 | RP4-646P11 | |
| ex10.26 | AE03RA01 | 57 | EST checked | H06312 | IMAGp998I02268 | |
| ex10.27 | AC01RA01 | 57 | EST checked | AL043174 | DKFZp434F2323Q2 | |
| ex10.28 | AG03RA01 | 57 | EST checked | AW468059 | IMAGp998B027200Q2 | |
| ex10.29 | AG08RA01 | 57 | EST checked | AW238577 | IMAGp998C056777Q2 | |
| ex10.30 | AG06RA03 | 57 | EST checked | AI537172 | IMAGp998E105403Q2 | |
| | AG06RA06 | 57 | EST checked | AI537172 | IMAGp998E105403Q2 | |
| | AG06RA09 | 57 | EST checked | AI537172 | IMAGp998E105403Q2 | |
| | AG11RA02 | 57 | EST checked | AI953463 | IMAGp998F196353Q2 | |
| | AG11RA05 | 57 | EST checked | AI953463 | IMAGp998F196353Q2 | |
| ex10.31 | AG02ra03 | 57 | EST checked | AL037724 | DKFZp564A057Q3 | |
| ex10.32 | AG02ra04 | 57 | EST checked | AL037724 | DKFZp564A057Q3 | |
| ex10.34 | AG02ra07 | 57 | EST checked | AL037724 | DKFZp564A057Q3 | |
| ex10.35 | AE1ra02 | 57 | EST checked | AW160820 | IMAGp998I196882 | |
| ex10.36 | AG9ra01 | 57 | EST checked | AI570017 | IMAGp998E065507Q2 | |
| ex10.37 | | 57 | cDNA | AL136890 | DKFZp434D177 | |
| ex11.1 | BRPEX1A | 57 | genomic DNA | | | = exon x |
| | AB06RA01 | 57 | EST checked | AL045522 | DKFZp434J205 | |
| | AB13RA01 | 57 | EST checked | AA705685 | IMAGp998I041023 | |
| | AB14RA01 | 57 | EST checked | AA701673 | IMAGp998HI01019 | |
| | AC03RA02 | 57 | EST checked | AL043132 | DKFZp434D0623 | |
| | AC03RA05 | 57 | EST checked | AL043132 | DKFZp434D0623 | |
| | AD02RA01 | 57 | EST checked | AA322028 | EST24684 | |
| | B01RA02 | 57 | BAC | AL354666.3 | RP11-87L17 | |
| | B02RA01 | 57 | BAC | AL356581.2 | RP5-1020C22 | |
| | B02RA04 | 57 | BAC | AL356581.2 | RPS-1020C22 | |
| | AB18RA03 | 57 | EST checked | AL044108 | DKFZp434M0628 | |
| | 2H2RA01 | 57 | 5′RACE TR14 | | | |
| | RT1c6ra1 | 57 | RT-PCR 2F53VIII | | | |
| | R1d10ra1 | 57 | RT-PCR 2F53VIII | | | |
| | RT1a7ra1 | 57 | RT-PCR 2F53VIII | | | |
| | RT1b2ra1 | 57 | RT-PCR 2F53VIII | | | |
| | RT1b7ra1 | 57 | RT-PCR 2F53VIII | | | |
| | R1a11ra1 | 57 | RT-PCR 2F53VIII | | | |
| | RT1a1ra1 | 57 | RT-PCR 2F53VIII | | | |
| | AB25RA03 | 57 | EST checked | AL042839 | DKFZp434G2022 | |
| | AB25RA06 | 57 | EST checked | AL042839 | DKFZp434G2022 | |
| | AB25RA09 | 57 | EST checked | AL042839 | DKFZp434G2022 | |
| | AG10RA03 | 57 | EST checked | AW173183 | IMAGp998J126649Q2 | |
| | | 57 | cDNA | AB051480 | KIAA1693 | |
| ex11.2 | E10R03LA | 57 | EST | AW173183 | xj84d06.x1 | |
| ex11.3 | E05R02LA | 57 | EST | AL037724 | zj17a04.s1 | |
| | E09R02LA | 57 | EST | AA570017 | nm48h10.s1 | |
| | E11R02LA | 57 | EST | AI537172 | tp01b05.x1 | |
| | AL117R3A | 57 | pred. cDNA | AL117237 | HS328E191 | |
| | AL117R7A | 57 | pred. cDNA | AL117237 | HS328E191 | |
| | AK00R03A | 57 | cDNA | AK00726 | HEP17004 | |
| | AL050R1A | 57 | cDNA | AL050141 | DKFZp586O031 | |
| | AL050R4A | 57 | cDNA | AL050141 | DKFZp586O031 | |
| | | 57 | BAC | AL022240.8 | RP3-328E19 | 2 X in this BAC |
| | | 57 | BAC | AL049715.25 | RP4-646P11 | 2 X in this BAC |
| | AE03RA02 | 57 | EST checked | H06312 | IMAGp998I02268 | |
| | AG01RA03 | 57 | EST checked | AL043174 | DKFZp434F2323Q2 | |
| | AG01RA06 | 57 | EST checked | AL043174 | DKFZp434F2323Q2 | |
| | AG03RA01 | 57 | EST checked | AW468059 | IMAGp998B027200Q2 | |
| | AG04RA04 | 57 | EST checked | AI049567 | IMAGp998M024318Q2 | |
| | AG08RA02 | 57 | EST checked | AW238577 | IMAGp998C056777Q2 | |
| | AG06RA02 | 57 | EST checked | AI537172 | IMAGp998E105403Q2 | |
| | AG06RA05 | 57 | EST checked | AI537172 | IMAGp998E105403Q2 | |
| | AG06RA08 | 57 | EST checked | AI537172 | IMAGp998E10540302 | |
| | AG06RA011 | 57 | EST checked | AI537172 | IMAGp998E105403Q2 | |
| | AGIIRA04 | 57 | EST checked | AI953463 | IMAGp998F196353Q2 | |
| | AG11RA07 | 57 | EST checked | AI953463 | IMAGp998F196353Q2 | |
| ex11.4 | E06R02LA | 57 | EST | AI909921 | IL-BT228-060599-008 | |
| ex11.5 | KIAR11A | 57 | cDNA | AB033071 | KIAA1245 | |
| | KIAR03A | 57 | cDNA | AB033071 | KIAA1245 | |
| | KIAR07A | 57 | cDNA | AB033071 | KIAA1245 | |
| | KIAR15A | 57 | cDNA | AB033071 | KIAA1245 | |
| ex11.6 | AF116R3A | 57 | mRNA | AF161426 | HSPC308 | |
| ex11.7 | RT1a4ra1 | 57 | RT-PCR 2F53VIII | | | |
| ex11.8 | RT1a8ra1 | 57 | RT-PCR 2F53VIII | | | |
| ex11.9 | AB06RA04 | 57 | EST checked | AL045522 | DKFZp434J205 | |
| ex11.10 | AG02ra11 | 57 | EST checked | AL037724 | DKFZp564A057Q3 | |
| ex11.11 | | 57 | cDNA | AL136890 | DKFZp434D177 | |
| ex12.1 | E17R01LB | 35 | EST | AW851278 | IL3-CT0220-170200-067-E01 | |
| | E06R02LB | 35 | EST | AI909921 | IL-BT228-060599-008 | |
| ex12.2 | KIAR03B | 37 | cDNA | AB033071 | KIAA1245 | |
| | KIAR07B | 37 | cDNA | AB033071 | KIAA1245 | |
| | KIAR11B | 37 | cDNA | AB033071 | KIAA1245 | |
| | AF131R1B | 37 | mRNA | AF131738 | 24976 | |
| | AG01RB07 | 37 | EST checked | AL043174 | DKFZp434F2323Q2 | |
| ex12.3 | E05R02LB | 37 | EST | AL037724 | zj17a04.s1 | |
| | AL117R3B | 37 | pred. cDNA | AL117237 | HS328E191 | |
| | AL117R7B | 37 | pred. cDNA | AL117237 | HS328E191 | |
| | AL050R1b | 37 | cDNA | AL050141 | DKFZp586O031 | |
| | 2c3RB01 | 37 | 5′RACE SK-N-SH | | | |
| | | 37 | BAC | AL022240.8 | RP3-328E19 | |
| | | 37 | BAC | AL049715.25 | RP4-646P11 | 2 X in this BAC |
| | AG01RB01 | 37 | EST checked | AL043174 | DKFZp434F2323Q2 | |
| | AC01RB04 | 37 | EST checked | AL043174 | DKFZp434F2323Q2 | |
| | AG04RB01 | 37 | EST checked | AI049567 | IMAGp998M024318Q2 | |
| | AG06RB02 | 37 | EST checked | AI537172 | IMAGp998E105403Q2 | |
| | AG06RB05 | 37 | EST checked | AI537172 | IMAGp998E105403Q2 | |
| | AG06RB08 | 37 | EST checked | AI537172 | IMAGp998E105403Q2 | |
| | AG11RB01 | 37 | EST checked | AI953463 | IMAGp998F196353Q2 | |
| | AG11RB04 | 37 | EST checked | AI953463 | IMAGp998F196353Q2 | |
| ex12.4 | E03R03LB | 38 | EST | AL043132 | DKFZp434D0623 | |
| | AB18RB04 | 38 | EST checked | AL044108 | DKFZp434M0628 | |
| | AC03RB03 | 38 | EST checked | AL043132 | DKFZp434D0623 | |
| | AC03RB06 | 38 | EST checked | AL043132 | DKFZp434D0623 | |
| ex12.5 | BRPEX2B | 38 | genomic DNA | | | = exon y |
| | AB06RB01 | 38 | EST checked | AL045522 | DKFZp434J205 | |
| | AB03RB01 | 38 | EST checked | AL040932 | DKFZp434K1515 | |
| | AD02RB01 | 38 | EST checked | AA322028 | EST24684 | |
| | B01RB01L | 38 | BAC | AL354666.3 | RP11-87L17 | |
| | B02RB02L | 38 | BAC | AL356581.2 | RP5-1020C22 | |
| | RT1b2RB2 | 38 | RT-PCR 2F53VIII | | | |
| | RT1a8RB2 | 38 | RT-PCR 2F53VIII | | | |
| | RT1a7RB2 | 38 | RT-PCR 2F53VIII | | | |
| | RT1a4RB2 | 38 | RT-PCR 2F53VIII | | | |
| | RT1a1RB2 | 38 | RT-PCR 2F53VIII | | | |
| | R1d10RB2 | 38 | RT-PCR 2F53VIII | | | |
| | R1a11RB2 | 38 | RT-PCR 2F53VIII | | | |
| | AB25RA03 | 38 | EST checked | AL042839 | DKFZp434G2022 | |
| | AB25RA06 | 38 | EST checked | AL042839 | DKFZp434G2022 | |
| | AB25RA09 | 38 | EST checked | AL042839 | DKFZp434G2022 | |
| ex12.6 | B02RB01L | 38 | BAC | AL356581.2 | RP5-1020C22 | |
| | AB06RB04 | 38 | EST checked | AL045522 | DKFZp434J205 | |
| ex12.7 | AB13RB01 | 25 | EST checked | AA705685 | IMAGp998I041023 | partial |
| | B14RB01 | 25 | EST checked | AA701673 | IMAGp998H101019 | partial |
| ex12.8 | AG07RB01 | 37 | EST checked | AA609104 | IMAGp998B132576Q2 | |
| ex12.9 | AG02RB08 | 37 | EST checked | AL037724 | DKFZp564A057Q3 | |
| ex12.10 | AG02RB05 | 37 | EST checked | AL037724 | DKFZp564A057Q3 | |
| ex13.1 | E03R04SB | 18 | EST | AL043132 | DKFZp434D0623 | diff. From EST checked |
| ex13.2 | E02R02SB | 18 | EST | AL045522 | DKFZp434J205 | |
| | E10R01SB | 18 | EST | AW173183 | xj84d06.x1 | |
| | KIAR13B | 18 | cDNA | AB033071 | KIAA1245 | |
| | KIAR12B | 18 | cDNA | AB033071 | KIAA1245 | |
| | KIAR09B | 18 | cDNA | AB033071 | KIAA1245 | |
| | KIAR08B | 18 | cDNA | AB033071 | KIAA1245 | |
| | KIAR05B | 18 | cDNA | AB033071 | KIAA1245 | |
| | AL117R5B | 18 | pred. cDNA | AL117237 | HS328E191 | |
| | KIAR01B | 18 | cDNA | AB033071 | KIAA1245 | |
| | AL117R1B | 18 | pred. cDNA | AL117237 | HS328E191 | |
| | AL117R4B | 18 | pred. cDNA | AL117237 | HS328E191 | |
| | KIAR04B | 18 | cDNA | AB033071 | KIAA1245 | |
| | AK00R1B | 18 | cDNA | AK00726 | HEP17004 | |
| | B01RB02S | 18 | BAC | AL354666.5 | RP11-87L17 | |
| | B02RB04S | 18 | BAC | AL356581.2 | RP5-1020C22 | |
| | AL050R2B | 18 | cDNA | AL050141 | DKFZp586O031 | |
| | 1b3RB02S | 18 | 5′RACE 2F53VIII | | | |
| | 1b7RB02S | 18 | 5′RACE 2F53VIII | | | |
| | 1b9RB02S | 18 | 5′RACE 2F53VIII | | | |
| | 1e2rRB01 | 18 | 5′RACE 7A | | | |
| | 1f4RBO1 | 18 | 5′RACE 7A | | | |
| | 2g2RB02 | 18 | 5′RACE 7A | | | |
| | AB18RB02 | 18 | EST checked | AL044108 | DKFZp434M0628 | |
| | AC03RB04 | 18 | EST checked | AL043132 | DKFZp434D0623 | |
| | 1h5RB02 | 18 | 5′RACE TR14 | | | |
| | 2a8RB01 | 18 | 5′RACE TR14 | | | |
| | 2a1RB02 | 18 | 5′RACE TR14 | | | |
| | 2b10RB02 | 18 | 5′RACE TR14 | | | |
| | 2c4RB02 | 18 | 5′RACE SK-N-SH | | | |
| | 2f1RB01 | 18 | 5′RACE SK-N-SH | | | |
| | 2d5RB02 | 18 | 5′RACE SK-N-SH | | | |
| | | 18 | BAC | AL109937.4 | HSJ322E17 | |
| | | 18 | BAC | AL390296.4 | RP11-555C20 | |
| | | 18 | BAC | AC027727.3 | RP11-18B10 | |
| | | 18 | BAC | AC023990.1 | RP11-510K4 | 2 X in this BAC |
| | | 18 | BAC | AL356581.4 | RP5-1020C22 | |
| | | 18 | BAC | AC024119.2 | RP11-542J23 | 3 X in this BAC |
| | | 18 | BAC | AC018381.3 | RP11-5G21 | 5 X in this BAC |
| | 18 | BAC | AL355800.5 | RP11-284O172 | 2X in this BAC | |
| | | 18 | BAC | AC026900.3 | RP11-798G11 | |
| | | 18 | BAC | AL359093.9 | RP11-35B4 | 8 X in this BAC |
| | | 18 | BAC | AC079074.1 | CTD-2386B2 | |
| | | 18 | BAC | AL355149.4 | RP5-875O13 | |
| | | 18 | BAC | AL356004.4 | RP11-94I2 | |
| | | 18 | BAC | AC073223.4 | RP11580F15 | 2 X in this BAC |
| | | 18 | BAC | AL138842.4 | RP4-679C16 | 2 X in this BAC |
| | | 18 | BAC | AL138796.3 | RP4-791M13 | |
| | | 18 | BAC | AC018729.3 | RP11-1F5 | 7 X in this BAC |
| | | 18 | BAC | AC015618.3 | RP11-45I3 | |
| | | 18 | BAC | AC007734.3 | RP11-44O1 | 6 X in this BAC |
| | | 18 | BAC | AL355505.7 | RP4-634B11 | 7 X in this BAC |
| | | 18 | BAC | AC084206.1 | RP11-305B17 | 2 X in this BAC |
| | | 18 | BAC | AL359176.4 | RP11-47D6 | 3 X in this BAC |
| | | 18 | BAC | AL139152.2 | RP4-704D21 | |
| | | 18 | BAC | AC060753.8 | RP11-285C11 | 5 X in this BAC |
| | R1a11RB3 | 18 | RT-PCR 2F53VIII | | | |
| | R1d10Rb3 | 18 | RT-PCR 2F53VIII | | | |
| | AC01RB01 | 18 | EST checked | AL042283 | DKFZp434L1620 | |
| | | 18 | BAC | AL022240.8 | RP3-328E19 | 3 X in this BAC |
| | 18 | BAC | AL049715.25 | RP4-646P11 | 2 X in this BAC | |
| | 18 | BAC | AL137798.8 | RP5-1182A14 | | |
| | R1a11rb3 | 18 | RT-PCR 2F53VIII | | | |
| | R1d10rb3 | 18 | RT-PCR 2F53VIII | | | |
| | AE03RB01 | 18 | EST checked | H06312 | IMAGp998I02268 | |
| | AB06RB02 | 18 | EST checked | AL045522 | DKFZp434J205 | |
| | AB25RA01 | 18 | EST checked | AL042839 | DKFZp434G2022 | |
| | AB25RA04 | 18 | EST checked | AL042839 | DKFZp434G2022 | |
| | AB25RA07 | 18 | EST checked | AL042839 | DKFZp434G2022 | |
| | AG01RB02 | 18 | EST checked | AL043174 | DKFZp434F2323Q2 | |
| | AG01RB05 | 18 | EST checked | AL043174 | DKFZp434F2323Q2 | |
| | AG03RB01 | 18 | EST checked | AW468059 | IMAGp998B027200Q2 | |
| | AG04RB02 | 18 | EST checked | AI049567 | IMAGp998M024318Q2 | |
| | AG06RB03 | 18 | EST checked | AI537172 | IMAGp998E105403Q2 | |
| | AG06RB06 | 18 | EST checked | AI537172 | IMAGp998E105403Q2 | |
| | AG06RB09 | 18 | EST checked | AI537172 | IMAGp998E105403Q2 | |
| | AG11RA02 | 18 | EST checked | AI953463 | IMAGp998F196353Q2 | |
| | AG11RA05 | 18 | EST checked | AI953463 | IMAGp998F196353Q2 | |
| | AG10RA01 | 18 | EST checked | AW173183 | IMAGp998J126649Q2 | |
| | | 18 | cDNA | AB051480 | KIAA1693 | |
| ex13.3 | AC03RB02 | 18 | EST checked | AL043132 | DKFZp434D0623 | |
| | AC03RB05 | 18 | EST checked | AL043132 | DKFZp434D0623 | |
| | KIAR02SB | 18 | cDNA | AB033071 | KIAA1245 | |
| | KIAR06B | 18 | cDNA | AB033071 | KIAA1245 | |
| | KIAR10B | 18 | cDNA | AB033071 | KIAA1245 | |
| | KIAR14B | 18 | cDNA | AB033071 | KIAA1245 | |
| | E10R02SB | 18 | EST | AW173183 | xj84d06.x1 | |
| | E11R01SB | 18 | EST | AI537172 | tp01b05.x1 | |
| | AL117R2B | 18 | pred. cDNA | AL117237 | HS328E191 | |
| | AL117R6B | 18 | pred. cDNA | AL117237 | HS328E191 | |
| | E09R01SB | 18 | EST | AA570017 | tr91b03.x1 | |
| | B01RB01′ | 18 | BAC | AL354666.3 | RP11-87L17 | |
| | AC03RB02 | 18 | EST checked | AL043132 | DKFZp434D0623 | |
| | AL050R3B | 18 | cDNA | AL050141 | DKFZp586O031 | |
| | 1b1RB01S | 18 | 5′RACE 2F53VIII | | | |
| | 1b3RB01S | 18 | 5′RACE 2F53VIII | | | |
| | 1b4RB01 | 18 | 5′RACE 2F53VIII | | | |
| | 1b7RB01S | 18 | 5′RACE 2F53VIII | | | |
| | 1b9RB01S | 18 | 5′RACE 2F53VIII | | | |
| | 1c11RB01S | 18 | 5′RACE 2F53VIII | | | |
| | 1a8RB01 | 18 | 5′RACE 2F53VIII | | | |
| | 2g2RB01 | 18 | 5′RACE 7A | | | |
| | 2g7RB01 | 18 | 5′RACE 7A | | | |
| | AB18R803 | 18 | EST checked | AL044108 | DKFZp434M0628 | |
| | 1h5RB01 | 18 | 5′RACE TR14 | | | |
| | 1h3RB01 | 18 | 5′RACE TR14 | | | |
| | 2b2RB01 | 18 | 5′RACE TR14 | | | |
| | 2a1RB01 | 18 | 5′RACE TR14 | | | |
| | 2c4RB01 | 18 | 5′RACE SK-N-SH | | | |
| | RT1a4RB1 | 18 | RT-PCR 2F53VIII | | | |
| | RT1b7RB1 | 18 | RT-PCR 2F53VIII | | | |
| | RT1a8RB1 | 18 | RT-PCR 2F53VIII | | | |
| | RT1a7RB1 | 18 | RT-PCR 2F53VIII | | | |
| | RT1b2RB1 | 18 | RT-PCR 2F53VIII | | | |
| | RT1a1RB1 | 18 | RT-PCR 2F53VIII | | | |
| | R1a11RB1 | 18 | RT-PCR 2F53VIII | | | |
| | R1d10RB1 | 18 | RT-PCR 2F53VIII | | | |
| | AC01RB02 | 18 | EST checked | AL042283 | DKFZp434L1620 | |
| | AE03RB02 | 18 | EST checked | H06312 | IMAGp998I02268 | |
| | RT1a4rb1 | 18 | RT-PCR 2F53VIII | | | |
| | RT1b7rb1 | 18 | RT-PCR 2F53VIII | | | |
| | RT1a8r1b | 18 | RT-PCR 2F53VIII | | | |
| | RT1a7rb1 | 18 | RT-PCR 2F53VIII | | | |
| | RT1b2rb1 | 18 | RT-PCR 2F53VIII | | | |
| | RT1a11rb1 | 18 | RT-PCR 2F53VIII | | | |
| | R1d10rb1 | 18 | RT-PCR 2F53VIII | | | |
| | AB25RA02 | 18 | EST checked | AL042839 | DKFZp434G2022 | |
| | AB25RA05 | 18 | EST checked | AL042839 | DKFZp434G2022 | |
| | AB25RA08 | 18 | EST checked | AL042839 | DKFZp434G2022 | |
| | AG03RB02 | 18 | EST checked | AW468059 | IMAGp998B027200Q2 | |
| | AG04RB01 | 18 | EST checked | AI049567 | IMAGp998M024318Q2 | |
| | AG08RB03 | 18 | EST checked | AW238577 | IMAGp9980056777Q2 | |
| | AG06RB01 | 18 | EST checked | AI537172 | IMAGp998E105403Q2 | |
| | AG06RB04 | 18 | EST checked | AI537172 | IMAGp998E105403Q2 | |
| | AG06RB07 | 18 | EST checked | AI537172 | IMAGp998E105403Q2 | |
| | AG06RB10 | 18 | EST checked | AI537172 | IMAGp998E105403Q2 | |
| | AG11RB03 | 18 | EST checked | AI953463 | IMAGp998F196353Q2 | |
| | AG11RB06 | 18 | EST checked | AI953463 | IMAGp998F196353Q2 | |
| | AG10RB02 | 18 | EST checked | AW173183 | IMAGp998J126649Q2 | |
| ex13.4 | AK00R2B | 18 | cDNA | AK00726 | HEP17004 | |
| | 1e10RB01 | 18 | 5′RACE 7A | | | |
| | 1e2RB01 | 18 | 5′RACE 7A | | | |
| | AG01RB03 | 18 | EST checked | AL043174 | DKFZp434F2323Q2 | |
| | AG01RB06 | 18 | EST checked | AL043174 | DKFZp434F2323Q2 | |
| ex13.5 | B02RB03S | 18 | BAC | AL356581.2 | RP5-1020C22 | |
| | 1f10RB01 | 18 | 5′RACE 7A | | | |
| | AB06RB03 | 18 | EST checked | AL045522 | DKFZp434J205 | |
| ex13.6 | AFI6R2B | 18 | mRNA | AF161426 | HSPC308 | |
| ex13.7 | 1e5RB01S | 18 | 5′RACE 7A | | | |
| ex13.8 | 1g1RB01S | 18 | 5′RACE 7A | | | |
| ex13.9 | 1g1RB02S | 18 | 5′RACE 7A | | | |
| | | 18 | BAC | AL109937.4 | HSJ322E17 | |
| ex13.10 | 2b10RB01 | 18 | 5′RACE TR14 | | | |
| ex13.11 | 1a8RB02S | 18 | 5′RACE 2F53VIII | | | |
| | | 18 | BAC | AC018381.3 | RP11-5G21 | |
| | | 18 | BAC | AC007734.3 | RP11-44O1 | 2 X in this BAC |
| | | 18 | BAC | AC053486.4 | RP11-100A19 | |
| ex13.12 | 2g7RB02 | 18 | 5′RACE 7A | | | |
| ex13.13 | 2c11RB01 | 18 | 5′RACE SK-N-SH | | | |
| ex13.14 | 2d5RB01 | 18 | 5′RACE SK-N-SH | | | |
| | | 18 | cDNA | AB051480 | KIAA1693 | |
| ex13.15 | ex13.AD | 18 | BAC | AC007734.3 | RP11-44O1 | |
| ex13.16 | ex13.AJ | 18 | BAC | AC026565.3 | RP11-702G12 | 2 X in this BAC |
| | ex13.AM | 18 | BAC | AL391068.7 | RP11-288L16 | 2 X in this BAC |
| ex13.17 | ex13.AQ | 18 | BAC | AL359258.4 | RP11-483I13 | |
| | ex13.AR | 18 | BAC | AC013627.3 | RP11-11L4 | |
| | ex13.AS | 18 | BAC | AL390038.5 | RP11-131J3 | |
| ex13.18 | ex13.F | 18 | BAC | AC023990.1 | RP11-510K4 | |
| | ex13.O | 18 | BAC | AC018381.3 | RP11-5G21 | |
| | ex13.Q | 18 | BAC | AL359093.9 | RP11-35B4 | |
| | ex13.U | 18 | BAC | AC073223.4 | RP11580F15 | 2 X in this BAC |
| | ex13.W | 18 | BAC | AC018729.3 | RP11-1F5 | |
| | ex13.AC | 18 | BAC | AC007734.3 | RP11-44O1 | 2 X in this BAC |
| | ex13.AF | 18 | BAC | AL355505.7 | RP4-634B11 | |
| | | 18 | BAC | AL049715.25 | RP4-646P11 | |
| ex13.19 | ex13.AK | 18 | BAC | AC026565.3 | RP11-702G12 | |
| | ex13.AN | 18 | BAC | AL391068.7 | RP11-288L16 | |
| ex13.20 | ex13.AI | 18 | BAC | AC053486.4 | RP11-100A19 | |
| ex13.21 | ex13.AB | 18 | BAC | AC007734.3 | RP11-44O1 | 1 stop codon |
| ex13.22 | ex13.G | 18 | BAC | AC023990.1 | RP11-510K4 | 1 stop codon |
| ex13.23 | ex13.AU | 18 | BAC | AC015706.3 | RP11-325P15 | |
| ex13.24 | ex13.L | 18 | BAC | AC024119.2 | RP11-542J23 | 2 X in this BAC |
| | ex13.N | 18 | BAC | AC018381.3 | RP11-5G21 | 2 X in this BAC |
| | | 18 | BAC | AL049715.25 | RP4-646P11 | |
| ex13.25 | ex13.AO | 18 | BAC | AC013673.2 | RP11-20I21 | |
| ex13.26 | ex13.B | 18 | BAC | AC027727.3 | RP11-18B10 | |
| ex13.27 | ex13.AT | 18 | BAC | AC015706.3 | RP11-325P15 | |
| ex13.28 | ex13.H | 18 | BAC | AC023990.1 | RP11-510K4 | |
| ex13.29 | ex13.I | 18 | BAC | AL356581.4 | RP5-1020C22 | 2 X in this BAC |
| ex13.30 | ex13.C | 18 | BAC | AL109937.4 | HSJ322E17 | |
| | | 18 | BAC | AC060753.8 | RP11-285C11 | 7 X in this BAC |
| ex13.31 | ex13.A | 18 | BAC | AL359176.4 | RP11-47D6 | 2 X in this BAC |
| | | 18 | BAC | AL139152.2 | RP4-704D21 | 2 X in this BAC |
| | | 18 | BAC | AL109937.4 | HSJ322E17 | 2 X in this BAC |
| | | 18 | BAC | AC027727.3 | RP11-18B10 | 2 X in this BAC |
| | | 18 | BAC | AC023990.1 | RP11-510K4 | 4 X in this BAC |
| | | 18 | BAC | AC024119.2 | RP11-542J23 | |
| | | 18 | BAC | AC018381.3 | RP11-5G21 | 5 X in this BAC |
| | | 18 | BAC | AL355800.5 | RP11-284O17 | 2 X in this BAC |
| | | 18 | BAC | AL354666.5 | RP11-87L17 | |
| | | 18 | BAC | AC026900.3 | RP11-798G11 | 2 X in this BAC |
| | | 18 | BAC | AL359093.9 | RP11-35B4 | 5 X in this BAC |
| | | 18 | BAC | AL356004.4 | RP11-94I2 | |
| | | 18 | BAC | AC073223.4 | RP11580F15 | 2 X in this BAC |
| | | 18 | BAC | AL138842.4 | RP4-679C16 | |
| | | 18 | BAC | AL138796.3 | RP4-791M13 | |
| | | 18 | BAC | AC018729.3 | RP11-1F5 | 4 X in this BAC |
| | | 18 | BAC | AC015618.3 | RP11-45I3 | 2 X in this BAC |
| | | 18 | BAC | AC007734.3 | RP11-44O1 | 6 X in this BAC |
| | | 18 | BAC | AC084206.1 | RP11-305B17 | |
| | | 18 | BAC | AC053486.4 | RP11-100A19 | |
| | | 18 | BAC | AC026565.3 | RP11-702G12 | |
| | AL050R0B | 18 | BAC | AL050141 | DKFZp586O031 | |
| | | 18 | BAC | AL022240.8 | RP3-328E19 | |
| | | 18 | BAC | AL022240.8 | RP3-328E19 | |
| ex13.32 | ex13.M | 18 | BAC | AC024119.2 | RP11-542J23 | |
| ex13.33 | ex13.X | 18 | BAC | AC018729.3 | RP11-1F5 | 3 X in this BAC |
| ex13.34 | ex13.S | 18 | BAC | AC079074.1 | CTD-2386B2 | |
| ex13.35 | ex13.V | 18 | BAC | AC018729.3 | RP11-1F5 | 1 stop codon |
| ex13.36 | AG9rb01 | 18 | EST checked | AI570017 | IMAGp998E065507Q2 | |
| ex13.37 | | 18 | cDNA | AL136890 | DKFZp434D177 | |
| ex14.1 | E16CT | 80 | EST | C15842 | GEN-181D02 | |
| | | 80 | BAC | AL138842.4 | RP4-679C16 | |
| | | 80 | BAC | AL355505.7 | RP4-634B11 | |
| | | 80 | BAC | AC018381.3 | RP11-5G21 | |
| | | 80 | BAC | AL359093.9 | RP11-35B4 | |
| | | 80 | BAC | AC007734.3 | RP11-44O1 | |
| | AG07CT | 80 | EST checked | AA609104 | IMAGp998B132576Q2 | |
| ex14.2 | E18CT | 80 | EST | AA609104 | af11c07.s1 | |
| ex14.3 | E21CT | 80 | EST | AA301198 | EST14111 | |
| | AE1CT | 80 | EST checked | AW160820 | IMAGp998I196882 | |
| | | 80 | BAC | AL138796.3 | RP4-791M13 | |
| ex14.5 | E24CT | 80 | EST | H29685 | ym61b08 | |
| ex14.6 | AL117R8C | 80 | pred. cDNA | AL117237 | HS328E191 | |
| | | 80 | BAC | AL022240.8 | RP3-328E19 | |
| ex14.7 | E17CT | 80 | EST | AW851278 | IL3-CT0220-170200-067-E01 | |
| ex14.8 | E19CT | 80 | EST | AA333188 | EST37291 | |
| ex14.9 | AD01CT | 80 | EST checked | AA350323 | EST57569 | |
| | E14CT | 80 | EST | AI372468 | EST175301 | |
| | E15CT | 80 | EST | F11837 | c-32c04 | |
| ex14.10 | E29CT | 80 | EST | T65179 | yc74b04.r1 | |
| ex14.11 | AB03CT | 80 | EST checked | AL040932 | DKFZp434K1515 | |
| | B01CT01 | 80 | BAC | AL354666.5 | RP11-87L17 | |
| | | 80 | BAC | AC084206.1 | RP11-305B17 | |
| ex14.12 | BRKPEX3L | 80 | genomic DNA | | | = exon z |
| | AB25CT | 80 | EST checked | AL042839 | DKFZp434G2022 | |
| ex14.13 | E23CT | 80 | EST | AA315005 | EST186812 | |
| ex14.14 | E27CT | 80 | EST | C17016 | GEN-537A10 | |
| ex14.15 | AF131CT | 80 | mRNA | AF131738 | 24976 | |
| | | 80 | BAC | AL359176.4 | RP11-47D6 | |
| | | 80 | BAC | AL139152.2 | RP4-704D21 | |
| ex14.16 | B02CT01 | 80 | BAC | AL356581.2 | RP5-1020C22 | |
| | AB06CT | 80 | EST checked | AL045522 | DKFZp434J205 | |
| ex14.17 | AD02CT | 80 | EST checked | AA322028 | EST24684 | |
| ex14.18 | AC03CT | 80 | EST checked | AL043132 | DKFZp434D0623 | |
| | AB18CT | 80 | EST checked | AL044108 | DKFZp434M0628 | |
| | | 80 | BAC | AL355800.5 | RP11-284O17 | |
| | | 80 | BAC | AL355149.4 | RP5-875O13 | |
| | | 80 | BAC | AC015618.3 | RP11-45I3 | |
| ex14.19 | ex14.A | 80 | BAC | AC027727.3 | RP11-18B10 | |
| ex14.20 | ex14.B | 80 | BAC | AC060753.8 | RP11-285C11 | |
| | AG01CT | 80 | EST checked | AL043174 | DKFZp434F2323Q2 | |
| ex14.21 | ex14.C | 80 | BAC | AC024119.2 | RP11-542J23 | |
| | ex14.D | 80 | BAC | AC073223.4 | RP11580F15 | |
| ex14.22 | ex14.E | 80 | BAC | AC027209.2 | RP11-277H3 | |
| ex14.23 | ex14.G | 80 | BAC | AL356004.4 | RP11-94I2 | |
| ex14.24 | ex14.H | 80 | BAC | AC053486.4 | RP11-100A19 | |
| ex14.25 | ex14.I | 80 | BAC | AL391068.7 | RP11-288L16 | |
| | ex14.J | 80 | BAC | AL391068.7 | RP11-288L16 | |
| | ex14.K | 80 | BAC | AC026565.3 | RP11-702G12 | |
| | ex14.L | 80 | BAC | AC026565.3 | RP11-702G12 | |
| ex14.26 | AG02ct | 80 | EST-checked | AL037724 | DKFZp564A057Q3 | |
| | | 80 | cDNA | AB051480 | KIAA1693 | |

Footnotes to Table 5:
¹Exons are classified in types according to homologies at the protein level (FIGS. 9 to 15)
²Exon symbols refer to Laboratory Notebooks of the Dept. of Molecular Biology
³Occurrence of exons in genomic and cDNA clones, as indicated (summarized in FIGS. 7, 8, 21, 22, 23)
⁴Among the clone types are expressed sequence tags (EST), whose sequence was either taken from the public domain or confirmed and completed in the authors' laboratory (labeled as ‘EST-checked’, see also Table 6).

TABLE 6
Sequenced NBG-related cDNA clones

| | Depicted in FIG./ | | | GenBank Acc. Nos. | |
|---|---|---|---|---|---|
| DMB-VIB name | Sequence type | cDNA Clone Name(s) | GenBank Acc. No. | EST sequences | |
| AB01 | 3′-UTR | DKFZp434B235 | AF379611 | AL045343 | AL045344 |
| AB02 | 3′UTR + poly-A | DKFZp434J2318 | AF379612 | AL048907 | |
| AB03 | | DKFZp434K1515 | AF379613 | AL040932 | |
| AB04 | 3′UTR + poly-A | IMAGp998O19822 | AF379614 | W95853 | |
| | | IMAGE: 385122 | | | |
| AB05 | 3′UTR + poly-A | IMAGp998L06676 | AF379615 | W17223 | |
| | | IMAGE: 301973 | | | |
| AB06 | | DKFZp434J205 | AF379616 | AL045522 | |
| AB10 | 3′UTR + poly-A | IMAGp998I18822 | AF379617 | W95142 | |
| | | IMAGE: 357977 | | | |
| AB11 | 3′UTR + poly-A | IMAGp998I10824 | AF379618 | W94444 | |
| | | IMAGE: 358737 | | | |
| AB13 | | IMAGp998I041023 | AF379619 | AA705685 | |
| | | IMAGE: 435147 | | | |
| AB14 | | IMAGp998H101019 | AF420437 | AA701673 | |
| | | IMAGE: 433594 | | | |
| AB16 | 3′UTR + poly-A | IMAGp998N141787 | AF379621 | AA398843 | |
| | | IMAGE: 728653 | | | |
| AB18 | | DKFZp434M0628 | AF379622 | AL044108 | |
| AB23 = AE04 | | IMAGp998N074517 | AF379623 | AI239884 | |
| | | IMAGE: 1846470 | | | |
| AB25 | | DKFZp434G2022 | AF379624 | AL042839 | |
| AC02 | Pseudogene? | DKFZp434K2231 | AF379625 | AL110415 | |
| AC03 | | DKFZp434D0623 | AF379626 | AL043132 | |
| AD01 | | EST57569 | AF379627 | AA350323 | |
| AD02 | | EST24684 | AF379628 | AA322028 | |
| AE01 | | IMAGp998I196882 | 5′: AF419618 | AW160820 | |
| | | IMAGE: 2782170 | 3′: AF419619 | | |
| AE02 | 5′UTR + start | IMAGp9988N14778 | AF379629 | W58508 | |
| | codon | | | | |
| | | IMAGE: 341197 | | | |
| AE03 | | IMAGp998I02268 | AF379630 | H06312 | |
| | | IMAGE: 44185 | | | |
| AE05 | | IMAGp998I071063 | AF379631 | AA704208 | |
| | | IMAGE: 450510 | | | |
| AG01 | | DKFZp434F2323 | AF380580 | AL043174 | |
| AG02 | | DKFZp564A057 | AF419616 | AL037724 | |
| AG03 | | IMAGp998B027200 | AF380581 | AW468059 | |
| | | IMAGE: 2920609 | | | |
| AG04 | | IMAGp998M024318 | AF379632 | AI049567 | |
| | | IMAGE: 1700501 | | | |
| AG06 | | IMAGp998E105403 | AF379633 | AI537172 | |
| | | IMAGE: 2186481 | | | |
| AG07 | | IMAGp998B132576 | AF379634 | AA609104 | |
| | | IMAGE: 1031340 | | | |
| AG08 | | IMAGp998C056777 | AF380582 | AW238577 | |
| | | IMAGE: 2741692 | | | |
| AG09 | | IMAGp998E065507 | AF419617 | AI570017 | |
| | | IMAGE: 2226413 | | | |
| AG10 | | IMAGp998J126649 | AF379635 | AW173183 | |
| | | IMAGE: 2663915 | | | |
| AG11 | | IMAGp998F196353 | AF379636 | AI953463 | |
| | | IMAGE: 2550162 | | | |

TABLE 7
Human genomic contig sequences comprising
NBG-related sequences

| | | | # of | # | |
|---|---|---|---|---|---|
| | | Chromosomal | exon | of exon | # of |
| Contig | Size (bp) | localization | 7 | 8 | exon 9 |
| NT_021932.4 | 1,279,026 bp | 1q12 | 3 | 3 | 3 |
| NT_004754.5 | 5,941,040 bp | 1q12 | 5 | 6 | 5 |
| NT_023400.4 | 373,558 bp | not placed | 1 | 1 | 1 |
| | | (chr 6?) | | | |
| NT_004644.5 | 1,210,659 bp | 1q42.11 | 1 | 1 | 1 |
| NT_028134.1 | 147,523 bp | not placed | 1 | 1 | 1 |
| | | (chr 3?) | | | |
| NT_004434.5 | 1,108,112 bp | 1q21 | 1 | 2 | 1 |
| NT_004873.5 | 4,329,220 bp | 1p36.13 | 2 | 2 | 2 |
| NT_004576.5 | 1,278,015 bp | 1p36.11 | 0 | 1 | 0 |
| NT_004966.5 | 5,599,926 bp | 1p13.1 | 0 | 0 | 0 |
| NT_021907.5 | 1,184,925 bp | 1q21.3 | 0 | 0 | 0 |
| NT_019284.5 | 2,180,645 bp | 1p21.3 | 0 | 0 | 1 |
| NT_027019.2 | 493,854 bp | 5q14 | 0 | 0 | 0 |
| NT_005580.5 | 1,740,243 bp | 3p22.1 | 0 | 0 | 0 |
| | | Total | 14 | 17 | 15 |

Result of a TBLASTN analysis (Sep. 16, 2001) to the Human Genome Data using a typical NBG-protein as query. Thirteen contigs contained NBG-related sequences. Their GenBank accesion numbers are given in the first column, their size (bp) in the second column, and their chromosomal localization as determined by MapView (NCBI: hhtp://www.ncbi.nlm.nih.gov) in the third column. The occurrence (#) of different variants of the exon types 7, 8 and 9 (generally represented by a single copy in NBG transcripts) is also determined by BLAST analysis to the Human Genome Data.

### References

Altschul, S. F., Madden, T. L., Schaffer, A. A., Zhang, J. H., Zhang, Z., Miller, W., and Lipman, D. J. (1997). Gapped BLAST and PSI-BLAST: a new generation of protein database search programs, Nucleic Acids Res. 25, 3389-3402.

Ambros, I. M., Rumpler, S., Luegmayr, A., Hattinger, C. M., Strehl, S., Kovar, H., Gadner, H. and Ambros, P. F. (1997). Neuroblastoma cells can actively eliminate supernumerary MYCN gene copies by micronucleus formation-sign of tumour cell revertance? Eur. J. Cancer 33, 2043-9.

Bader, S. A., Fasching, C., Brodeur, G. M., and Stanbridge, E. J. (1991). Dissociation of suppression of tumorigenicity and differentiation in vitro effected by transfer of single human chromosomes into human neuroblastorma cells, Cell Growth Differ. 2, 245-55.

Bieche, I., Khodja, A., and Lidereau, R. (1998). Deletion mapping in breast tumor cell lines points to two distinct tumor-suppressor genes in the 1p32-pter region, one of deleted regions (1p36.2) being located within the consensus region of LOH in neuroblastoma, Oncol. Rep. 5, 267-272.

Biegel, J. A., White, P. S., Marshall, H. N., Fujimori, M., Zackai, E. H., Scher, C. D., Brodeur, G. M., and Emanuel, B. S. (1993). Constitutional 1p36 deletion in a child with neuroblastoma, Am. J. Hum. Genet. 52, 176-82.

Bonfield, J. K., Smith, K. F., and Staden, R. (1995). A new DNA sequence assembly program, Nucleic Acids Res. 23, 4992-4999.

Bown, N., Cotterill, S., Lastowska, M., O'Neill, S., Pearson, A. D., Plantaz, D., Meddeb, M., Danglot, G., Brinkschmidt, C., Christiansen, H., Laureys, G., and Speleman, F. (1999). Gain of chromosome arm 17q and adverse outcome in patients with neuroblastoma [see comments], N. Engl. J. Med. 340, 1954-61.

Brinkschmidt, C., Christiansen, H., Terpe, H. J., Simon, R., Boecker, W., Lampert, F., and Stoerkel, S. (1997). Comparative genomic hybridization (CGH) analysis of neuroblastomas-an important methodological approach in paediatric tumour pathology, J. Pathol. 181, 394-400.

Brodeur, G. M. (1998). Clinical and biological aspects of neuroblastoma. In The Genetic Basis of Human Cancer, B. Vogelstein, and K. W. Kinzler, eds. (New York, N.Y., McGraw-Hill), pp. 691-711.

Brodeur, G. M., Sekhon, G., and Goldstein, M. N. (1977). Chromosomal aberrations in human neuroblastomas, Cancer 40, 2256-63.

Buroker N., Bestwick R., Haight G., Magenis R. E., and Litt M. (1987). A hypervariable repeated sequence on human chromosome 1p36, Hum. Genet. 77, 175-81.

Cai D .X., Banerjee R., Scheithauer B. W., Lohse C. M., Kleinschmidt-Demasters B. K., Perry A. (2001). Chromosome 1p and 14q FISH analysis in clinicopathologic subsets of meningioma: diagnostic and prognostic implications, J. Neuropathol. Exp. Neurol. 60, 628-636.

Canute, G. W., Longo, S. L., Longo, J. A., Winfield, J. A., Nevaldine, B. H. and Hahn, P. J. (1996). Hydroxyurea accelerates the loss of epidermal growth factor receptor genes amplified as double-minute chromosomes in human glioblastoma multiforme, Neurosurgery 39, 976-83.

Caron, H. (1995). Allelic loss of chromosome 1 and additional chromosome 17 material are both unfavourable prognostic markers in neuroblastoma, Med. Pediatr. Oncol. 24, 215-21.

Caron, H., van Sluis, P., van Hoeve, M., de Kraker, J., Bras, J., Slater, R., Mannens, M., Voute, P. A., Westerveld, A., and Versteeg, R. (1993). Allelic loss of chromosome 1p36 in neuroblastoma is of preferential maternal origin and correlates with N-myc amplification (with published erratum in Nat. Genet. 1993 4:4311, Nat. Genet. 4, 187-90.

Collins C., Kuo W. L., Segraves R., Fuscoe J., Pinkel D., and Gray J. W. (1991). Construction and characterization of plasmid libraries enriched in sequences from single human chromosomes, Genomics 11, 997-1006.

De Angelis, P. M., Stokke, T., Beigi, M., Mjaland, O., and Clausen, O. P. (2001). Prognostic significance of recurrent chromosomal aberrations detected by comparative genomic hybridization in sporadic colorectal cancer. Int. J. Colorectal Dis. 16, 38-45.

Dorschner M. O., Sybert V. P., Weaver M., Pletcher B. A., Stephens K. (2000). NF1 microdeletion breakpoints are clustered at flanking repetitive sequences, Hum Mol Genet 9, 35-46.

Doz, F., Peter, M., Schleiermacher, G., Vielh, P., Validire, P., Putterman, M., Blanquet, V., Desjardins, L., Dufier, J. L., Zucker, J. M., Mosseri, V., Thomas, G., Magdelenat, H., and Delattre, O. (1996). N-MYC amplification, loss of heterozygosity on the short arm of chromosome 1 and DNA ploidy in retinoblastoma, Eur. J. Cancer 32A, 645-649.

Eckhardt, S. G., Dai, A., Davidson, K. K., Forseth, B. J., Wahl, G. M. and Von Hoff, D. D. (1994). Induction of differentiation in HL60 cells by the reduction of extrachromosomally amplified c-myc, Proc. Natl. Acad. Sci. U.S.A. 91, 6674-8.

Fong, C. T., Dracopoli, N. C., White, P. S., Merrill, P. T., Griffith, R. C., Housman, D. E., and Brodeur, G. M. (1989). Loss of heterozygosity for the short arm of chromosome 1 in human neuroblastomas: correlation with N-myc amplification, Proc. Natl. Acad. Sci. U.S.A. 86, 3753-7.

Fong, C. T., White, P. S., Peterson, K., Sapienza, C., Cavenee, W. K., S. E., K., Vogelstein, B., Cantor, A. B., Look, A. T., and Brodeur, G. M. (1992). Loss of heterozygosity for chromosomes 1 or 14 defines subsets of advanced neuroblastomas, Cancer Res. 52, 1780-1785.

Gilbert, F., Feder, M., Balaban, G., Brangman, D., Lurie, D. K., Podoisky, R., Rinaldt, V., Vinikoor, N., and Weisband, J. (1984). Human neuroblastomas and abnormalities of chromosomes 1 and 17, Cancer Res. 44, 5444-9.

Igarashi, J., Nimura, Y., Fujimori, M., Mihara, M., Adachi, W., Kageyama, H., and Nakagawara, A. (2000). Allelic loss of the region of chromosome 1p35-pter is associated with progression of human gastric carcinoma, Jpn. J. Cancer Res. 91, 797-801.

Ioannou, P. A., Amemiya, C. T., Games, J., Kroisel, P. M., Shizuya, H., Chen, C., Batzer, M. A., and de Jong, P. J. (1994). A new bacteriophage P1-derived vector for the propagation of large human DNA fragments, Nat. Genet. 6, 84-89.

Kanda, T., Otter, M., and Wahl, G. M. (2001). Mitotic segregation of viral and cellular acentric extrachromosomal molecules by chromosome tethering, J. Cell Sci. 114, 49-58.

Knuutila, S., Bjorkqvist, A. M., Autio, K., Tarkkanen, M., Wolf, M., Monni, O., Szymanska, J., Larramendy, M. L., Tapper, J., Pere, H. et al. (1998). DNA copy number amplifications in human neoplasms: review of comparative genomic hybridization studies, Am. J. Pathol. 152, 1107-23.

Lastowska, M., Nacheva, E., McGuckin, A., Curtis, A., Grace, C., Pearson, A., and Bown, N. (1997). Comparative genomic hybridization study of primary neuroblastoma tumors. United Kingdom Children's Cancer Study Group, Gene Chromosome Canc. 18, 162-9.

Lastowska, M., Van Roy, N., Brown, N., Speleman, F., Lunec, J., Strachan, T., Pearson, A. D. J., and Jackson, M. S. (1998). Molecular cytogenetic delineation of 17q translocation. breakpoints in neuroblastoma cell lines, Gene Chromosome Canc. 23, 116-122.

Laureys, G., Speleman, F., Opdenakker, G., Benoit, Y., and Leroy, J. (1990). Constitutional translocation t(1;17)(p36;q12-21) in a patient with neuroblastoma, Gene Chromosome Canc. 2, 252-4.

Laureys, G., Speleman, F., Versteeg, R., van der Drift, P., Chan, A., Leroy, J., Francke, U., Opdenakker, G., and Van Roy, N. (1995). Constitutional translocation t(1;17)(p36.31-p36.13; q11.2q12.1) in a neuroblastoma patient. establishment of somatic cell hybrids and identification of PND/a12m2 on chromosome 1 and NF1/SCYA7 on chromosome 17 as breakpoint flanking single copy markers, Oncogene 10, 1087-1093.

Levan, A. and Levan, G. (1978). Have double minutes functioning centromeres? Hereditas 88, 81-92.

Nelson, D. L., Ledbetter, S. A., Corbo, L., Victoria, M. F., Ramirez-Solis, R., Webster, T. D., Ledbetter, D. H., and Caskey, C. T. (1989). Alu polymerase chain reaction: a method for rapid isolation of human-specific sequences from complex DNA sources, Proc. Natl. Acad. Sci. U.S.A. 86, 6686-90.

Plantaz, D., Mohapatra, G., Matthay, K. K., Pellarin, M., Seeger, R. C., and Feuerstein, B. G. (1997). Gain of chromosome 17 is the most frequent abnormality detected in neuroblastoma by comparative genomic hybridization, Am. J. Pathol. 150, 81-9.

Ragnarsson, G., Eiriksdottir, G., Johannsdottir, J. T., Jonasson, J. G., Egilsson, V., and Ingvarsson, S. (1999). Loss of heterozygosity at chromosome 1p in different solid human tumours: association with survival, Br. J. Cancer 79, 1468-1474.

Raymond, E., Faivre, S., Weiss, G., McGill, J., Davidson, K., Izbicka, E., Kuhn, J. G., Allred, C., Clark, G. M. and Von Hoff, D. D. (2001). Effects of hydroxyurea on extrachromosomal DNA in patients with advanced ovarian carcinomas, Clin. Cancer Res. 7, 1171-80.

Riley, J., Butler, R., Ogilvie, D., Finniear, R., Jenner, D., Powell, S., Anand, R., Smith, J. C., and Markham, A. F. (1990). A novel, rapid method for the isolation of terminal sequences from yeast artificial chromosome (YAC) clones, Nucleic Acid Res. 18, 2887-2890.

Roth, M. B., Zahler, A. M. and Stolk, J. A. (1991). A conserved family of nuclear phosphoproteins localized to sites of polymerase 11 transcription, J. Cell Biol. 115, 587-596.

Sambrook, J., Fritsch, E. F., and Maniatis, T. (1989). Molecular Cloning. A Laboratory Manual (Cold Spring Harbor, N.Y., Cold Spring Harbor Laboratory Press).

Sanger, F. (1981). Determination of nucleotide sequences in DNA, Science 214, 1205-1210.

Savelyeva, L., Corvi, R., and Schwab, M. (1994). Translocation involving 1p and 17q is a recurrent genetic alteration of human neuroblastoma cells, Am. J. Hum. Genet. 55, 334-40.

Schleiermacher, G., Peter, M., Michon, J., Hugot, J. P., Vielh, P., Zucker, J. M., Magdelenat, H., Thomas, G., and Delattre, O. (1994). Two distinct deleted regions on the short arm of chromosome 1 in neuroblastoma, Gene Chromosome Canc. 10, 275-81.

Schwab, M., Praml, C., and Amler, L. C. (1996). Genomic instability in 1p and human malignancies, Gene Chromosome Canc. 16, 211-29.

Shimizu, N., Nakamura, H., Kadota, T., Kitajima, K., Oda, T., Hirano, T. and Utiyama, H. (1994). Loss of amplified c-myc genes in the spontaneously differentiated HL-60 cells, Cancer Res. 54, 3561-7.

Shimizu, N., Itoh, N., Utiyama, H. and Wahl, G. M. (1998). Selective entraprnent of extrachromosomally amplified DNA by nuclear budding and micronucleation during S phase, J. Cell Biol. 140, 1307-20.

Shimizu, N., Shimura, T. and Tanaka, T. (2000). Selective elimination of acentric double minutes from cancer cells through the extrusion of micronuclei, Mutat. Res. 448, 81-90.

Siebert, P. D., Chenchik, A., Kellogg, D. E., Lukyanov, K. A., and Lukyanov, S. A. (1995). An improved PCR method for walking in uncloned genomic DNA, Nucleic Acids Res. 23, 1087-1088.

Speleman, F., Van Gele, M., Maertens, L., and Van Roy, N. (1997). Improved protocol for chromatin fibre preparation from fixed cells. Technical Tips online, Trends Genet., T01123.

Takeda, O., Homma, C., Maseki, N., Sakurai, M., Kanda, N., Schwab, M., Nakamura, Y., and Kaneko, Y. (1994). There may be two tumor suppressor genes on chromosome arm 1p closely associated with biologically distinct subtypes of neuroblastoma, Gene Chromosome Canc. 10, 30-9.

Tanaka, K., Yanoshita, R., Konishi, M., Oshimura, M., Maeda, Y., Mori, T., and Miyaki, M. (1993). Suppression of tumourigenicity in human colon carcinoma cells by introduction of normal chromosome 1p36 region, Oncogene 8, 2253-2258.

van der Drift, P., Chan, A., Laureys, G., van Roy, N., Sickmann, G., den Dunnen, J., Westerveld, A., Speleman, F., and Versteeg, R. (1995). Balanced translocation in a neuroblastoma patient disrupts a cluster of small nuclear RNA U1 and tRNA genes in chromosomal band 1p36, Gene Chromosome Canc. 14, 35-42.

van der Drift, P., Chan, A., van Roy, N., Laureys, G., Westerveld, A., Speleman, F., and Versteeg, R. (1994). A multimegabase cluster of snRNA and tRNA genes on chromosome 1p36 harbours an adenovirus/SV40 hybrid virus integration site, Hum. Mol. Genet. 3, 2131-6.

Von Hoff, D. D., Waddelow, T., Forseth, B., Davidson, K., Scott, J. and Wahl, G. (1991). Hydroxyurea accelerates loss of extrachromosomally amplified genes from tumor cells. Cancer Res. 51, 6273-9.

Von Hoff, D. D., McGill, J. R., Forseth, B. J., Davidson, K. K., Bradley, T. P., Van Devanter, D. R. and Wahl, G. M. (1992). Elimination of extrachromosomally amplified MYC genes from human tumor cells reduces their tumorigenicity, Proc. Natl. Acad. Sci. U.S.A. 89, 8165-9.

Van Roy, N., Cheng, N. C., Laureys, G., Opdenakker, G., and Speleman, F. (1995). Molecular cytogenetic analysis of 1,17 translocations in neuroblastoma, Eur. J. Cancer 31A, 530-535.

Van Roy, N., Jauch, A., Van Gele, M., Laureys, G., Versteeg, R., De Paepe, A., Cremer, T., and Speleman, F. (1997a). Comparative genomic hybridization analysis of human neuroblastomas: Detection of distal 1p deletions and further molecular genetic characterization of neuroblastoma cell lines, Cancer Genet. Cytogenet. 97, 35-142.

Van Roy, N., Laureys, G., Cheng, N. C., Willem, P., Opdenakker, G., Versteeg, R., and Speleman, F. (1994). 1;17 translocations and other chromosome 17 rearrangements in human primary neuroblastoma tumors and cell lines, Gene Chromosome Canc. 10, 103-114.

Van Roy, N., Laureys, G., Van Gele, M., Opdenakker, G., Miura, R., vanderDrift, P., Chan, A., Versteeg, R., and Speleman, F. (1997b). Analysis of 1;17 translocation breakpoints in neuroblastoma: Implications for mapping of neuroblastoma genes, Eur. J. Cancer 33, 1974-1978.

Vandesompele, J., Van Roy, N., Van Gele, M., Laureys, G., Ambros, P., Heimann, P., Devalck, C., Schuuring, E., Brock, P., Otten, J., Gyselinck, J., De Paepe, A., and Speleman, F. (1998). Genetic heterogeneity of neuroblastoma studied by comparative genomic hybridization, Gene Chromosome Canc. 23, 141-152.

Versteeg, R., Caron, H., Cheng, N. C., Van der Drift, P., Slater, R., Westerveld, A., Voute, P. A., Delattre, O., Laureys, G., Van Roy, N., and Speleman, F. (1995). 1p36: every subband a suppressor?, Eur. J. Cancer 31A, 538-541.

White, P. S., Thompson, P. M., Seifreid, B. A., Sulman, E. P., Jensen, S. J., Guo, G., Maris, J. M., Hogarty, M. D., Allen, C., Biegel, J. A., Matise, T. C., Gregory, S. G., Reynolds, C. P., Brodeur, G. M. (2001). Detailed molecular analysis of 1p36 in neuroblastoma, Med Ped Oncol 36, 37-41.

Zehetner, G., and Lehrach, H. (1994). The reference library system—sharing biological material and experimental data, Nature (London) 367, 489-491.

Zhang, J., Glaffelter, A. A., Taetle, R., and Trent, J. M. (1999). Frequent alterations of evolutionarily conserved regions of chromosome 1 in human malignant melanoma, Cancer Genet Cytogenet. 111I, 119-123.

1. An isolated tumor suppressor gene product, comprising SEQ ID N° 202 or a functional fragment, variant or fusion protein thereof.

2. A functional fragment of a tumor suppressor gene product according to claim 1, whereby said functional fragment comprises at least SEQ ID N° 2 or SEQ ID N° 161.

3. A variant of a tumor suppressor gene product according to claim 1, whereby said variant comprises at least a sequence selected from the group consisting of SEQ ID N° 167, 169, 171, 173, 175, 177, 179, 181, 183, 185, 187, 189, 191, 193, 195, 197, 198 and 200.

4. A fusion protein of a tumor suppressor gene product according to claim 1, whereby said fusion product comprises SEQ ID N° 163 or SEQ ID N° 165.

5. An isolated nucleic acid encoding a tumor suppressor gene product or a tumor suppressor gene product fragment, variant, or fusion product according to claim any of the claims 1-4.

6. An isolated nucleic acid according to claim 5, comprising the sequence presented in SEQ ID N° 1.

7. An isolated nucleic acid according to claim 5, comprising the sequence presented in SEQ ID N° 3.

8. An isolated nucleic acid according to claim 5, comprising the sequence presented in SEQ ID N° 4.

9. An isolated nucleic acid according to claim 5, comprising the sequence presented in SEQ ID N° 201.

10. An isolated nucleic acid according to claim 5, comprising the sequence presented in SEQ ID N° 203.

11. An isolated nucleic acid according to claim 5, comprising the sequence presented in SEQ ID N° 160.

12. An isolated nucleic acid according to claim 5, comprising the sequence selected from the group consisting of SEQ ID N° 166, 168, 172, 174, 176, 178, 180, 182, 184, 186, 188, 190, 192, 194, 196 and 199.

13. An isolated nucleic acid according to claim 5, comprising the sequence presented in SEQ ID N° 162 or SEQ ID N° 164.

14. The use of a nucleic acid according to any of the claims 5-13, or a nucleic acid with at least 60% identity to said nucleic acid, or a functional fragment thereof in diagnosis of cancer and/or prediction of the likelihood of developing cancer.

15. The use of a nucleic acid according to any of the claims 5-12, or a nucleic acid with at least 60% identity to said nucleic acid, or a functional fragment thereof in treatment of cancer.

16. The use of a tumor suppressor gene product, or a functional fragment or a variant thereof according to any of the claims 1 to 4, or a protein with at least 60% identity to said tumor suppressor gene product, for the manufacture of a medicament to treat cancer.

17. The use according to any of the claims 14-16, whereby said cancer is meningioma, colorectal cancer, gastric carcinoma and/or breast cancer.

18. The use according to any of the claims 14-16, whereby said cancer is neuroblastoma.

19. The use of a tumor suppressor gene product, or a functional fragment or a variant thereof according to any of the claims 1 to 4, or a protein with at least 60% identity to said tumor suppressor gene product for the generation of micronuclei and/or the removal of amplified DNA

20. A method for the production of antibodies against a tumor suppressor gene product, using a tumor suppressor gene product, or a functional fragment, variant or fusion product thereof according to any of the claims 1-4, or a protein with at least 60% identity to said tumor suppressor gene product, or an isolated nucleic acid encoding such polypeptide.

21. An antibody obtainable by the use according to claim 20.

22. The use of an antibody according to claim 21 in diagnosis of cancer and/or prediction of the likelihood of developing cancer.

23. The use of an antibody according to claim 22, whereby the cancer is meningioma, colorectal cancer, gastric carcinoma and/or breast cancer.

24. The use of an antibody according to claim 22, whereby the cancer is neuroblastoma

25. The use of a tumor suppressor gene product or a functional fragment, variant or fusion product thereof according to any of the claims 1-4, or a protein with at least 60% identity to said tumor suppressor gene product, for the isolation of an interacting compound.
